(19) United States
(12) Patent Application Publication
(10) Pub. No.: US 2013/0078276 A1
(43) Pub. Date: Mar. 28, 2013

(54) VECTORS EXPRESSING HIV ANTIGENS AND GM-CSF AND RELATED METHODS OF GENERATING AN IMMUNE RESPONSE

(71) Applicants: Harriet L. Robinson, Atlanta, GA (US); Rama R. Amara, Decatur, GA (US); Michael Hellerstein, Atlanta, GA (US); Lilin Lai, Decatur, GA (US)

(72) Inventors: Harriet L. Robinson, Atlanta, GA (US); Rama R. Amara, Decatur, GA (US); Michael Hellerstein, Atlanta, GA (US); Lilin Lai, Decatur, GA (US)

(21) Appl. No.: 13/579,667

(22) PCT Filed: Feb. 18, 2011
(86) PCT No.: PCT/US11/25422

§ 371 Date: Dec. 11, 2012

Related U.S. Application Data
(60) Provisional application No. 61/305,936, filed on Feb. 18, 2010.
(60) Provisional application No. 61/387,801, filed on Sep. 29, 2010.

(51) Int. Cl. C12N 15/85

(52) U.S. Cl. CPC C12N 15/85
USPC 424/208.1; 435/320.1; 536/23.5

(57) ABSTRACT

The disclosure provides vectors encoding one or more HIV antigens and GM-CSF. Also provided are methods of inducing an immune response in a subject, methods of treating a subject having HIV, and methods of manufacturing a medicament for inducing an immune response that require the use of these vectors and vaccine inserts.

### TECHNICAL FIELD

This disclosure relates to vectors and vaccine inserts useful for inducing an immune response to a human immunodeficiency virus (HIV) in a subject and methods of inducing an immune response to a HIV in a subject using one or more of the provided vectors and vaccine inserts.

### BACKGROUND OF THE DISCLOSURE

Vaccines have had profound and long lasting effects on world health. Smallpox has been eradicated, polio is near elimination, and diseases such as diphtheria, measles, mumps, pertussis, and tetanus are contained.

The prevalence of HIV1 infection has made vaccine development for this recently emergent agent a high priority for world health. The development of safe and effective vaccines against existing and emerging pathogens is a major focus of medical research. Considerable effort has been directed to making a vaccine that will protect against human immunodeficiency virus-1 (HIV). An effective vaccine is thought to require the induction of cellular and Immoral responses (Douek et at, 2006).

### SUMMARY

Described herein is a vector comprising: a prokaryotic origin of replication; and an eukaryotic transcription cassette comprising a vaccine insert encoding HIV Gag, HIV Pol lacking the integrase domain, HIV Tat, HIV Rev, HIV Vpu, HIV Env and GM-CSF. Thus, the vector comprises sequences encoding HIV Gag, HIV Pol lacking the integrase domain, HIV Tat, HIV Rev, HIV Vpu, HIV Env and GM-CSF (e.g., human GM-CSF) and operably linked sequences for expressing HIV Gag, HIV Pol lacking the integrase domain, HIV Tat, HIV Rev, HIV Vpu, HIV Env and GM-CSF in a eukaryotic (e.g., human) cell. In various embodiments: the HIV Gag comprises or consists of an amino acid sequence that is at least 80%, 85% 90%, 95%, or 98% identical to an HIV Gag amino acid sequence depicted herein below; the HIV Pol comprises or consists of an amino acid sequence that is at least 80%, 85% 90%, 95%, or 98% identical to an HIV Pol amino acid sequence depicted herein below; the HIV Tat comprises or consists of an amino acid sequence that is at least 80%, 85% 90%, 95%, or 98% identical to an HIV Pol amino acid sequence depicted herein below; the HIV Tat comprises or consists of an amino acid sequence that is at least 80%, 85% 90%, 95%, the HIV Rev comprises or consists of an amino acid sequence that is at least 80%, 85% 90%, 95%, or 98% identical to an HIV Rev amino acid sequence depicted herein below; the HIV Vpu comprises or consists of an amino acid sequence that is at least 80%, 85% 90%, 95%, or 98% identical to an HIV Vpu amino acid sequence depicted herein below; the HIV Env comprises or consists of an amino acid sequence that is at least 80%, 85% 90%, 95%, or 98% identical to an HIV Env amino acid sequence depicted herein below; and the GM-CSF comprises or consists of an amino acid sequence that is at least 80%, 85% 90%, 95%, or 98% identical to an GM-CSF amino acid sequence depicted herein below.

In various embodiments: the eukaryotic transcription cassette comprises a eukaryotic promoter operably linked to the nucleic acid sequence encoding HIV Gag, HIV Pol lacking the integrase domain, HIV Tat, HIV Rev, HIV Vpu, HIV Env and GM-CSF; the HIV Gag, HIV Pol lacking the integrase domain, HIV Tat, HIV Rev, HIV Vpu, and HIV Env are from one or more HIV clades; the HIV Gag, HIV Pol lacking the integrase domain, HIV Tat, HIV Rev, HIV Vpu, and HIV Env are from the same HIV clade; the one or more HIV clades are selected from the group consisting of HIV clades A, B, C, D, E, F, G, H, I, J, K, and L; expression of the eukaryotic expression cassette in human cells produces a pre-mRNA encoding HIV Gag, HIV Pol lacking the integrase domain, HIV Tat, HIV Rev, HIV Vpu, HIV Env and GM-CSF; the eukaryotic expression cassette comprises an internal ribosome entry site positioned for translation of GM-CSF; the eukaryotic expression cassette further comprises one or more of: a leader sequence, an intron sequence and a polyadenylation sequence; the eukaryotic expression cassette further comprises one or more of: the tissue plasminogen activator leader sequence, CMV intron A sequence and bovine growth hormone polyadenylation sequence; the

HIV Gag has a mutation in a zinc finger domain that reduces RNA packaging activity; the HIV Pol has a mutation that reduces protease activity; the HIV Pol has a mutation that reduces polymerase activity; the HIV Pol has a mutation that reduces strand transfer activity; the HIV Pol has a mutation that reduces RNaseH activity; the HIV Pol has HIV Pol has a mutation that reduces protease activity, a mutation that reduces polymerase activity, a mutation that reduces strand transfer activity, and a mutation that reduces RNaseH activity; the vector comprises a sequence encoding a prokaryotic selectable marker; the vector further comprises a prokaryotic transcriptional terminator operably linked to the sequence encoding the prokaryotic selectable marker; the encoded GM-CSF is a full-length human GM-CSF; the sequence encoding GM-CSF comprises the sequence of: nucleotides 6633-7067 of SEQ ID NO: 7, nucleotides 6648-7082 of SEQ ID NO: 8, or nucleotides 7336-7770 of SEQ ID NO: 9; the encoded GM-CSF is a truncated human GM-GSF or a mutant human GM-GSF that is capable of stimulating macrophage differentiation and proliferation, or activating antigen presenting cells; the GM-CSF comprises the amino acid sequence of SEQ ID NO: 10; the HIV Gag comprises an amino acid sequence that is at least 95% identical to SEQ ID NO:A, the HIV Pol lacking the integrase domain comprises an amino acid sequence that is at least 95% identical to SEQ ID NO:B, the HIV Tat comprises an amino acid sequence that is at least 95% identical to SEQ ID NO:C, HIV Rev comprises an amino acid sequence that is at least 95% identical to SEQ ID NO:D, the HIV Vpu comprises an amino acid sequence that is at least 95% identical to the amino acid sequence of SEQ ID NO:E, and HIV Env comprises an amino acid sequence that is at least 95% identical to the amino acid sequence of SEQ ID NO:E; the vector comprises or consists of the sequence of GEO-D03 (SEQ ID NO: 7) or a sequence at least 85%, 90%, 95% or 98% identical thereto; the vector comprises or consists if the sequence of GEO-D06 (SEQ ID NO: 8) or a sequence at least 85%, 90%, 95% or 98% identical thereto; and the vector comprises the sequence of GEO-D07 (SEQ ID NO: 9) or a sequence at least 85%, 90%, 95% or 98% identical thereto.

Also described is a method of eliciting an immune response (e.g., a cellular immune response and/or a humoral immune response) in a subject, the method comprising administering to a subject one or more doses of a composition comprising a vector described herein. In various embodiments: at least two doses of the composition comprising the vector are administered to the subject; at least two doses of the composition comprising the vector are administered at least two months apart; the method further comprises the step of administering one or more doses of a composition comprising recombinant MVA virus expressing an HIV Gag, HIV Pol and HIV Env; the HIV Gag, HIV Pol and HIV Env expressed by the MVA are from the same clade as the HIV Gag, HIV Pol lacking the integrase domain, HIV Tat, HIV Rev, HIV Vpu, and HIV Env encoded by the DNA vector; at least one dose of a composition comprising recombinant MVA virus expressing an HIV Gag, HIV Pol and HIV Env is administered to the subject after the administration of at least one dose of a composition comprising of the vector; the administering results in an increase in the avidity of immunogen-specific antibodies, an increase in immunogen-specific antibody titers, an increase in immunogen specific IgA levels, or an increase in resistance to HIV infection.

Also described is a method of treating a subject infected with HIV, comprising administering to the subject one or more doses of a composition comprising a vector described herein. In various embodiments: the vector comprises or consists of the sequence of GEO-D03 (SEQ ID NO: 7) or a sequence at least 85%, 90%, 95% or 98% identical thereto; the vector comprises or consists if the sequence of GEO-D06 (SEQ ID NO: 8) or a sequence at least 85%, 90%, 95% or 98% identical thereto; and the vector comprises the sequence of GEO-D07 (SEQ ID NO: 9) or a sequence at least 85%, 90%, 95% or 98% identical thereto; the method further comprises a step of administering one or more doses of composition comprising recombinant MVA virus expressing an HIV Gag, HIV Pol and HIV Env; the HIV Gag, HIV Pol and HIV Env expressed by the MVA are from the same clade as the HIV Gag, HIV Pol lacking the integrase domain, HIV Tat, HIV Rev, HIV Vpu, and HIV Env encoded by the DNA vector; the at least one dose of a composition comprising recombinant MVA virus expressing an HIV Gag, HIV Pol and HIV Env is administered to the subject after the administration of at least one dose a of a composition comprising of a vector described herein to the subject; and the administering results in an increase in the avidity of immunogen-specific antibodies, an increase in immunogen-specific antibody titers, an increase in immunogen specific IgA levels, or an increase in resistance to HIV infection.

The present disclosure provides plasmid vectors that expresses one or more (e.g., two, three, four, five, six, seven, eight, nine, or ten) HIV antigens and human GM-CSF (granulocyte-macrophage colony stimulating factor; GenBank NP_{—}000749). Also provided are methods for using such vectors alone or in combination with MVA vectors expressing one or more (e.g., two, three, four, five, six, seven, eight, nine, or ten) HIV antigens, and methods for using a combination of a DNA vector encoding one or more (e.g., two, three, four, five, six, seven, eight, nine, or ten) HIV antigens together with a DNA vector encoding GM-CSF. This combination can be used in methods that also entail administration of a MVA vector encoding one or more (e.g., two, three, four, five, six, seven, eight, nine, or ten) HIV antigens.

Plasmid or viral vectors can include nucleic acids representing one or more (e.g., two, three, four, five, six, seven, eight, nine, or ten) genes found in one or more HIV clades or any fragments or derivatives thereof that, when expressed, elicit an immune response against the virus (or viral clade) from which the nucleic acid was derived or obtained. The nucleic acids may be purified from HIV or they may have been previously cloned, subcloned, or synthesized and, in any event, can be the same as or different from a naturally-occurring nucleic acid sequence. The plasmid vectors of the present disclosure may be referred to herein as, inter alia, expression vectors, expression constructs, plasmid vectors or, simply, as plasmids, regardless of whether or not they include a vaccine insert (i.e., a nucleic acid sequence that encodes an antigen or immunogen). Similar variations of the term “viral vector” may appear as well (e.g., we may refer to the “viral vector” as a “poxvirus vector,” a “vaccinia vector,” a “modified vaccinia Ankara vector,” or an “MVA vector”). The viral vector may or may not include a vaccine insert.

The disclosure provides compositions (including pharmaceutically or physiologically acceptable compositions) that contain, but are not limited to, a DNA vector, having a vaccine insert and a sequence encoding GM-CSF. The insert can include one or more of the sequences described herein (the features of the inserts and representative sequences are described at length below; any of these, or any combination of these, can be used as the insert). When the insert is expressed, the expressed protein(s) may generate an immune response against one or more (e.g., two, three, four, five, or six) HIV clades. One can increase the probability that the immune response will be effective against more than one clade by including sequences from more than one clade in the insert of a single vector (multi-vector vaccines are also useful and are described further below). For example, the vaccine inserts of any of the vectors, or the vectors described herein, may contain one or more (e.g., two, three, four, five, or six) designer sequences (e.g., mosaic sequences that contain a sequence from one or more HIV clades as described herein, for e.g., by using the Mosaic Vaccine Designer tool available from the Los Alamos website). The vaccine inserts of any of the vectors, or the vectors described herein, may also contain one or more (e.g., two, three, four, five, or six) sequences that encode one or more (e.g., two, three, four, five, or six) conserved protein sequences (for example, those sequences described in Rolland et al., PloS Pathogen 3:e157, 2007; Jiang et al., Nature Struct. Mol. Biol. 17:955-961, 2010; Mullins et al., AIDS Vaccine 2010, Oral Abstract No. OA01.01; and U.S. Patent Application Publication No. 20090092628, incorporated by reference) present in one or more HIV clades as described herein.

The disclosure also features compositions (including pharmaceutically or physiologically acceptable compositions) that contain, but are not limited to, at least one (e.g., two, three, four, five, or six) vector that encodes one or more (e.g., two, three, four, five, six, seven, eight, nine, or ten) antigens (i.e., a vector that includes a vaccine insert and/or a sequence expressing GM-CSF) that elicit (e.g., induces or enhances) an immune response against an HIV. A DNA vector can encode Gag-Pol or a modified form thereof. In addition, it can encode Gag-Pol and Env or modified forms thereof. The encoded HIV antigen can be a variant of a natural-occurring HIV antigen that includes one or more point mutations, insertions, or deletions. Particularly useful HIV antigen sequences include one or more (e.g., at least two, three, four, or five) safety mutations (e.g., deletion of the LTRs and of sequences encoding integrase (IN), Vif, Vpr, and Nef). The nucleic acids can encode one or more (e.g., two, three, four, five, six, or seven) of Gag, PR, RT, IN, Env, Tat, Rev, and Vpu proteins, one or more (e.g., two, three, four, five, six, or seven) of which may contain safety mutations (particular mutations are described at length below). Moreover, the isolated nucleic acids can be of any HIV clade and nucleic acids from different clades can be used in combination (as described further below). In the work described herein, clade B inserts are designated JS (e.g., JS2, JS7, and JS7.1), clade AG inserts are designated IC (e.g., IC2, IC25, IC48, and IC90), and clade C inserts are designated IN (e.g., IN2 and IN3). These inserts are within the scope of the present disclosure, as are vectors (whether plasmid or viral) containing them (particular vector/insert combinations are referred to below as, for example, pGA1/JS2, pGA2/JS2 etc. The DNA vectors can also encode human GM-CSF (mwlqsllllg tvacsisapa rspspstqpw ehvnaiqear rllnlsrdta aemnetvevi semfdlqept clqtrlelyk qglrgsltkl kgpltmmash ykqhcpptpe tscatqiitf esfkenlkdf llvipfdcwe pvqe; SEQ ID NO: 10). A non-limiting example of a location for insertion of the GM-CSF is shown in FIG. 1. The GM-CSF coding sequence can replace nef coding sequence and thus transcription will produce a full length mRNA that encodes a spliced mRNA that encodes Tat, a spliced mRNA that encodes Rev, a spliced mRNA that encodes Vpu-Env, and a spliced mRNA that encodes GM-CSF (produced using nef splicing sequences). In additional embodiments of the vaccine inserts and vectors of the disclosure, the GM-CSF coding sequence may contain an IRES (internal ribosom entry site). For example, the IRES sequence may be located 5′ of the nucleic acid sequence encoding GM-CSF. In additional embodiments of the vaccine inserts and vectors of the disclosure, the GM-CSF protein that is translated from a nucleic acid sequence encoding GM-CSF may be part of a polyprotein (e.g., a protein that contains one or more (e.g., at least 10, 15, 20, 25, 30, 35, 40, 45, 50, 60, 70, 80, 90, or 100) amino acids in addition to the polypeptide sequence of GM-CSF (e.g., the full-length protein or a fragment that has one or more biological activities GM-CSF). If a GM-CSF is expressed as a polyprotein, the full length GM-CSF or fragment of GM-CSF with one or more biological activities of GM-CSF may be produced following internal proteolytic cleavage using one or more (e.g., two proteases).

The DNA vectors of the present disclosure can include a termination sequence that improves stability. The termination sequence and other regulatory components (e.g., promoters and polyadenylation sequences) are discussed below.

The compositions of the disclosure can be administered to humans, including children. Accordingly, the disclosure features methods of immunizing a patient (or of eliciting an immune response in a patient, which may include multi-epitope CD8⁺ T cell responses) by administering one or more (e.g., two, three, four, five, or six) types of vectors (e.g., one or more plasmids, which may or may not have identical sequences, components, or inserts (e.g., sequences that can encode antigens) and/or one or more (e.g., two, three, four, five, or six) viral vectors, which may or may not be identical or express identical antigens). As noted above, the vectors, whether plasmid or viral vectors, can include one or more (e.g., two, three, four, five, or six) nucleic acids obtained from or derived from (e.g., a mutant sequence is a derivative sequence) one or more HIV clades. When these sequences are expressed, they produce an antigen or antigens that elicit an immune response to one or more (e.g., two, three, four, five, six, seven, eight, nine, ten, eleven, or twelve) epitopes from one or more (e.g., two, three, four, five, or six) HIV clades.

Where the compositions contain vectors that differ either in their backbone, regulatory elements, or insert(s), the ratio of the vectors in the compositions, and the routes by which they are administered, can vary. The ratio of one type of vector to another can be equal or roughly equal (e.g., roughly 1:1 or 1:1:1, etc.). Alternatively, the ratio can be in any desired proportion (e.g., 1:2, 1:3, 1:4 . . . 1:10; 1:2:1, 1:3:1, 1:4:1 . . . 1:10:1; etc.). Thus, the disclosure features compositions containing a variety of vectors, the relative amounts of antigen-expressing vectors being roughly equal or in a desired proportion. While preformed mixtures may be made (and may be more convenient), one can, of course, achieve the same objective by administering two or more (e.g., three, four, five, or six) vector-containing compositions (on, for example, the same occasion (e.g., within minutes of one another) or nearly the same occasion (e.g., on consecutive days)).

Plasmid vectors can be administered alone (i.e., a plasmid can be administered on one or several occasions with or without an alternative type of vaccine formulation (e.g., with or without administration of protein or another type of vector, such as a viral vector)) and, optionally, with an adjuvant or in conjunction with (e.g., prior to) an alternative booster immunization (e.g., a live-vectored vaccine such as a recombinant modified vaccinia Ankara vector (MVA)) comprising an insert that may be distinct from that of the “prime” portion of the immunization or may be a related vaccine insert(s). For example, the viral vector can contain at least some of the sequence contained with the plasmid administered as the “prime” portion of the inoculation protocol (e.g., sequences encoding one or more, and possibly all, of the same antigens). The adjuvant can be a “genetic adjuvant” (i.e., a protein delivered by way of a DNA sequence). Similarly, as described further below, one can immunize a patient (or elicit an immune response, which can include multi-epitope CD8⁺ T cell responses) by administering a live-vectored vaccine (e.g., an MVA vector) without administering a plasmid-based (or “DNA”) vaccine. Thus, in alternative embodiments, the disclosure features compositions having only viral vectors (with, optionally, one or more (e.g., two, three, four, five, or six) of any of the inserts described here, or inserts having their features) and methods of administering them. The viral-based regimens (e.g., “MVA only” or “MVA-MVA” vaccine regimens) are the same as those described herein for “DNA-MVA” regimens, and the MVAs in any vaccine can be in any proportion desired. For example, in any case (whether the immunization protocol employs only plasmid-based immunogens, only viral-carried immunogens, or a combination of both), one can include an adjuvant and administer a variety of antigens, including those obtained from any HIV clade, by way of the plurality of vectors administered.

As implied by the term “immunization” (and variants thereof), the compositions of the disclosure can be administered to a subject who has not yet become infected with a pathogen (thus, the terms “subject” or “patient,” as used herein encompasses apparently healthy or non-HIV-infected individuals), but the disclosure is not so limited; the compositions described herein can also be administered to treat a subject or patient who has already been exposed to, or who is known to be infected with, a pathogen (e.g., an HIV of any clade, including those presently known as clades A-L or mutant or recombinant forms thereof). In either infected or uninfected patients, the vectors can elicit a beneficial immune response that either decreases (e.g., by at least 10%, 15%, 20%, 25%, 30%, 35%, 40%, 45%, 50%, 55%, 60%, 65%, 70%, 75%, 80%, 85%, 90%, 95%, or 100%) the risk or rate of infection (in the case of uninfected patients) or provides a therapeutic benefit in patients that are infected.

An advantage of DNA and rMVA immunizations is that the immunogen may be presented by both MHC class I and class II molecules. Endogenously synthesized proteins readily enter processing pathways that load peptide epitopes onto MHC I as well as MHC II molecules. MHC I-presented epitopes raise CD8 cytotoxic T cell (Tc) responses, whereas MHC II-presented epitopes raise CD4 helper T cells (Th). By contrast, immunogens that are not synthesized in cells are largely restricted to the loading of MHC II epitopes and therefore raise CD4 Th but not CD8 Tc. In addition, DNA plasmids express only the immunizing antigens in transfected cells and can be used to focus the immune response on only those antigens desired for immunization. In contrast, live virus vectors express many antigens (e.g., those of the vector as well as the immunizing antigens) and prime immune responses against both the vector and the immunogen. Thus, these vectors could be highly effective at boosting a DNA-primed response by virtue of the large amounts of antigen that can be expressed by a live vector preferentially boosting the highly targeted DNA-primed immune response. The live virus vectors also stimulate the production of pro-inflammatory cytokines that augment immune responses. Thus, administering one or more of the DNA vectors described herein (as a “prime”) and subsequently administering one or more of the viral vectors (as a “boost”), could be more effective than DNA-alone or live vectors-alone at raising both cellular and humoral immunity. Insofar as these vaccines may be administered by DNA expression vectors and/or recombinant viruses, there is a need for plasmids that are stable in bacterial hosts and safe in animals. Plasmid-based vaccines that may have this added stability are disclosed herein, together with methods for administering them to animals, including humans.

The antigens encoded by DNA or rMVA are necessarily proteinaceous. The terms “protein,” “polypeptide,” and “peptide” are generally interchangeable, although the term “peptide” is commonly used to refer to a short sequence of amino acid residues or a fragment of a larger protein. In any event, serial arrays of amino acid residues, linked through peptide bonds, can be obtained by using recombinant techniques to express DNA (e.g., as was done for the vaccine inserts described and exemplified herein), purified from a natural source, or synthesized. Other advantages of DNA-based vaccines (and of viral vectors, such as pox virus-based vectors) are described below.

Accordingly, the disclosure provides vectors containing a prokaryotic origin of replication, a promoter sequence, a eukaryotic transcription cassette containing a vaccine insert encoding one or more (e.g., two, three, four, five, six, seven, eight, nine, or ten) immunogens and GM-CSF, a polyadenylation sequence, and a transcription termination sequence. In additional embodiments of the vector, the prokaryotic origin of replication is ColE1 or the promoter sequence is CMVIE-intron A or CMV promoter. In additional embodiments of the vectors, the polyadenylation sequence is bovine growth hormone polyadenylation sequence or the transcription termination sequence is lambda T0 terminator. Additional embodiments of the above vectors further contain a selectable marker gene. In additional embodiments, the vector contains the sequence of GEO-D03 (SEQ ID NO: 7), GEO-D06 (SEQ ID NO: 8), or GEO-D07 (SEQ ID NO: 9).

As noted above, the disclosure also provides vaccine inserts encoding one or more (e.g., two, three, four, five, six, seven, eight, nine, or ten) immunogens and GM-CSF. In additional embodiments of the vaccine inserts, the insert contains the sequence of nucleotides 106 to 7067 of GEO-D03 (SEQ ID NO: 7), the sequence of nucleotides 99 to 7082 of GEO-D06 (SEQ ID NO: 8), or nucleotides 787 to 7770 of GEO-D07 (SEQ ID NO: 9).

In any of the above described vectors or vaccine inserts, the vaccine insert can contain a sequence that encodes one or more (e.g., two, three, four, five, six, seven, eight, nine, or ten) immunogens selected from the group of: Gag, gp160, gp120, gp41, pol, env, Tat, Rev, Vpu, Nef, Vif, and Vpr. In additional embodiments of all the above vectors and vaccine inserts, the one or more (e.g., two, three, four, five, six, seven, eight, nine, or ten) immunogens are from one or more (e.g., two, three, four, five, six, seven, eight, nine, ten, eleven, or twelve) HIV clades (e.g., HIV clades A, B, C, D, E, F, G, H, I, J, K, and L). In additional embodiments of the above vectors and vaccine inserts, the one or more (e.g., two, three, four, five, six, seven, eight, nine, or ten) immunogens are from the same HIV clade (e.g., HIV clades A, B, C, D, E, F, G, H, I, J, K, or L). In additional embodiments of all the above vectors and vaccine inserts, the one or more (e.g., two, three, four, five, six, seven, eight, nine, or ten) immunogens (e.g., Gag, Env (e.g., gp120, gp41, or gp160), Pol, Tat, Rev, Vpu, Nef, Vif, and Vpr) is a mutant or a natural variant (e.g., an immunogen that is a result of recombination or alternative splicing). For example, in any of the above vectors or vaccine inserts, the mutant immunogen is gag, and a mutation is in a sequence encoding matrix protein (p17), capsid protein (p24), nucleocapsid protein (p7), or C-terminal peptide (p6). In any of the above vectors or vaccine inserts, the mutant immunogen can be pol, and a mutation can be present in a sequence encoding protease protein (p10), reverse transcriptase (p66/p51), or integrase protein (p32). In any of the above vectors and vaccine inserts, the insert can contain a sequence that encodes Gag, Pol, Tat, Rev, and Env. In additional embodiments of the above vectors and vaccine inserts, the insert can contain a sequence that encodes Gag, Pol, Tat, Rev, Env, and Vpu.

In any of the above vectors or vaccine inserts, the encoded GM-CSF can be full-length human GM-CSF. In additional embodiments of the vectors and vaccine inserts, the sequence encoding GM-CSF can contain the sequence of: nucleotides 6633-7067 of SEQ ID NO: 7, nucleotides 6648-7082 of SEQ ID NO: 8, or nucleotides 7336-7770 of SEQ ID NO: 9. In any of the above vectors or vaccine inserts, the encoded GM-CSF can be a truncated human GM-CSF or a mutant human GM-CSF that is capable of stimulating macrophage differentiation and proliferation, or activating antigen presenting dendritic cells. In additional embodiments of the above vectors and vaccine inserts, the translated GM-CSF polypeptide encoded by the vaccine does not contain a polypeptide sequence of an immunogen (e.g., a HIV immunogen).

The disclosure further provides methods of inducing an immune response in a subject requiring administering to a subject one or more (e.g., two, three, four, five, or six) doses of any of the above described vectors. In additional embodiments of these methods, the subject has HIV or is at risk of developing HIV infection. In further examples of these methods, the administering results in an increase (e.g., at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, or 50%) in the avidity of immunogen-specific antibodies, no increase or an increase (e.g., by at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 300-fold, 400-fold, 500-fold) in immunogen-specific antibody titers, an increase (e.g., at least 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold) in immunogen-specific IgA levels (e.g., IgA levels in rectal secretions), a level of between 0.03 to 0.3 ng of immunogen-specific IgA per μg of total IgA, a level of between 0.1 to 0.3 ng of immunogen-specific IgA per μg of total IgA, a level of between 0.2 to 0.3 ng of immunogen-specific IgA per μg of total IgA, an increase (e.g., at least 10%, 15%, 20%, 25%, 30%, 35%, 40%, 45%, 50%, 55%, 60% 65%, 70%, 75%, 80%, 85%, 90%, 95%, or 100%) in resistance to HIV infection, an increase (e.g., by at least 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 40-fold, 50-fold, 100-fold, 200-fold, 300-fold, 400-fold, 500-fold) in neutralizing antibody titers, an increase (e.g., by at least 10%, 15%, 20%, 25%, 30%, 35%, 40%, 45%, 50%, 55%, 60% 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, or 300%) in antibody-dependent cellular cytotoxicity, an increase (e.g., at least 5%, 10%, 15%, 20%, 25%, 30%, 35%, 40%, 45%, 50%, 55%, 60%, 65%, 70%, 75%, 80%, 85%, 90%, 95%, or 100%) in immunogen-specific CD4 helper T cells, and/or an increase (e.g., at least 5%, 10%, 15%, 20%, 25%, 30%, 35%, 40%, 45%, 50%, 55%, 60%, 65%, 70%, 75%, 80%, 85%, 90%, 95%, or 100%) in immunogen-specific CD8 cytotoxic T cells.

The disclosure further provides methods of treating a subject having HIV requiring administrating to the subject one or more (e.g., two, three, four, five, or six) doses of any of the above described vectors. In additional embodiments of these methods, the administering results in an increase (e.g., by at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, or 50%) in the avidity of immunogen-specific antibodies, an increase (e.g., at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 300-fold, 400-fold, 500-fold) in immunogen-specific antibody titers, an increase (e.g., at least 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold) in immunogen-specific IgA levels (e.g., IgA levels in rectal secretions), a level of between 0.03 to 0.3 ng of immunogen-specific IgA per μg of total IgA, a level of between 0.1 to 0.3 ng of immunogen-specific IgA per μg of total IgA, a level of between 0.2 to 0.3 ng of immunogen-specific IgA per μg of total IgA, an increase (e.g., by at least 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 40-fold, 50-fold, 100-fold, 200-fold, 300-fold, 400-fold, 500-fold) in neutralizing antibody titers, an increase (e.g., by at least 10%, 15%, 20%, 25%, 30%, 35%, 40%, 45%, 50%, 55%, 60% 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, or 300%) in antibody-dependent cellular cytotoxicity, an increase (e.g., at least 5%, 10%, 15%, 20%, 25%, 30%, 35%, 40%, 45%, 50%, 55%, 60%, 65%, 70%, 75%, 80%, 85%, 90%, 95%, or 100%) in immunogen-specific CD4 helper T cells, and/or an increase (e.g., at least 5%, 10%, 15%, 20%, 25%, 30%, 35%, 40%, 45%, 50%, 55%, 60%, 65%, 70%, 75%, 80%, 85%, 90%, 95%, or 100%) in immunogen-specific CD8 cytotoxic T cells.

In any of the above methods, the vector contains a vaccine insert that contains a sequence that encodes one or more (e.g., two, three, four, five, six, seven, eight, nine, or ten) immunogens selected from the group of: Gag, Pol, Env (e.g., gp160, gp120, and gp41), Tat, Rev, Vpu, Nef, Vif, and Vpr. In any of the above methods, the vector can contain the sequence of GEO-D03 (SEQ ID NO: 7), GEO-D06 (SEQ ID NO: 8), or GEO-D07 (SEQ ID NO: 9). In additional embodiments of the above methods, at least one (e.g., two, three, four, five, or six) doses of at least one (e.g., two, three, four, five, or six) vector is administered to the subject. In additional examples of the above methods, at least two doses of at least one (e.g., two, three, four, five, or six) vector is administered at least 1 week (e.g., at least 2 weeks, 3 weeks, 1 month, 5 weeks, 6 weeks, 7 weeks, 2 months, 3 months, 4 months, 5 months, 6 months, 7 months, 8 months, 9 months, 10 months, 11 months, 1 year, 13 months, 14 months, 15 months, 16 months, 17 months, or 18 months) apart. In further examples, the above methods further include the step of administering one or more (e.g., two, three, four, five, or six) doses of a MVA vaccine encoding one or more (e.g., two, three, four, five, six, seven, eight, nine, ten, eleven, twelve, or thirteen) immunogens (e.g., Gag, gp41, gp120, gp160, pol, env, Tat, Rev, Vpu, Nef, Vif, Vpr, pr, rt, and in (integrase)). In additional embodiments of the above methods, the one or more immunogens encoded by the MVA are from one or more (e.g., two, three, four, five, six, seven, eight, nine, ten, eleven, or twelve) HIV clades. In additional examples of the above methods, the one or more immunogens encoded by the MVA are from the same HIV clade.

In any of the above methods, the at least one (e.g., two, three, four, five, or six) dose of the MVA vaccine is administered to the subject after (e.g., at least 1 day, 2 days, 3 days, 4 days, 5 days, 6 days, 1 week, 2 weeks, 3 weeks, 4 weeks, 5 weeks, 6 weeks, 7 weeks, 8 weeks, 10 weeks, 12 weeks, 16 weeks, 20 weeks, or 24 weeks after) the administration of at least one (e.g., two, three, four, five, or six) dose of any of the above described vectors. In additional embodiments of the above methods, the at least one dose (e.g., two, three, four, five, or six) of the MVA vaccine is administered to the subject at the same time as administration of a dose of any of the above described vectors. In any of the above described methods, the subject can be human.

The disclosure further provides methods of manufacturing a medicament for inducing an immune response in a subject using any of the above described vectors. In additional embodiments of these methods, the subject has or is at risk of developing a HIV infection. In additional embodiments of these methods, the vector contains the sequence of GEO-D03 (SEQ ID NO: 7), GEO-D06 (SEQ ID NO: 8), or GEO-D07 (SEQ ID NO: 9).

By the term “inducing an immune response” is meant at least an increase (e.g., at least 1%, 2%, 3%, 4%, 5%, 6%, 7%, 9%, 10%, 11%, 12%, 13%, 14%, 15%, 16%, 17%, 18%, 19%, 20%, 30%, 40%, or 50%) in the avidity of immunogen-specific antibodies, an increase (e.g., at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 300-fold, 400-fold, or 500-fold) in immunogen-specific antibody titers, an increase (e.g., at least 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold) in immunogen-specific IgA levels (e.g., IgA levels in rectal secretions), a level of between 0.03 to 0.3 ng of immunogen-specific IgA per μg of total IgA, a level of between 0.1 to 0.3 ng of immunogen-specific IgA per μg of total IgA, a level of between 0.2 to 0.3 ng of immunogen-specific IgA per μg of total IgA, an increase (e.g., by at least 10%, 15%, 20%, 25%, 30%, 35%, 40%, 45%, 50%, 55%, 60% 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, or 300%) in antibody-dependent cellular cytotoxicity, an increase (e.g., at least 5%, 10%, 15%, 20%, 25%, 30%, 35%, 40%, 45%, 50%, 55%, 60%, 65%, 70%, 75%, 80%, 85%, 90%, 95%, or 100%) in immunogen-specific CD4 helper T cells, or an increase (e.g., at least 5%, 10%, 15%, 20%, 25%, 30%, 35%, 40%, 45%, 50%, 55%, 60%, 65%, 70%, 75%, 80%, 85%, 90%, 95%, or 100%) in immunogen-specific CD8 cytotoxic T cells.

By the term “natural variant” is meant a sequence that is naturally found in a subject or a virus. For example, human genes often contain single nucleotide polymorphisms that are present in certain individuals within a population. Viruses often acquire spontaneous mutations in their nucleic acid after serial passage in vitro or upon replication in an infected subject. Mutations within HIV sequences may confer resistance to drug treatment or alter the rate of infection or replication of the virus in a subject. Several natural variant sequences of HIV clades are known in the art (see, for example, the Los Alamos DNA Database website).

By the term “mutant” is meant at least one (e.g., at least two, three, four, five, six, seven, eight, nine, or ten) amino acid or nucleotide change in a sequence when compared to a wild type or predominant polypeptide or nucleotide sequence. A mutation may occur naturally in a cell or may be introduced by molecular biology techniques into a target sequence. The term mutant can include one or more (e.g., two, three, four, five, six, seven, eight, nine, or ten) amino acid or nucleotide deletions, additions, or substitutions.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1. Schematic drawing of a DNA vector expressing HIV antigens and GM-CSF.

FIG. 2. Immunization schedule of macaques.

FIG. 3. Intrarectal challenge conditions.

FIG. 4. Schematics of SIV239 DNA and recombinant MVA vaccines. D, SIV239 DNA vaccine; Dg, GM-CSF co-expressing SIV239 DNA vaccine; M, SIV239 MVA vaccine. Transcriptional control elements are shaded. For the DNA vaccines, transcription is initiated by the cytomegalovirus immediate early promoter (CMVIE) including intron A and terminated by the bovine growth hormone polyadenylation sequence (BGHpA). For the MVA vaccine, transcription is under the control of the p7.5 (env) and mH5 (gag-pol) promoters. gag, Pr, RT, tat, rev, env are sequences encoding the group specific antigens, protease, reverse transcriptase, transcriptional activator, regulatory protein, and envelope glycoprotein respectively of SIV239. Xs indicates inactivating point mutations in reverse transcriptase and packaging sequences in gag.

FIG. 5. Humoral immune responses elicited by the GM-CSF-adjuvanted and nonadjuvanted DNA/MVA vaccines. DNA priming immunizations were administered at weeks 0 and 8 and MVA booster immunizations at weeks 16 and 24. A, Env-specific IgG responses measured in serum at pre-immunization, 2, 10, 18, 21, 26, and 37 weeks in the trial. Micrograms of IgG are estimated relative to a standard curve of rhesus IgG. Values are medians±interquartile ranges. B, Tukey plots presenting Env-specific IgA responses in rectal secretions at pre-immunization, 2 weeks after the indicated immunizations, and pre-challenge. IgA is presented as Env-specific IgA divided by total IgA. C, Avidity indices for elicited IgG for the SIV239 Env of the immunogen and the SIVE660 Env of the challenge measured at 2 weeks after the second MVA immunization. Avidity indices increased with time in the trial and further increased post infection. D, Neutralization titers for pseudotypes with two Envs molecularly cloned from the genetically diverse SIVE660 stock. Titers for SIVE660.11 were determined at 2 weeks post the second MVA boost; and, for SIVE660.17, at 13 weeks after the second MVA boost. Titers are the reciprocal for the dilution of serum achieving an inhibitory dose 50 (ID50) in the TZM-bl assay. E, ADCC titers for SIVmac239 gp120 coated CEM.NKRCCR5 cells at two weeks following the second MVA boost. In panels C-E, Boxplots present median and 25th and 75th percentiles for responses. Target Envs and the significance for differences between the DDMM and DgDgMM regimens are indicated above boxplots. Statistical comparisons were made using a two-sided Wilcoxon's rank-sum test.

FIG. 6. SIVmac251 Env-specific IgA antibodies in rectal secretions of M11 macaques.

FIG. 7. SIV Gag/Pol-specific antibodies in rectal secretions of M11 macaques.

FIG. 8. Cellular immune responses elicited by the GM-CSF-adjuvanted and non-adjuvanted DNA/MVA vaccines. DNA priming immunizations were administered at weeks 0 and 8 and MVA booster immunizations at weeks 16 and 24. A: Vaccine-elicited CD4. B: CD8 T cell responses at preimmunization and 2, 10, 17, 21, 25, and 37 weeks in the trial. Responses are IFN-γ secreting cells scored by ICS following Gag and Env peptide stimulation of PBMCs. Grey boxes represent the background for detection. C: Breadth of vaccine-elicited IFN-γ secreting CD4 responses. D: CD8 T cell responses measured by ICS of PBMC stimulated with 13 Gag and 11 Env peptide pools at one week post the 1st and 2nd MVA immunizations. E and F: Polyfunctionality for cytokine production by elicited CD4 and CD8 T cell responses at one week following the 2nd MVA immunization. Boolean analyses were used to determine the frequencies of IFN-γ, IL-2, and TNF-α producing cells responding to Gag and Env. Only those responses that were >0.07% of total cytokine positive cells were considered for analysis. The boxplots present the median and interquartile ranges for the percent of responding cells (as a proportion of total cytokine positive cells) producing 1, 2, or 3 cytokines. Patterns of cytokine production for individual subsets of single or double producers were overall similar (data not shown).

FIG. 9. Co-expressed GM-CSF enhances protection against infection. A: Kaplan-Meier curve for number of challenges to infection. Animals that were not infected by the 12 challenges are plotted at 14 challenges. P=0.003 is the significance for the difference in number of challenges to infection between the DgDgMM and unvaccinated group (log-rank Mandel-Cox test). B: Temporal post-challenge viremia in animals that became infected. Infection dates are adjusted with week one being the 1st week an infection was detected. Data are presented as means±one standard deviation to show the differences in overall levels of viremia in the groups. Differences between groups are not significant due to small group sizes and variability in responses. The grey box represents the background for detection.

FIG. 10. Absence of anamnestic Ab responses in repeatedly challenged animals that did not become infected. A: Absence of a detectable anamnestic Env-specific IgA response in uninfected rhesus macaques at various weeks post the last challenge. B: Strong anamestic IgA responses for Env in vaccinated animals that became infected. C: Absence of a detectable anamnestic IgG response for Env in uninfected rhesus macaques at various weeks post the last challenge. D: Strong anamnestic IgG responses for Env in vaccinated animals that became infected. Data are presented as medians±interquartile ranges. The grey boxes represent backgrounds for detection.

FIG. 11. Post challenge humoral and cellular immune responses. A: Titers of SIV239 Env-specific IgG in vaccinated macaques who did become infected. Note the strong IgG response in the infected animals. Titers of IgG are estimated relative to a standard curve of macaque IgG. D: T cells post-challenge in infected animals.

FIG. 12. Avidity of the vaccine-elicited IgG for the Env of the challenge virus correlates with protection. A: Significant correlation between avidity of the elicited IgG for the SIVE660 Env of the challenge virus and the number of challenges to infection. Data are presented as the mean±one standard deviation for 3 independent assays. Animals that did not become infected by the 12 challenges are plotted at 14 challenges. Correlations were done using the two sided Spearman rank order statistical analysis. B: The TRIM5α genotype of vaccinated rhesus macaques does not restrict the number of challenges to infection r, restrictive TRIM5α genotype (homozygous or heterozygous for TRIM5α TFP or CYPA); s, susceptible genotype (homozygous for TRIM5αQ); m, moderately susceptible (heterozygous for a restrictive and permissive allele). Animals that were not infected by the 12 challenges are plotted at challenge 14.

FIG. 13. Avidity of the vaccine-elicited IgG for the Env of the challenge virus correlates with protection. A: Lack of correlation between the avidity of the elicited IgG for the SIV239 Env and the number of challenges to infection. In A, data are means±standard deviations for 3 independent assays. Animals that did not become infected by the 12 challenges are plotted at 14 challenges. Correlations were done using the two sided Spearman rank order statistical analysis. B: Lack of correlation between the Trim5α genotype of vaccinated macaques and the height of peak viremia. r, restrictive Trim5α genotype (homozygous or heterozygous for Trim5αTFP or CYPA); s, susceptible genotype (homozygous for Trim5αQ); m, moderately susceptible (heterozygous for a restrictive and permissive allele). Horizontal lines indicate median number of challenges to infection. Note how the unvaccinated controls, but not the vaccinated animals, are sensitive to the Trim5α restriction for both the number of challenges to infection and the height of peak viremia.

FIG. 14. Sequence of the GEO-D03 DNA vector (SEQ ID NO: 7) expressing HIV antigens and GM-CSF.

FIG. 15. Sequence of the GEO-D06 DNA vector (SEQ ID NO: 8) expressing HIV antigens and GM-CSF.

FIG. 16. Sequence of the GEO-D07 DNA vector (SEQ ID NO: 9) expressing HIV antigens and GM-CSF.

### DETAILED DESCRIPTION

This disclosure encompasses a wide variety of vectors and types of vectors (e.g., plasmid and viral vectors), each of which can, but do not necessarily, include one or more nucleic acid sequences that encode one or more antigens that elicit (e.g., that induce or enhance) an immune response against the pathogen from which the antigen was obtained or derived (the sequences encoding proteins that elicit an immune response may be referred to herein as “vaccine inserts” or, simply, “inserts”; when a mutation is introduced into a naturally occurring sequence, the resulting mutant is “derived” from the naturally occurring sequence). We point out that the vectors do not necessarily encode antigens to make it clear that vectors without “inserts” are within the scope of the disclosure and that the inserts per se are also compositions of the disclosure.

Accordingly, the disclosure features the nucleic acid sequences disclosed herein, analogs thereof, and compositions containing those nucleic acids (whether vector plus insert or insert only; e.g., physiologically acceptable solutions, which may include carriers such as liposomes, calcium, particles (e.g., gold beads) or other reagents used to deliver DNA to cells). The analogs can be sequences that are not identical to those disclosed herein, but that include the same or similar mutations (e.g., the same point mutation or a similar point mutation) at positions analogous to those included in the present sequences (e.g., any of the JS, IC, or IN sequences disclosed herein). A given residue or domain can be identified in various HIV clades even though it does not appear at precisely the same numerical position. The analogs can also be sequences that include mutations that, while distinct from those described herein, similarly inactivate an HIV gene product. For example, a gene that is truncated to a greater or lesser extent than one of the genes described here, but that is similarly inactivated (e.g., that loses a particular enzymatic activity) is within the scope of the present disclosure.

The pathogens and antigens, which are described in more detail in US-2003-0175292-A1 (incorporated by reference), include human immunodeficiency viruses of any clade (e.g. from any known clade or from any isolate (e.g., clade A, AG, B, C, D, E, F, G, H, I, J, K, or L). Additional HIV sequences and mutant sequences are known in the art (e.g., the HIV Sequence Database in Los Alamos and the HIV RT/Protease Sequence Database in Stanford). When the vectors include sequences from a pathogen, they can be administered to a patient to elicit an immune response. Thus, methods of administering antigen-encoding vectors, alone or in combination with one another, are also described herein. These methods can be carried out to either immunize patients (thereby reducing the patient's risk of becoming infected) or to treat patients who have already become infected; when expressed, the antigens may elicit both cell-mediated and humoral immune responses that may substantially prevent the infection (e.g., immunization can protect against subsequent challenge by the pathogen) or limit the extent of the impact of an infection on the patient's health. While in many instances the patient will be a human patient, the disclosure is not so limited. Other animals, including non-human primates, domesticated animals, and livestock can also be treated.

The compositions described herein, regardless of the pathogen or pathogenic subtype (e.g., the HIV clade(s)) they are directed against, can include a nucleic acid vector (e.g., a plasmid). As noted herein, vectors having one or more of the features or characteristics (particularly the oriented termination sequence and a strong promoter) of the plasmids designated pGA1, pGA2 (including, of course, those vectors per se), can be used as the basis for a vaccine or therapy. Such vectors can be engineered using standard recombinant techniques (several of which are illustrated in the examples, below) to include sequences that encode antigens that, when administered to, and subsequently expressed in, a patient will elicit (e.g., induce or enhance) an immune response that provides the patient with some form of protection against the pathogen from which the antigens were obtained or derived (e.g., protection against infection, protection against disease, or amelioration of one or more of the signs or symptoms of a disease). The encoded antigens can be of any HIV clade or subtype or any recombinant form thereof. With respect to inserts from immunodeficiency viruses, different clades exhibit clustal diversity, with each isolate within a clade having overall similar diversity from the consensus sequence for the clade (see, e.g., Subbarao et al., AIDS 10(Suppl A):513-23, 1996). Thus, most isolates can be used as a reasonable representative of sequences for other isolates of the same clade. Accordingly, the compositions of the disclosure can be made with, and the methods described herein can be practiced with, natural variants of genes or nucleic acid molecules that result from recombination events, alternative splicing, or mutations (these variants may be referred to herein simply as “recombinant forms” of HIV).

Moreover, one or more of the inserts within any construct can be mutated to decrease their natural biological activity (and thereby increase their safety) in humans.

At least one of the two or more sequences can be mutant or mutated so as to limit the encapsidation of viral RNA (preferably, the mutation(s) limit encapsidation appreciably). One can introduce mutations and determine their effect (on, for example, expression or immunogenicity) using techniques known in the art; antigens that remain well expressed (e.g., antigens that are expressed about as well as or better than their wild type counterparts), but are less biologically active than their wild type counterparts, are within the scope of the disclosure. Techniques are also available for assessing the immune response. One can, for example, detect anti-viral antibodies or virus-specific T cells. Desirably, the mutant vectors or vaccine inserts provided result in an increase (e.g., at least 1%, 2%, 3%, 4%, 5%, 6%, 7%, 8%, 9%, 10%, 11%, 12%, 13%, 14%, 15%, 16%, 17%, 18%, 19%, 20%, 30%, 40%, or 50%) in the avidity of immunogen-specific antibodies, an increase (e.g., by at least 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 300-fold, 400-fold, or 500-fold) in immunogen-specific antibody titers, an increase (e.g., at least 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold) in immunogen-specific IgA levels (e.g., IgA levels in rectal secretions), a level of between 0.03 to 0.3 ng of immunogen-specific IgA per μg of total IgA, a level of between 0.1 to 0.3 ng of immunogen-specific IgA per μg of total IgA, a level of between 0.2 to 0.3 ng of immunogen-specific IgA per μg of total IgA, an increase (e.g., at least 10%, 15%, 20%, 25%, 30%, 35%, 40%, 45%, 50%, 55%, 60% 65%, 70%, 75%, 80%, 85%, 90%, 95%, or 100%) in resistance to HIV infection, an increase (e.g., by at least 10%, 15%, 20%, 25%, 30%, 35%, 40%, 45%, 50%, 55%, 60% 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, or 300%) in antibody-dependent cellular cytotoxicity, an increase (e.g., at least 5%, 10%, 15%, 20%, 25%, 30%, 35%, 40%, 45%, 50%, 55%, 60%, 65%, 70%, 75%, 80%, 85%, 90%, 95%, or 100%) in immunogen-specific CD4 helper T cells, and/or an increase (e.g., at least 5%, 10%, 15%, 20%, 25%, 30%, 35%, 40%, 45%, 50%, 55%, 60%, 65%, 70%, 75%, 80%, 85%, 90%, 95%, or 100%) in immunogen-specific CD8 cytotoxic T cells, and/or an increase (e.g., by at least 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 40-fold, 50-fold, 100-fold, 200-fold, 300-fold, 400-fold, 500-fold) in neutralizing antibody titers.

The mutant constructs (e.g., a vaccine insert) can include sequences encoding one or more of the substitution mutants described herein (see, e.g. the Examples) or an analogous mutation in another HIV clade. In addition to, or alternatively, HIV antigens can be rendered less active by deleting part of the gene sequences that encode them. Thus, the compositions of the disclosure can include constructs that encode antigens that, while capable of eliciting an immune response, are mutant (whether encoding a protein of a different length or content than a corresponding wild type sequence) and thereby less able to carry out their normal biological function when expressed in a patient. As noted above, expression, immunogenicity, and activity can be assessed using standard techniques in molecular biology and immunology.

The DNA vectors express HIV-1 antigens and GM-CSF, and those constructs can be administered to patients as described herein. The GM-CSF sequence can be introduced into a variety of different DNA vectors expressing HIV-1 antigens. JS7-like inserts, described below, and in US-2003-0175292-A1 are particularly useful. Any plasmid within the scope of the disclosure can be tested for expression by transfecting cells, such as 293T cells (a human embryonic kidney cell line) and assessing the level of antigen expression (by, for example, an antigen-capture ELISA or a Western blot).

The GM-CSF sequence included in the vectors and the vaccine inserts may be a full-length human GM-CSF (SEQ ID NO: 10) or may be a polypeptide that includes a sequence that is at least 95% identical to GM-CSF (SEQ ID NO: 10) and has one or more (e.g., two or three) biological activities of GM-CSF (e.g., capable of stimulating macrophage differentiation and proliferation, or activating antigen presenting dendritic cells). The GM-CSF may include one or more mutations (e.g., one or more (e.g., at least two, three, four, five, or six) amino acid substitutions, deletions, or additions)). Desirably, any mutant GM-CSF proteins also have one or more (e.g., two or three) biological activities of GM-CSF (as described above). Assays for the measurement of the biological activity of GM-CSF proteins are known in the art (see, e.g., U.S. Pat. No. 7,371,370; incorporated herein by reference in its entirety).

The nucleic acid vectors of the disclosure encode GM-CSF and at least one antigen (which may also be referred to as an immunogen) obtained from, or derived from, any HIV clade or isolate (i.e., any subtype or recombinant form of HIV). The antigen (or immunogen) may be: a structural component of an HIV virus; glycosylated, myristoylated, or phosphorylated; one that is expressed intracellularly, on the cell surface, or secreted (antigens that are not normally secreted may be linked to a signal sequence that directs secretion). More specifically, the antigen can be all, or an antigenic portion of, Gag, Pol, Env (e.g., gp160 or gp120, or a CCR5-using Env), Tat, Rev, Vpu, Nef, Vif, Vpr, or a VLP (e.g., a polypeptide derived from a VLP that is capable of forming a VLP, including an Env-defective HIV VLP).

Particular inserts and insert-bearing compositions include the following. Where the composition includes either a vector with an insert or an insert alone, and that insert encodes a single antigen, the antigen can be a wild type or mutant gag sequence (e.g., a gag sequence having a mutation in one or more of the sequences encoding a zinc finger at one or more of the cysteine residues at positions 392, 395, 413, or 416 to another residue (e.g., serine) or the mutation can change one or more of the cysteine residues at positions 390, 393, 411, or 414 to another residue (e.g., serine).

Where the composition includes either a vector with an insert or an insert alone, and that insert encodes multiple protein antigens, one of the antigens can be a wild type or mutant gag sequence, including those described above. Similarly, where a composition includes more than one type of vector or more than one type of insert, at least one of the vectors or inserts (whether encoding a single antigen or multiple antigens) can include a wild type or mutant gag sequence, including those described above or analogous sequences from other HIV clades. For example, where the composition includes first and second vectors, the vaccine insert in either or both vectors (whether the insert encodes single or multiple antigens) can encode gag; where both vectors encode gag, the gag sequence in the first vector can be from one HIV clade (e.g., clade B) and that in the second vector can be from another HIV clade (e.g., clade C).

Where the composition includes either a vector with an insert or an insert alone, and that insert encodes a single antigen, the antigen can be wild type or mutant Pol. The sequence can be mutated by deleting or replacing one or more nucleic acids, and those deletions or substitutions can result in a Pol gene product that has less enzymatic activity than its wild type counterpart (e.g., less integrase activity, less reverse transcriptase (RT) activity, or less protease activity). For example, one can inhibit RT by inactivating the polymerase's active site or by ablating strand transfer activity. Alternatively, or in addition, one can inhibit the polymerase's RNase H activity. Where the composition includes either a vector with an insert or an insert alone, and that insert encodes multiple protein antigens, one of the antigens can be a wild type or mutant pol sequence, including those described above (these multi-protein-encoding inserts can also encode the wild type or mutant gag sequences described above). Similarly, where a composition includes more than one type of vector or more than one type of insert, at least one of the vectors or inserts (whether encoding a single antigen or multiple antigens) can include a wild type or mutant pol sequence, including those described above (and, optionally, a wild type or mutant gag sequence, including those described above (i.e., the inserts can encode Gag-Pol). For example, where the composition includes first and second vectors, the vaccine insert in either or both vectors (whether the insert encodes single or multiple antigens) can encode Pol; where both vectors encode Pol, the Pol sequence in the first vector can be from one HIV clade (e.g., clade B) and that in the second vector can be from another HIV clade (e.g., clade A or G).

Where an insert includes some or all of the pol sequence, another portion of the pol sequence that can optionally be altered is the sequence encoding the protease activity (regardless of whether or not sequences affecting other enzymatic activities of Pol have been altered). Where the composition includes either a vector with an insert or an insert alone, and that insert encodes a single antigen, the antigen can be a wild type or mutant Env, Tat, Rev, Nef, Vif, Vpr, or Vpu. Where the composition includes either a vector with an insert or an insert alone, and that insert encodes multiple protein antigens, one of the antigens can be a wild type or mutant Env. For example, multi-protein expressing inserts can encode wild type or mutant Gag-Pol and Env; they can also encode wild type or mutant Gag-Pol and Env and one or more of Tat, Rev, Nef, Vif, Vpr, or Vpu (each of which can be wild type or mutant). As with other antigens, Env, Tat, Rev, Nef, Vif, Vpr, or Vpu can be mutant by virtue of a deletion, addition, or substitution of one or more amino acid residues (e.g., any of these antigens can include a point mutation). With respect to Env, one or more mutations can be in any of the domains shown in FIG. 19. For example, one or more amino acids can be deleted from the gp120 surface and/or gp41 transmembrane cleavage products of Env. With respect to Gag, one or more amino acids can be deleted from one or more of: the matrix protein (p17), the capsid protein (p24), the nucleocapsid protein (p7) and the C-terminal peptide (p6). For example, amino acids in one or more of these regions can be deleted (this may be especially desired where the vector is a viral vector, such as MVA). With respect to Pol, one or more amino acids can be deleted from the protease protein (p10), the reverse transcriptase protein (p66/p51), or the integrase protein (p32).

More specifically, the compositions of the disclosure can include a vector (e.g., a plasmid or viral vector) that encodes: (a) a Gag protein in which one or more of the zinc fingers has been inactivated to limit the packaging of viral RNA; (b) a Pol protein in which (i) the integrase activity has been inhibited by deletion of some or all of the pol sequence and (ii) the polymerase, strand transfer, and/or RNase H activity of reverse transcriptase has been inhibited by one or more point mutations within the pol sequence; and (c) Env, Tat, Rev, and Vpu, with or without mutations. In this embodiment, as in others, the encoded proteins can be obtained or derived from a subtype A, B or C HIV (e.g., HIV-1) or recombinant forms thereof. Where the compositions include non-identical vectors, the sequence in each type of vector can be from a different HIV clade (or subtype or recombinant form thereof). For example, the disclosure features compositions that include plasmid vectors encoding the antigens just described (Gag-Pol, Env etc.), where some of the plasmids include antigens that are obtained from, or derived from, one clade and other plasmids include antigens that are obtained (or derived) from another clade. Mixtures representing two, three, four, five, six, or more clades (including all clades) are within the scope of the disclosure.

Where first and second vectors are included in a composition, either vector can be pGA1/JS2, pGA1/JS7, pGA1/JS7.1, pGA2/JS2, pGA2/JS7, pGA2/JS7.1 (pGA1.1, pGA1.2 or the pGA vectors with other permutations in restrictions sites used for addition of vaccine inserts can be used in place of pGA1, and pGA2.1 or pGA2.2 can be used in place of pGA2). Similarly, either vector can be pGA1/IC25, pGA1/IC2, pGA1/IC48, pGA1/IC90, pGA2/IC25, pGA2/IC2, pGA2/IC48, or pGA2/IC90 (here again, pGA1.1 or pGA1.2 can be used in place of pGA1, and pGA2.1 or pGA2.2 can be used in place of pGA2). In alternative embodiments, the encoded proteins can be those of, or those derived from, a subtype C HIV (e.g., HIV1) or a recombinant form thereof. For example, the vector can be pGA1/IN2, pGA1.1/IN2, pGA1.2/IN2, pGA1/IN3, pGA1.1/IN3, pGA1.2/IN3, pGA2/IN2, pGA2.1/IN2, pGA2.2/IN2, pGA2/IN3, pGA2.1/IN3, or pGA2.2/IN3.

The encoded proteins can also be those of, or those derived from, any of HIV clades (or subtypes) E, F, G, H, I, J, K or L or recombinant forms thereof. An HIV-1 classification system has been published by Los Alamos National Laboratory (HIV Sequence Compendium-2001, Kuiken et al, published by Theoretical Biology and Biophysics Group T-10, Los Alamos, NM, (2001)), more recent HIV sequences are available on the Los Alamos HIV sequence database website.

The compositions of the disclosure can also include a vector (e.g., a plasmid vector) encoding: (a) a Gag protein in which one or both zinc fingers have been inactivated; (b) a Pol protein in which (i) the integrase activity has been inhibited by deletion of some or all of the pol sequence, (ii) the polymerase, strand transfer, and/or RNase H activity of reverse transcriptase has been inhibited by one or more point mutations within the pol sequence and (iii) the proteolytic activity of the protease has been inhibited by one or more point mutations; and (c) Env, Tat, Rev, and Vpu, with or without mutations. As noted above, proteolytic activity can be inhibited by introducing a mutation at positions 1641-1643 of SEQ ID NO:8 or at an analogous position in the sequence of another HIV clade. For example, the plasmids can contain the inserts described herein as JS7, IC25, and IN3. As is true for plasmids encoding other antigens, plasmids encoding the antigens just described can be combined with (e.g., mixed with) other plasmids that encode antigens obtained from, or derived from, a different HIV clade (or subtype or recombinant form thereof). The inserts per se (sans vector) are also within the scope of the disclosure. As described herein, the inserts may contain sequences that encode one or more conserved protein sequences and/or may contain one or more designer sequences (e.g., mosaic sequences that contain a sequence from one or more HIV clades).

Other vectors of the disclosure include plasmids encoding a Gag protein (e.g., a Gag protein in which one or both of the zinc fingers have been inactivated); a Pol protein (e.g., a Pol protein in which integrase, RT, and/or protease activities have been inhibited); a Vpu protein (which may be encoded by a sequence having a mutant start codon); and Env, Tat, and/or Rev proteins (in a wild type or mutant form). As is true for plasmids encoding other antigens, plasmids encoding the antigens just described can be combined with (e.g., mixed with) other plasmids that encode antigens obtained from, or derived from, a different HIV clade (or subtype or recombinant form thereof). The inserts per se (sans vector) are also within the scope of the disclosure.

The plasmids described above, including those that express the JS2 or JS7 series of clade B HIV-1 sequences, can be administered to any subject, but may be most beneficially administered to subjects who have been, or who are likely to be, exposed to an HIV of clade B (the same is true for vectors other than plasmid vectors). Similarly, plasmids or other vectors that express an IN series of clade C HIV-1 sequences can be administered to a subject who has been, or who may be, exposed to an HIV of clade C. As vectors expressing antigens of various clades can be combined to elicit an immune response against more than one clade (this can be achieved whether one vector expresses multiple antigens, or mosaic or conserved element antigens from different clades or multiple vectors express single antigens from different clades), one can tailor the vaccine formulation to best protect a given subject. For example, if a subject is likely to be exposed to regions of the world where clades other than clade B predominate, one can formulate and administer a vector or vectors that express an antigen (or antigens) that will optimize the elicitation of an immune response to the predominant clade or clades.

The antigens they express are not the only parts of the plasmid vectors that can vary. Useful plasmids may or may not contain a terminator sequence that substantially inhibits transcription (the process by which RNA molecules are formed upon DNA templates by complementary base pairing). Useful terminator sequences include the lambda T0 terminator and functional fragments or variants thereof. The terminator sequence is positioned within the vector in the same orientation and at the C terminus of any open reading frame that is expressed in prokaryotes (i.e., the terminator sequence and the open reading frame are operably linked). By preventing read through from the selectable marker into the vaccine insert as the plasmid replicates in prokaryotic cells, the terminator stabilizes the insert as the bacteria grow and the plasmid replicates.

Selectable marker genes are known in the art and include, for example, genes encoding proteins that confer antibiotic resistance on a cell in which the marker is expressed (e.g., resistance to kanamycin, ampicillin, or penicillin). The selectable marker is so-named because it allows one to select cells by virtue of their survival under conditions that, absent the marker, would destroy them. The selectable marker, the terminator sequence, or both (or parts of each or both) can be, but need not be, excised from the plasmid before it is administered to a patient. Similarly, plasmid vectors can be administered in a circular form, after being linearized by digestion with a restriction endonuclease, or after some of the vector “backbone” has been altered or deleted.

The nucleic acid vectors can also include an origin of replication (e.g., a prokaryotic origin of replication) and a transcription cassette that, in addition to containing one or more restriction endonuclease sites, into which an antigen-encoding insert can be cloned, optionally includes a promoter sequence and a polyadenylation signal. Promoters known as strong promoters can be used and may be preferred. One such promoter is the cytomegalovirus (CMV) intermediate early promoter, although other (including weaker) promoters may be used without departing from the scope of the present disclosure. Similarly, strong polyadenylation signals may be selected (e.g., the signal derived from a bovine growth hormone (BGH) encoding gene, or a rabbit β globin polyadenylation signal (Bohm et al., J. Immunol. Methods 193:29-40, 1996; Chapman et al., Nucl. Acids Res. 19:3979-3986, 1991; Hartikka et al., Hum. Gene Therapy 7:1205-1217, 1996; Manthorpe et al., Hum. Gene Therapy 4:419-431, 1993; Montgomery et al., DNA Cell Biol. 12:777-783, 1993)).

The vectors can further include a leader sequence (a leader sequence that is a synthetic homolog of the tissue plasminogen activator gene leader sequence (tPA) is optional in the transcription cassette) and/or an intron sequence, such as a cytomegalovirus (CMV) intron A or an SV40 intron. The presence of intron A increases the expression of many antigens from RNA viruses, bacteria, and parasites, presumably by providing the expressed RNA with sequences that support processing and function as a eukaryotic mRNA. Expression can also be enhanced by other methods known in the art including, but not limited to, optimizing the codon usage of prokaryotic mRNAs for eukaryotic cells (Andre et al., J. Virol. 72:1497-1503, 1998; Uchijima et al., J. Immunol. 161:5594-5599, 1998). Multi-cistronic vectors may be used to express more than one immunogen or an immunogen and an immunostimulatory protein (Iwasaki et al., J. Immunol. 158:4591-4601, 1997a; Wild et al., Vaccine 16:353-360, 1998). Thus (and as is true with other optional components of the vector constructs), vectors encoding one or more antigens from one or more HIV clades or isolates may, but do not necessarily, include a leader sequence and an intron (e.g., the CMV intron A).

The vectors of the present disclosure differ in the sites that can be used for accepting antigen-encoding sequences and in whether the transcription cassette includes intron A sequences in the CMVIE promoter. Accordingly, one of ordinary skill in the art may modify the insertion site(s) or cloning site(s) within the plasmid without departing from the scope of the disclosure. Both intron A and the tPA leader sequence have been shown in certain instances to enhance antigen expression (Chapman et al., Nucleic Acids Research 19:3979-3986, 1991).

As described further below, the vectors of the present disclosure can be administered with an adjuvant, including a genetic adjuvant. Accordingly, the nucleic acid vectors, regardless of the antigen they express, can optionally include such genetic adjuvants as GM-CSF, IL-15, IL-2, interferon response factors, secreted forms of flt-3, CD40 ligand and mutated caspase genes. Genetic adjuvants can also be supplied in the form of fusion proteins, for example by fusing one or more C3d gene sequences (e.g., 1-3 (or more) C3d gene sequences) to an expressed antigen.

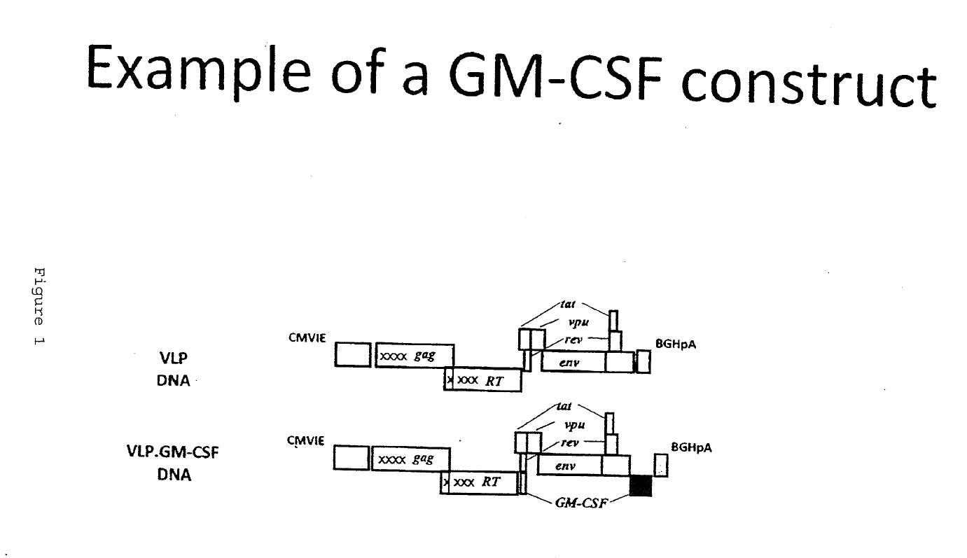

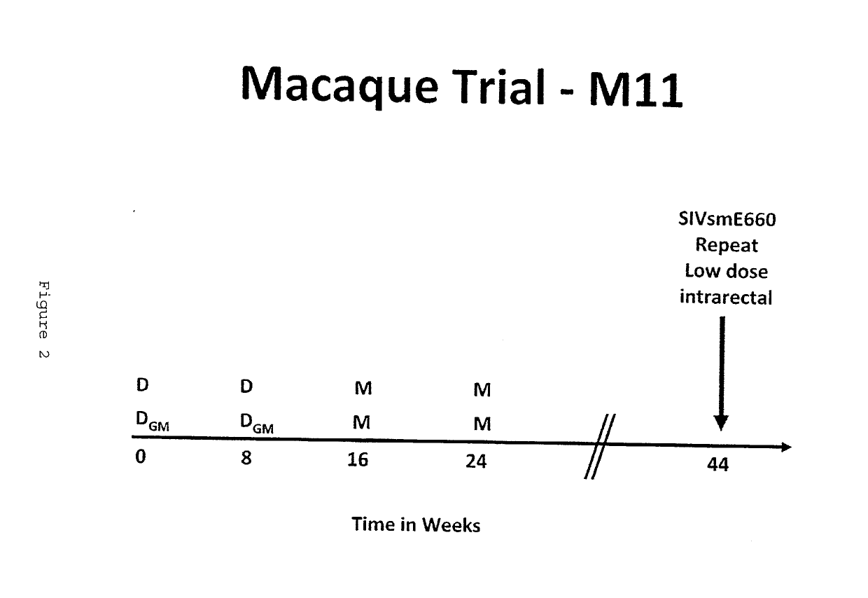

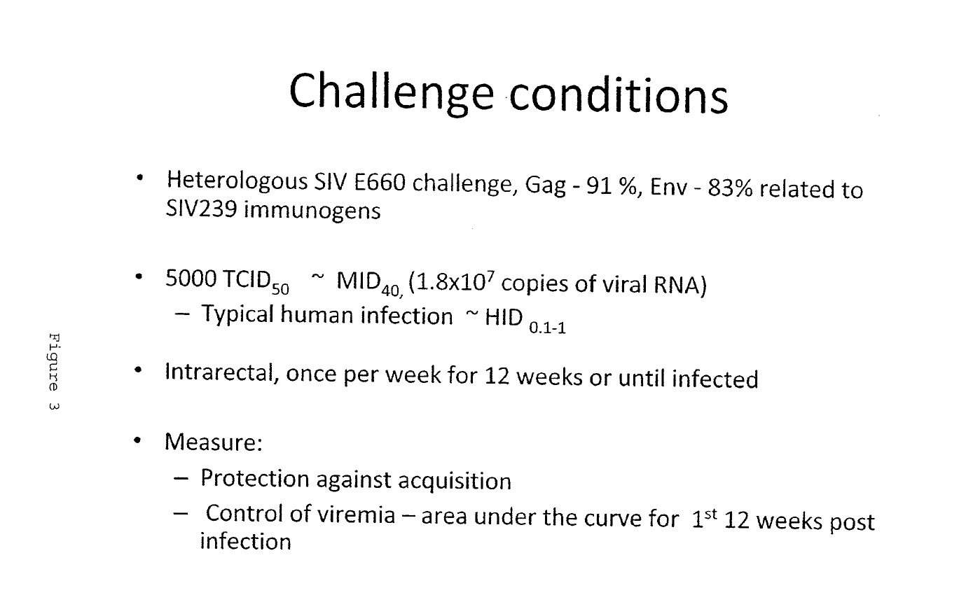

In the event the vector administered is a pGA vector, it can comprise the sequence of, for example, pGA1 (SEQ ID NO:1) or derivatives thereof (e.g., SEQ ID NOs:2 and 3), or pGA2 (SEQ ID NO:4) or derivatives thereof (e.g., SEQ ID NOs:5 and 6). The pGA vectors are described in more detail here (see also Examples 1-8). pGA1 is a 3897 bp plasmid that includes a promoter (bp 1-690), the CMV-intron A (bp 691-1638), a synthetic mimic of the tPA leader sequence (bp 1659-1721), the bovine growth hormone polyadenylation sequence (bp 1761-1983), the lambda T0 terminator (bp 1984-2018), the kanamycin resistance gene (bp 2037-2830) and the ColEI replicator (bp 2831-3890). The DNA sequence of the pGA1 construct (SEQ ID NO:1) is shown in FIG. 2. In FIG. 1, the indicated restriction sites are useful for cloning antigen-encoding sequences. The Cla I or BspD I sites are used when the 5′ end of a vaccine insert is cloned upstream of the tPA leader. The Nhe I site is used for cloning a sequence in frame with the tPA leader sequence. The sites listed between Sma I and Bln I are used for cloning the 3′ terminus of an antigen-encoding sequence.

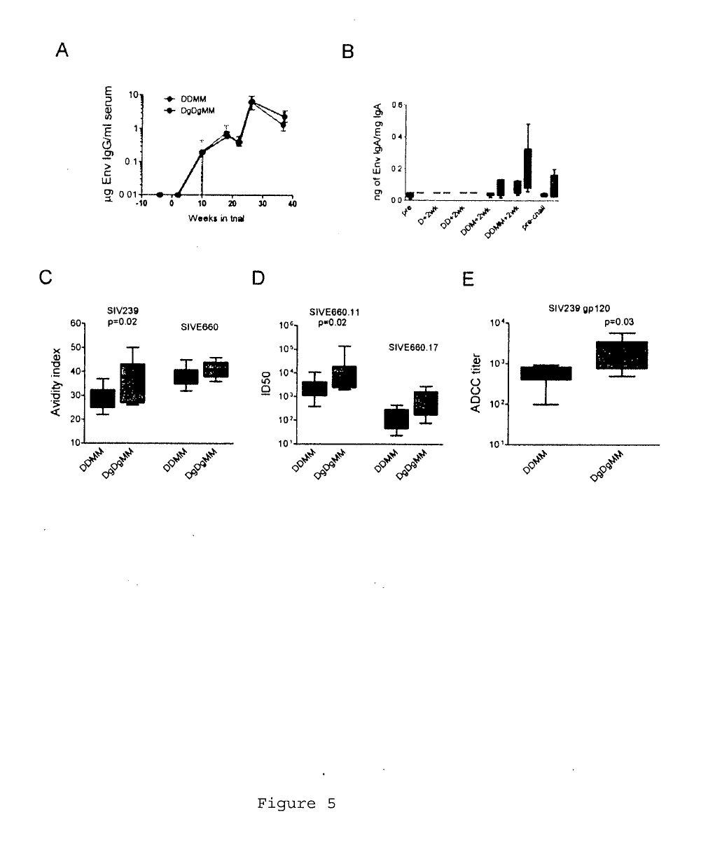

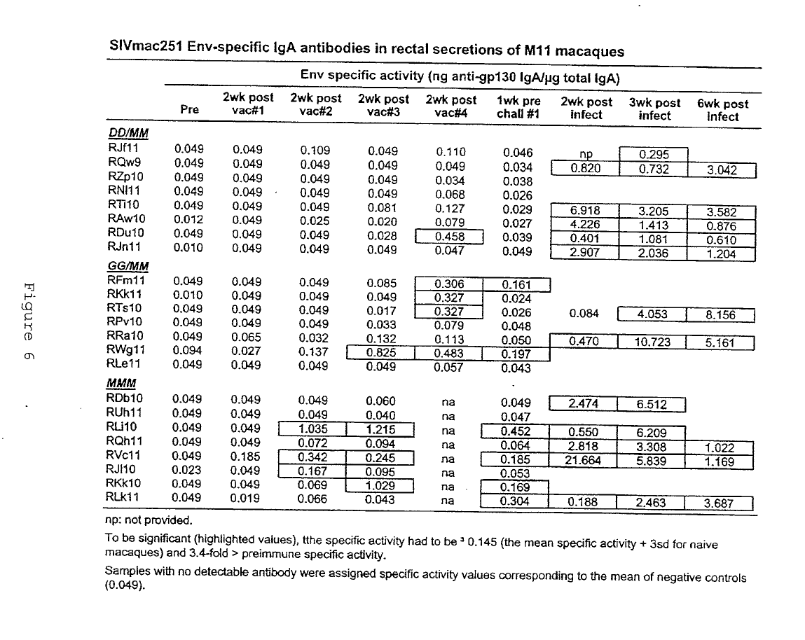

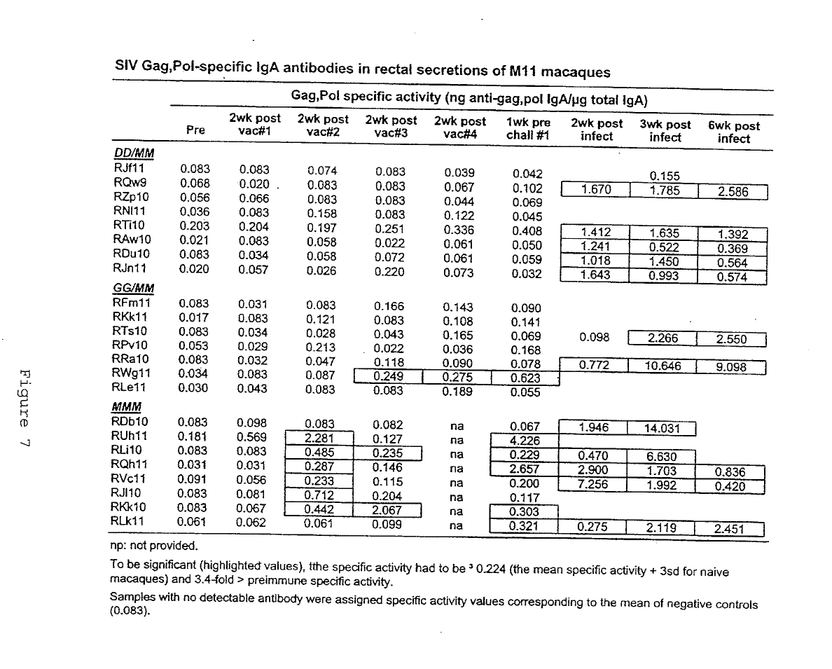

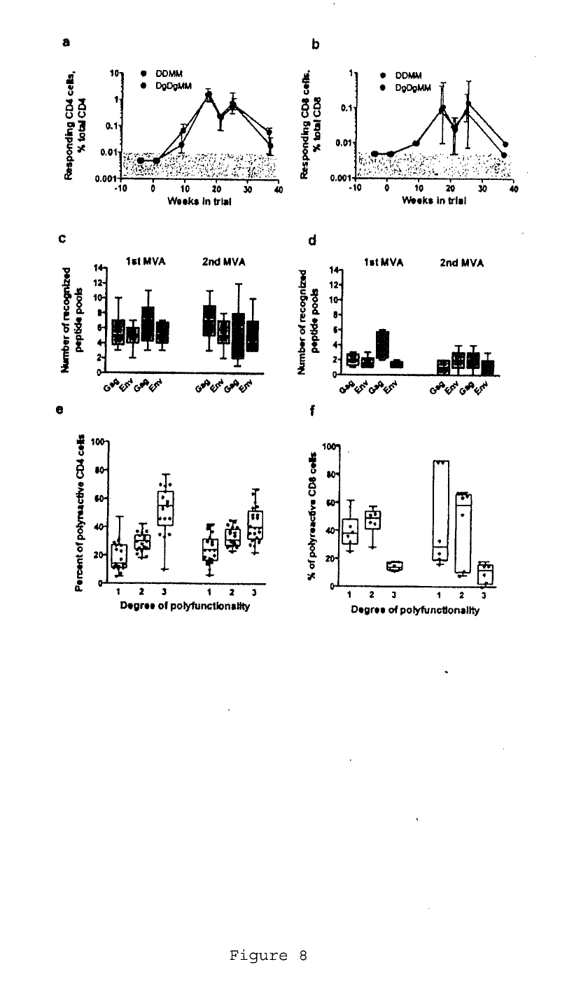

pGA2 is a 2947 bp plasmid lacking the 947 bp of intron A sequences found in pGA1. pGA2 is the same as pGA1, except for the deletion of intron A sequences. pGA2 is valuable for cloning sequences which do not require an upstream intron for efficient expression, or for cloning sequences in which an upstream intron might interfere with the pattern of splicing needed for good expression. FIG. 5 presents a schematic map of pGA2 with useful restriction sites for cloning vaccine inserts. FIG. 6a shows the DNA sequence of pGA2 (SEQ ID NO:2). The use of restriction sites for cloning vaccine inserts into pGA2 is the same as that used for cloning fragments into pGA1. pGA2.1 and pGA2.2 are multiple cloning site derivatives of pGA2. FIGS. 7a and 8a show the DNA sequence of pGA2.1 (SEQ ID NO:5) and pGA2.2 (SEQ ID NO:6) respectively.

pGA plasmids having “backbone” sequences that differ from those disclosed herein are also within the scope of the disclosure so long as the plasmids retain substantially all of the characteristics necessary to be therapeutically effective (e.g., one can substitute nucleotides, add nucleotides, or delete nucleotides so long as the plasmid, when administered to a patient, induces or enhances an immune response against a given or desired pathogen). For example, 1-10, 11-20, 21-30, 31-40, 41-50, 51-60, 61-70, 71-80, 81-90, 91-100, or more than 100 nucleotides can be deleted or replaced.

In one embodiment, the methods of the disclosure (e.g., methods of eliciting an immune response in a patient) can be carried out by administering to the patient a therapeutically effective amount of a physiologically acceptable composition that includes a vector, which can contain a vaccine insert that encodes one or more antigens that elicit an immune response against an HIV. The vector can be a plasmid vector having one or more of the characteristics of the pGA constructs described above (e.g., a selectable marker gene, a prokaryotic origin of replication, a termination sequence (e.g., the lambda T0 terminator) and operably linked to the selectable gene marker, and a eukaryotic transcription cassette comprising a promoter sequence, a nucleic acid insert encoding at least one antigen derived from an immunodeficiency virus, and a polyadenylation signal sequence). Of course, the vaccine inserts of the disclosure may be delivered by plasmid vectors that do not have the characteristics of the pGA constructs (e.g., vectors other than pGA1 or pGA2). Alternatively, the composition can include any viral or bacterial vector that includes an insert described herein. The disclosure, therefore, also encompasses administration of at least two (e.g., three, four, five, or six) vectors (e.g., plasmid or viral vectors that contain the same vaccine insert (i.e., an insert encoding the same antigens). As is made clear elsewhere, the patient may receive two types of vectors, and each of those vectors can elicit an immune response against an HIV of a different clade. For example, the disclosure features methods in which a patient receives a composition that includes (a) a first vector comprising a vaccine insert encoding one or more antigens that elicit an immune response against a human immunodeficiency virus (HIV) of a first subtype or recombinant form and (b) a second vector comprising a vaccine insert encoding one or more antigens that elicit an immune response against an HIV of a second subtype or recombinant form. The first and second vectors can be any of those described herein. Similarly, the inserts in the first and second vectors can be any of those described herein.

A therapeutically effective amount of a vector (whether considered the first, second, third, etc. vector) can be administered by an intramuscular, a mucosal, or an intradermal route, together with a physiologically acceptable carrier, diluent, or excipient, and, optionally, an adjuvant. A therapeutically effective amount of the same or a different vector can subsequently be administered by an intramuscular or an intradermal route, together with a physiologically acceptable carrier, diluent, or excipient, and, optionally, an adjuvant to boost an immune response. Such components can be readily selected by one of ordinary skill in the art, regardless of the precise nature of the antigens incorporated in the vaccine or the vector by which they are delivered.

The methods of eliciting an immune response can be carried out by administering only the plasmid vectors of the disclosure, by administering only the viral vectors of the disclosure, or by administering both (e.g., one can administer a plasmid vector (or a mixture or combination of plasmid vectors)) to “prime” the immune response and a viral vector (or a mixture or combination of viral vectors)) to “boost” the immune response. Where plasmid and viral vectors are administered, their inserts may be “matched.” To be “matched,” one or more of the sequences of the inserts (e.g., the sequences encoding Gag, or the sequences encoding Env, etc.) within the plasmid and viral vectors may be identical, but the term is not so limited. “Matched” sequences can also differ from one another. For example, inserts expressed by viral vectors are “matched” to those expressed by DNA vectors when the sequences used in the DNA vector are mutated or further mutated to allow (or optimize) replication of a viral vector that encodes those sequences and expression of the encoded antigens (e.g., Gag, Gag-Pol, or Env) in cells infected with the viral vector.

In certain embodiments of the methods, a subject is administered one or more (e.g., two, three, four, five, or six) doses of a vector containing a prokaryotic origin of replication, a promoter sequence, a eurkaryotic expression cassette containing a vaccine insert encoding one or more immunogens and GM-CSF, a polyadenylation sequence, and a transcription termination sequence. If two or more doses of the vectors described herein are administered to a subject, two of such doses may be administered at least 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 10 weeks, 12 weeks, 16 weeks, 20 weeks, or 24 weeks apart. The one or more (e.g., two, three, four, five, or six) doses of a vector (as described herein) may further be administered with one or more (e.g., two, three, four, five, or six) doses of a MVA vaccine encoding one or more (e.g., two, three, four, five, six, seven, eight, nine, or ten) HIV immunogens. In such embodiments, the MVA vaccine may be administered to the subject after (e.g., 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 10 weeks, 12 weeks, 16 weeks, 20 weeks, or 24 weeks apart. The one or more (e.g., two, three, four, five, or six) administration of at least one (e.g., two, three, four, five, or six) doses of one of the vectors described herein. In additional embodiments, at least one dose of a MVA vaccine is administered to the subject at the same time at least one vector described herein in administered to the subject. Additional doses of one or more of the vectors described herein and/or the MVA vaccines described herein may be administered to a subject following an assessment of the immune response in a subject (e.g., medical assessment by a physician).

At least some of the immunodeficiency virus vaccine inserts of the present disclosure were designed to generate non-infectious VLPs (a term that can encompass true VLPs as well as aggregates of viral proteins) from a single DNA. This was achieved using the subgenomic splicing elements normally used by immunodeficiency viruses to express multiple gene products from a single viral RNA. The subgenomic splicing patterns are influenced by (i) splice sites and acceptors present in full length viral RNA, (ii) the Rev responsive element (RRE) and (iii) the Rev protein. The splice sites in retroviral RNAs use the canonical sequences for splice sites in eukaryotic mRNAs. The RRE is an approximately 200 bp RNA structure that interacts with the Rev protein to allow transport of viral RNAs from the nucleus to the cytoplasm. In the absence of Rev, the approximately 10 kb RNA of immunodeficiency virus mostly undergoes splicing to the mRNAs for the regulatory genes Tat, Rev, and Nef. These genes are encoded by exons present between RT and Env and at the 3′ end of the genome. In the presence of Rev, the singly spliced mRNA for Env and the unspliced mRNA for Gag and Pol are expressed in addition to the multiply spliced mRNAs for Tat, Rev, and Nef.

The expression of non-infectious VLPs from a single DNA affords a number of advantages to an immunodeficiency virus vaccine. The expression of a number of proteins from a single DNA affords the vaccinated host the opportunity to respond to the breadth of T- and B-cell epitopes encompassed in these proteins. The expression of proteins containing multiple epitopes allows epitope presentation by diverse histocompatibility types. By using whole proteins, one offers hosts of different histocompatibility types the opportunity to raise broad-based T cell responses. This may be essential for the effective containment of immunodeficiency virus infections, whose high mutation rate supports ready escape from immune responses (Evans et al., Nat. Med. 5:1270-1276, 1999; Poignard et al., Immunity 10:431-438, 1999, Evans et al., 1995). In the context of the present vaccination scheme, just as in drug therapy, multi-epitope T cell responses that require multiple mutations for escape will provide better protection than single epitope T cell responses (which require only a single mutation for escape).

Immunogens can also be engineered to be more or less effective for raising antibody or Tc by targeting the expressed antigen to specific cellular compartments. For example, antibody responses are raised more effectively by antigens that are displayed on the plasma membrane of cells, or secreted therefrom, than by antigens that are localized to the interior of cells (Boyle et al., Int. Immunol. 9:1897-1906, 1997; Inchauspe et al., DNA Cell. Biol. 16:185-195, 1997). Tc responses may be enhanced by using N-terminal ubiquitination signals which target the DNAencoded protein to the proteosome causing rapid cytoplasmic degradation and more efficient peptide loading into the MHC I pathway (Rodriguez et al., J. Virol. 71:8497-8503, 1997; Tobery et al., J. Exp. Med. 185:909-920, 1997; Wu et al., J. Immunol. 159:6037-6043, 1997). For a review on the mechanistic basis for DNA-raised immune responses, refer to Robinson and Pertmer, Advances in Virus Research, vol. 53, Academic Press (2000).

Another approach to manipulating immune responses is to fuse immunogens to immunotargeting or immunostimulatory molecules. To date, the most successful of these fusions have targeted secreted immunogens to antigen presenting cells (APCs) or lymph nodes (Boyle et al., Nature 392:408-411, 1998). Accordingly, the disclosure features the HIV antigens described herein fused to immunotargeting or immunostimulatory molecules such as CTLA-4, L-selectin, or a cytokine (e.g., an interleukin such as IL-1, IL-2, IL-4, IL-7, IL10, IL-15, or IL-21). Nucleic acids encoding such fusions and compositions containing them (e.g., vectors and physiologically acceptable preparations) are also within the scope of the present disclosure.

DNA can be delivered in a variety of ways, any of which can be used to deliver the plasmids of the present disclosure to a subject. For example, DNA can be injected in, for example, saline (e.g., using a hypodermic needle) delivered as a ballistic (by, for example, a gene gun that accelerates DNA-coated beads) or delivered by electroporation. Saline injections deliver DNA into extracellular spaces, whereas gene gun deliveries bombard DNA directly into cells. Electroporations transiently disrupt the integrity of cellular membranes, thereby allowing entry of the DNA. The saline injections require much larger amounts of DNA (typically 100-1000 times more) than the gene gun (Fynan et al., Proc. Natl. Acad. Sci. U.S.A. 90:11478-11482, 1993). These types of delivery also differ in that saline injections and electroporations bias responses towards type 1 T-cell help, whereas gene gun deliveries bias responses towards type 2 T-cell help (Feltquate et al., J. Immunol. 158:2278-2284, 1997; Pertmer et al., J. Virol. 70:6119-6125, 1996). DNAs injected in saline rapidly spread throughout the body. DNAs delivered by the gun are more localized at the target site. Following either method of inoculation, extracellular plasmid DNA has a short half life of about 10 minutes (Kawabata et al., Pharm. Res. 12:825-830, 1995; Lew et al., Hum. Gene Ther. 6:553, 1995). Vaccination by saline injections can be intramuscular (i.m.), intradermal (i.d.), or mucosal (as described below in more detail); gene gun deliveries can be administered to the skin or to surgically exposed tissue such as muscle.

While other routes of delivery are generally less favored, they can nevertheless be used to administer the compositions of the disclosure. For example, the DNA can be applied to the mucosa or by a parenteral route of inoculation. Intranasal administration of DNA in saline has met with both good (Asakura et al., Scand. J. Immunol. 46:326-330, 1997; Sasaki et al., Infect. Immun. 66:823-826, 1998b) and limited (Fynan et al., Proc. Natl. Acad. Sci. U.S.A. 90:11478-82, 1993) success. The gene gun has successfully raised IgG following the delivery of DNA to the vaginal mucosa (Livingston et al., Ann. New York Acad. Sci. 772:265-267, 1995). Some success at delivering DNA to mucosal surfaces has also been achieved using liposomes (McCluskie et al., Antisense Nucleic Acid Drug Dev. 8:401-414, 1998), microspheres (Chen et al., J. Virol. 72:5757-5761, 1998a; Jones et al., Vaccine 15:814-817, 1997), and recombinant Shigella vectors (Sizemore et al., Science 270:299-302, 1995; Sizemore et al., Vaccine 15:804-807, 1997). Agents such as these (liposomes, microspheres, and recombinant Shigella vectors) can be used to deliver the nucleic acids of the present disclosure.

The dose of DNA needed to raise a response depends upon the method of delivery, the host, the vector, and the encoded antigen. The method of delivery may be the most influential parameter. From 10 μg to 5 mg of DNA is generally used for saline injections of DNA, whereas from 0.2 μg to 20 μg of DNA is used more typically for gene gun deliveries of DNA. In general, lower doses of DNA are used in mice (10-100 μg for saline injections and 0.2 μg to 2 μg for gene gun deliveries), and higher doses in primates (100 μg to 1 mg for saline injections and 2 μg to 20 μg for gene gun deliveries). The much lower amount of DNA required for gene gun deliveries reflect the gold beads directly delivering DNA into cells.

In addition to the DNA vectors described above, a number of different poxviruses can be used either alone (i.e., without a nucleic acid or DNA prime) or as the boost component of a vaccine regimen. MVA has been particularly effective in mouse models (Schneider et al., Nat. Med. 4:397-402, 1998). MVA is a highly attenuated strain of vaccinia virus that was developed toward the end of the campaign for the eradication of smallpox, and it has been safety tested in more than 100,000 people (Mahnel et al., Berl. Munch Tierarztl Wochenschr 107:253-256, 1994; Mayr et al., Zentralbl. Bakteriol. 167:375-390, 1978). During over 500 passages in chicken cells, MVA lost about 10% of its genome and the ability to replicate efficiently in primate cells. Despite its limited replication, MVA has proved to be a highly effective expression vector (Sutter et al., Proc. Natl. Acad. Sci. U.S.A. 89:10847-10851, 1992), raising protective immune responses in primates for parainfluenza virus (Durbin et al. J. Infect. Dis. 179:1345-1351, 1999), measles (Stittelaar et al. J. Virol. 74:4236-4243, 2000), and immunodeficiency viruses (Barouch et al., J. Virol. 75:5151-5158, 2001; Ourmanov et al., J. Virol. 74:2740-2751, 2000; Amara et al., J. Virol. 76:7625-7631, 2002). The relatively high immunogenicity of MVA has been attributed in part to the loss of several viral anti-immune defense genes (Blanchard et al., J. Gen. Virol. 79:1159-1167, 1998).

Vaccinia viruses have been used to engineer viral vectors for recombinant gene expression and as recombinant live vaccines (Mackett et al., Proc. Natl. Acad. Sci. U.S.A. 79:7415-7419; Smith et al., Biotech. Genet. Engin. Rev. 2:383-407, 1984). DNA sequences, which may encode any of the HIV antigens described herein, can be introduced into the genomes of vaccinia viruses. If the gene is integrated at a site in the viral DNA that is non-essential for the life cycle of the virus, it is possible for the newly produced recombinant vaccinia virus to be infectious (i.e., able to infect foreign cells) and to express the integrated DNA sequences. Preferably, the viral vectors featured in the compositions and methods of the present disclosure are highly attenuated. Several attenuated strains of vaccinia virus were developed to avoid undesired side effects of smallpox vaccination. The modified vaccinia Ankara (MVA) virus was generated by long-term serial passages of the Ankara strain of vaccinia virus on chicken embryo fibroblasts (CVA; see Mayr et al., Infection 3:6-14, 1975). The MVA virus is publicly available from the American Type Culture Collection (ATCC; No. VR-1508; Manassas, Va.). The desirable properties of the MVA strain have been demonstrated in clinical trials (Mayr et al., Zentralbl. Bakteriol. 167:375-390, 1978; Stickl et al., Dtsch. Med. Wschr. 99:2386-2392, 1974; see also, Sutter and Moss, Proc. Natl. Acad. Sci. U.S.A. 89:10847-10851, 1992). During these studies in over 120,000 humans, including high-risk patients, no side effects were associated with the use of MVA vaccine.

The MVA vectors can be prepared as follows. A DNA construct that contains a DNA sequence that encodes a foreign polypeptide (e.g., any of the HIV antigens described herein) and that is flanked by MVA DNA sequences adjacent to a naturally occurring deletion within the MVA genome (e.g., deletion III or other non-essential site(s); six major deletions of genomic DNA (designated deletions I, II, III, IV, V, and VI) totaling 31,000 base pairs have been identified (Meyer et al., J. Gen. Virol. 72:1031-1038, 1991)) is introduced into cells infected with MVA under conditions that permit homologous recombination to occur. Insertions may also be introduced into naturally-occurred deletions with modified deletion sites to enhance stability of the insertion or introduced between essential genes using sequences flanking the insertion site. One site between essential genes that has proven useful is 18G1 (see, for e.g., Wyatt et al., Retrovirology 6:416, 2009). Once the DNA construct has been introduced into the eukaryotic cell and the foreign DNA has recombined with the viral DNA, the recombinant vaccinia virus can be isolated by methods known in the art (isolation can be facilitated by use of a detectable marker). The DNA constructed to be inserted can be linear or circular (e.g., a plasmid, linearized plasmid, gene, gene fragment, or modified HIV genome). The foreign DNA sequence is inserted between the sequences flanking the naturally-occurring deletion, between the sequences of a modified naturally occurring deletion, or between the sequences marking the boundaries of two essential genes. For better expression of a DNA sequence, the sequence can include regulatory sequences (e.g., a promoter, such as the promoter of the vaccinia 11 kDa gene or the 7.5 kDa gene). The DNA construct can be introduced into MVA-infected cells by a variety of methods, including calcium phosphate-assisted transfection (Graham et al., Virol. 52:456-467, 1973 and Wigler et al., Cell 16:777-785, 1979), electroporation (Neumann et al., EMBO J. 1:841-845, 1982), microinjection (Graessmann et al., Meth. Enzymol. 101:482-492, 1983), by means of liposomes (Straubinger et al., Meth. Enzymol. 101:512-527, 1983), by means of spheroplasts (Schaffner, Proc. Natl. Acad. Sci. U.S.A. 77:2163-2167, 1980), or by other methods known in the art.

One can arrive at an appropriate dosage when delivering DNA by way of a viral vector, just as one can when a plasmid vector is used. For example, one can deliver 1×10⁸ pfu of an MVA-based vaccine, and administration can be carried out intramuscularly, intradermally, intravenously, or mucosally.

Accordingly, the disclosure features a composition comprising: (a) a first viral vector comprising a vaccine insert encoding one or more antigens that elicit an immune response against a human immunodeficiency virus (HIV) of a first subtype or recombinant form and (b) a second viral vector comprising a vaccine insert encoding one or more antigens that elicit an immune response against an HIV of a second subtype or recombinant form. The viral vector can be a recombinant poxvirus or a modified vaccinia Ankara (MVA) virus, and the insert can be any of the HIV antigens described herein from any clade (e.g., one can administer a prophylactically or therapeutically effective amount of an MVA that encodes a clade A, B, or C HIV (e.g., HIV-1 antigen). Moreover, when administered in conjunction with a plasmid vector (e.g., when administered subsequent to a “DNA prime”), the MVA-borne sequence can be “matched” to the plasmid-borne sequence. For example, a vaccinia virus (e.g., MVA) that expresses a recombinant clade B sequence can be matched to the JS series of plasmid inserts. Similarly, a vaccinia virus (e.g., MVA) that expresses a recombinant clade A sequence can be matched to the IC series of plasmid inserts; a vaccinia virus (e.g., MVA) that expresses a recombinant clade C sequence can be matched to the IN series of plasmid inserts. While particular clades are exemplified below, the disclosure is not so limited. The compositions that contain a viral vector, can include viral vectors that express an HIV antigen from any known clade (including clades A, B, C, D, E, F, G, H, I, J, K, or L). Methods of eliciting an immune response can, of course, be carried out with compositions expressing antigens from any of these clades as well, or with designer HIV genes, such as mosaic genes (e.g., containing sequences from one or more (e.g., two, three, four, five, or six) HIV clades), or conserved epitope genes (e.g., nucleic acid sequences that encode one or more (e.g., two, three, four, five, or six) conserved protein epitope sequences).

Either the plasmid, or viral vectors, described here can be administered with an adjuvant (i.e., any substance that is added to a vaccine to increase the vaccine's immunogenicity) and they can be administered by any conventional route of administration (e.g., intramuscular, intradermal, intravenous or mucosally; see below). The adjuvant used in connection with the vectors described here (whether DNA or viral-based) can be one that slowly releases antigen (e.g., the adjuvant can be a liposome), or it can be an adjuvant that is strongly immunogenic in its own right (these adjuvants are believed to function synergistically). Accordingly, the vaccine compositions described here can include known adjuvants or other substances that promote DNA uptake, recruit immune system cells to the site of the inoculation, or facilitate the immune activation of responding lymphoid cells. These adjuvants or substances include oil and water emulsions, Corynebacterium parvum, Bacillus Calmette Guerin, aluminum hydroxide, glucan, dextran sulfate, iron oxide, sodium alginate, Bacto-Adjuvant, certain synthetic polymers such as poly amino acids and co-polymers of amino acids, saponin, REGRESSIN (Vetrepharm, Athens, Ga.), AVRIDINE (N,N-dioctadecyl-N′,N′-bis(2-hydroxyethyl)-propanediamine), paraffin oil, and muramyl dipeptide. Adjuvants being developed by Smith Kline designated AS01, AS02, AS03, AS04 and by Novartis, designated MF59, that combine agents such as MPL and QS21 are also valuable. AS02 contains MPLTM and QS-21 in an oil-in-water emulsion. AS04 also is composed of MPL, but in combination with alum. MPL is composed of a series of 4′-monophosphoryl lipid A species that vary in the extent and position of fatty acid substitution. It is prepared from lipopolysaccharide (LPS) of Salmonella Minnesota R595 by treating LPS with mild acid and base hydrolysis followed by purification of the modified LPS. Genetic adjuvants, which encode immunomodulatory molecules on the same or a co-inoculated vector, can also be used. For example, GM-CSF, IL-15, IL-2, interferon response factors, and mutated caspase genes can be included on a vector that encodes a pathogenic immunogen (such as an HIV antigen) or on a separate vector that is administered at or around the same time as the immunogen is administered. Expressed antigens can also be fused to an adjuvant sequence such as one, two, three or more copies of C3d.

The compositions described herein can be administered in a variety of ways including through any parenteral or topical route. For example, an individual can be inoculated by intravenous, intraperitoneal, intradermal, subcutaneous or intramuscular methods. Inoculation can be, for example, with a hypodermic needle, needleless delivery devices such as those that propel a stream of liquid into the target site, or with the use of a gene gun that bombards DNA on gold beads into the target site. The vector comprising the pathogen vaccine insert can be administered to a mucosal surface by a variety of methods including, but not limited to, electroporation, intranasal administration (e.g., nose drops or inhalants), or intrarectal or intravaginal administration by solutions, gels, foams, or suppositories. Alternatively, the vector comprising the vaccine insert can be orally administered in the form of a tablet, capsule, chewable tablet, syrup, emulsion, or the like. In an alternate embodiment, vectors can be administered transdermally, by passive skin patches, iontophoretic means, and the like.

Any physiologically acceptable medium can be used to introduce a vector (whether nucleic acid-based or live-vectored) comprising a vaccine insert into a patient. For example, suitable pharmaceutically acceptable carriers known in the art include, but are not limited to, sterile water, saline, glucose, dextrose, or buffered solutions. The media may include auxiliary agents such as diluents, stabilizers (i.e., sugars (glucose and dextrose were noted previously) and amino acids), preservatives, wetting agents, emulsifying agents, pH buffering agents, additives that enhance viscosity or syringability, colors, and the like. Preferably, the medium or carrier will not produce adverse effects, or will only produce adverse effects that are far outweighed by the benefit conveyed.

The present disclosure is further illustrated by the following examples, which are provided by way of illustration and should not be construed as limiting. The contents of all references, published patent applications and patents cited throughout the present application are hereby incorporated by reference in their entirety. A number of embodiments of the disclosure have been described. Nevertheless, it will be understood that various modifications may be made without departing from the spirit and scope of the disclosure.

### EXAMPLES

### Example 1

### DNA Vectors Expressing GM-CSF Induce an Immune Response

Described below are studies showing the results of studies comparing immunization with two DNA vectors, one expressing GM-CSF and one not expressing GM-CSF. FIG. 1 shows suitable a DNA vector expressing HIV antigens and GM-CSF.

### Detailed Discussion of Challenge Experiments

We tested the ability of a SIVmac239 (SIV239)-based vaccine that induces both antibody and T cells to prevent infection by a heterologous SIVsmE660 (SIVE660) challenge. The vaccine consisted of a recombinant DNA used to prime immune responses and a recombinant MVA used to boost responses. Both the DNA and MVA components of the vaccine expressed the three major proteins of immunodeficiency viruses: Gag, Pol, and Env, and produced non-infectious virus like particles. The SIV vaccine was tested in the presence and absence of GM-CSF co-expressed with the SIV immunogens.

This study was designed using repeated moderate dose rectal challenges to better mimic human exposures (McDermott et al., J. Virol. 78:3140-3144, 2004; Keele et al., J. Exp. Med. 206:1117-1134, 2009). Also, to better represent human exposures, the study included the use of a challenge virus that was heterologous to the immunogen. Specifically, SIVmac239 sequences were used in the vaccine, and SIVsmE660, a virus 91% related in Gag and 83% related in Env, for the challenge (Yeh et al., J. Virol. 83:2686-2696, 2009; Reynolds et al., J. Exp. Med. 205:2537-2550, 2008). This level of variation is comparable to that observed between clade B isolates in the current pandemic (Yeh et al., 2009; Reynolds et al., 2008). Our primary objectives were to test the effect of the immunizations on the number of challenges to infection; and then, for animals that became infected, to test the effect of the immunizations on control of post-challenge virus replication. A secondary objective was to identify potential correlates for protection.

As shown in FIG. 2, macaques were immunized at time 0 and at 8 and 16 weeks with either the DNA HIV antigen vector (D) or the DNA HIV antigen/GM-CSF vector (D_{GM}). They were then immunized with a MVA vector at 16 and 24 weeks. The macaques were subjected to an intrarectal (see, FIG. 3) challenge once per week for 12 weeks or until infection was observed with heterologous SIV E660 challenge. The Gag encoded by this virus is 91% related to the immunogen and the Env encoded by this virus is 83% related. The challenge was carried out at 5000 TCID₅₀ (˜MID₃₀1.8×10⁷ copies of viral RNA).

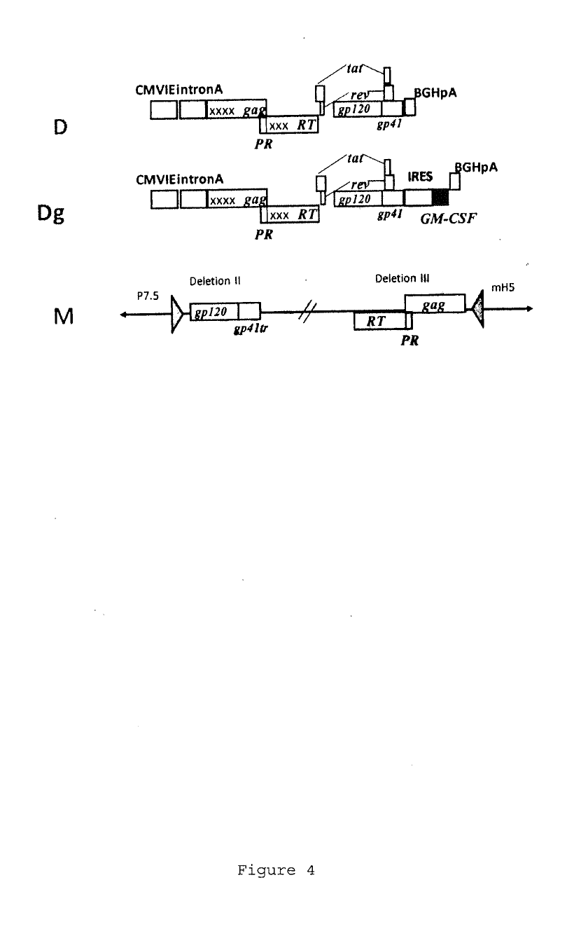

FIG. 4 shows schematics of the SIV239 DNA and recombinant MVA vaccines used in these studies. The GM-CSF co-expressing DNA vaccine (SIV239 DNA) was constructed by inserting rhesus macaque GM-CSF sequences in a plasmid that expressed SIV239 Gag, Pol and Env sequences. The GM-CSF co-expressing DNA expressed about 200 ng of GM-CSF per 106 transiently transfected 293T cells, a level of expression that has been found to be associated with enhanced immune responses for cellular cancer vaccines. A single recombinant MVA also expressed Gag, Pol and Env, but did not co-express GM-CSF (Van Rompay et al., J. Virol. 83:2686-2696, 2009). Both vaccines expressed membrane-bound trimeric forms of the envelope glycoprotein with the goal of eliciting Ab to the form of Env found on virions and infected cells. The MVA vaccine expressed virus like particles whereas the over-expressed Gag-pol sequence in the DNA vaccine formed intracellular aggregates, as well as virus-like particles. The co-expression of GM-CSF in the DNA immunogen did not cause changes in hematology or blood chemistries or elicit detectable antibody to GM-CSF (data not shown).

The DDMM and DgDgMM regimens elicited similar temporal patterns and magnitudes of Env-specific serum IgG, but different patterns of Env-specific IgA in rectal secretions (FIG. 5A,B). In both groups, the IgG responses rose subsequent to the MVA boosts and declined to about 20% of their peak values by the time of challenge. IgA, measured as a specific activity (ng of Env IgA per μg total IgA) was detected following the first MVA boost, and increased in both frequency of detection and height following the 2nd MVA boost. At the time of challenge, IgA titers had contracted by about 50%. At peak IgA responses, Env-specific IgA was detected in 57% of the animals in the DgDgMM group as opposed to 12% of the animals in the DDMM group. The specific activity of Env IgA in secretions was greater than that in blood, indicating that the rectal IgA had originated from local mucosal synthesis.

Further analysis of the elicited IgG response revealed that the Env-specific IgG elicited by the DgDgMM regimen was qualitatively different from that elicited by the DDMM regimen. Co-expression of GM-CSF in the immunogen increased the avidity of the Env-specific IgG response (FIG. 5C). This enhancement was significant for the SIV239 Env of the immunogen and showed a trend for the SIVE660 Env of the challenge virus. Consistent with the higher avidity, the Env-specific Ab in the GM-CSF adjuvanted group had higher neutralizing activity and higher ADCC activity (FIGS. 5D and E) (Xiao et al., J. Virol. 84:7161-7163, 2010). Titers of neutralizing activity were increased for two easy to neutralize isolates derived from the genetically diverse SIVE660 challenge stock: SIV660.11 and SIVE660.17, and achieved significance for SIVE660.11. Neutralizing Ab for a more difficult to neutralize isolate SIVE660.CR54 was below the level of detection in the TZM-bl assay (data not shown). ADCC activity was also tested and sera from both the DDMM and DgDgMM groups contained antibodies capable of mediating ADCC activity. Sera from the DgDgMM had significantly higher ADCC activity than the DDMM sera (FIG. 5E). FIGS. 6 and 7 present data on the level of Env-specific and Gag/Pol-specific IgA antibodies in rectal secretions of the M11 macaques.

Elicited T cell responses were analyzed for their magnitude, breadth, and cytokine co-expression using intracellular cytokine staining (ICS) of peripheral blood mononuclear cells (PBMC) stimulated with peptide pools representing SIV239 Gag and Env (FIG. 8). ICS assays tested for patterns of expression of interferon (IFN)-γ, interleukin (IL)-2, and tumor necrosis factor (TNF)-α. In contrast to the elicited antibody responses, where differences were found between the two groups, differences in T cell responses were not detected. Both vaccine regimens elicited similar temporal magnitudes of CD4 and CD8 T cell responses (FIGS. 8A and B), similar breadths of CD4 and CD8 T cell responses (FIGS. 8C and D), and similar patterns of polyfunctionality in responding CD4 and CD8 T cells (FIGS. 8E and F, and data not shown). Differences were also not found in proliferation assays conducted throughout the immunization phase (data not shown).

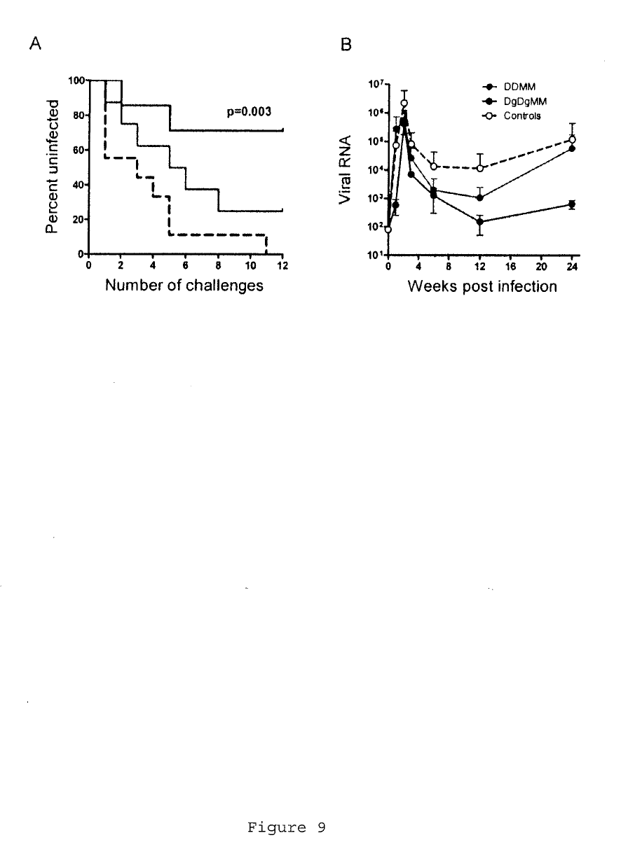

The repeated rectal challenge was initiated at 6 months following the last MVA immunization, a time when vaccine-elicited responses had contracted into memory. Infection was delayed in both the DDMM and DgDgMM vaccine groups with the DgDgMM group resisting infection at a highly significant level (FIG. 9A). As noted above, seventy-one percent of the animals in the DgDgMM group (5/7) were protected against the 12 challenges, whereas only 25% of the DDMM group (2/8) were protected. All but one of the 9 control animals were infected by the 5th challenge and the remaining animal was infected at the 11th challenge. The difference in protection for the GM-CSF adjuvanted and unvaccinated group was highly significant (p=0.003, Mantel Cox test). Differences between the adjuvanted and non-adjuvanted groups and between the non-adjuvanted and unvaccinated groups showed trends that did not achieve significance within our group sizes. Temporal levels of post-challenge viremia suggested a more stringent and sustained control in the GM-CSF-adjuvanted group (FIG. 9B). Despite the encouraging control in the DgDgMM group, the pattern in this group was not significantly different from the other groups because of the small number of infected animals in the DgDgMM group and the variable levels of post challenge control in the other groups.

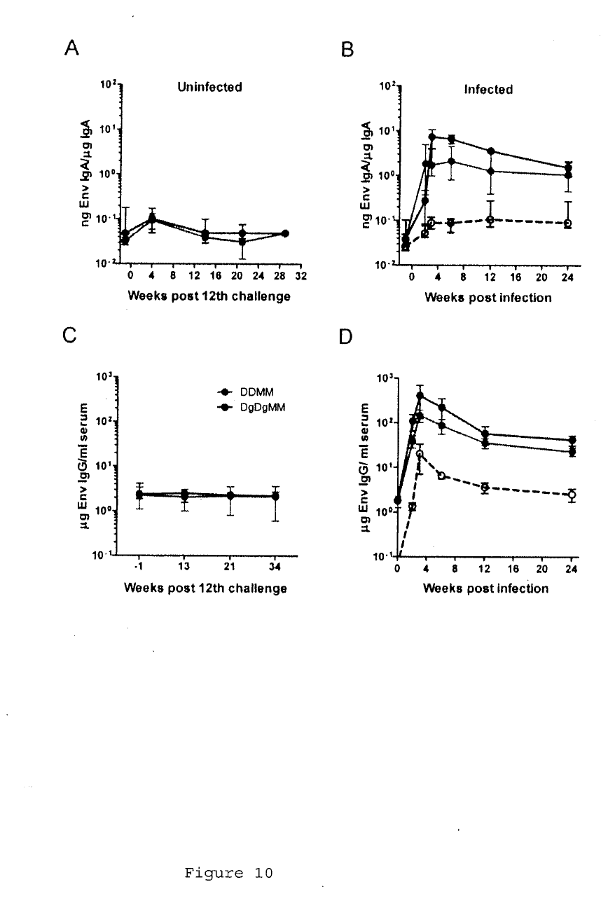

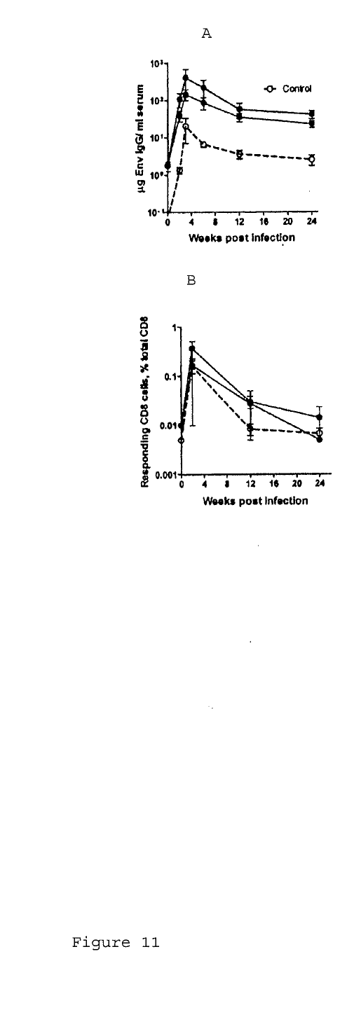

In both vaccine groups, the prevention of infection was complete. No evidence of viral replication or evolving SIV-specific immune responses were found during the 12 months post the last challenge. This includes the lack of anamnestic Env-specific IgA responses in rectal secretions (FIG. 10A), anamnestic Env-specific IgG responses in blood (FIG. 10C), and responding T cells for Nef; a protein present in the challenge virus, but not the vaccine. This is in strong contrast to the vaccinated and infected animals where strong anamnestic IgA responses in rectal secretions (FIG. 10B), anamnestic IgG responses in blood (FIG. 10D), and responding T cells to Nef (data not shown) were clearly evident. Vaccinated and infected animals also demonstrate strong anamestic IgG responses in blood (FIG. 11B).

Titers of neutralizing and ADCC antibody activities and the presence of anti-Env IgA, which were higher in the GM-CSF-adjuvanted group, did not correlate with protection (Table 1). Elicited T cell responses also did not correlate with the number of challenges to infection (Table 1). The correlation with avidity was specific for the Env of the challenge virus and was not observed for the Env of the SIV239 immunogen (Table 1).

TABLE 1
Correlations between vaccine-elicited responses and number of
challenges to infection¹

| Correlation | Spearman r | P value (two sided) |
|---|---|---|
| IFN-γ + CD4+ T cells | 0.1 | 0.8 |
| IFN-γ + CD4+ T cells | 0.2 | 0.6 |
| CD4+ proliferation | 0.2 | 0.5 |
| CD8+ proliferation | 0.2 | 0.5 |
| CD4 breadth, 1st MVA | 0.4 | 0.1 |
| CD4 breadth, 2nd MVA | 0.5 | 0.1 |
| CD8 breadth 1st MVA | −0.1 | 0.8 |
| CD8 breadth, 2nd MVA | −0.1 | 0.8 |
| Binding Ab, SIV239 Env | −0.3 | 0.3 |
| Binding Ab, SIVE660 Env | −0.5 | 0.1 |
| Avidity Index, SIV239 Env | 0.2 | 0.4 |
| Avidity Index SIVE660 Env | 0.9 | <0.0001 |
| Neutralizing Ab, E660.11 | 0.0 | 0.9 |
| Neutralizing Ab, E660.17 Env | 0.0 | 1.0 |
| ADCC titer | 0.2 | 0.4 |
| Rectal Secretions, Env - specific IgA | 0.0 | 1.0 |

¹Correlations for T cells were done at one week after the second MVA boost, except for breadth that was done at one week after the first and second MVA boosts. Correlations for Ab responses were done at two weeks post the second MVA boost with the exception of neutralizing Ab for SIVE660.17 that was done at 13 weeks post the second MVA boost. All correlations were for 15 XY pairs except for those for breadthy after the second MVA, which were for 13 XY pairs. The correlations used a two-tailed non-parametric Spearman test. P values are shown without the Bonferoni correction for multiple comparisons. The Bonferoni correction for a significance of 0.05 for the 16 analyses had a P value of 0.003 for the correlation between avidity for the E660 Env and the prevention of infection.

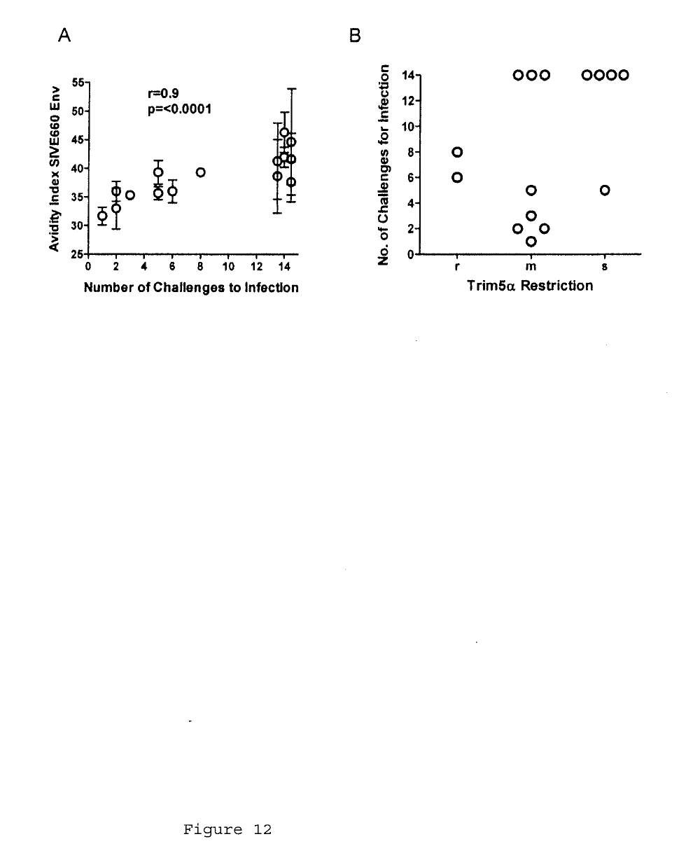

Analyses for correlates for prevention of infection revealed a strong correlation between the avidity of Env-specific IgG for the E660 Env of the challenge and the number of challenges to infection (r=0.9, p=<0.0001) (FIG. 12A). The avidity correlation suggested that animals with an avidity index of greater than 40 were largely protected against infection during the 12 challenges.

To test whether TRIM5α, an innate restriction factor that is polymorphic in rhesus macaques might have played a role in our findings, rhesus macaques were typed for TRIM5α. As noted above, the results of these analyses revealed no correlation in the vaccinated animals between the number of challenges to infection and the presence of restrictive (r), moderately restrictive (m), or susceptible (s) TRIM5α genotypes (FIG. 12B). Of the seven protected animals, four had the susceptible TRIM5α genotype; three, a moderately susceptible genotype, and none had the restrictive genotype. Thus, no evidence could be found for TRIM5α restricting infection in the vaccinated and protected animals.

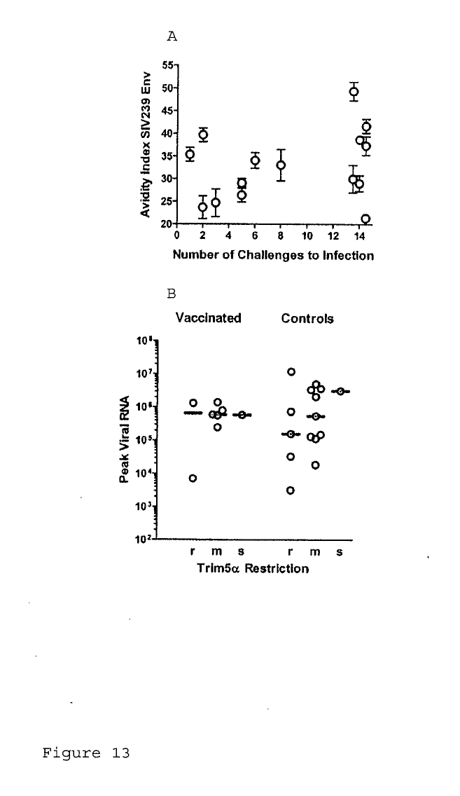

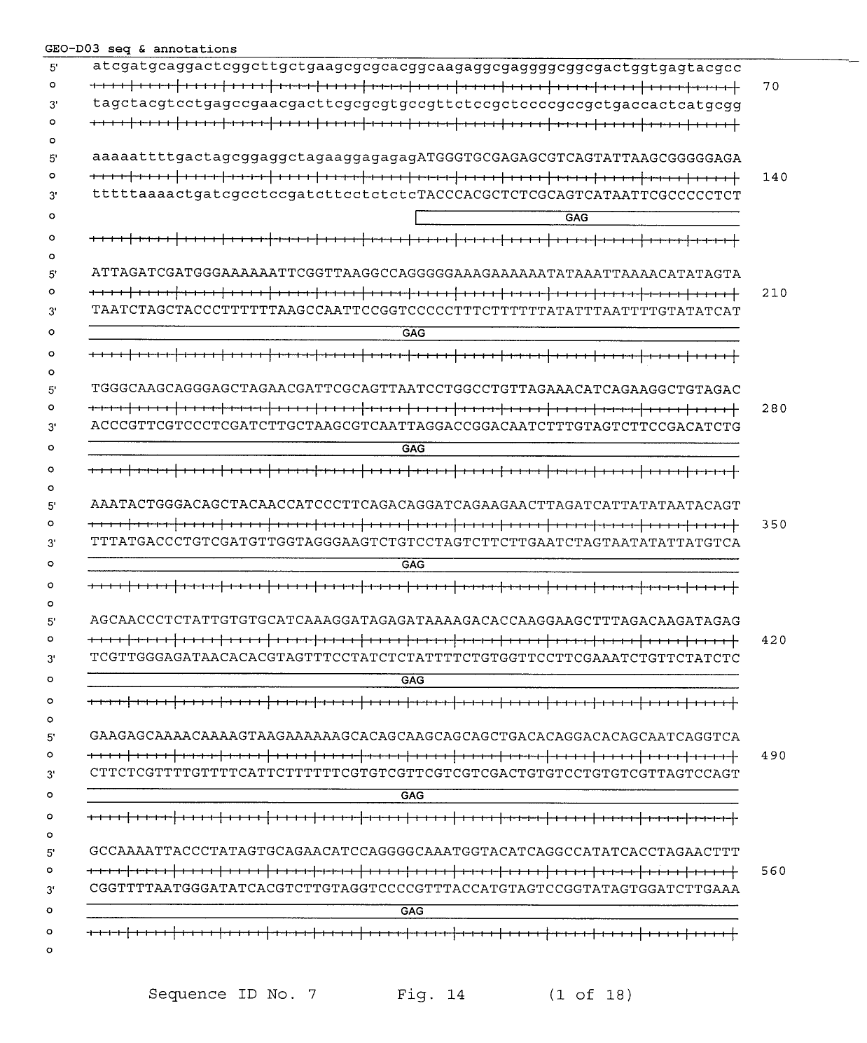

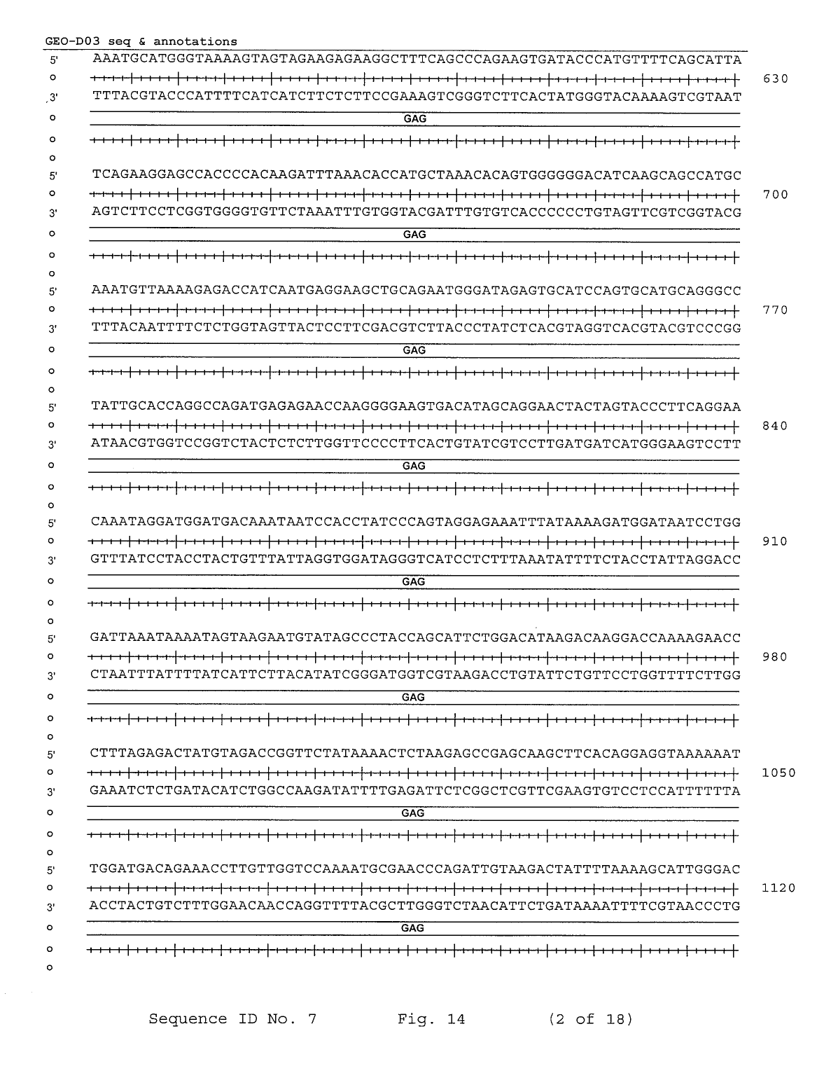

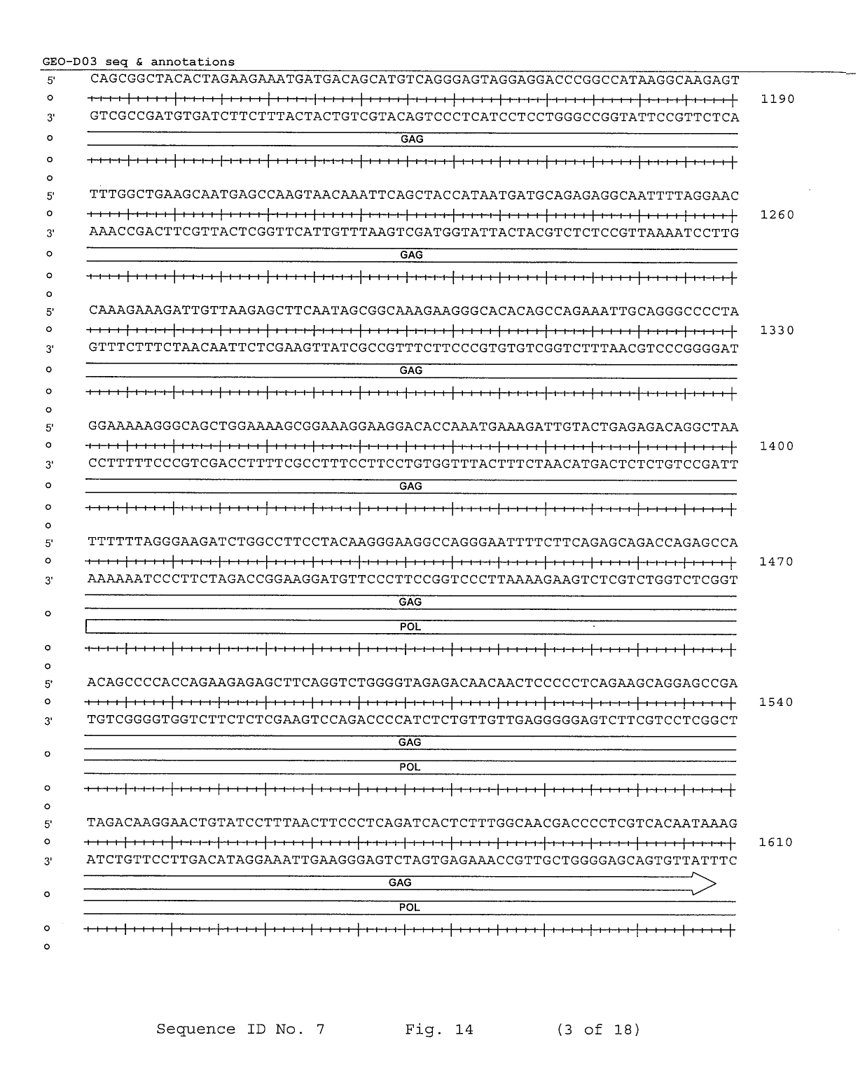

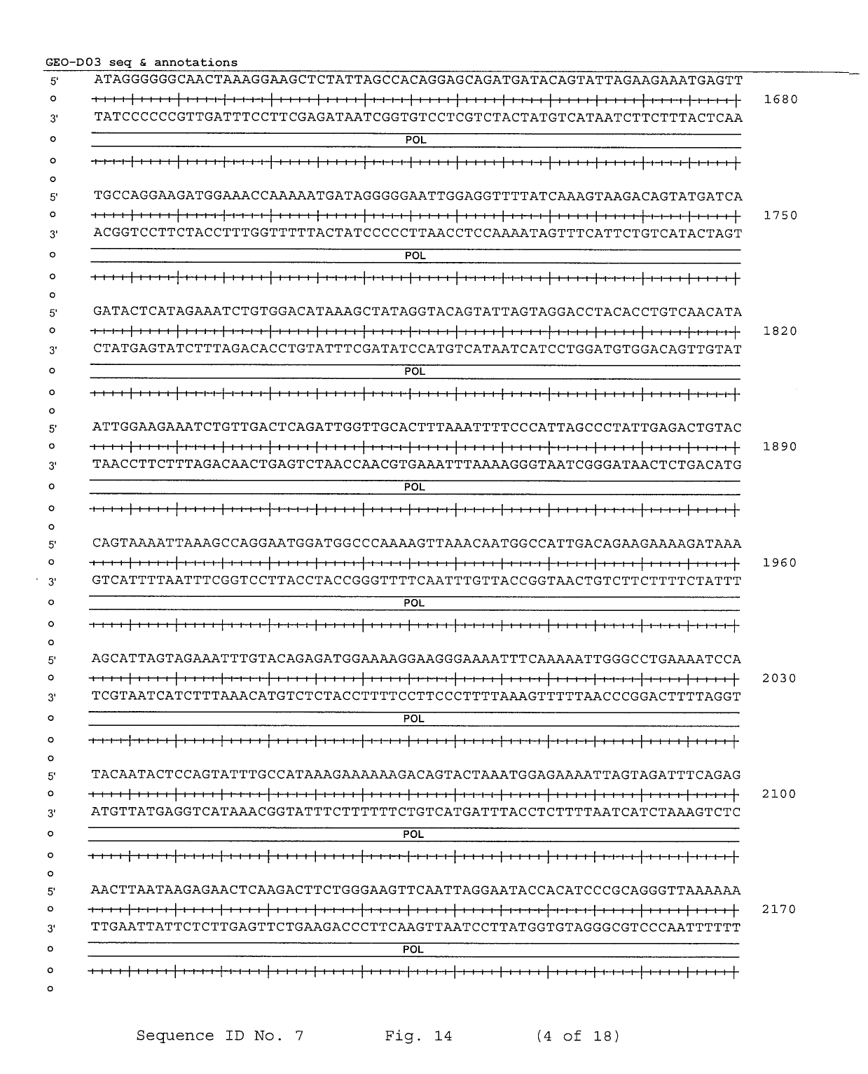

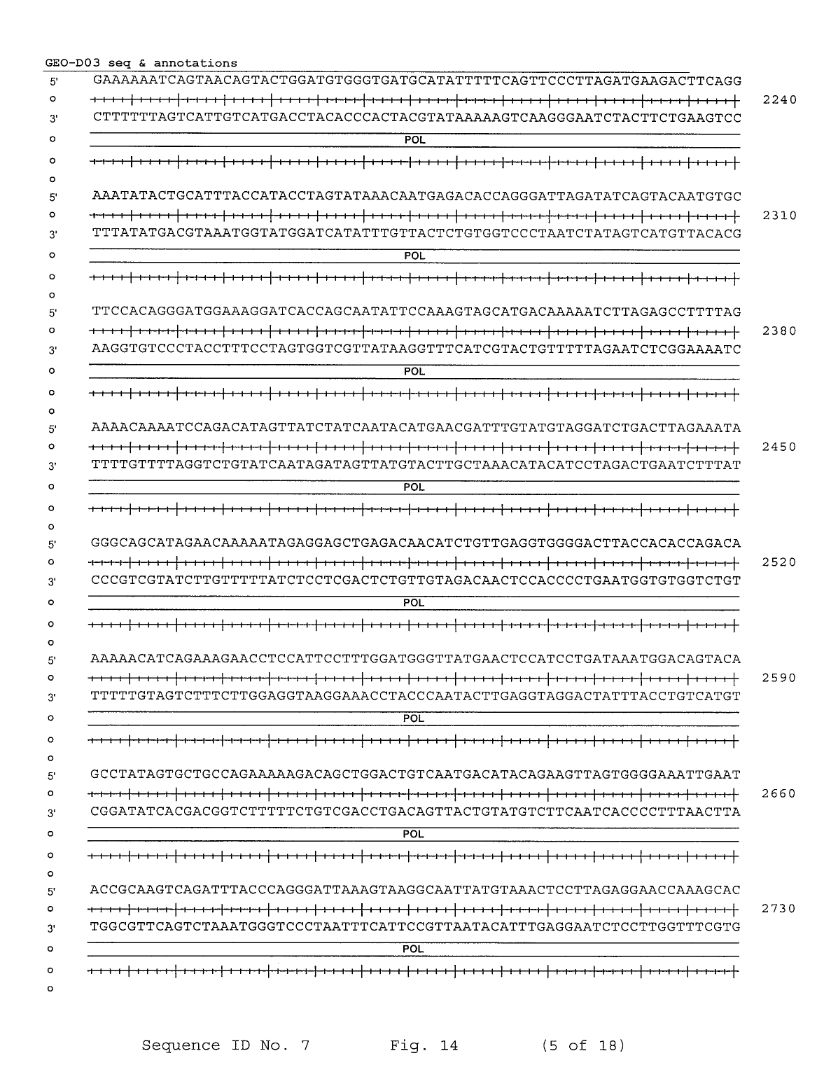

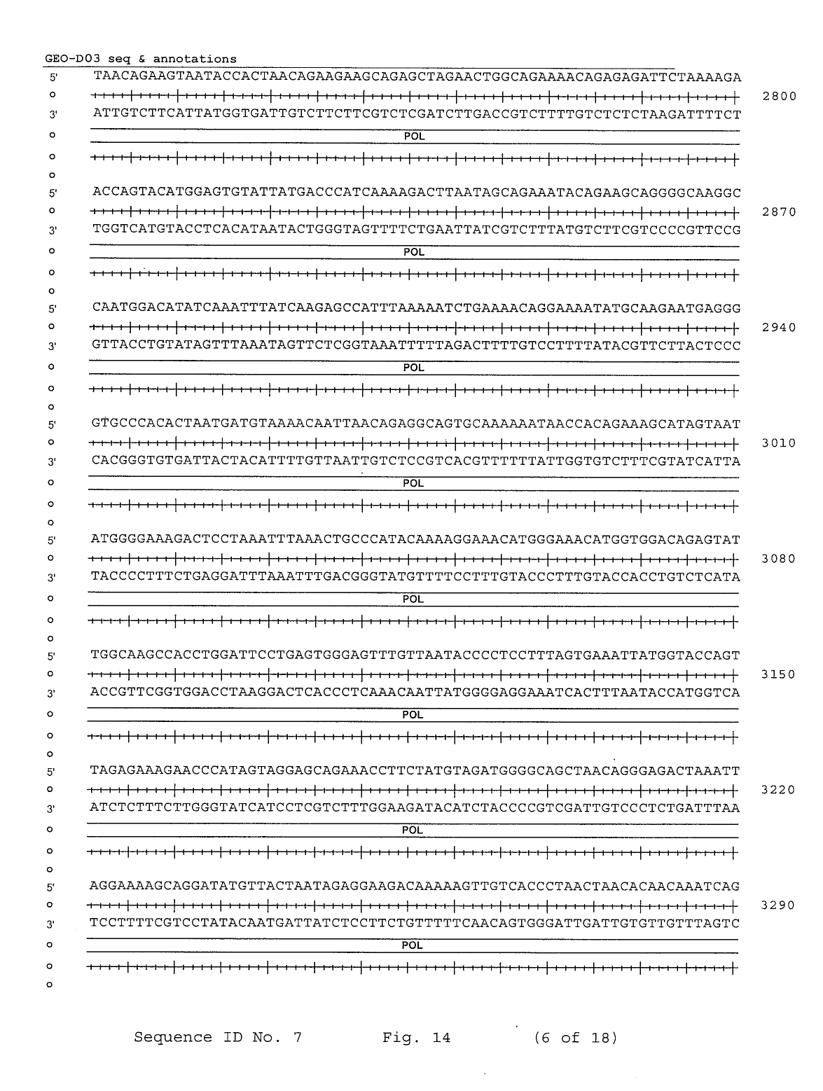

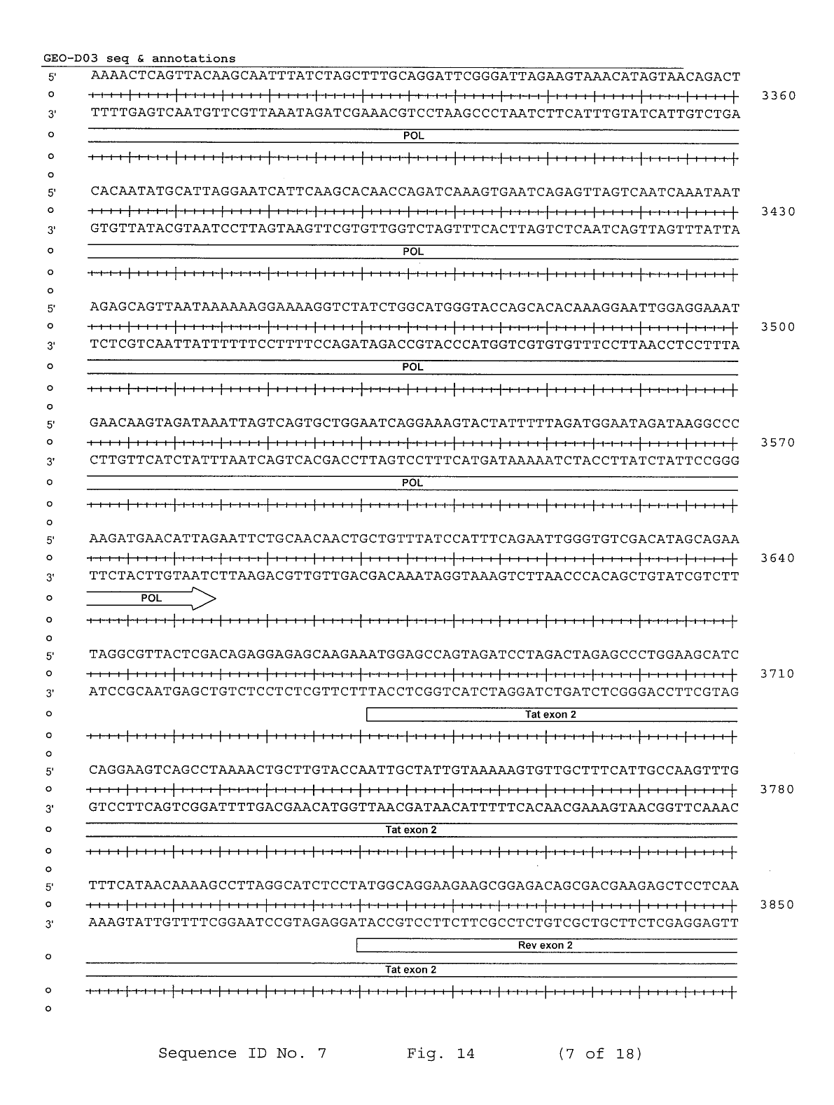

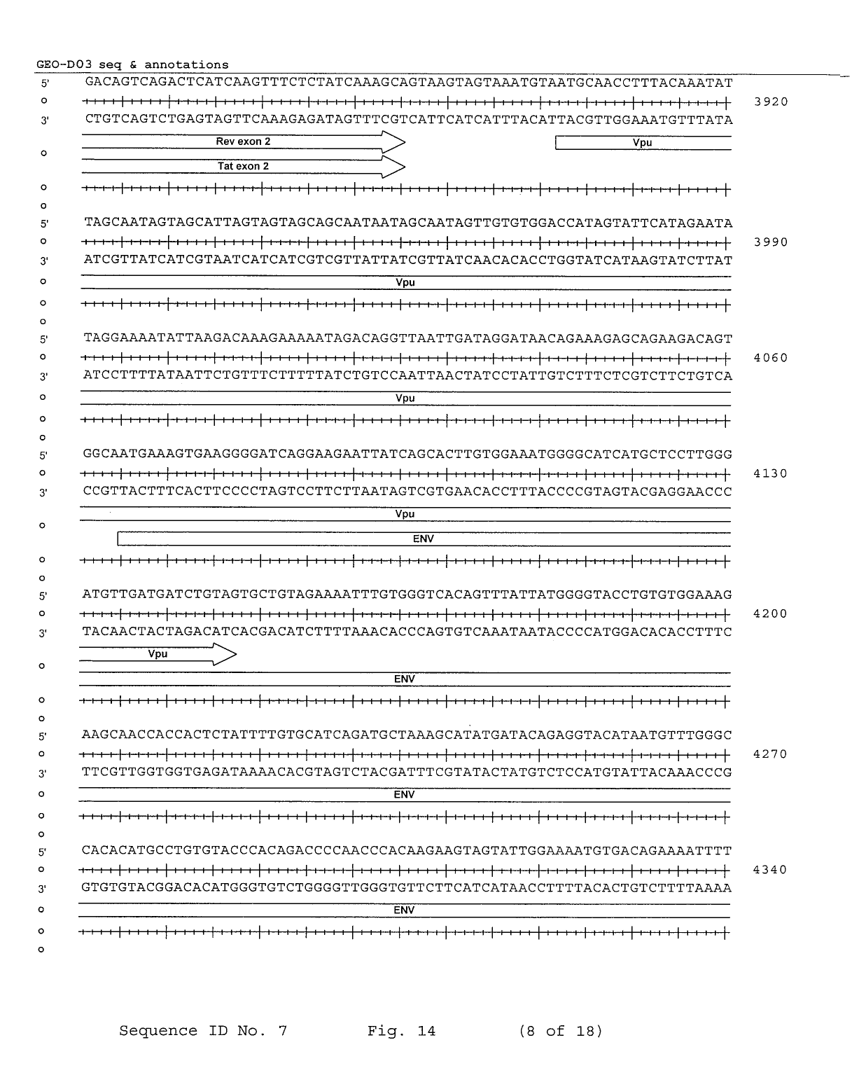

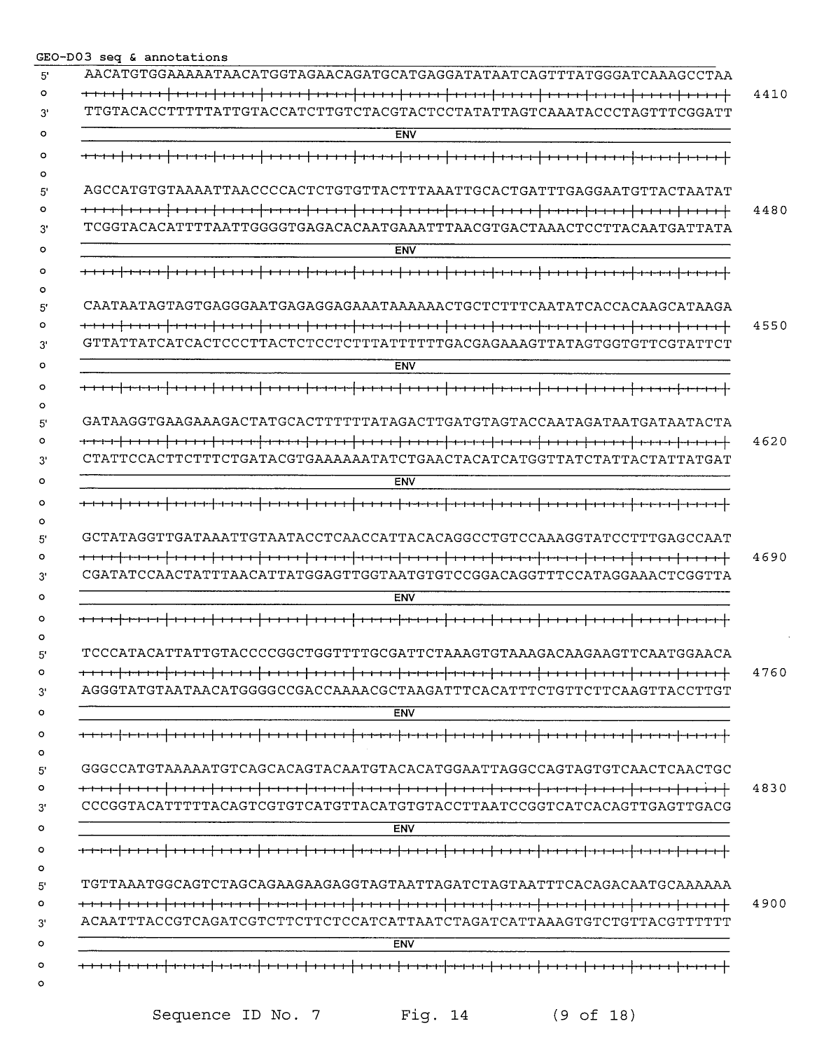

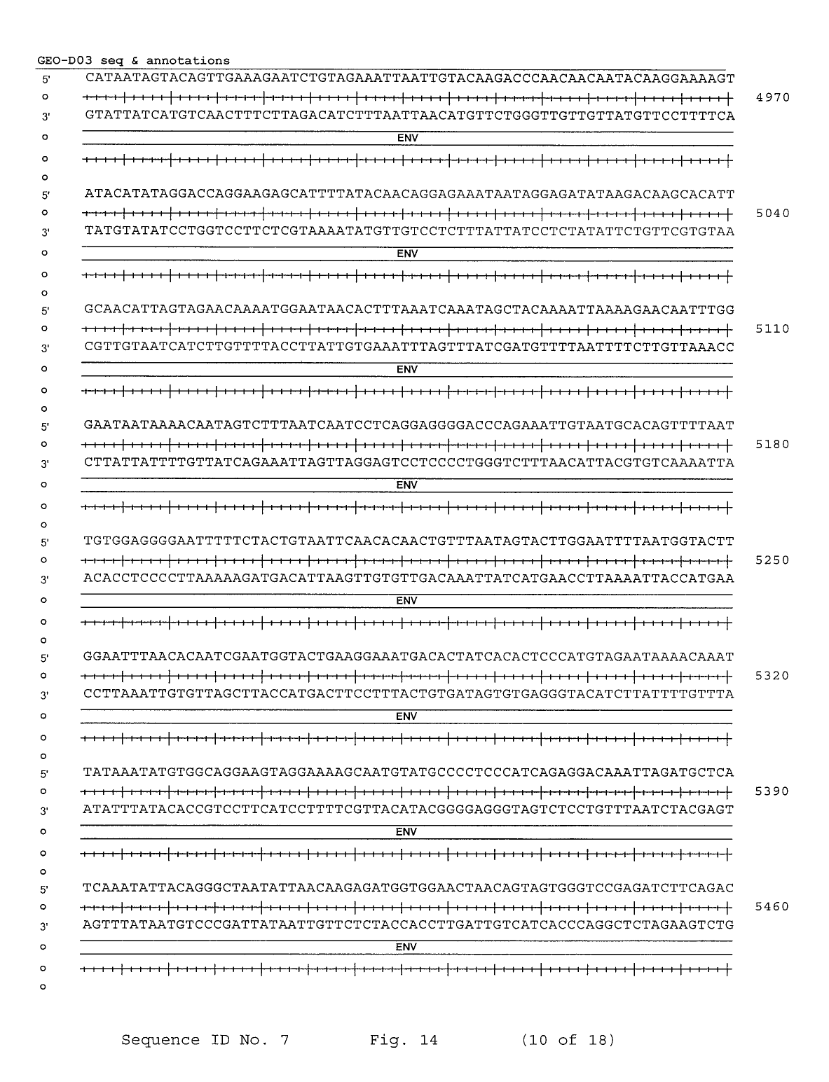

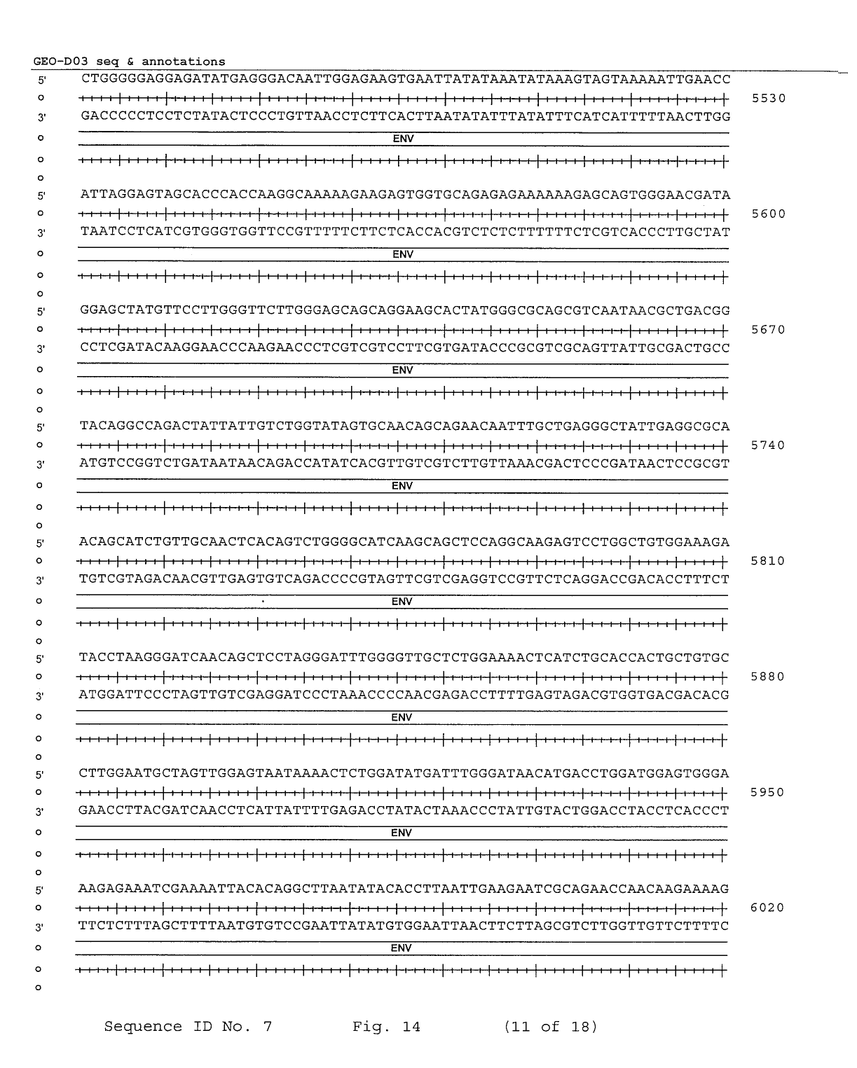

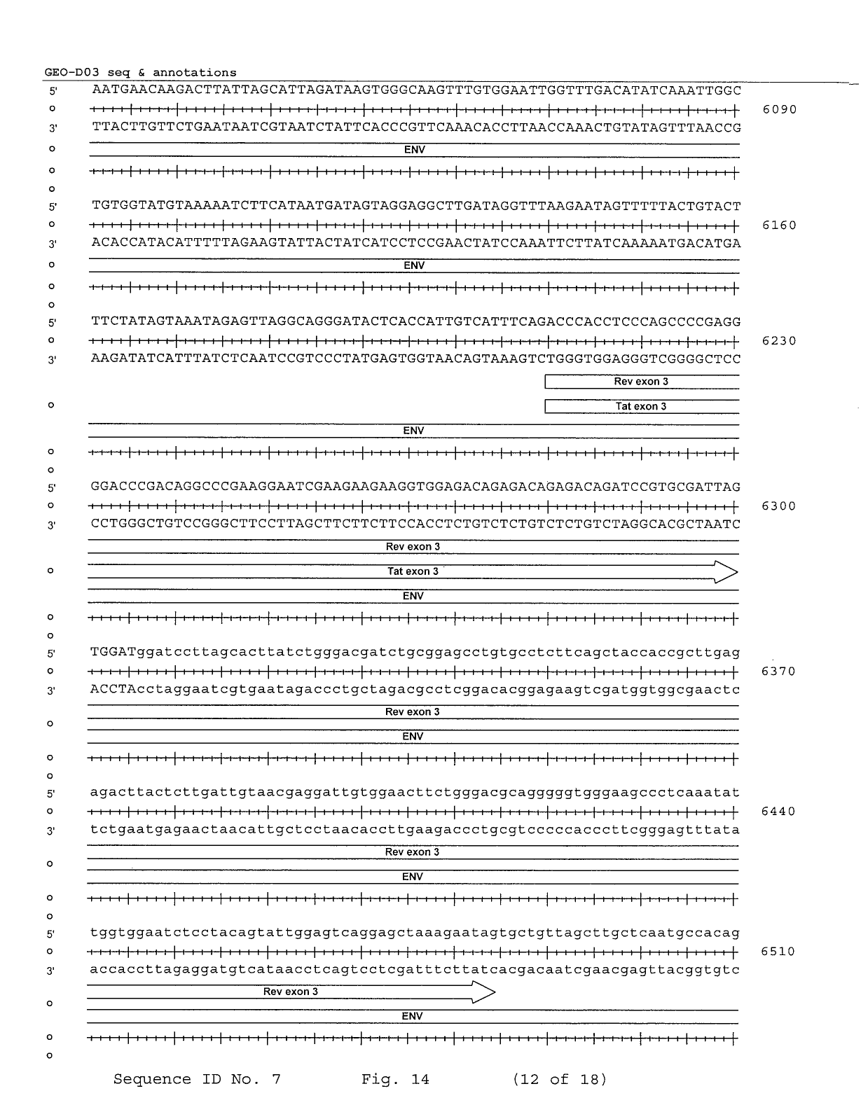

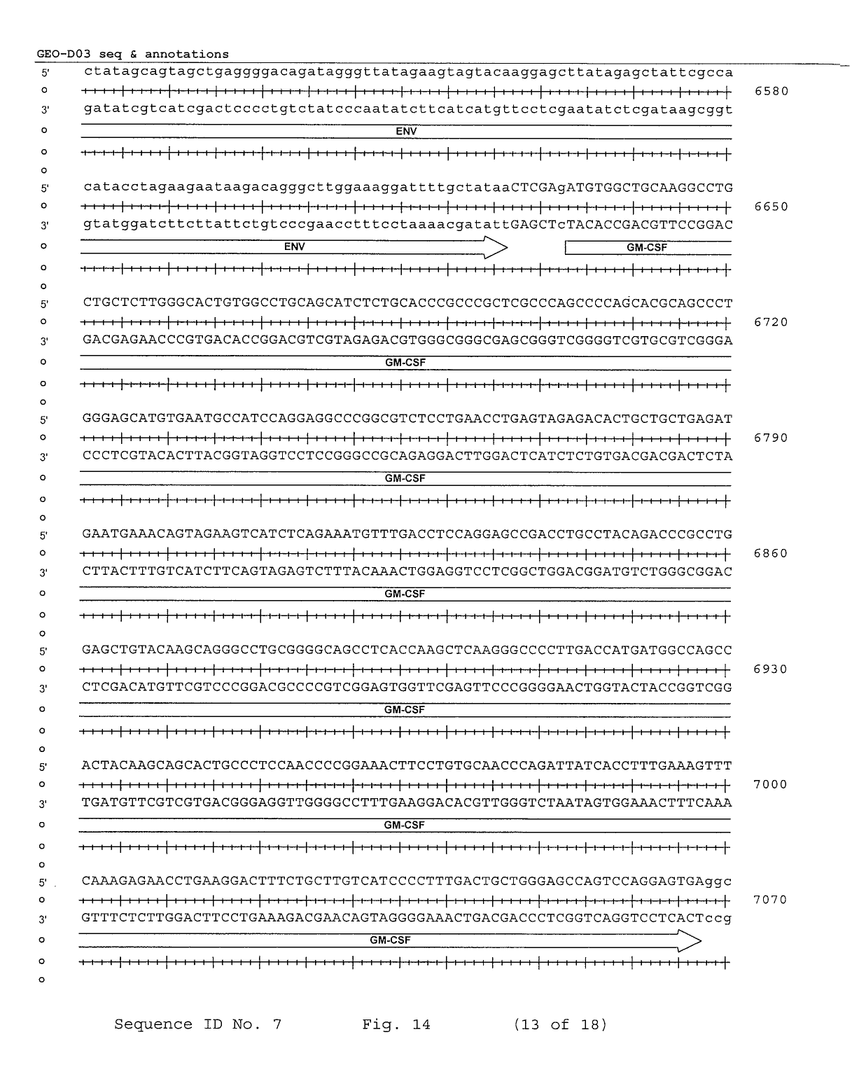

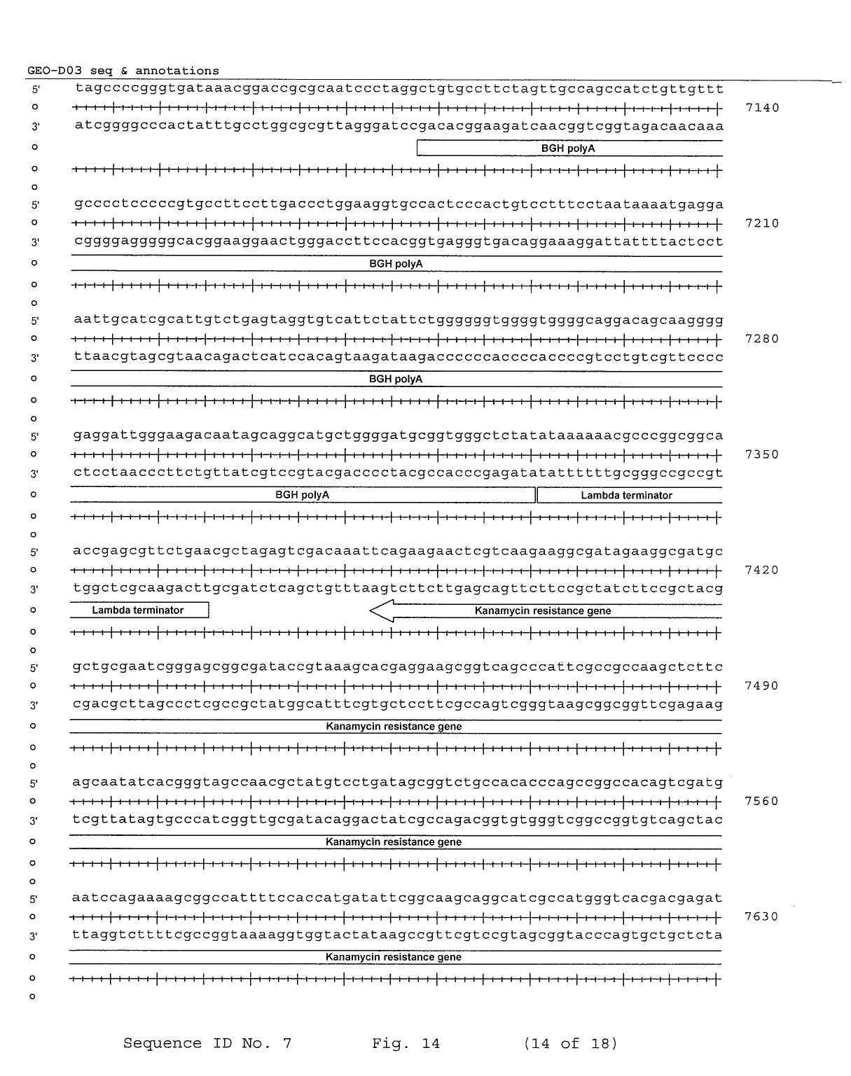

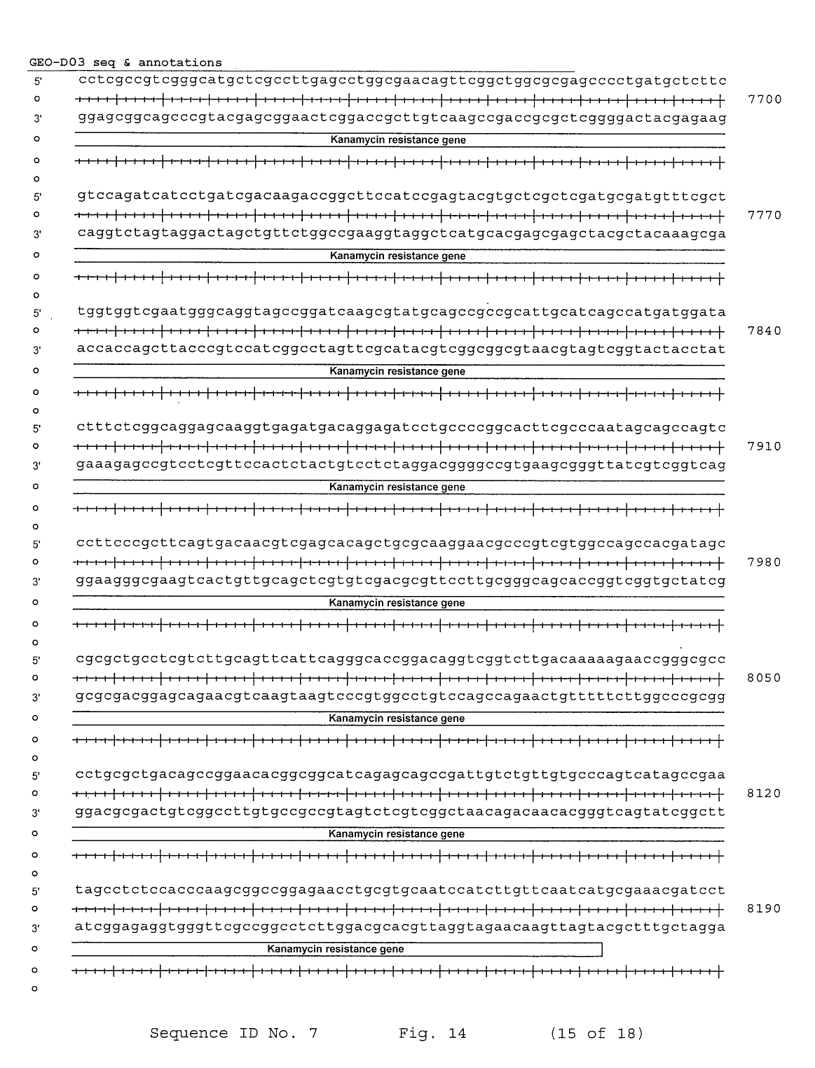

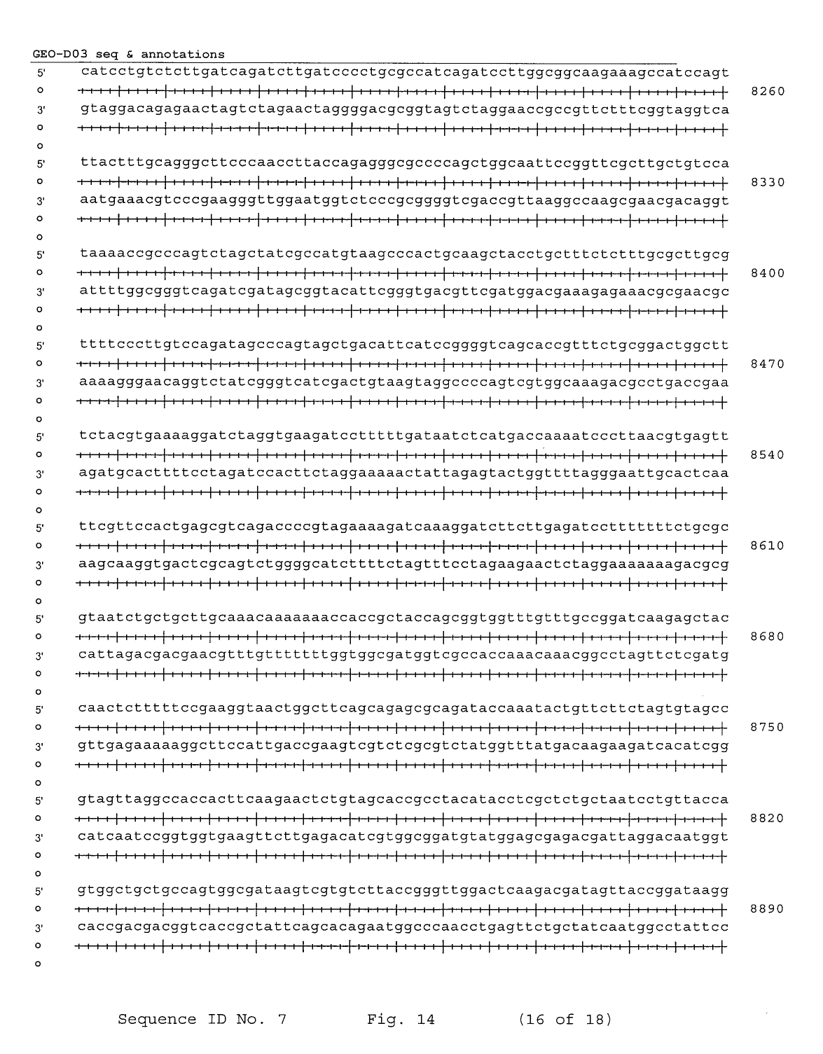

The correlation between avidity and prevention of infection was not observed for the avidity of vaccine-elicited IgG for the SIV239 Env of the immunogen (FIG. 13A). Also, no correlations were observed between neutralizing titers of E660.11 or E660.17, the presence of anti-Env IgA in rectal secretions, or the tested T cell responses and prevention of infection (data not shown). The strong correlation between avidity of vaccine-elicited Ab for the E660 Env and prevention of infection also was observed in an overlapping trial, which tested CD40 ligand as an adjuvant for the 239 vaccine. The correlations for the trial testing CD40 ligand directly overlie those for the trial testing GM-CSF, strengthening and repeating the findings for a non-neutralizing activity of Ab being a strong correlate for prevention of infection.

The co-expressed GM-CSF in the DNA prime for an MVA boost achieved highly significant protection against a repeated rectal challenge, whereas the vaccine without the coexpressed GM-CSF showed only a trend towards prevention of infection. In the presence of the co-expressed GM-CSF, 71% of the vaccinated animals were protected against 12 repeated rectal challenges; whereas in the absence of the co-expressed GM-CSF, only 25% of the group was protected. These results suggest that targeting low levels of GM-CSF expression to the site of DNA immunization can serve as a strong adjuvant for preventing immunodeficiency virus infections. A strong correlate for the prevention of infection was the avidity of the vaccine-elicited Env specific IgG. Animals that had avidity indices of approximately 40, or higher, were not infected whereas those with indices below 40 showed a strong correlation between their avidity index and the number of challenges required for infection. Given these results, we suggest that Ab elicited by trimeric membrane-bound Env might recognize Env on virions and infected cells, and that if this Ab has sufficient avidity, it can initiate Fc-mediated mechanisms of protection such as complement (C′)-mediated lysis, opsonization, ADCC, and antibody dependent cell-mediated virus inhibition (ADCVI) (Xiao et al., J. Virol. 84:7161-7173m 2010; Huber et al., J. Intern. Med. 262:5-25, 2007). The Fc region of the Ab can also bind to cervical mucus providing an Ab trap for viral infections (Hope et al., Program and Abstracts of AIDS Vaccine 2010, Abstract S04.01). In studies in rhesus monkeys with our HIV vaccines, we have shown that the Env-specific Ab elicited by our clade B vaccine has broad avidity for incident clade B, but not incident clade C isolates; and, that Ab elicited by our clade C vaccine has broad avidity for the Envs of incident clade C, but not incident clade B isolates (Zhao et al., J. Virol. 83:4102-4111, 2009). Thus, we suggest that high avidity Ab can have broad intraclade activity. This suggestion is consistent with studies on complement and Fc-mediated mechanisms of Ab-mediated protection which show patient sera having good breadth for mediating these activities against patient isolates.

Co-expression of GM-CSF in the DNA vaccine augmented avidity for both the Env of the SIV239 immunogen and the Env of the SIVE660 challenge. However, to observe the correlation between avidity and number of challenges to infection, avidity needed to be measured for the SIVE660 Env of the challenge stock. The SIV239 Env could elicit protective avidity for the SIVE660 Env, but the targets for this protection needed to be assessed using the challenge Env. These results indicate that there are multiple conserved targets for high avidity Ab on Env and suggest that each isolate will display different constellations of conserved targets.

In contrast to Ab responses, where four out of the five features we measured were enhanced by the co-expressed GM-CSF; none of the features we measured for T cell responses were changed. This likely reflected our assays having focused on responses characteristic of type 1 T cell help and not the follicular CD4+ T cell help that support the maturation of Ab responses in germinal centers or the elicitation of type 2 T cell help favored by GM-CSF-stimulated dendritic cells. GM-CSF stimulates the expansion and differentiation of myeloid dendritic cells, which display the receptor for GM-CSF. Myeloid dendritic cells preferentially migrate to the marginal zone of lymph nodes where germinal centers for the maturation of B cells undergo formation. The GM-CSF-stimulated myeloid dendritic cells produce IL-6, an important cytokine for the formation of germinal centers and the growth and differentiation of B cells in germinal centers. Also, GM-CSF-stimulated myeloid dendritic cells favor the elicitation of type 2 T cell help, a type of help that does not display the CCR5 chemokine receptor that is used as a co-receptor by HIV. Thus the GM-CSF adjuvant may facilitate prevention of infection by eliciting types of T cell help that do not seed mucosal surfaces with preferred targets for infection.

The strong correlation between the avidity of vaccine-elicited IgG and the number of challenges to infection is the first demonstration that avidity can provide a serological correlate for prevention of infection by an immunodeficiency virus challenge. This demonstration introduces a new concept for HIV vaccine development, non-neutralizing but tightly binding Ab can mediate prevention of a mucosal infection. The ability to elicit broadly neutralizing Ab has eluded vaccine developers, and is rare in natural infections. In contrast, binding Ab for the native form of Env is elicited in virtually all infections. Thus, vaccines that elicit high avidity binding Ab for the native form of Env may be able to provide a protective humoral component for a vaccine.

Prior examples of vaccines for which the avidity of an Ab response was found to be important for protection include the conjugate vaccines. These vaccines convert T-cell independent to T-cell-dependent immunogens and allow Ab stimulated by polysaccharides to undergo affinity maturation in children under two years of age. For example, the avidity of the Ab responses elicited by vaccines for Haemophilus influenzae type B (Hib)(Scgkesubger et al., JAMA 267:1489-1494, 1992) and Streptococcus pneumononiae (pneumococcus) (Anttila et al., J. Infect. Dis. 177:1614-1621, 1998) are key to their protective activities. Failed measles and respiratory syncytial viral vaccines elicit non-protective low-avidity Ab (Polack et al., Nat. Med. 9:1209-1213, 2003; Delgado et al., Nat. Med. 15:34-41, 2009). The measurement of avidity for HIV-1 immunogens may be of particular importance because of the slow maturation of Ab to the highly glycosylated Env (Parekh et al., AIDS Res. Human Retroviruses 17:137-146, 2001).

In sum, our data show a GM-CSF co-expressing DNA prime for a MVA boost eliciting immune responses that prevented infection in 71% of macaques receiving 12 repeated intrarectal challenges with doses of a heterologous SIV that are transmitted 30 to 300 times more frequently than HIV-1 during human heterosexual intercourse (Royce et al., New Eng. J. Med. 336:1072-1078, 1997). The SIVE660 challenge had the same tropism as typical HIV infections (Margolis et al., Nat. Rev. Microbiol. 4:312-317, 2006) and a similar genetic distance from the SIV239 vaccine strain as HIV-1 clade-specific vaccines have for within clade isolates. Provocatively, a non-neutralizing serological marker, avidity of the elicited IgG for the Env of the challenge virus, was identified as a correlate for prevention of infection.

The extent of the enhancement of the prevention of infection found for the GM-CSF co-expressing vaccine was not anticipated. Prior studies using high dose challenges had shown that GM-CSF co-expressing vectors could enhance vaccine-mediated reductions in peak viremia (Lai et al., GM-CSF DNA: an adjuvant for higher avidity IgG, rectal IgA, and increased protection against the acute phase of a SHIV-89.6P challenge by a DNA/MVA immunodeficiency virus vaccine. Virology 369:153-67, 2007; Zhao et al. Preclinical studies of human immunodeficiency virus/AIDS vaccines: inverse correlation between avidity of anti-Env antibodies and peak postchallenge viremia. J Virol 83:4102-11, 2009). In this study, using a repeated moderate dose challenge, actual prevention of infection (not just control of peak viremia) was found, with the GM-CSF increasing this prevention from 25 to 71%. This prevention correlated with the avidity of the Env-specific antibody response for the Env of the challenge virus. Studies being conducted at the same time using co-expressed CD40 ligand as an adjuvant also enhanced the avidity of the Env-specific antibody response but did not enhance prevention of infection to the same extent as observed for the GM-CSF-co-expressing vaccine. Thus, GM-CSF co-expression appeared to being providing protection by mechanisms in addition to the binding of high avidity antibody. We suggest that this may reflect GM-CSF co-expression favoring the vaccine eliciting type 2 T cell (Th2) help, instead of type 1 T cell (Th1) help. Saline injections of DNA tend to prime Th1 help (Feltquate et al. Different T helper cell types and antibody isotypes generated by saline and gene gun DNA immunization. Journal of Immunology 158:2278-84, 1997; Oran et al. DNA vaccines, combining form of antigen and method of delivery to raise a spectrum of IFN-gamma and IL-4 CD4+ and CD8+ T cells. Journal of Immunology 171:1995-2005, 2003). Th1 help displays the CCR5 chemokine receptor that also serves as the co-receptor for HIV infection on its surface (Bonecchi et al. Differential expression of chemokine receptors and chemotactic responsiveness of type 1 T helper cells and type 2 T helper cells. Journal of Experimental Medicine 187:129-34, 1998). In contrast, in the absence of other stimulatory signals, GM-CSF stimulates myeloid dendritic cells (DC) to elicit Th2 help, but requires signals in addition to GM-CSF (such as CD40 ligand, TNF-α) to elicit Th1 cells (Faith et al. Functional plasticity of human respiratory tract dendritic cells: GM-CSF enhances T(H)2 development. J Allergy Clin Immunol 116:1136-43, 2005; Stumbles et al. Resting respiratory tract dendritic cells preferentially stimulate T helper cell type 2 (Th2) responses and require obligatory cytokine signals for induction of Th1 immunity. Journal of Experimental Medicine 188:2019-31, 1998). Th2 cells display CCR4 and CCR3 and not the CCR5 chemokine receptor displayed by Th1 cells (Sallusto et al. The role of chemokine receptors in directing traffic of naive, type 1 and type 2 T cells. Curr Top Microbiol Immunol 246:123-8, 1999). It is possible that the GM-CSF adjuvant, especially when provided by a DNA that expands myeloid DC without providing stimulation of other pattern recognition receptors may minimize the elicitation of CCR5-displaying CD4 T cells. This is desirable for an HIV vaccine because, anti-viral CCR5CD4 T cells are preferential targets for infection (Douek et al. HIV preferentially infects HIV-specific CD4+ T cells. Nature 417:95-8, 2002). Also, the elicitation of high levels of virus-specific CCR5-displaying CD4 T cells by vaccination has been shown to reduce vaccine efficacy (Kannanganat et al. Preexisting Vaccinia Virus Immunity Decreases SIV-Specific Cellular Immunity but Does Not Diminish Humoral Immunity and Efficacy of a DNA/MVA Vaccine. J Immunol; 185:7262-73).

### Methods

Vaccines. The GM-CSF co-expressing DNA vaccine was constructed by inserting rhesus macaque GM-CSF sequences into the pGA1/SIV239 DNA plasmid (termed D) that expresses SIV239 Gag, PR, RT, Env, Tat, and Rev to create the GM-CSF co-expressing plasmid (termed Dg) (FIG. 4). The DNA vaccines express multiple SIV proteins from a single RNA by subgenomic splicing and frameshifting. GM-CSF is expressed by the same mRNA as Env using the encephalomyocarditis virus internal ribosome entry site (IRES). Dg expressed approximately 200 ng of GM-CSF per 106 transiently transfected 293T cells.

A single recombinant MVA (previously designated DR1 or MVASIVgpe and designated M here) expressed Gag, Pol, and Env, but did not co-express GM-CSF (FIG. 20). The MVA vaccine encodes gag and RT sequences in deletion III and env sequences in deletion II of MVA. The MVA vaccine expressed VLP whereas the over-expressed Gag in the DNA vaccine formed intracellular aggregates as well as VLP. The DNA vaccine expressed the complete gp160 form of Env and the MVA vaccine encoded a gp150 form which was truncated to remove 146 amino acids at the C-terminus of the gp41 subunit to enhance expression on the plasma membrane of infected cells and stabilize the insert (Wyatt et al., Virology 372:260-272, 2008). Both vaccines expressed membrane bound trimeric forms of the envelope glycoprotein.

Study Design. Animal studies were conducted at the Yerkes National Primate Research Center and approved by the Emory University Animal Care and Use Committee. Young adult male rhesus macaques were pre-screened to preclude the use of animals with the Mamu-A*01 histocompatibility type and to limit the use of animals with Mamu-B*08 and B*17 types to one per group because these histocompatability types are correlated with enhanced control of SIV infections (Kirmaier et al., PloS Biology 8, 2010). Animals were randomized to adjuvanted and non-adjuvanted vaccine groups of 8 each. Three mg of the DNA vaccines were administered at weeks 0 and 8 and 1×108 plaque forming units of the MVA vaccine at weeks 16 and 24. All vaccinations were delivered intramuscularly by needle injection. The control group, added at the time of challenge, consisted of 9 young adult male animals, similarly selected to be Mamu A*01, B*08 and B*17 negative.

A repeat dose intrarectal challenge was administered starting 6 months after the final MVA immunization using 5000 tissue culture infectious doses 50 (1.8×10⁷ copies of viral RNA) of SIVE660 (Keele et al., J. Exp. Med. 206:1117-1134, 2009). In three independent trials, this dose infected approximately 30% of vaccinated animals at each exposure independent of Mamu type, sex, age and institutional environment (data not shown, B Felber and G. Pavlakis, personal communication). Prior to challenge, one animal in the GM-CSF-adjuvanted group was euthanized because of self-mutilation. Throughout the study hematology and clinical chemistry testing was performed to assess any potential toxicological effects associated with the use of the GM-CSF. TRIM5 genotype was determined by sequence analysis of PCR fragments representing the TRIM5 TFP, CYPA and Q alleles as described (Kirmaier et al., 2010).

Antibody assays. Titers of Env-specific IgG in serum and Env-specific IgA in rectal secretions collected with Weck-Cel sponges were determined using SIV239 VLP or rgp130mac251 (Immunodiagnostics, Woburn, Mass.) as a source of Env antigen in assays for IgG and IgA, respectively (Lai et al., Virology 369:153-167, 2007). Avidity indices, or the fraction of retained Ab following a 1.5 M NaSCN wash×100, were determined using duplicate ELISAs (Lai et al., 2007). SIV239 Env captured from VLP produced by transient transfection of 293T cells and SIVE660 ENV captured from the challenge stock following one round of amplification on rhesus PBMC were used as antigen substrates. Pooled serum from vaccinated rhesus was used as a reference standard in each assay. This sample had a mean avidity index of 38 and a standard deviation of 3. Neutralization assays were conducted using HIV pseudovirions with Envs representing isolates from the genetically diverse SIVE660 stock and a luciferase reporter gene assay in TZM-bl cells (Montefiori, Evaluating neutralizing antibodies against HIV, SIV and SHIV in a luciferase reporter gene assay, New York: John Wiley and Sons, 2004). Assays for antibody dependent cellular cytotoxicity (ADCC) were conducted by adapting a previously published method (Packard et al., J. Immunol. 179:3812-3820, 2007). Briefly, recombinant SIVmac239 gp120 (Immune Tech Corp) was used to coat CEM.NKRCCR5 cells as targets and leukopheresis samples from an uninfected human healthy donor were used as effectors at an effector to target ratio of 30:1. The target cells were preloaded with a substrate that undergoes fluorescence following cleavage with granzymeB. Following one hour of incubation at 37° C. the % of target cells that had received granzyme B from the effector cells and scored as fluorescence positive were reported as % Granzyme B (% GzB) activity. A serum dilution is considered positive if % GzB was >9% after subtraction of the % GzB for effector and target cells incubated without serum.

Cellular immune assays. Cellular immune assays and breadth of responses were conducted using pools of peptides (15 mers overlapping by 11) matched to the SIV239 immunogen for stimulation of PBMC (Lai et al., Virology 369:153-167, 2007). Responding cells were measured using intracellular cytokine staining (ICS). Breadth of responses was tested using 13 Gag and 11 Env peptide pools. Boolean analysis was performed to measure polyfunctionality (Kannanganat et al., J. Virol. 81:12071-12076, 2007). Proliferation was tested using loss of carboxyfluorscein succinmidyl ester (CFSE) staining (Velu et al., J. Virol. 81:5819-5828, 2007).

Statistics. Statistics were conducted using Graphpad Prism and TIBCO Spotfire SPLUS 8.1.

### Example 2

### DNA Vectors Encoding HIV Immunogens and Human GM-CSf

Three exemplary DNA vectors that contain a prokaryotic origin of replication, a promoter sequence, a eurkaryotic transcription cassette comprising a vaccine insert encoding one or more immunogens and GM-CSF, a polyadenylation sequence, and a transcription termination sequence were generated. The DNA vector GEO-D03 is shown in FIG. 17 (SEQ ID NO: 7). The DNA vector GEO-D06 is shown in FIG. 18 (SEQ ID NO: 8). The DNA vector GEO-D07 is shown in FIG. 19 (SEQ ID NO: 9).

The GEO-D03, GEO-D06, and GEO-D07 vectors may be used to induce an immune response in a subject (e.g., a subject that has HIV or a subject that is at risk of developing HIV), to treat a subject having HIV, or to manufacture a medicament for inducing an immune response in a subject (e.g., a subject that has HIV or a subject that is at risk of developing HIV), as described herein.

### Example 3

### Phase I Clinical Study

Described below is a phase 1 clinical study to evaluate the safety and immunogenicity of a prime-boost vaccine of GEO-D03 DNA (SEQ ID NO: 7) and MVA/HIV 62 in healthy uninfected vaccinia naïve adult participants.

This phase 1 trial is a dose escalation study in which 0.3 mg of GEO-D03 and then 3 mg of GEO-D03 DNA will be used to prime a constant MVA62B boost (1×108 TCID50). This dose escalation will allow a careful assessment of the reactogenicity and tolerability of GEO-D03 as it is introduced into humans for the first time.

Inclusion criteria for subjects include: age of 18 to 50 years, good general health, hemoglobin≧11/0 g/dL, WBC of 3,000 to 12,000 cells/mm³, total lymphocyte count≧800 cells/mm³, willingness to receive HIV test results, plates between 125,000 to 550,000 mm³, ALT<1.25 times the institutional upper limit of normal, creatinine≦institutional upper limit of normal, cardiac troponin I or T does not exceed the institutional upper limit of normal, negative HIV-1 or -2 blood test, and negative hepatitis B surface antigen.

This phase 1 trial of GEO-D03/MVA62B will test two DNA primes at weeks 0 and 8 followed by 3 MVA boosts at weeks 16, 24, and 32 in a DDMMM regimen. The results of HVTN 065 indicate that two DNA primes are needed for maximal T cell responses. The results of HVTN 065 also suggest that three MVA inoculations may be needed for optimal Ab responses. Temporal studies on Ab responses showed the 3rd MVA in the MMM regimen increasing anti-Env Ab titers by about 4-fold.

Two products are described. The first is a plasmid DNA vaccine, GEO-D03 (SEQ ID NO: 7), which is manufactured under cGMP/GLP conditions. The second product, MVA/HIV62B (MVA62B) is a recombinant vaccinia virus manufactured under cGMP/GLP conditions by BioReliance Ltd, Glasgow, Scotland.

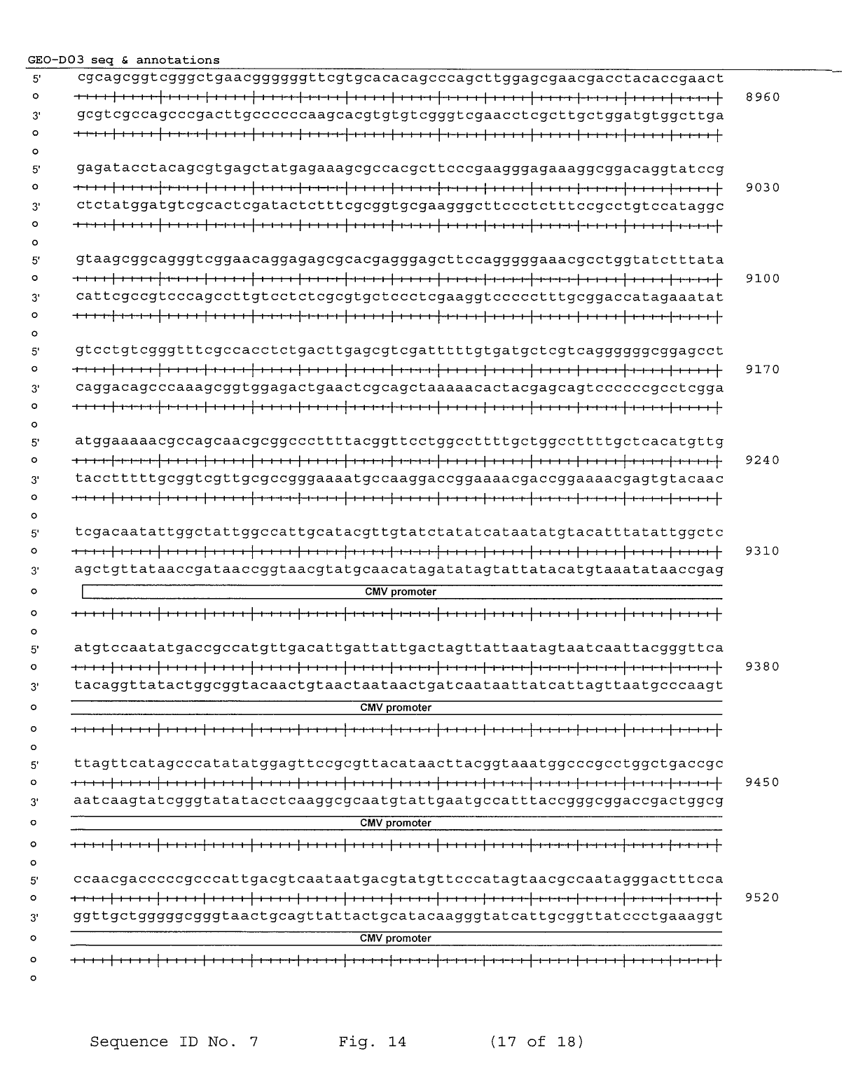

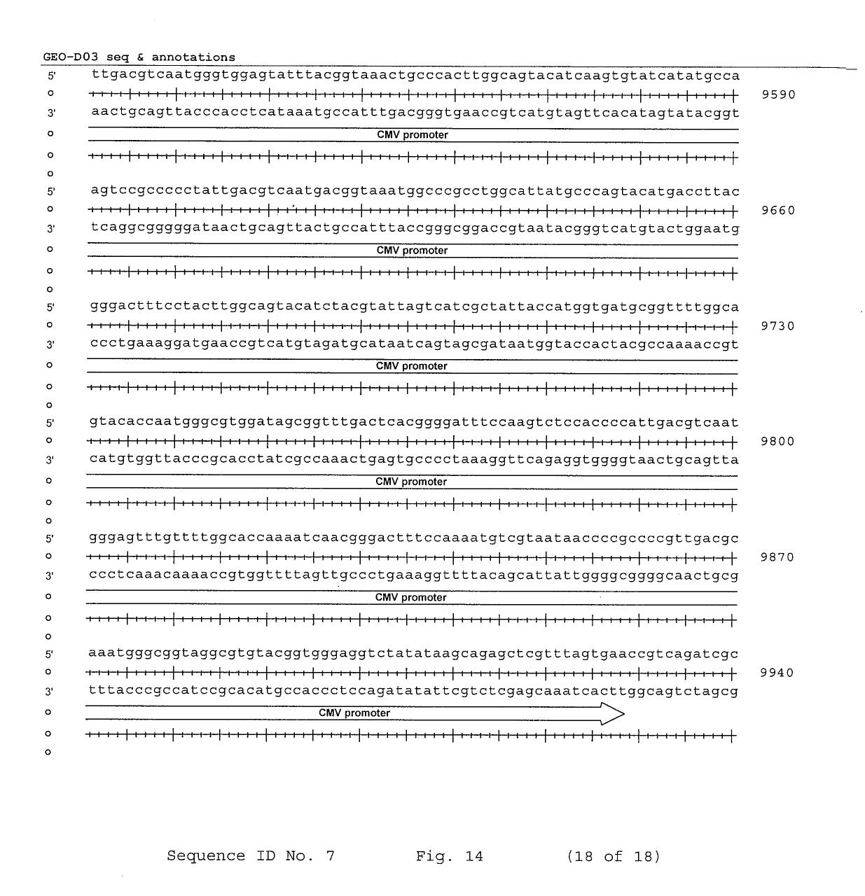

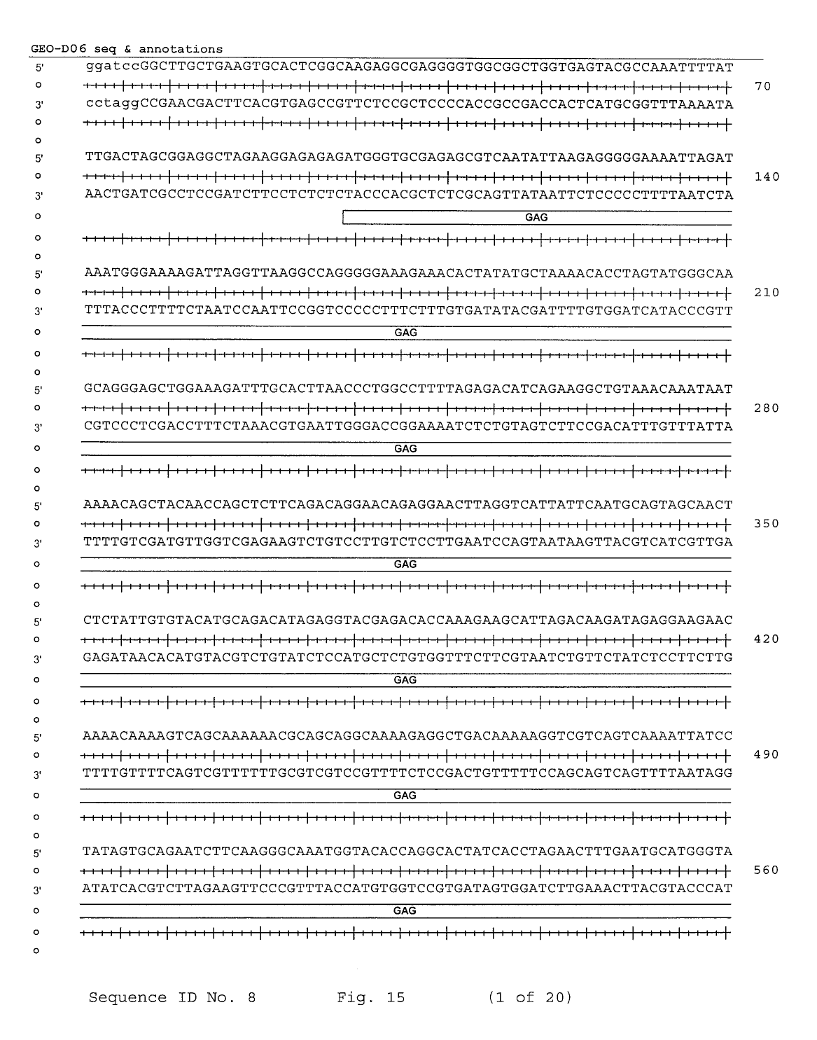

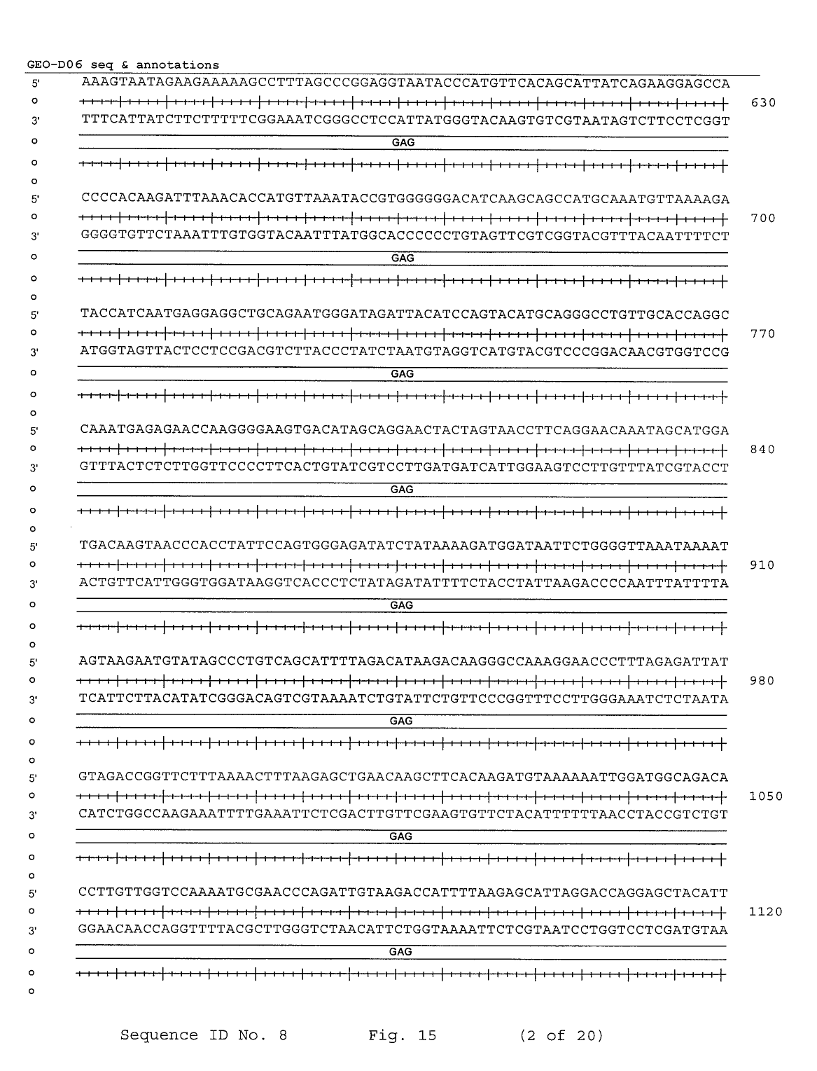

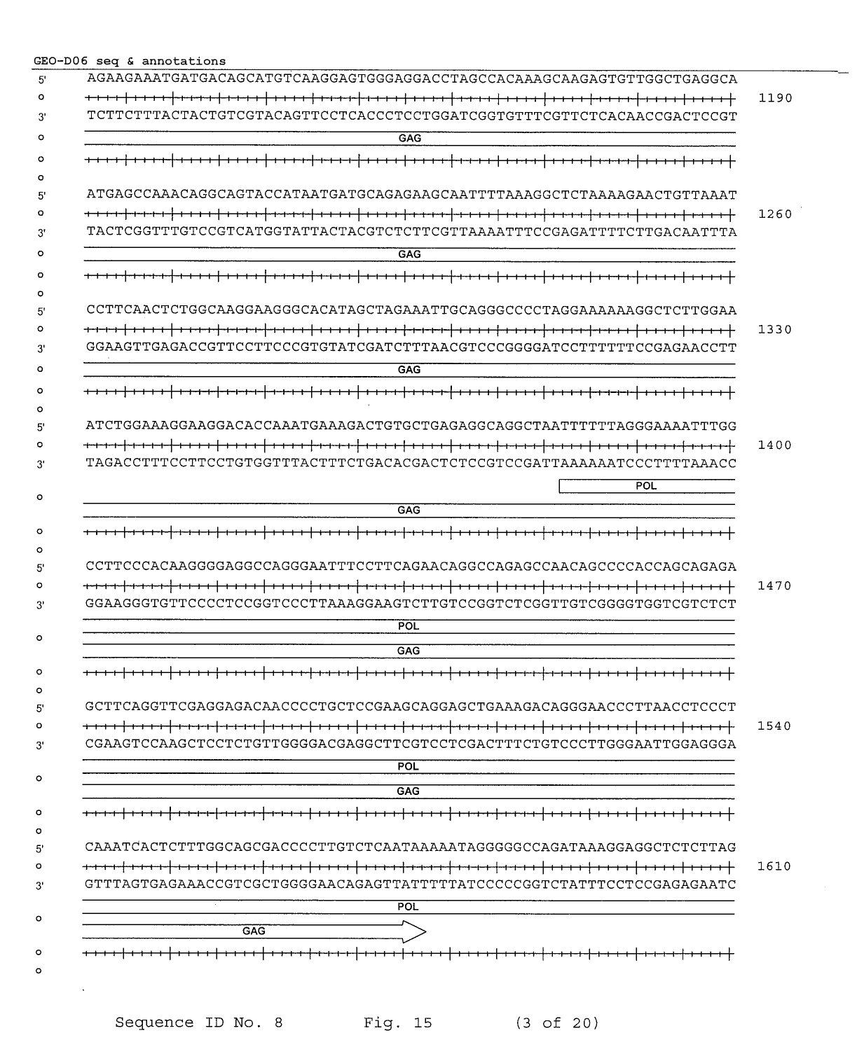

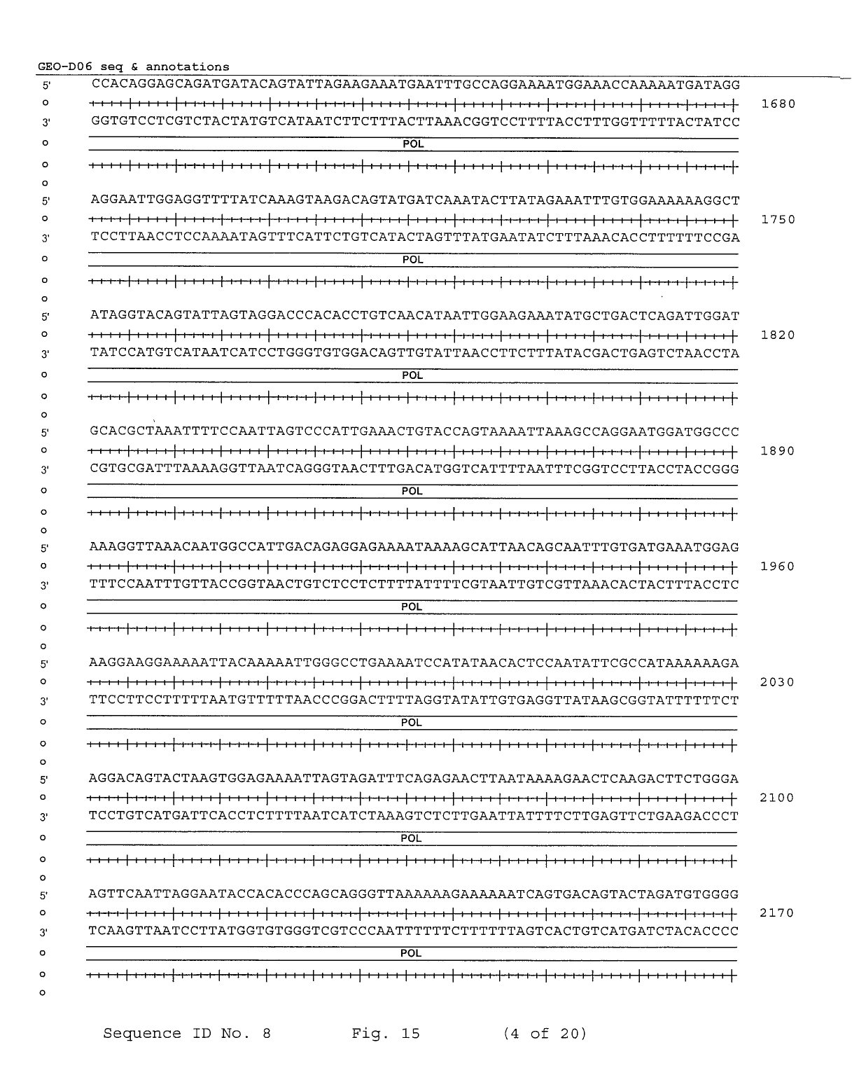

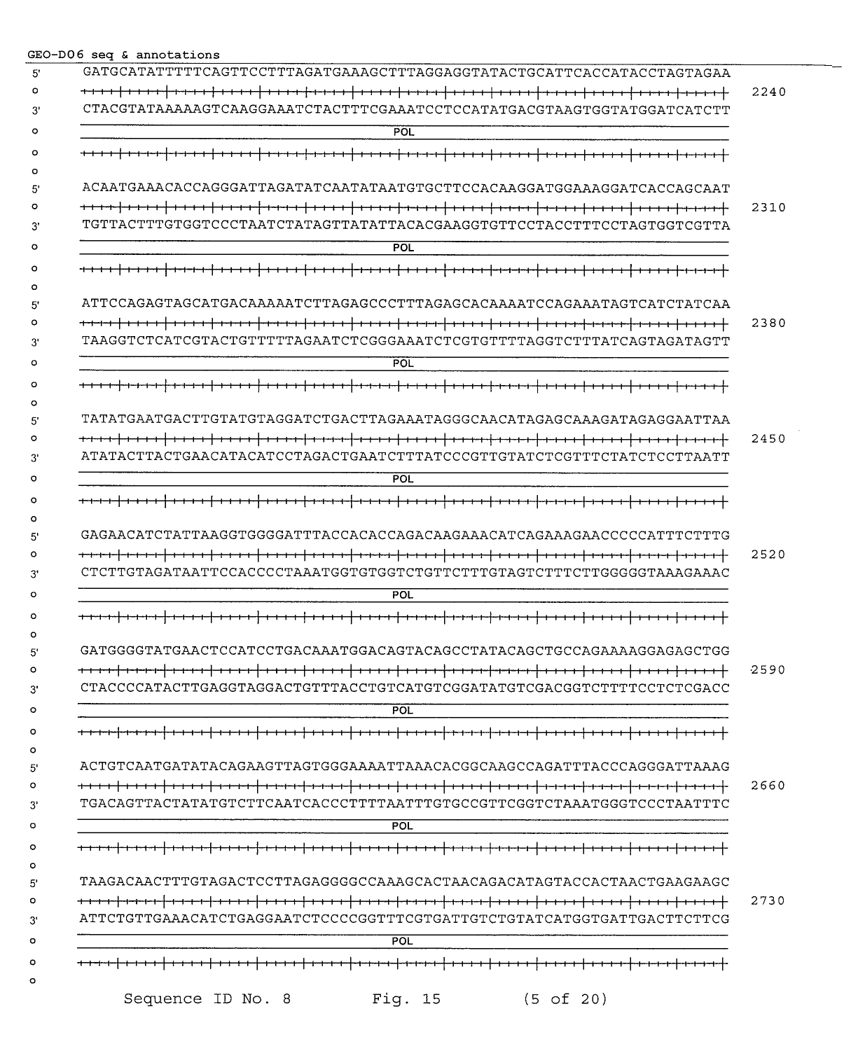

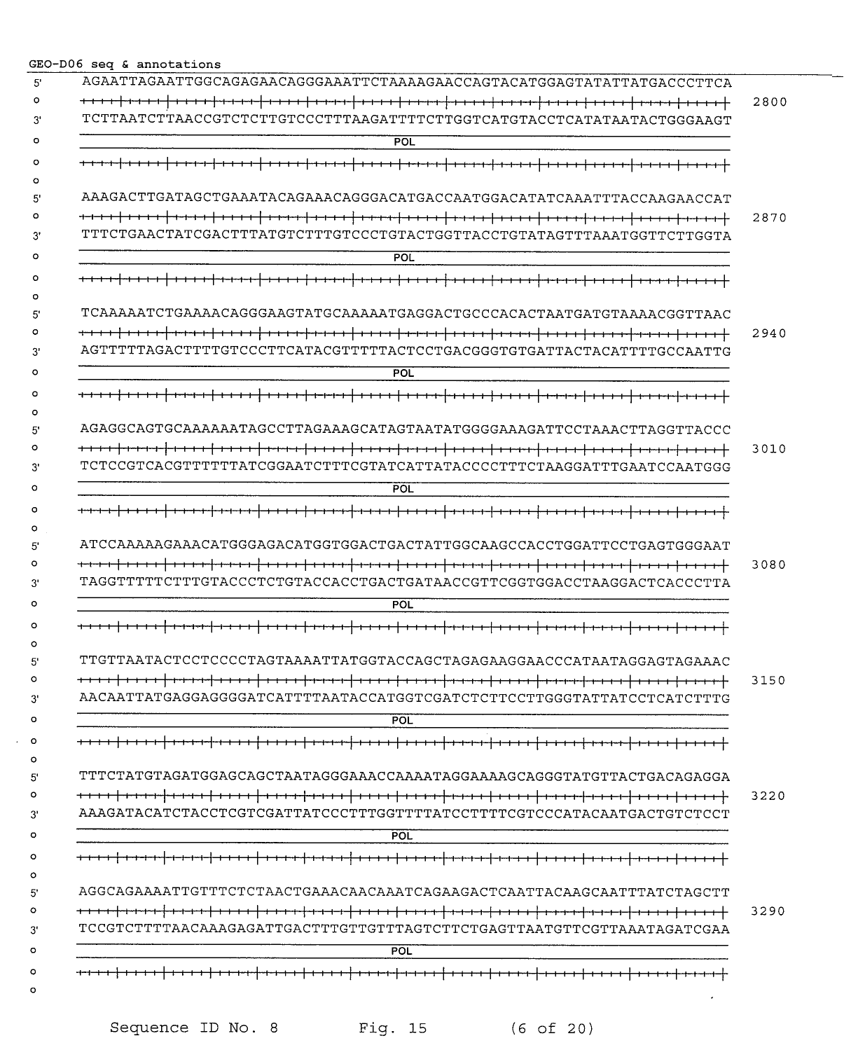

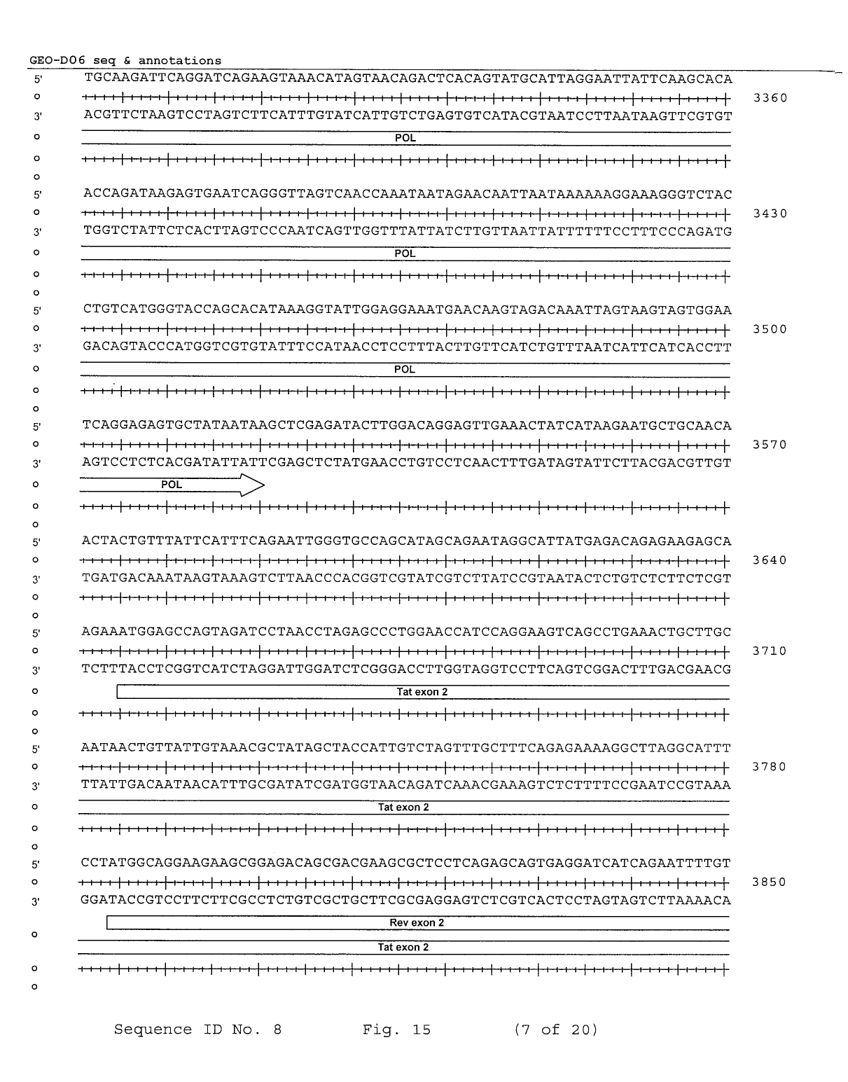

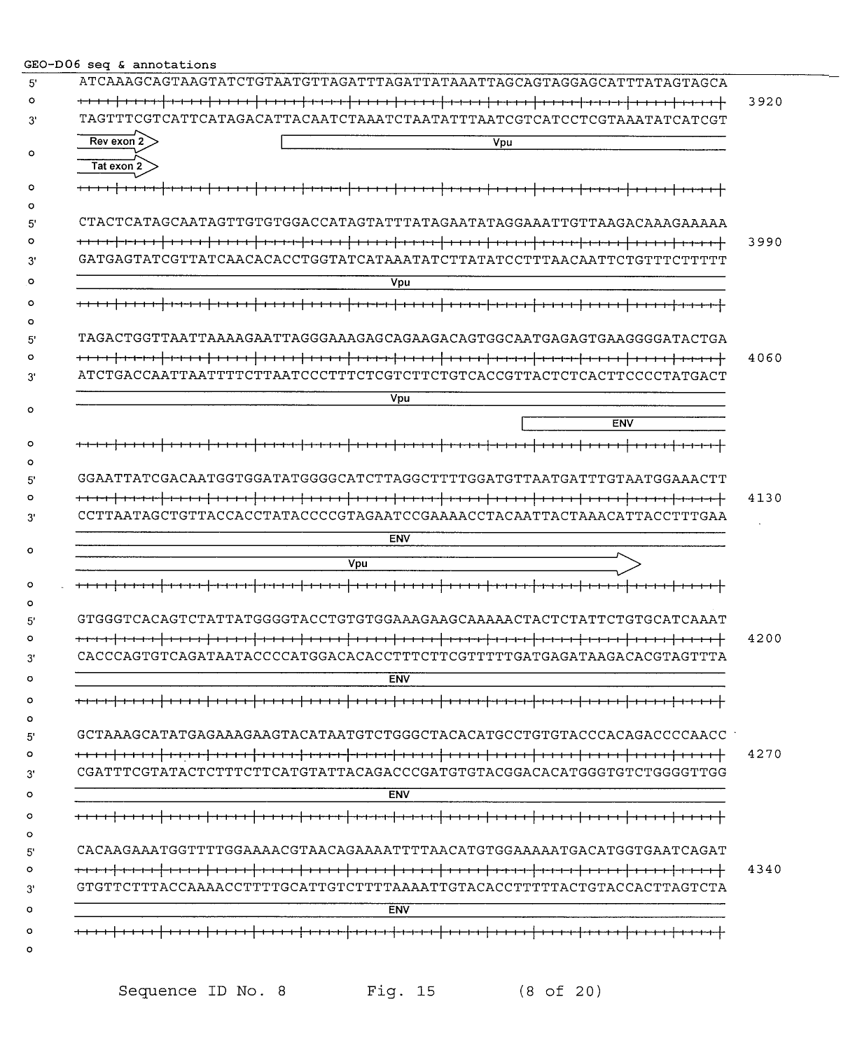

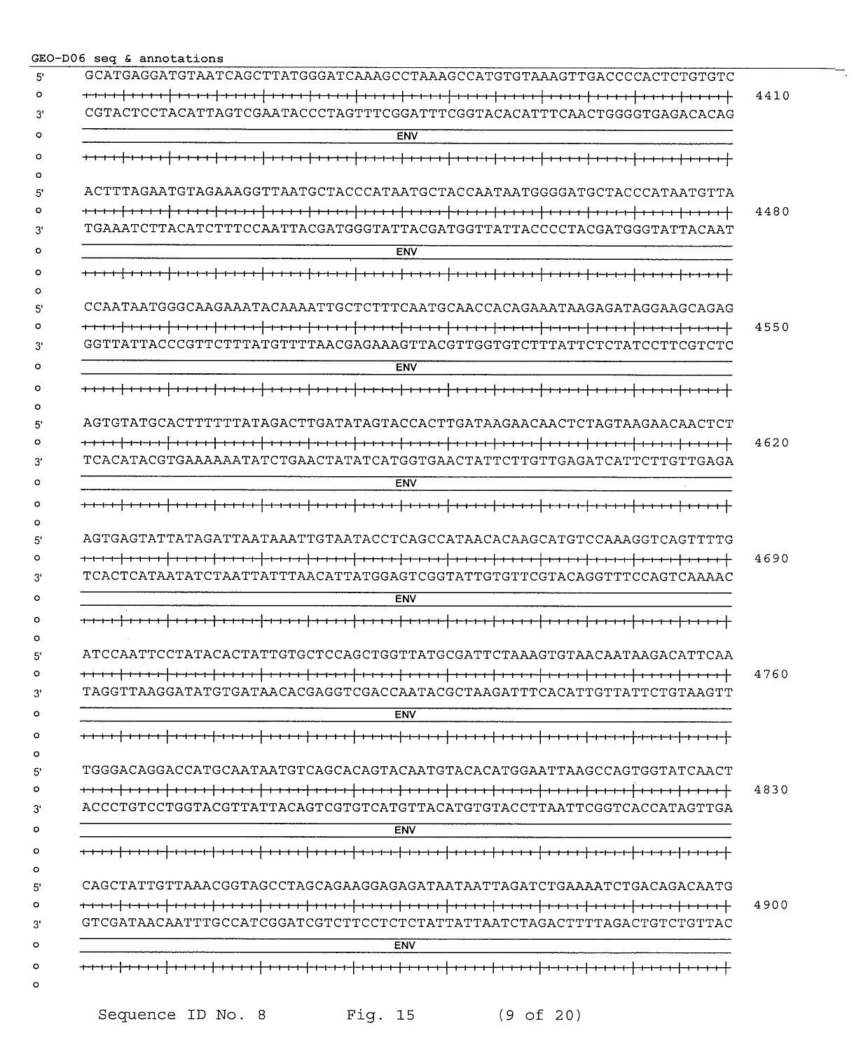

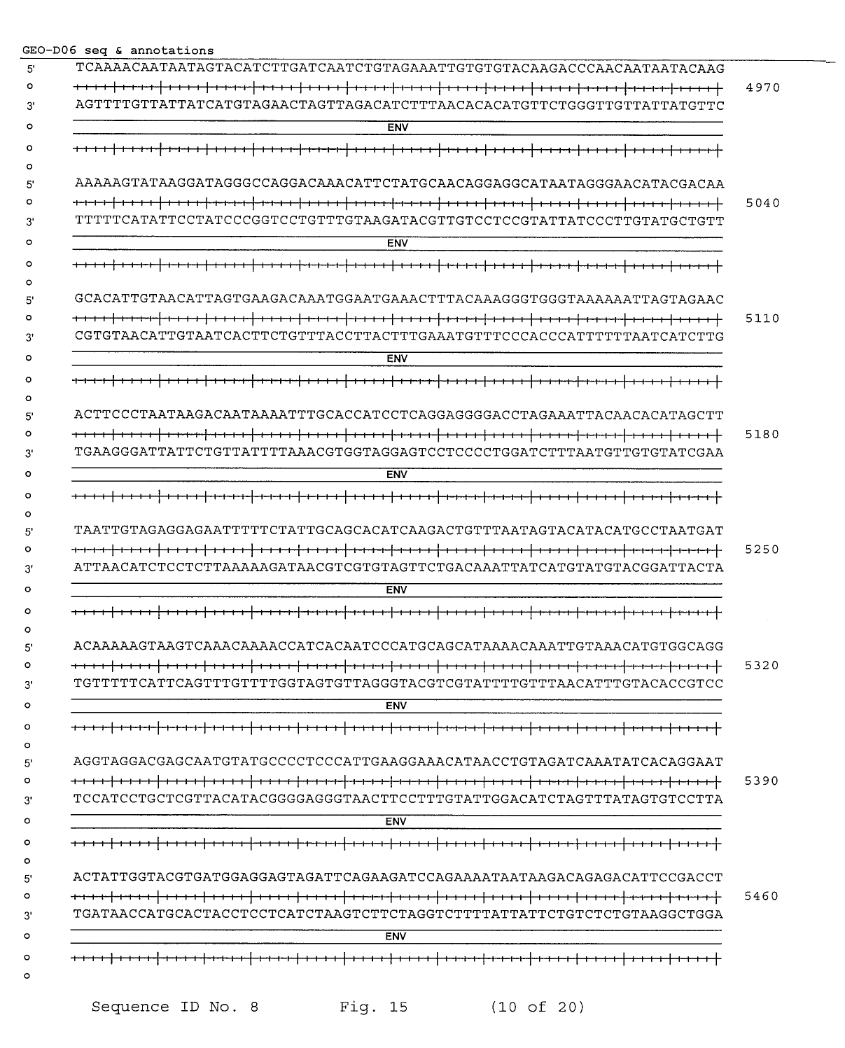

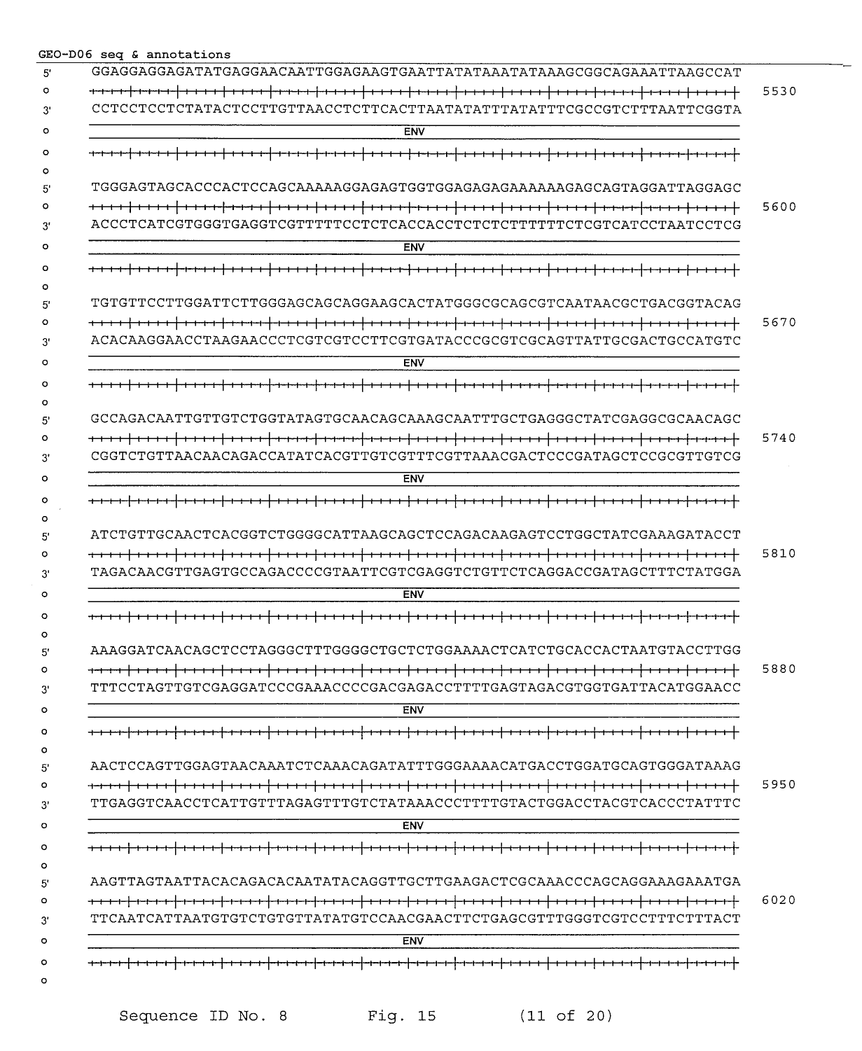

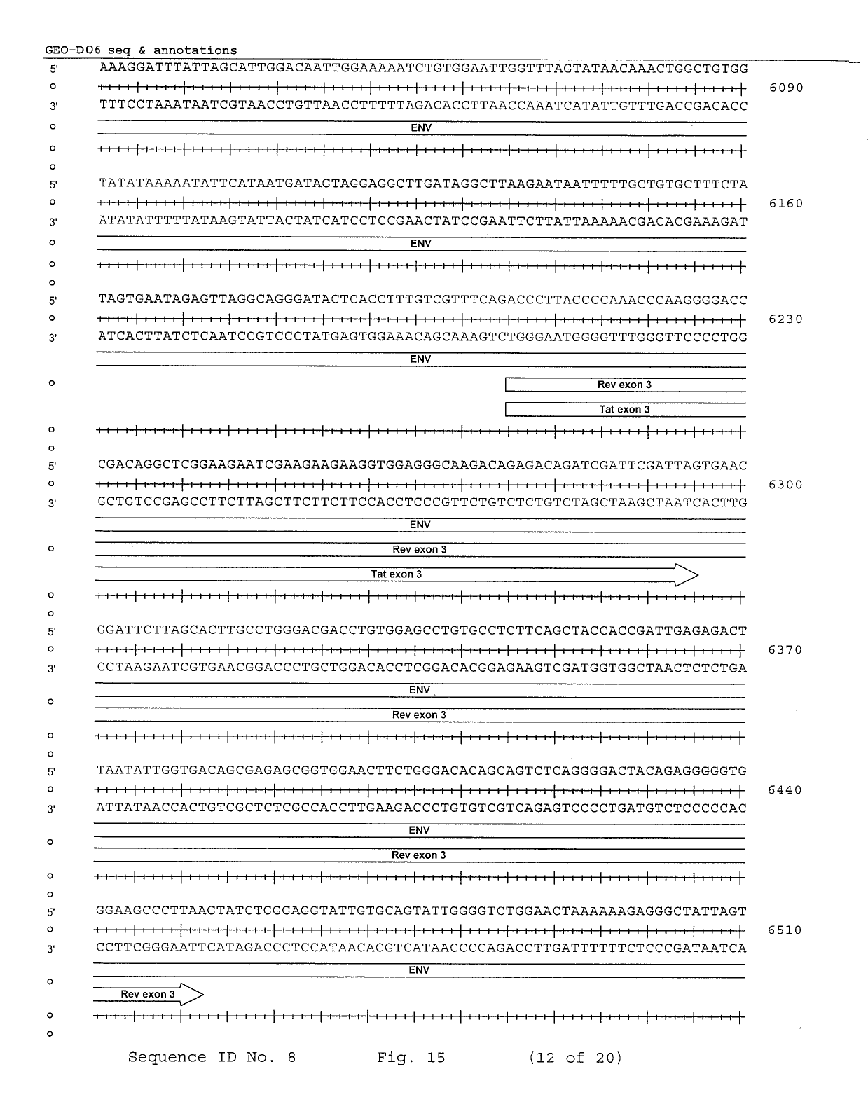

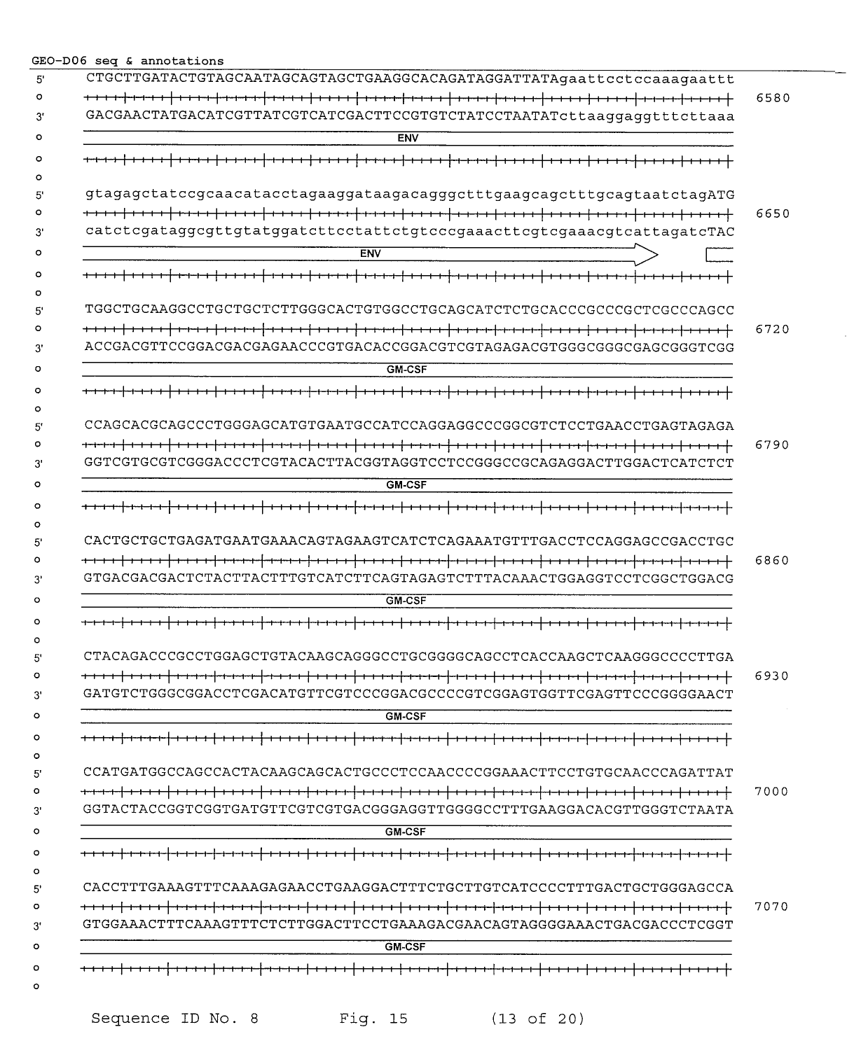

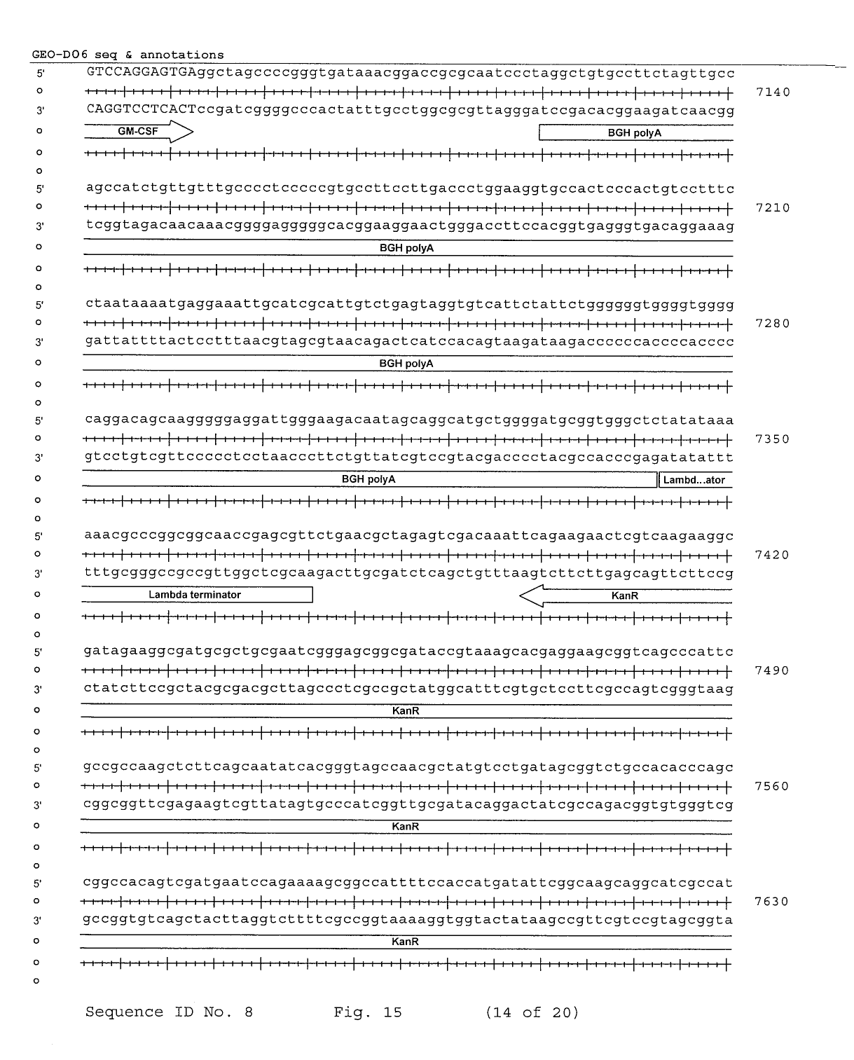

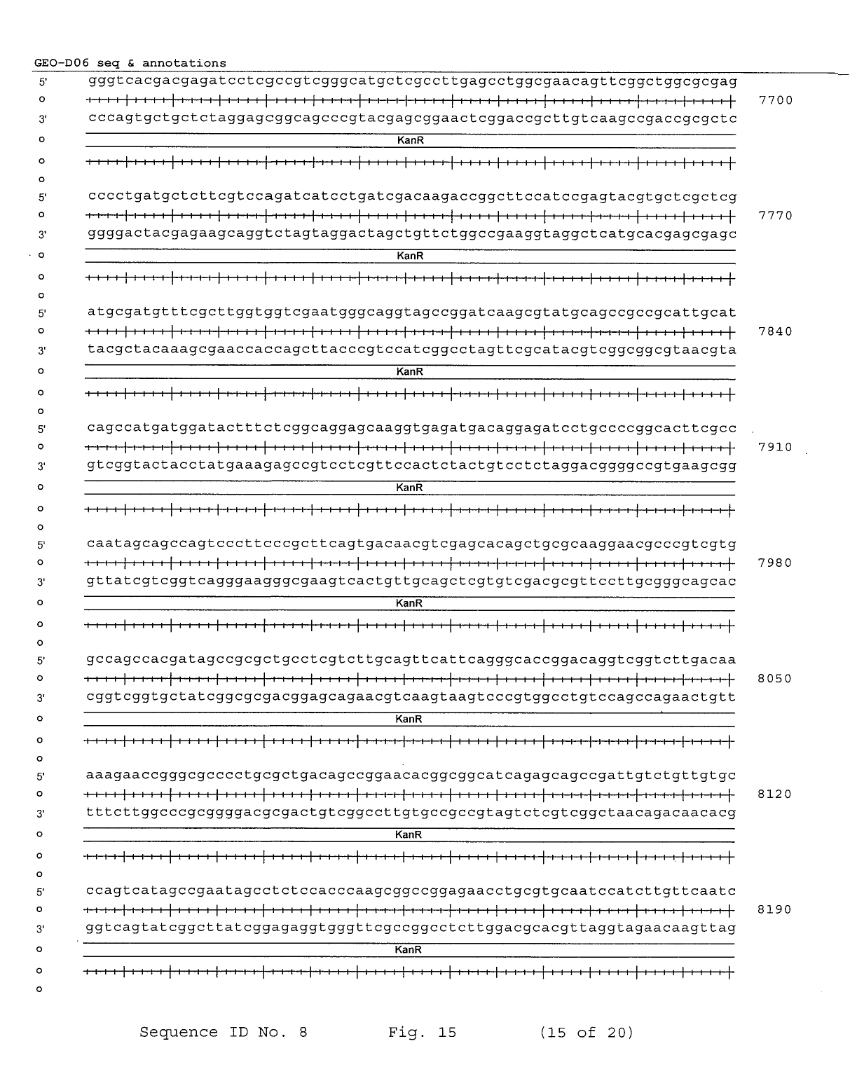

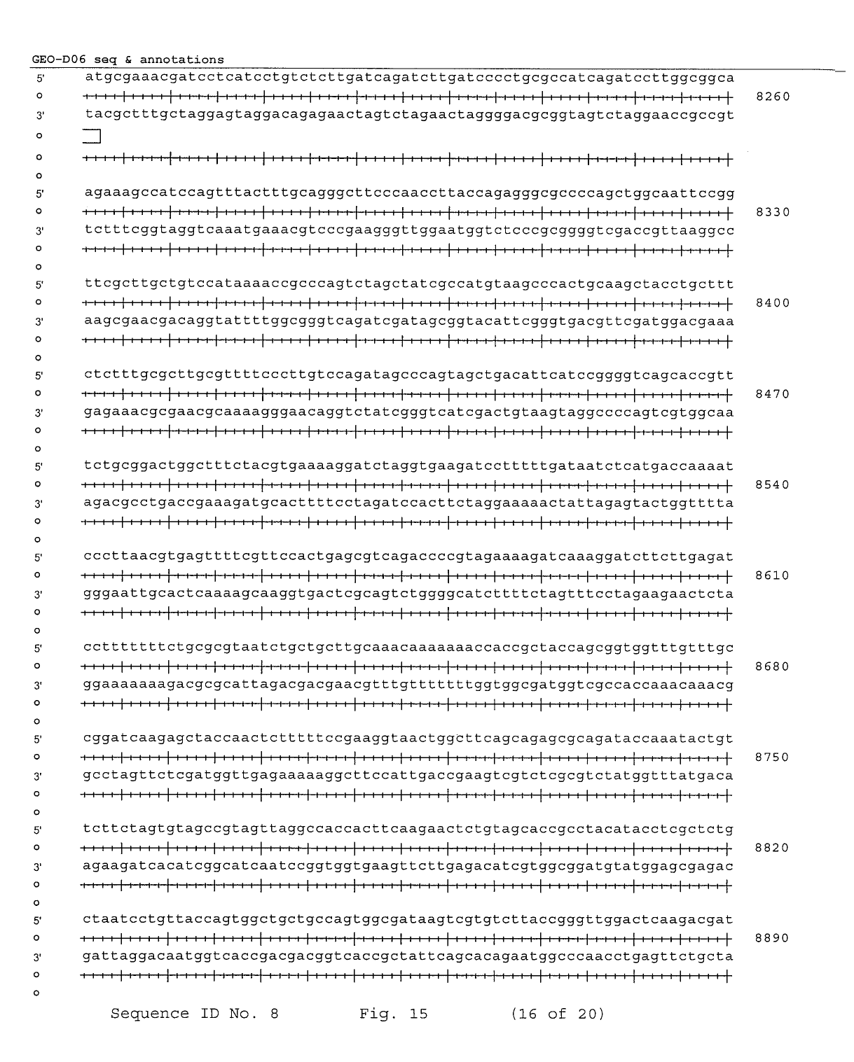

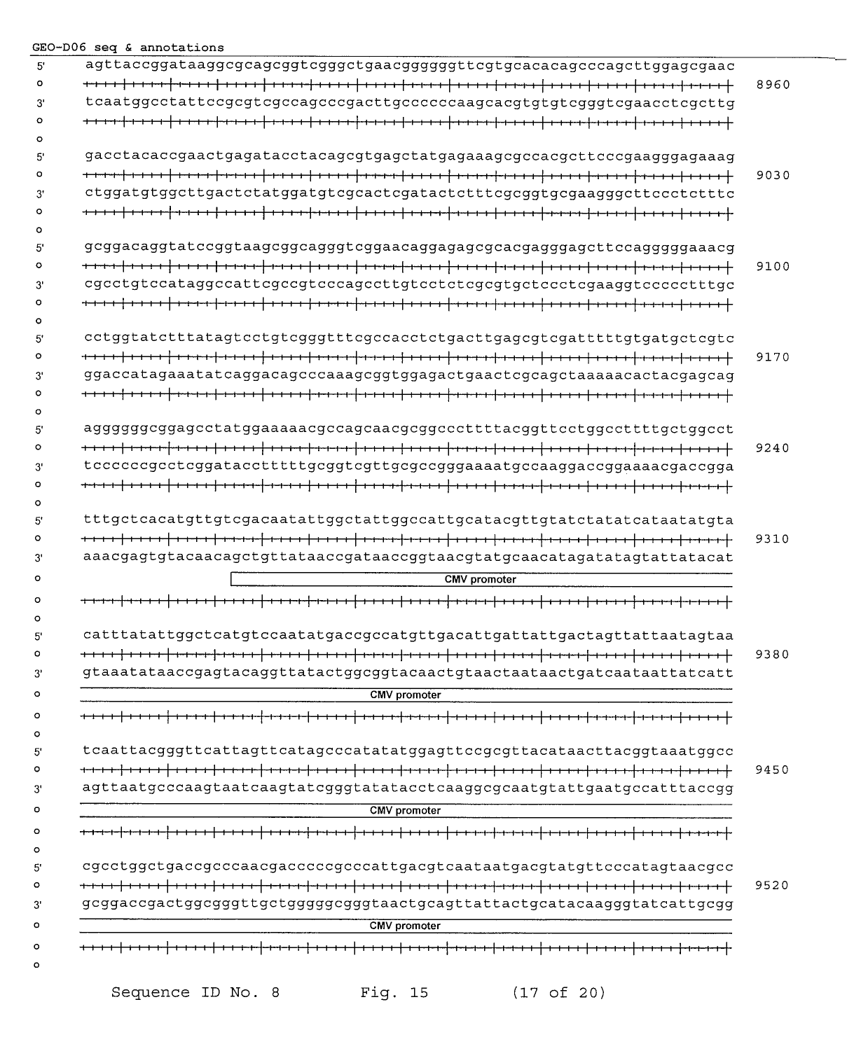

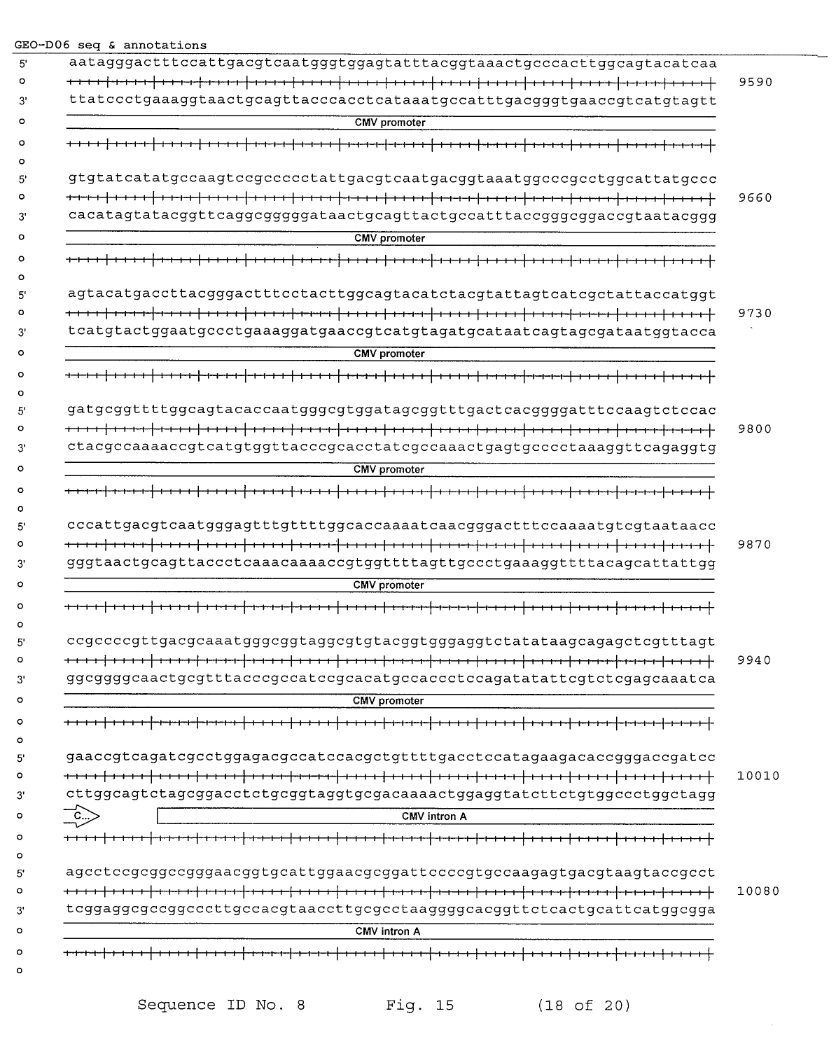

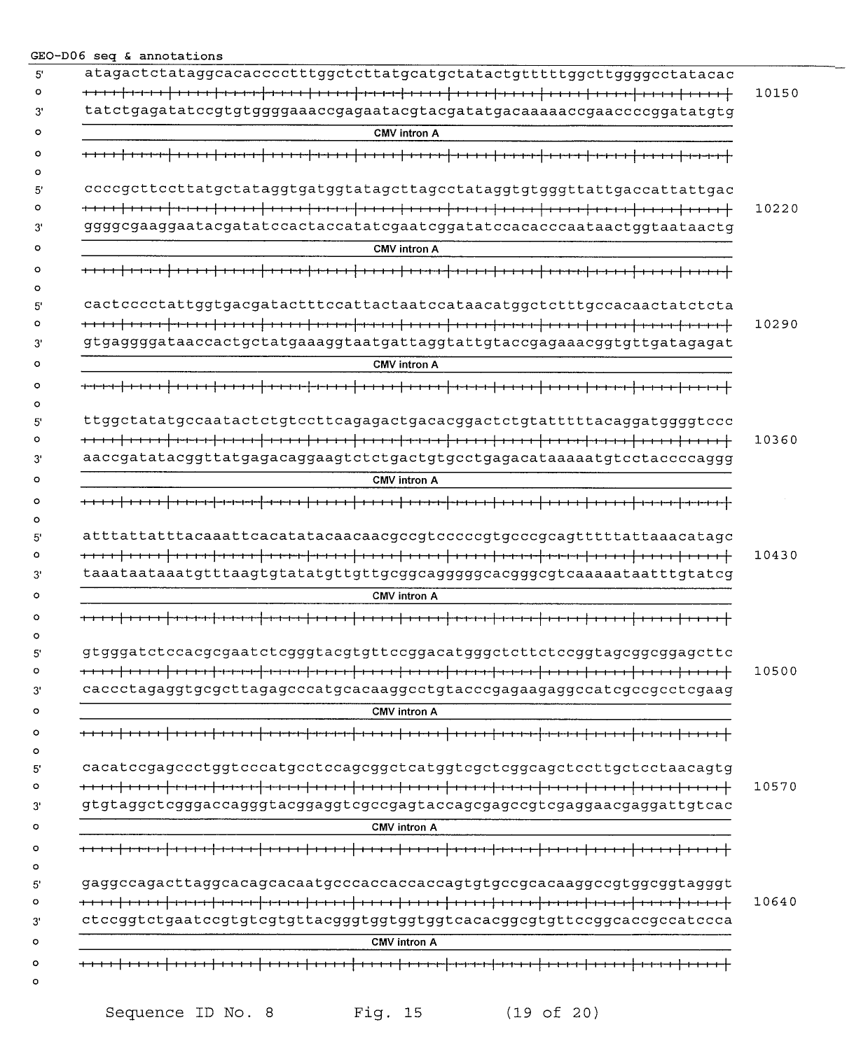

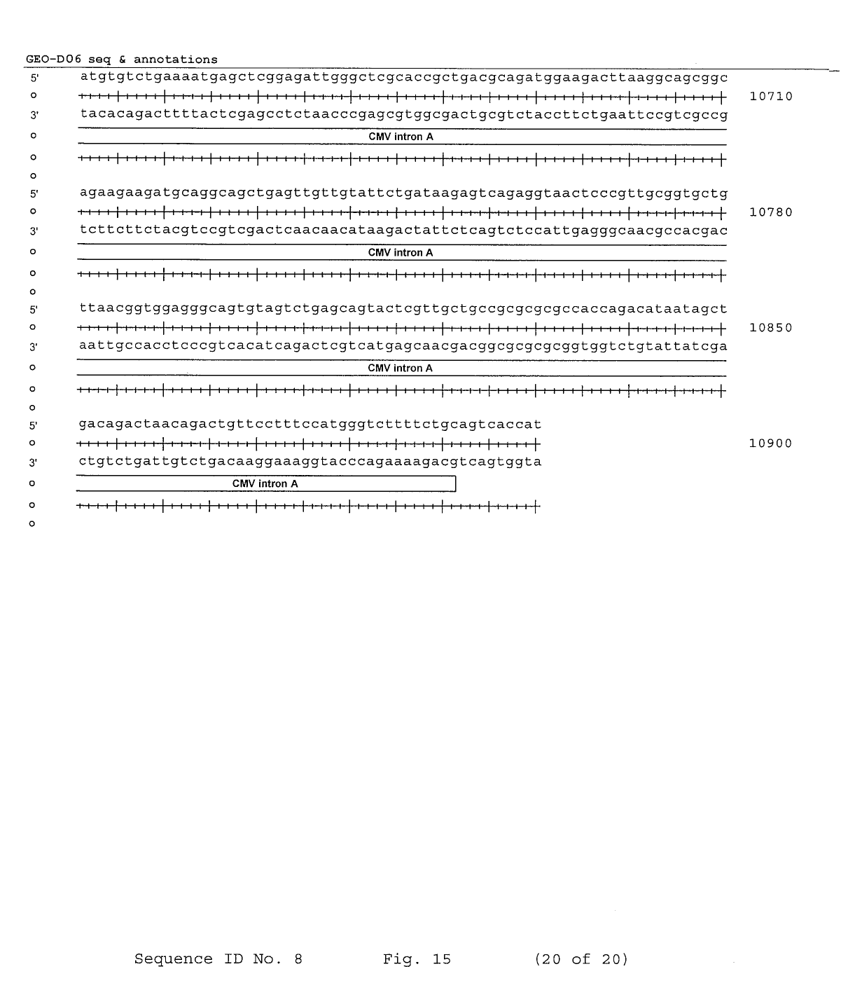

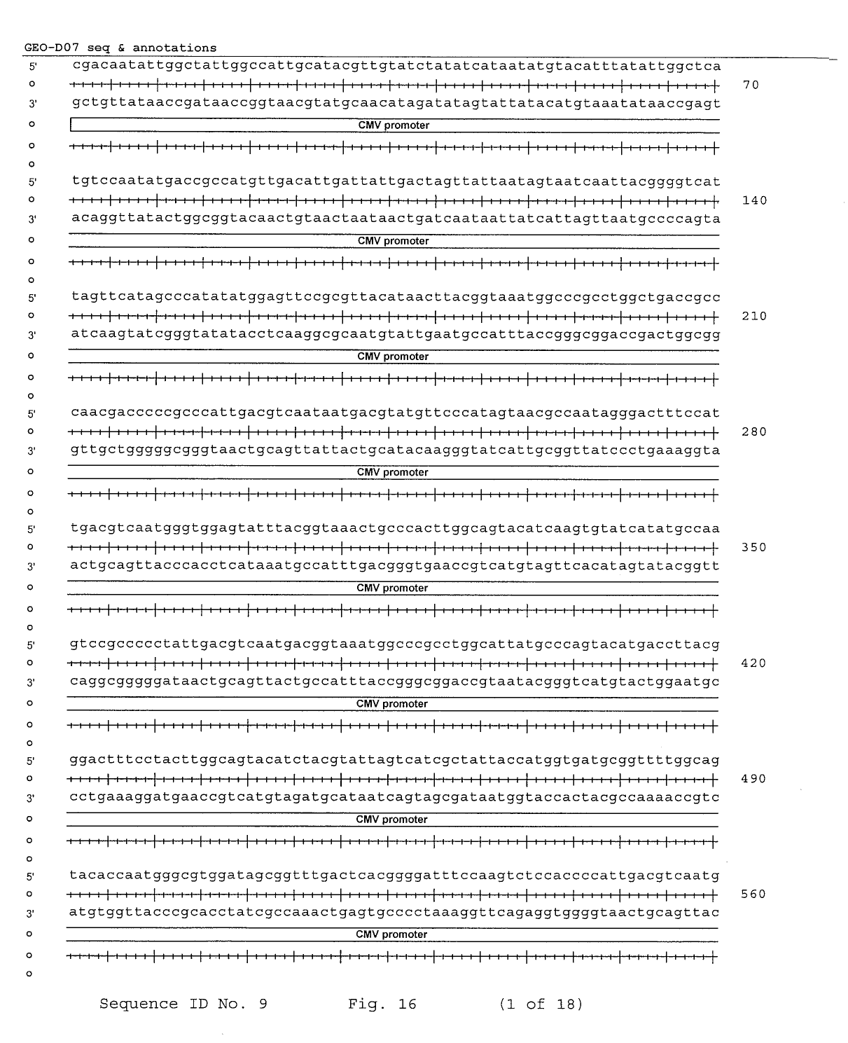

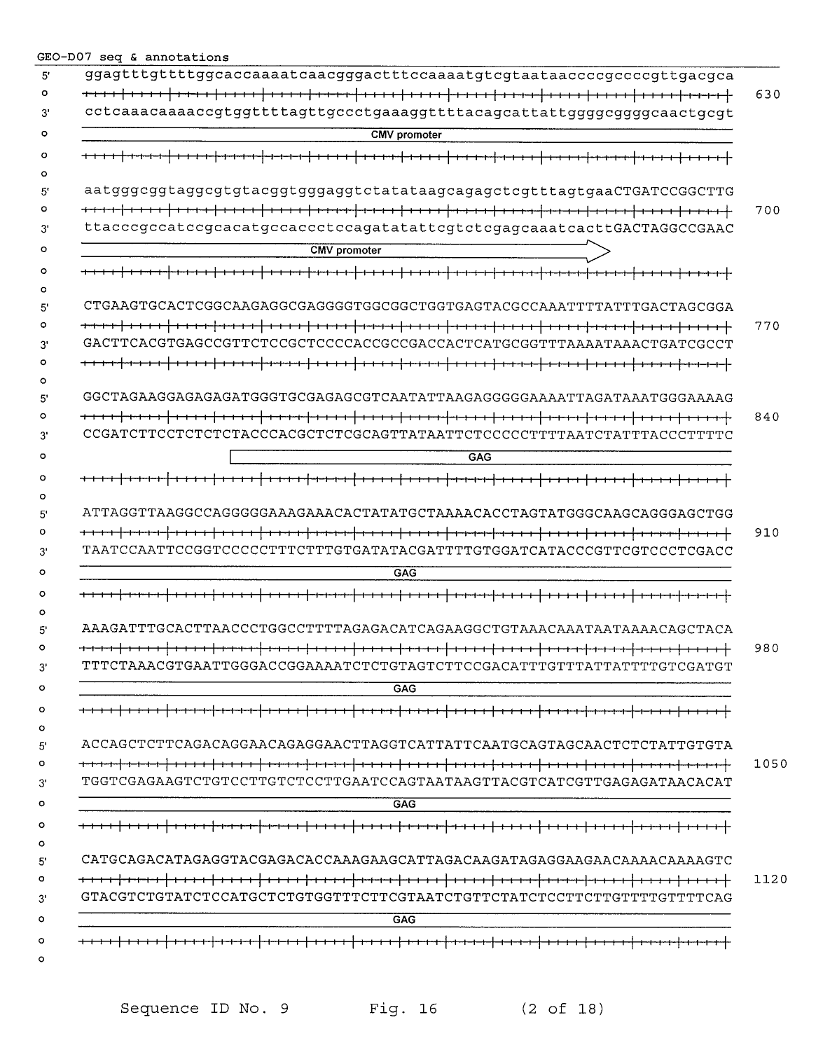

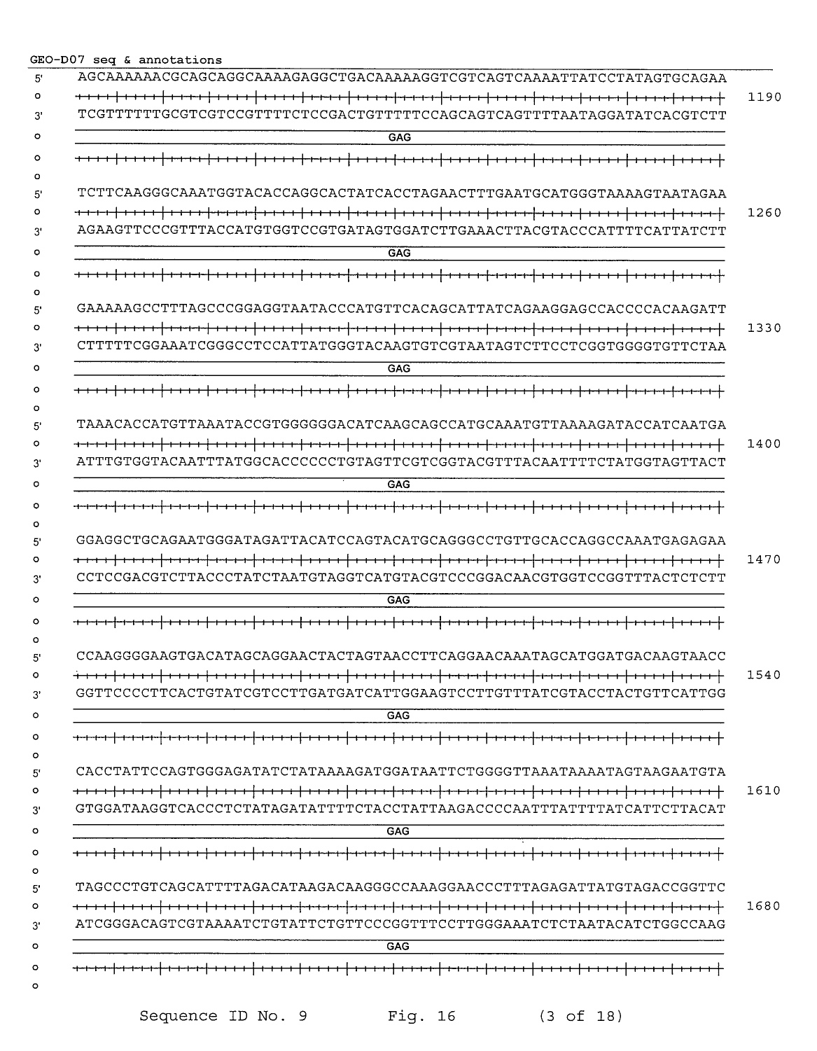

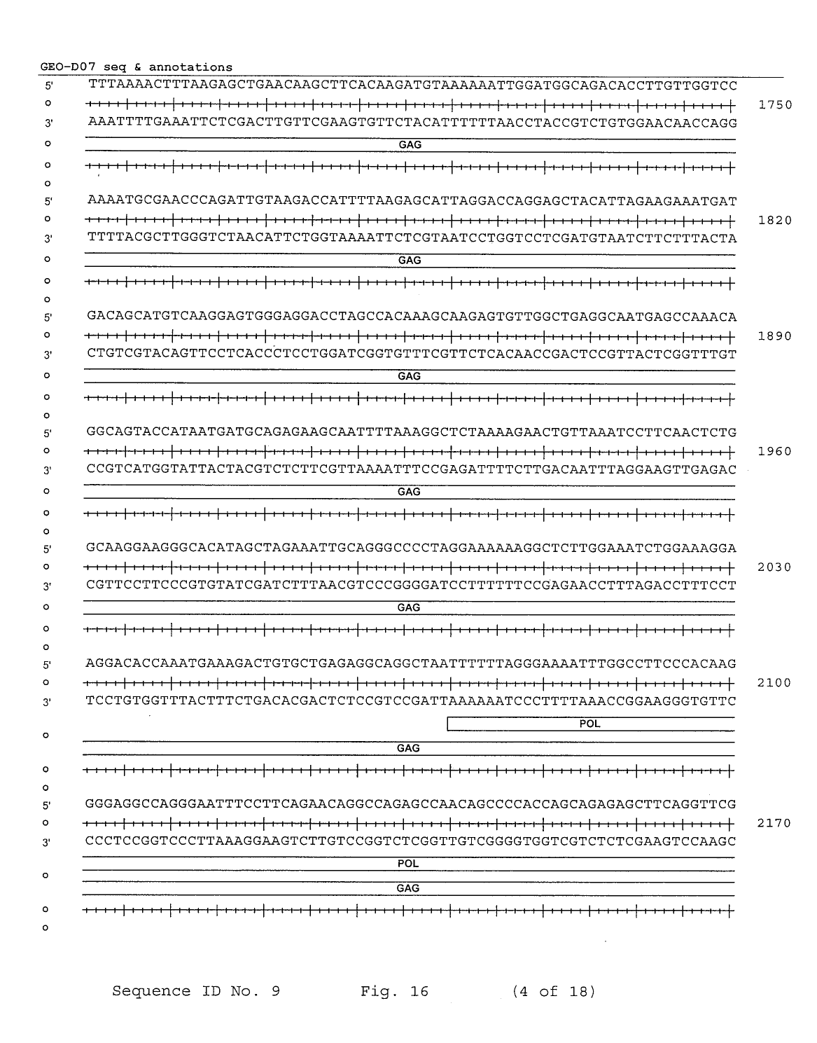

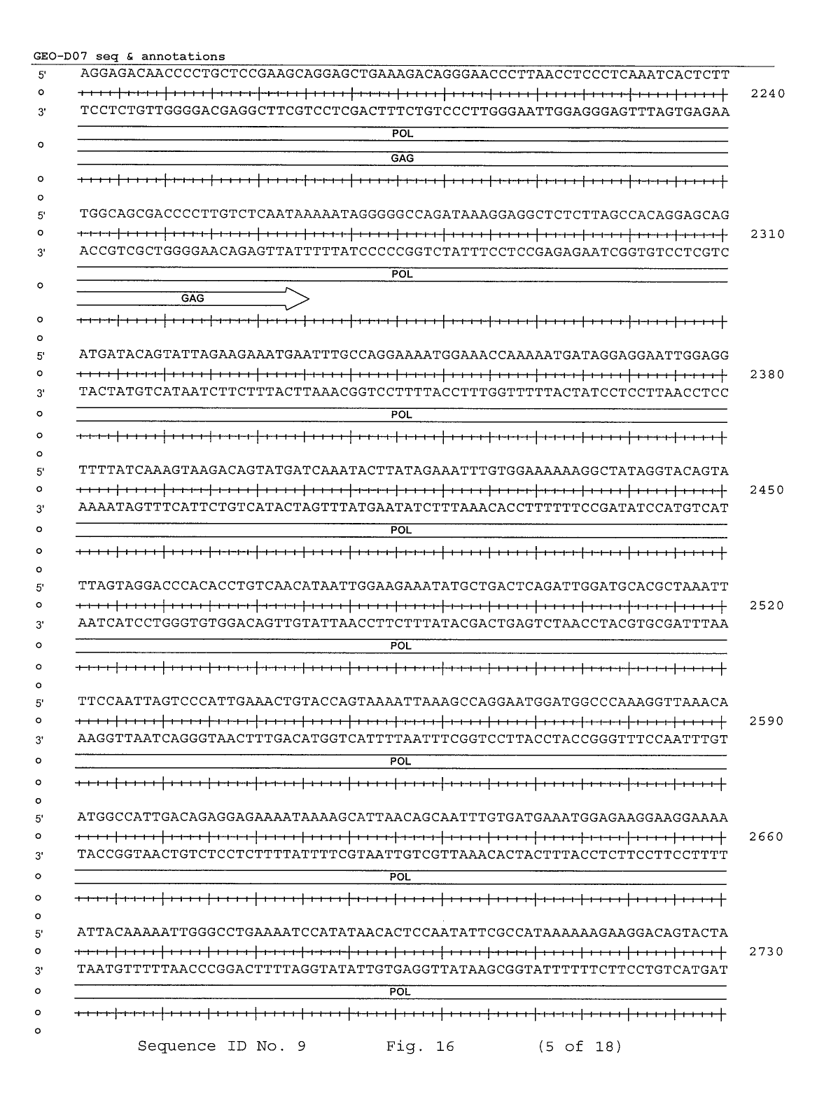

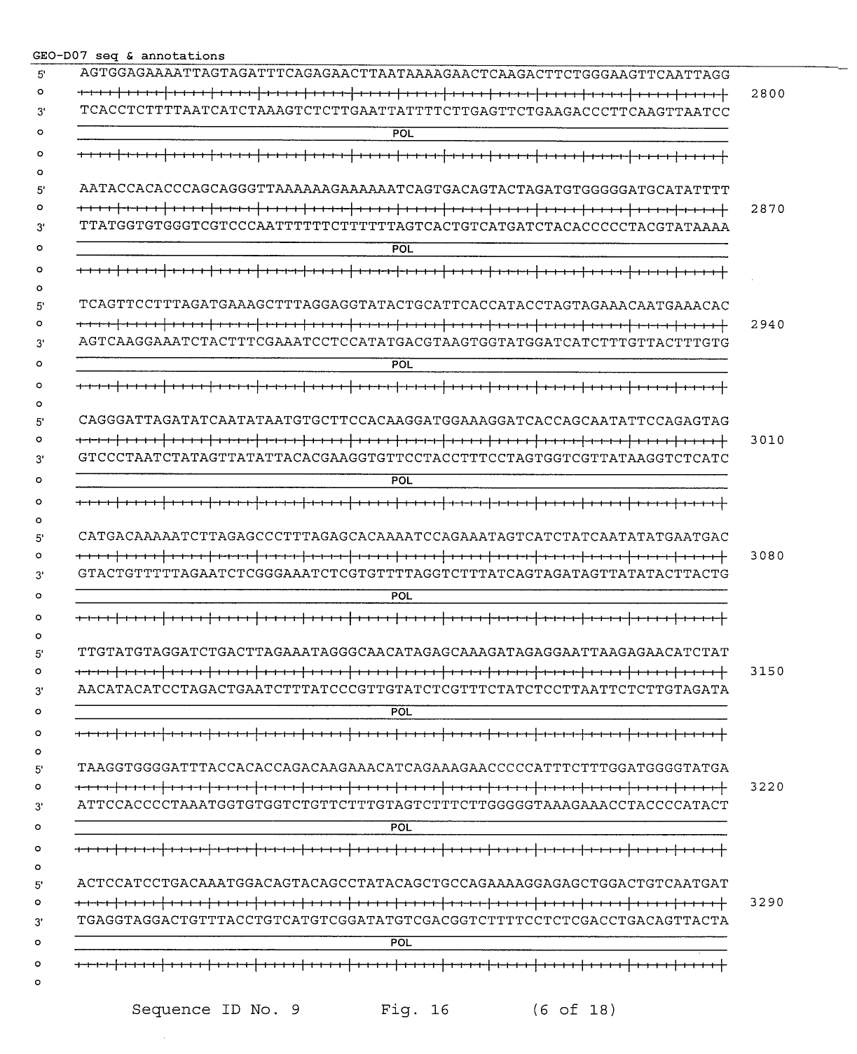

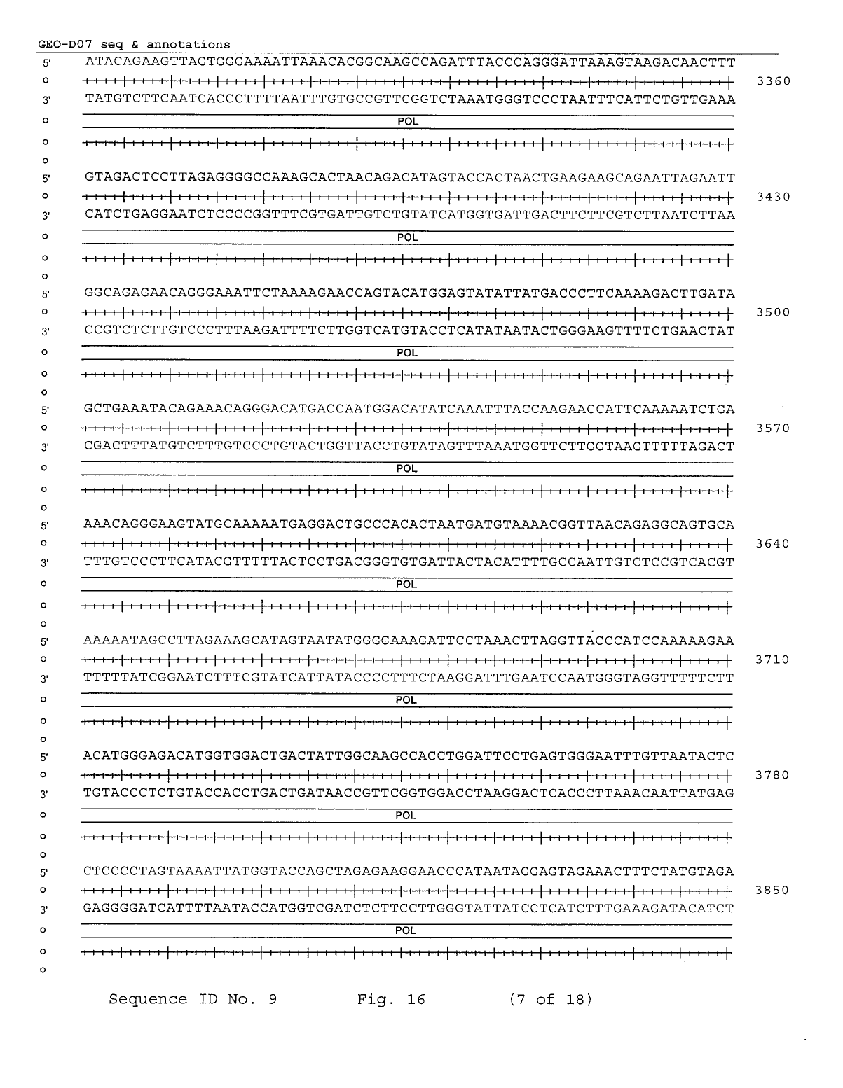

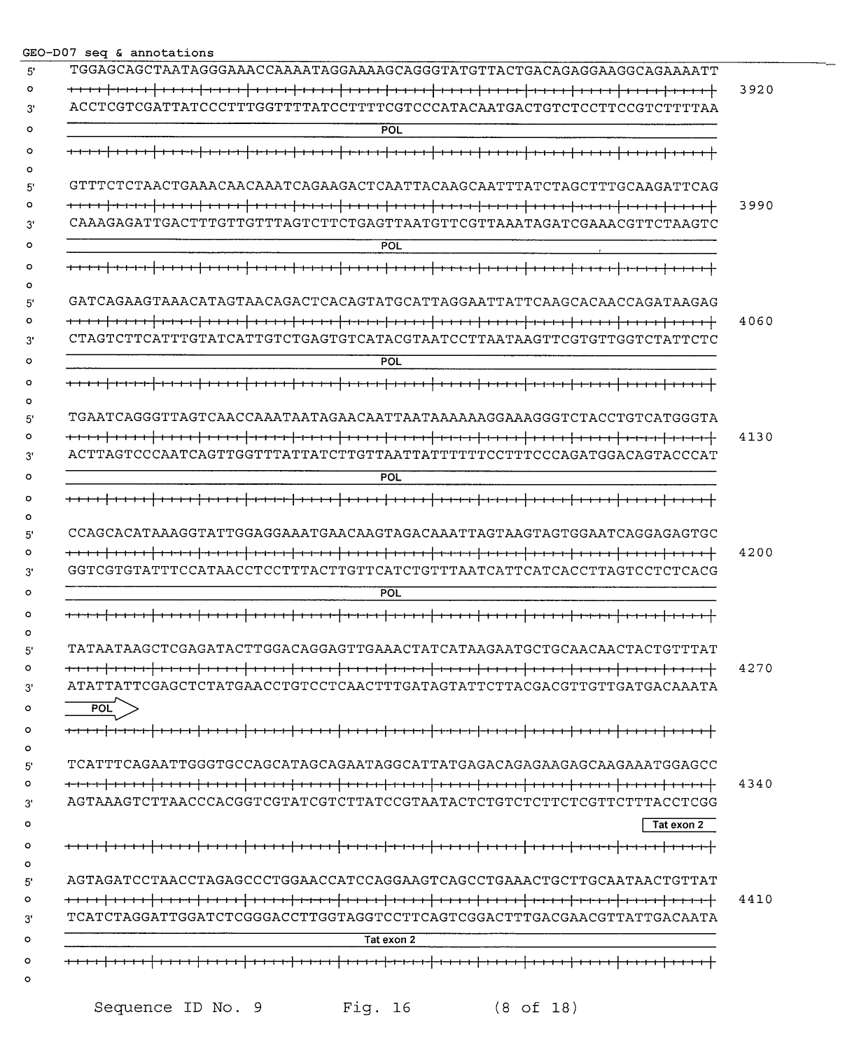

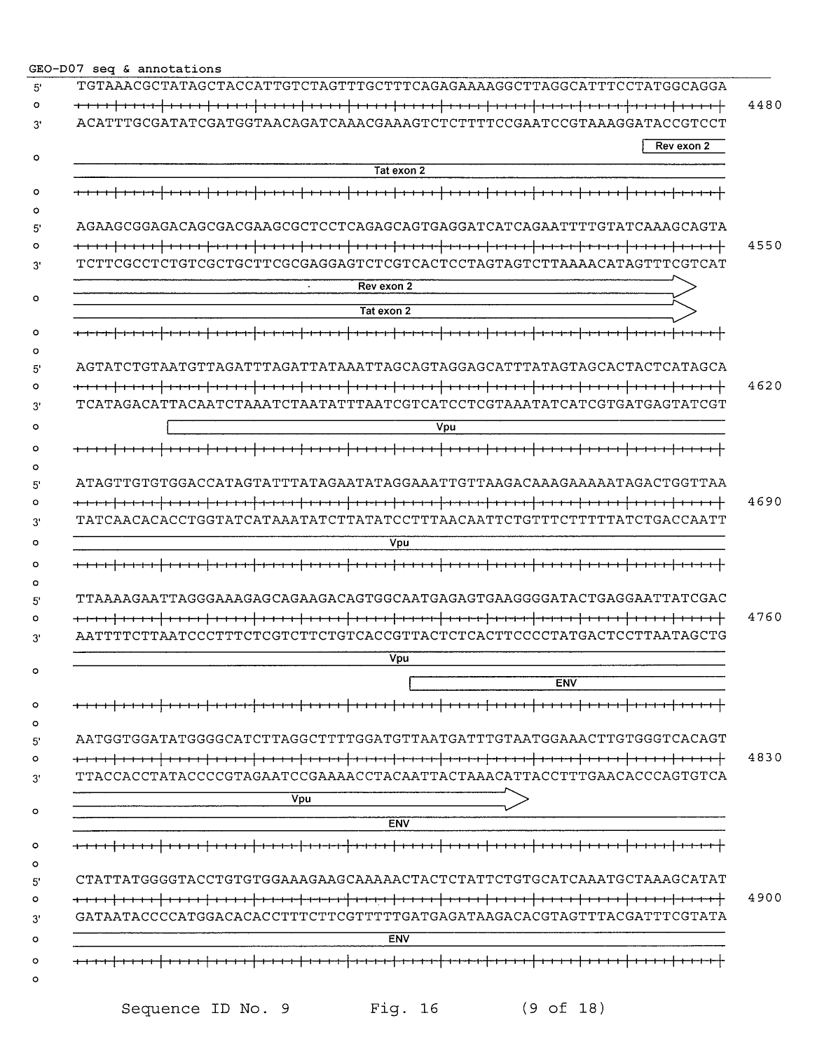

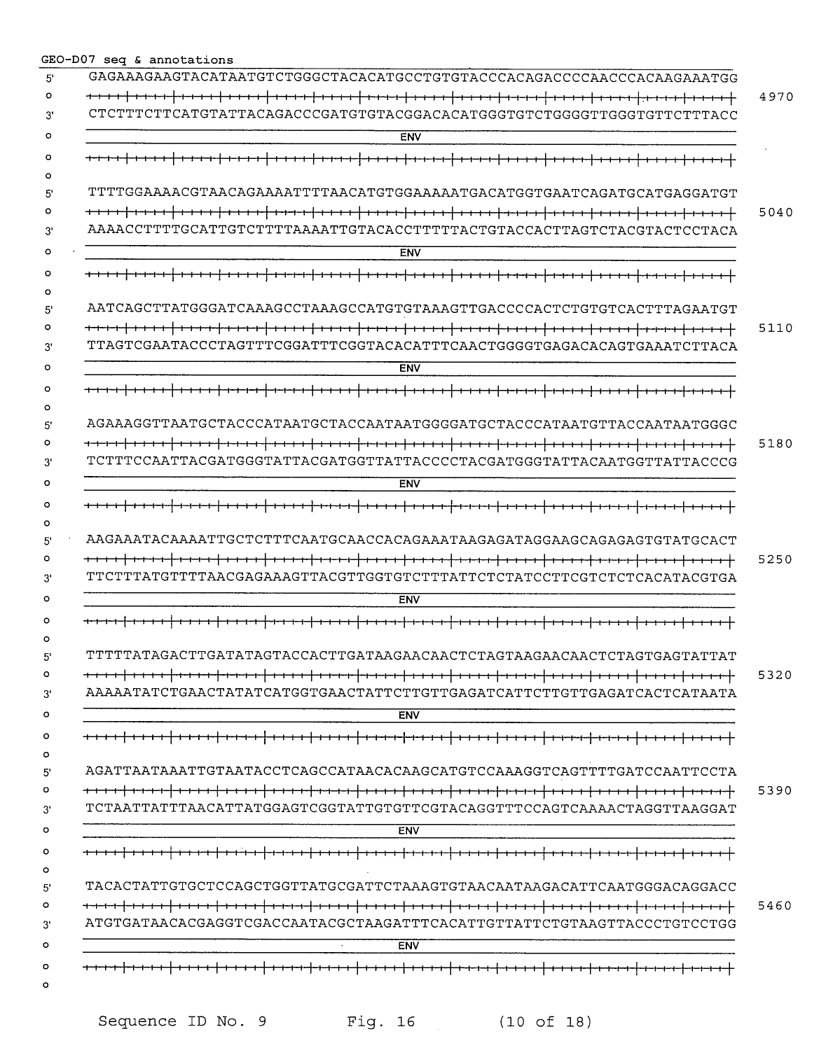

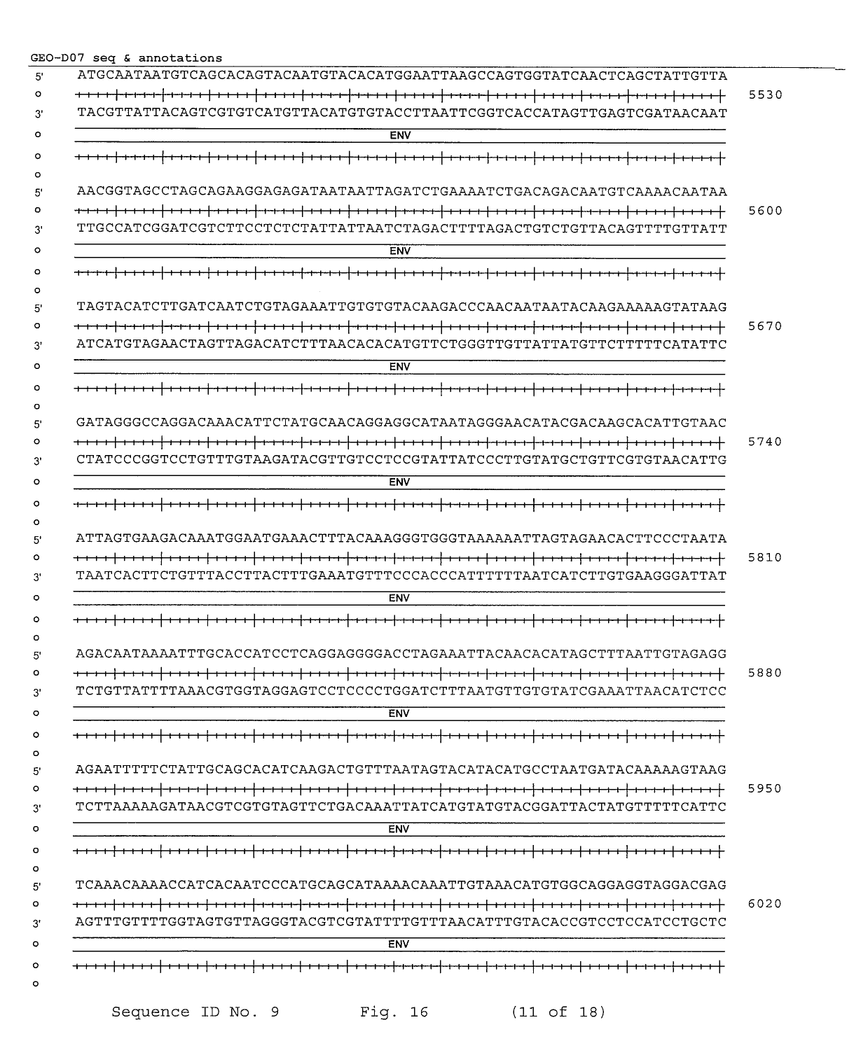

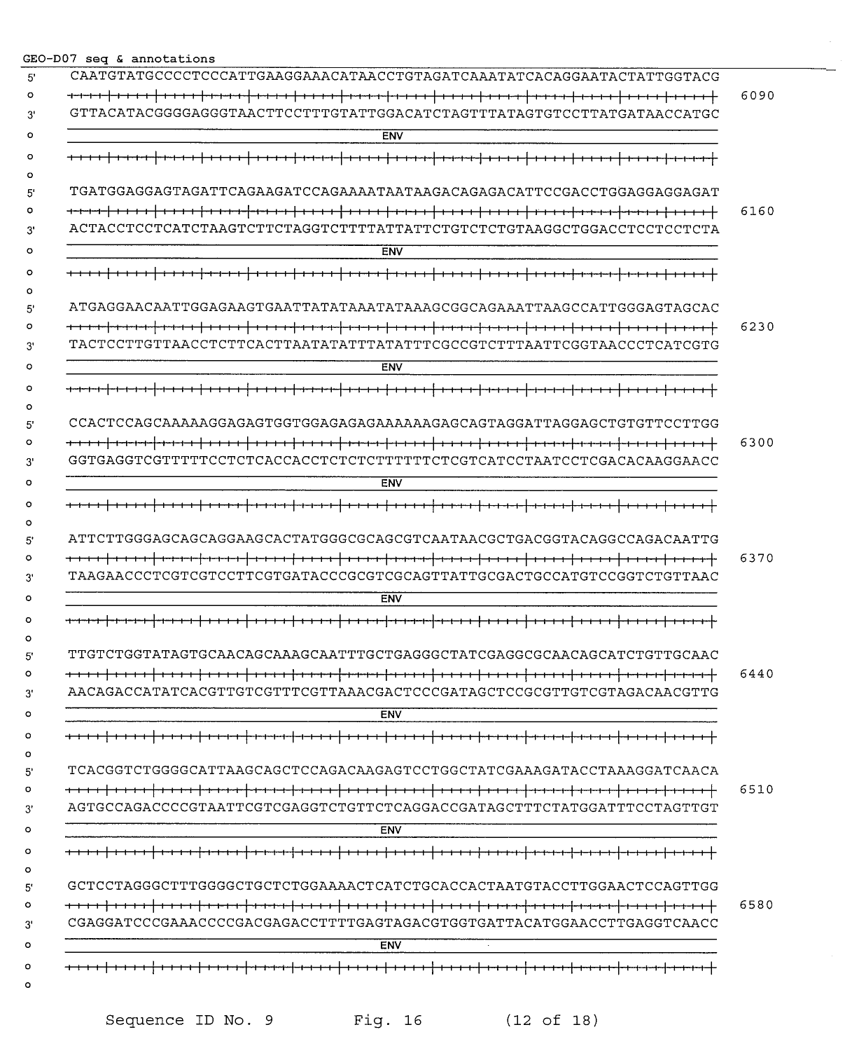

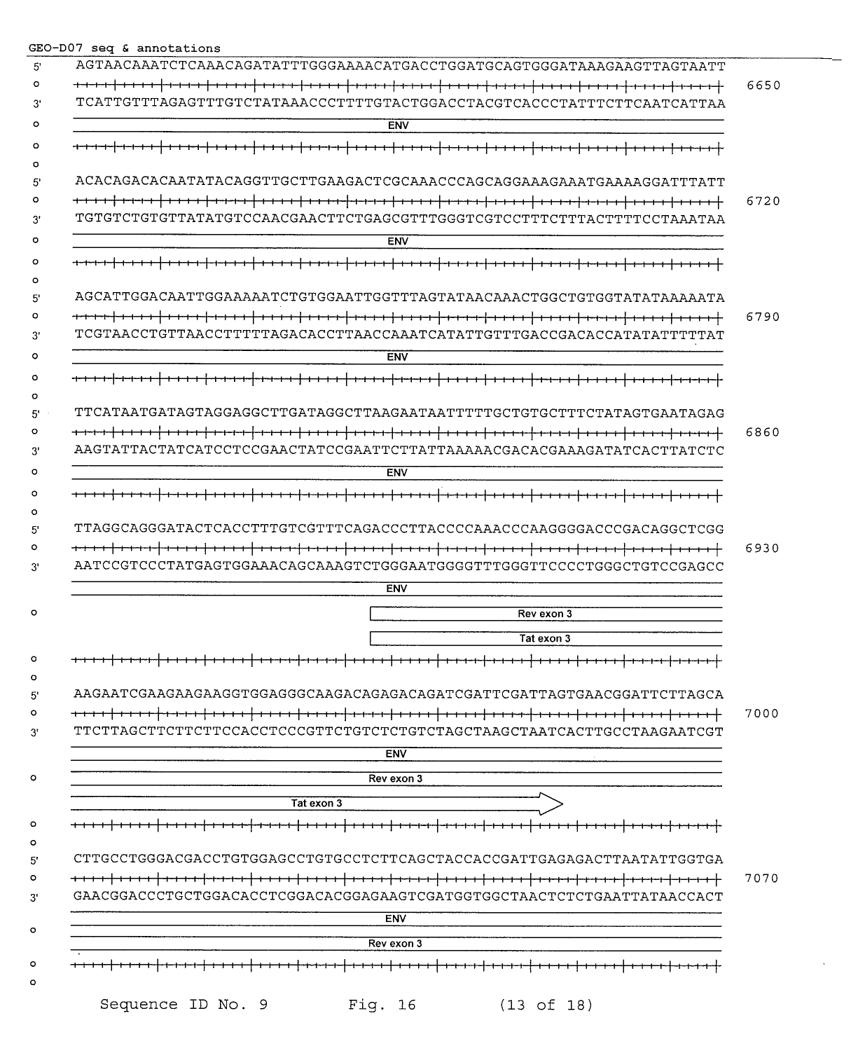

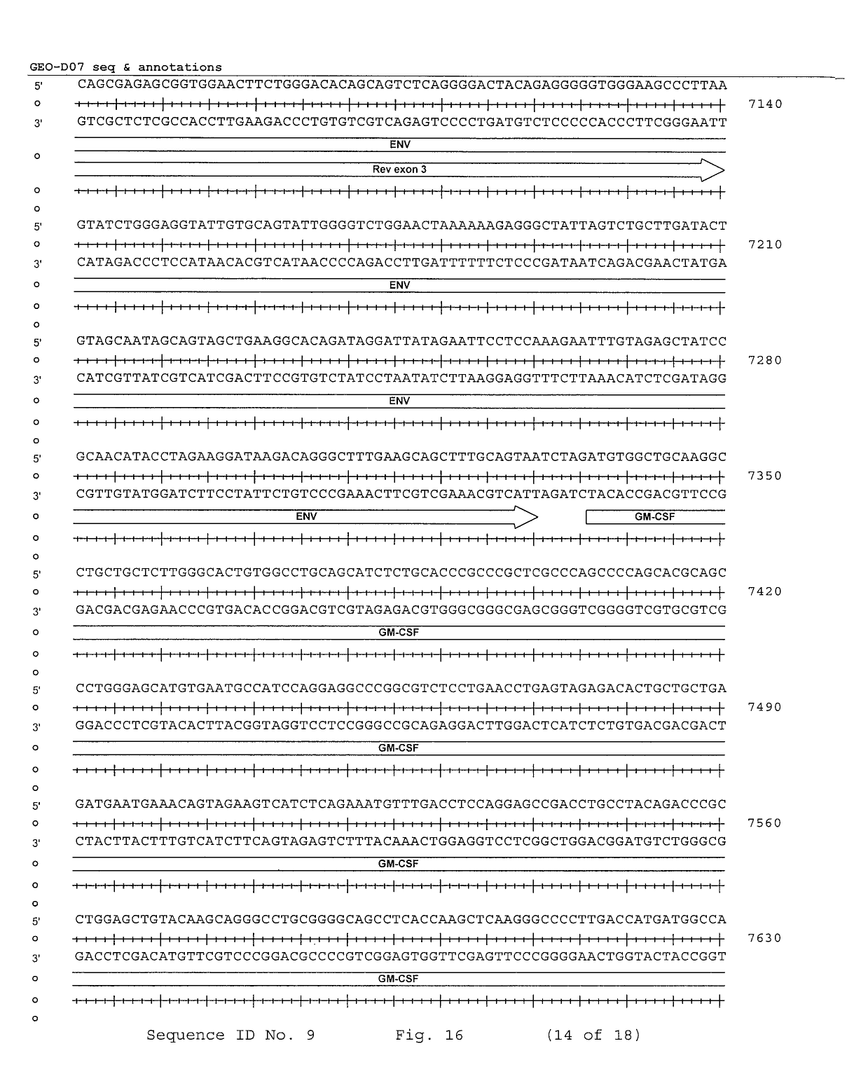

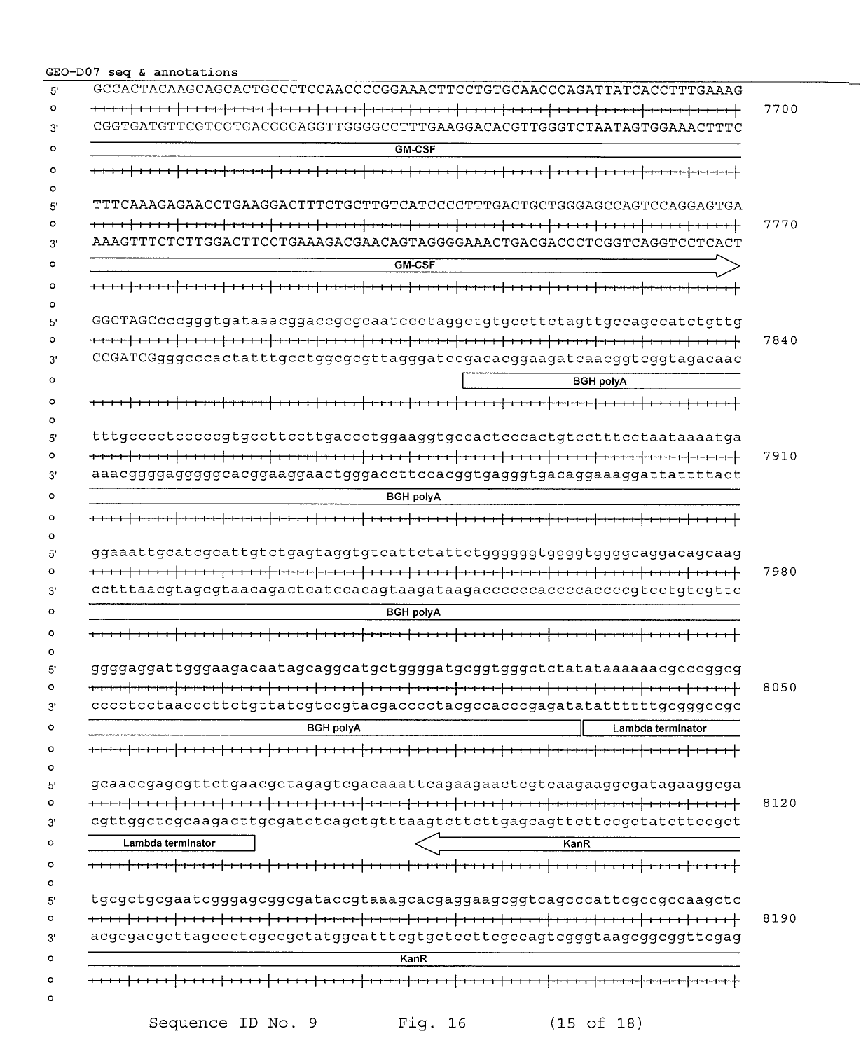

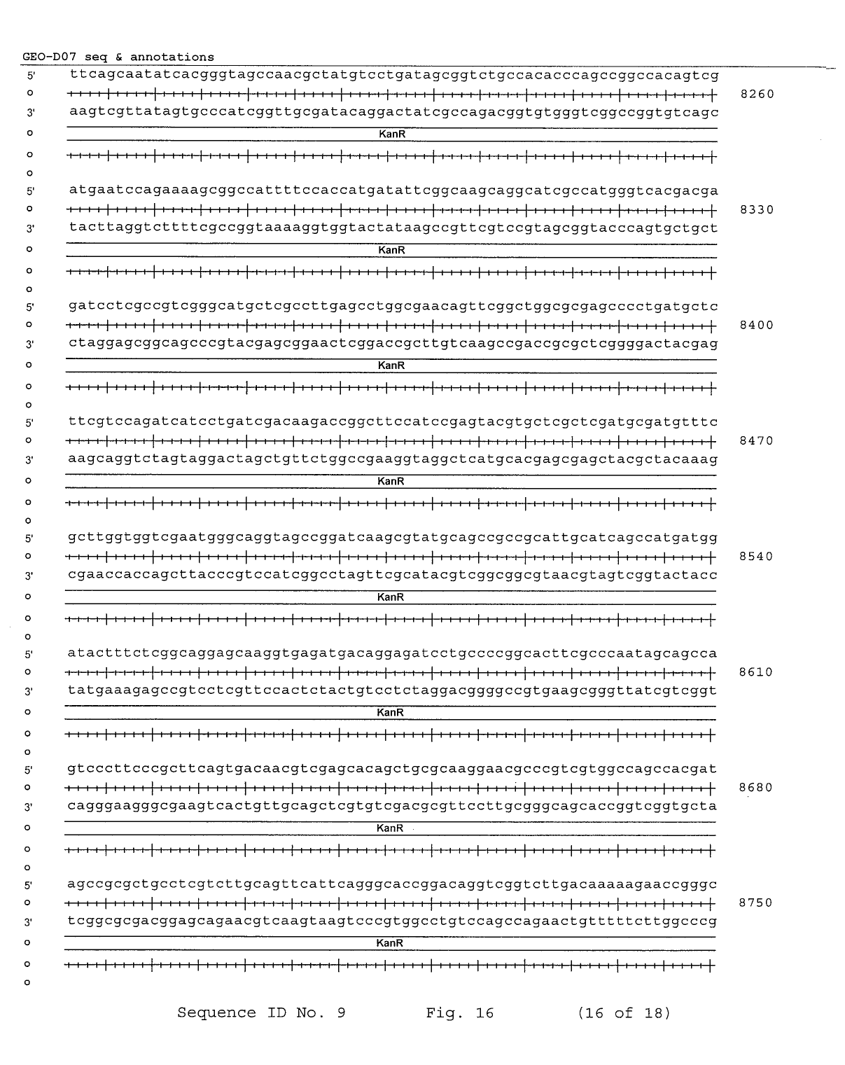

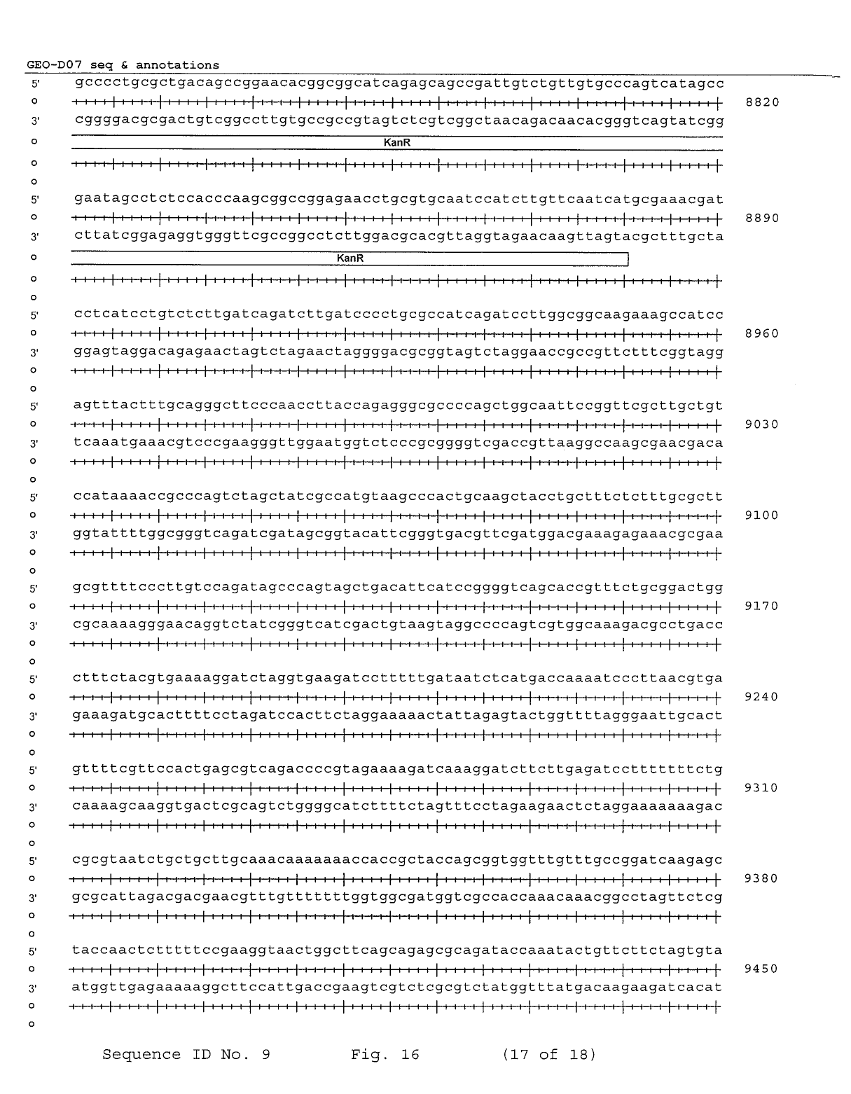

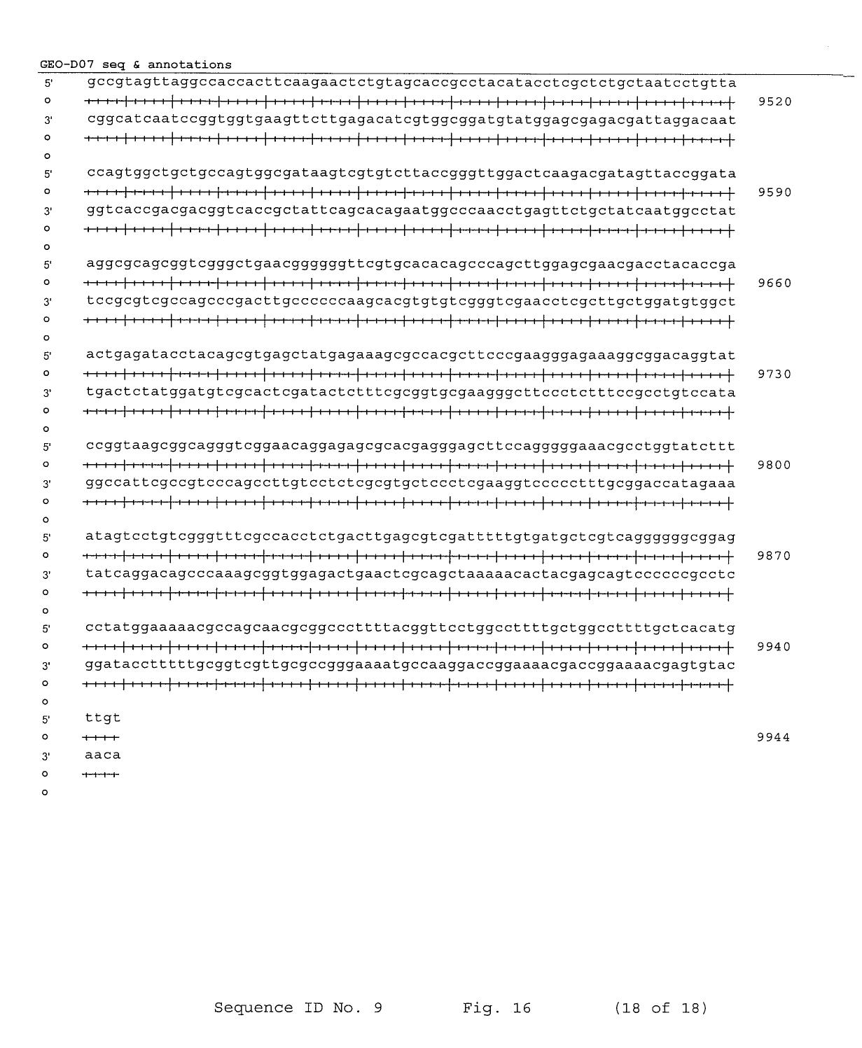

GEO-D03 was developed from the pGA2/JS7 (J57) plasmid DNA vaccine that was administered to normal volunteers in HVTN 065 and 205 under BB-IND 12930. GEO-D03 differs from JS7 by the insertion of a 435 base pair open reading frame for human GM-CSF in the position of a deleted nef sequence (FIG. 14; SEQ ID NO: 7). J57 is a 9.5 kb plasmid DNA composed of a 2.9 kb expression vector named pGA2 and a 6.6 kb vaccine insert expressing multiple HIV-1 clade B proteins from a single transcript that undergoes subgenomic splicing1. The vaccine insert expresses Protease (PR) and Reverse Transcriptase (RT) sequences of the BH10 strain of HIV-1; tat, rev, vpu, and env from a recombinant of HXB-2 and ADA HIV-1 sequences; and gag from HIV-1 HXB-2. The vaccine is rendered non-infectious by deletion of the long terminal repeat (LTR), vif, vpr, and nef and the region of pol encoding integrase; and by the introduction of inactivating point mutations into packaging sequences for viral RNA and the protease, reverse transcriptase, strand transfer and RNase H domains of Pol. Addition of GM-CSF was achieved through insertion of a synthetic gene using standard recombinant DNA technology. With the addition of the GM-CSF gene, the size of the new plasmid (GEO-D03) is 9.9 kb. With the exception of the HIV-1 sequences, there are no known viral or oncogenic protein coding sequences within the GEO-D03 plasmid DNA. In transient transfections in 293T cells, GEO-D03 expresses approximately 200 ng of human GM-CSF per 106 cells per 24 hours.

MVA/HIV62B (MVA62B) is a highly attenuated vaccinia virus expressing HIV-1 gag, pol, and env genes from the same sequences used to construct the JS7 DNA. Mayr and colleagues first produced MVA in Germany in 1975 as a smallpox vaccine for individuals considered to be poor risks for the standard vaccinia inoculation-2,3.

MVA originated from the dermovaccinia strain chorioallantois vaccinia Ankara (CVA) that was retained for many years at the Ankara Vaccination Station via donkey-calf-donkey passages. In 1953, Mayr and colleagues purified CVA and passaged it twice through cattle. In 1954/55, this purified product was used in the Federal Republic of Germany as a smallpox vaccine. In 1958, attenuation experiments with CVA were begun by terminal dilutions in chick embryo fibroblasts (CEF). After 360 passages, the virus was cloned by 3 successive plaque purifications and maintained in CEF to 570 passages. After 570 passages, the virus was plaque purified on cells from a recognized leucosis-free flock of chickens.

In the process of serial passages in CEF, 9% of the DNA was lost from the original CVA strain and the virulence for mammalian cells was greatly reduced. In particular, the resulting MVA strain undergoes an abortive infection in human cells 2-4. After 516 passages, the virus was called “modified vaccinia virus Ankara” and was given to the German State Institution, Bayerische Landesimpfanstalt, where human clinical trials with a dose as high as 2×106 pfu were conducted.

A sample of freeze dried MVA virus from the 572nd passage in primary CEF which had been harvested on Feb. 22, 1974, was sent directly from Dr. Mayr in Germany to Dr. Bernard Moss at NIAID in August 2001. The reconstituted virus was plaque purified 3 times by terminal dilutions in CEF (made from 10-day-old specific pathogen free [SPF] fertile chicken eggs, distributed by B and E Egg Company, York Springs, Pa.) using certified reagents including gamma irradiated fetal calf serum (from sources free of bovine spongiform encephalopathy) and trypsin. Sterility and mycoplasma tests were done and were negative. This MVA virus was used to prepare the current recombinant MVA/HIV62 construct.

MVA/HIV62 was constructed by introducing a Gag-Pol expression cassette into deletion III of MVA and an Env expression cassette into deletion 115. Both expression cassettes use the mH5 early/late promoter for expression of vaccine inserts. The pol gene in MVA/HIV62 contains the same mutations as found in the JS7 DNA vaccine with the exception of not including the inactivating point mutation in PR. The Env expression cassette contains an upstream start codon that has the potential for expressing a 33 amino acid fusion protein comprised of 7 amino acid residues encoded by a multiple cloning site and the 26 C-terminal amino acids of Vpu. The upstream start codon attenuates the expression of Env. The sequences in the fusion protein have no matches in the genome database for the 7 amino acid sequence and its fusion outside of the known Vpu match.

The MVA62 was manufactured in SPF CEF and is formulated in a buffer consisting of PBS and 7.5% sucrose. The placebo for both the DNA and MVA vaccines is Sodium Chloride for Injection USP, 0.9%.

Primary endpoint 1 is to determine the frequency of severe local (pain, tenderness, erythema, induration, and maximum severity) and systemic (fever, malaise/fatigue, myalgia, headache, nausea, vomiting, chills, arthralgia, and maximum severity) reactogenicity within the 1st 72 hours of vaccination. Primary endpoint 2 is the distribution of local laboratory values using boxplots by treatment group. Primary endpoint 3 is the frequency of all other adverse events by treatment arm throughout the trial.

Secondary endpoint 1 is to assess HIV-1 specific anti-Env antibody responses at 2 weeks post the last MVA boost: the frequency and titer of HIV binding Ab for ADA gp140 and the frequency and titer of neutralizing antibody assays for HIV-1-MN and the breadth of neutralizing Ab for tier 1 and tier 2 isolates. Secondary endpoint 2 is to evaluate HIV-1 specific CD4+ and CD8+ T cell responses: the frequency of CD4+ T cell responses measured by IFN-γ and/or IL-2, at two weeks after the last MVA vaccination to HIV peptides representing Gag, Pol and Env proteins expressed by the HIV-1 immunogens; and the frequency of CD8+ T cell responses measured by IFN-γ and/or IL-2, at two weeks after the last MVA vaccination to HIV peptides representing Gag, Pol and Env proteins expressed by the HIV immunogens.

Exploratory objective 1 will assess safety by testing for the elicitation of anti-GM-CSF Ab by the DNA vaccine. Exploratory endpoint 1 will determine the frequency and the titer of anti-GM-CSF Ab at 2 weeks after the 2nd GEO-D03 vaccination.

Exploratory Objective 2 will assess the avidity of Env-specific anti-Env elicited binding Ab. Exploratory endpoint 2 will determine the avidity index of Env-specific anti-Env binding Ab at 2 weeks after the 3rd MVA inoculation using biacore analyses (conducted at Duke) and duplicate ELISAs treated with either a phosphate-buffered saline or a sodium thiocyanate wash.

Exploratory objective 3 will assess the frequency of vaccine-induced positive results with end of study HIV serological testing by commercial assays. Exploratory Endpoint 3 will determine the frequency of HIV-positive Ab responses using commercial Ab and where appropriate western blot testing.

Exploratory Objective 4 will test for the presence of GM-CSF in blood at 3,5,7 and 14 days post each DNA immunization. Exploratory Endpoint 4 will determine the titers of GM-CSF in blood at preimmunization, 3, 5, and 7 days post each DNA immunization.

Exploratory Objective 5 will assess the production of Th1 and Th2 cytokines by responding T cells using luminex assays. Exploratory Endpoint 5 will determine the titers of IFN-γ, IL-2, TNF-α, IL-4, IL10 and IL-13 produced by peptide stimulated PBMCs at 48 hours post stimulation.

Exploratory Objective 6 will assess signatures for the GM-CSF adjuvanted response following the 1st, 2nd and 3rd MVA boosts. Exploratory Endpoint 6 will conduct microarray analyses on PBMC at days 1, 3, and 7 after the 1st, 2nd, and 3rd MVA boosts.

Exploratory objective 7 will assess temporal titers of anti-Env Ab responses to assess the importance of the 3rd MVA boost.

Exploratory Endpoint 3 will determine titers of Env-Specific Ab against various substrates after the 1^{st}, 2^{nd}, and 3^{rd} MVA boosts.

### Example 4

### Exemplary HIV Immunogen Sequences Used in Vectors

Provided below are non-limiting examples of immunogen nucleic acid sequences that may be included in any of the vectors or vaccine inserts described herein. Also provided are non-limiting examples of immunogen protein sequences that may be encoded by a sequence present in any of the vectors or vaccine inserts described herein. One or more (e.g., two, three, four, five, six, seven, eight, nine, ten, eleven, or twelve) of the immunogen sequences listed below may be included in (if a nucleic acid sequence) or encoded by (if a protein sequence) any of the vectors and vaccine inserts provided.

(SEQ ID NO: 11)
env HIV Clade B DNA Sequence (sequence present in GEO-D03)
ATGAAAGTGAAGGGGATCAGGAAGAATTATCAGCACTTGTGGAAATGGGGCATCATGCTCCTTGGGATGTTG
ATGATCTGTAGTGCTGTAGAAAATTTGTGGGTCACAGTTTATTATGGGGTACCTGTGTGGAAAGAAGCAACC
ACCACTCTATTTTGTGCATCAGATGCTAAAGCATATGATACAGAGGTACATAATGTTTGGGCCACACATGCCT
GTGTACCCACAGACCCCAACCCACAAGAAGTAGTATTGGAAAATGTGACAGAAAATTTTAACATGTGGAAAA
ATAACATGGTAGAACAGATGCATGAGGATATAATCAGTTTATGGGATCAAAGCCTAAAGCCATGTGTAAAAT
TAACCCCACTCTGTGTTACTTTAAATTGCACTGATTTGAGGAATGTTACTAATATCAATAATAGTAGTGAGGGA
ATGAGAGGAGAAATAAAAAACTGCTCTTTCAATATCACCACAAGCATAAGAGATAAGGTGAAGAAAGACTAT
GCACTTTTTTATAGACTTGATGTAGTACCAATAGATAATGATAATACTAGCTATAGGTTGATAAATTGTAATAC
CTCAACCATTACACAGGCCTGTCCAAAGGTATCCTTTGAGCCAATTCCCATACATTATTGTACCCCGGCTGGTT
TTGCGATTCTAAAGTGTAAAGACAAGAAGTTCAATGGAACAGGGCCATGTAAAAATGTCAGCACAGTACAAT
GTACACATGGAATTAGGCCAGTAGTGTCAACTCAACTGCTGTTAAATGGCAGTCTAGCAGAAGAAGAGGTAG
TAATTAGATCTAGTAATTTCACAGACAATGCAAAAAACATAATAGTACAGTTGAAAGAATCTGTAGAAATTAA
TTGTACAAGACCCAACAACAATACAAGGAAAAGTATACATATAGGACCAGGAAGAGCATTTTATACAACAGG
AGAAATAATAGGAGATATAAGACAAGCACATTGCAACATTAGTAGAACAAAATGGAATAACACTTTAAATCA
AATAGCTACAAAATTAAAAGAACAATTTGGGAATAATAAAACAATAGTCTTTAATCAATCCTCAGGAGGGGAC
CCAGAAATTGTAATGCACAGTTTTAATTGTGGAGGGGAATTTTTCTACTGTAATTCAACACAACTGTTTAATAG
TACTTGGAATTTTAATGGTACTTGGAATTTAACACAATCGAATGGTACTGAAGGAAATGACACTATCACACTCC
CATGTAGAATAAAACAAATTATAAATATGTGGCAGGAAGTAGGAAAAGCAATGTATGCCCCTCCCATCAGAG
GACAAATTAGATGCTCATCAAATATTACAGGGCTAATATTAACAAGAGATGGTGGAACTAACAGTAGTGGGT
CCGAGATCTTCAGACCTGGGGGAGGAGATATGAGGGACAATTGGAGAAGTGAATTATATAAATATAAAGTA
GTAAAAATTGAACCATTAGGAGTAGCACCCACCAAGGCAAAAAGAAGAGTGGTGCAGAGAGAAAAAAGAGC
AGTGGGAACGATAGGAGCTATGTTCCTTGGGTTCTTGGGAGCAGCAGGAAGCACTATGGGCGCAGCGTCAA
TAACGCTGACGGTACAGGCCAGACTATTATTGTCTGGTATAGTGCAACAGCAGAACAATTTGCTGAGGGCTAT
TGAGGCGCAACAGCATCTGTTGCAACTCACAGTCTGGGGCATCAAGCAGCTCCAGGCAAGAGTCCTGGCTGT
GGAAAGATACCTAAGGGATCAACAGCTCCTAGGGATTTGGGGTTGCTCTGGAAAACTCATCTGCACCACTGCT
GTGCCTTGGAATGCTAGTTGGAGTAATAAAACTCTGGATATGATTTGGGATAACATGACCTGGATGGAGTGG
GAAAGAGAAATCGAAAATTACACAGGCTTAATATACACCTTAATTGAAGAATCGCAGAACCAACAAGAAAAG
AATGAACAAGACTTATTAGCATTAGATAAGTGGGCAAGTTTGTGGAATTGGTTTGACATATCAAATTGGCTGT
GGTATGTAAAAATCTTCATAATGATAGTAGGAGGCTTGATAGGTTTAAGAATAGTTTTTACTGTACTTTCTATA
GTAAATAGAGTTAGGCAGGGATACTCACCATTGTCATTTCAGACCCACCTCCCAGCCCCGAGGGGACCCGACA
GGCCCGAAGGAATCGAAGAAGAAGGTGGAGACAGAGACAGAGACAGATCCGTGCGATTAGTGGATggatcct
tagcacttatctgggacgatctgcggagcctgtgcctcttcagctaccaccgcttgagagacttactcttgattgtaac
gaggattgtggaacttctgggacgcagggggtgggaagccctcaaatattggtggaatctcctacagtattggagtcag
gagctaaagaatagtgctgttagcttgctcaatgccacagctatagcagtagctgaggggacagatagggttatagaag
tagtacaaggagcttatagagctattcgccacatacctagaagaataagacagggcttggaaaggattttgctataa
(SEQ ID NO: 12)
Env HIV Clade B Protein Sequence (sequence encoded by GEO-D03)
MKVKGIRKNYQHLWKWGIMLLGMLMICSAVENLWVTVYYGVPVWKEATTTLFCASDAKAYDTEVHNVWATH
ACVPTDPNPQEVVLENVTENFNMWKNNMVEQMHEDIISLWDQSLKPCVKLTPLCVTLNCTDLRNVTNINNSSE
GMRGEIKNCSFNITTSIRDKVKKDYALFYRLDVVPIDNDNTSYRLINCNTSTITQACPKVSFEPIPIHYCTPAGFAILKC
KDKKFNGTGPCKNVSTVQCTHGIRPVVSTQLLLNGSLAEEEVVIRSSNFTDNAKNIIVQLKESVEINCTRPNNNTRKS
IHIGPGRAFYTTGEIIGDIRQAHCNISRTKWNNTLNQIATKLKEQFGNNKTIVFNQSSGGDPEIVMHSFNCGGEFFY
CNSTQLFNSTWNFNGTWNLTQSNGTEGNDTITLPCRIKQIINMWQEVGKAMYAPPIRGQIRCSSNITGLILTRDG
GTNSSGSEIFRPGGGDMRDNWRSELYKYKVVKIEPLGVAPTKAKRRVVQREKRAVGTIGAMFLGFLGAAGSTMG
AASITLTVQARLLLSGIVQQQNNLLRAIEAQQHLLQLTVWGIKQLQARVLAVERYLRDQQLLGIWGCSGKLICTTAV
PWNASWSNKTLDMIWDNMTWMEWEREIENYTGLIYTLIEESQNQQEKNEQDLLALDKWASLWNWFDISNWL
WYVKIFIMIVGGLIGLRIVFTVLSIVNRVRQGYSPLSFQTHLPAPRGPDRPEGIEEEGGDRDRDRSVRLVDGSLALIW
DDLRSLCLFSYHRLRDLLLIVTRIVELLGRRGWEALKYWWNLLQYWSQELKNSAVSLLNATAIAVAEGTDRVIEVVQ
GAYRAIRHIPRRIRQGLERILL
(SEQ ID NO: 13)
env HIV Clade C DNA Sequence (sequence present in GEO-D06)
ATGAGAGTGAAGGGGATACTGAGGAATTATCGACAATGGTGGATATGGGGCATCTTAGGCTTTTGGATGTTA
ATGATTTGTAATGGAAACTTGTGGGTCACAGTCTATTATGGGGTACCTGTGTGGAAAGAAGCAAAAACTACTC
TATTCTGTGCATCAAATGCTAAAGCATATGAGAAAGAAGTACATAATGTCTGGGCTACACATGCCTGTGTACC
CACAGACCCCAACCCACAAGAAATGGTTTTGGAAAACGTAACAGAAAATTTTAACATGTGGAAAAATGACAT
GGTGAATCAGATGCATGAGGATGTAATCAGCTTATGGGATCAAAGCCTAAAGCCATGTGTAAAGTTGACCCC
ACTCTGTGTCACTTTAGAATGTAGAAAGGTTAATGCTACCCATAATGCTACCAATAATGGGGATGCTACCCAT
AATGTTACCAATAATGGGCAAGAAATACAAAATTGCTCTTTCAATGCAACCACAGAAATAAGAGATAGGAAG
CAGAGAGTGTATGCACTTTTTTATAGACTTGATATAGTACCACTTGATAAGAACAACTCTAGTAAGAACAACTC
TAGTGAGTATTATAGATTAATAAATTGTAATACCTCAGCCATAACACAAGCATGTCCAAAGGTCAGTTTTGATC
CAATTCCTATACACTATTGTGCTCCAGCTGGTTATGCGATTCTAAAGTGTAACAATAAGACATTCAATGGGACA
GGACCATGCAATAATGTCAGCACAGTACAATGTACACATGGAATTAAGCCAGTGGTATCAACTCAGCTATTGT
TAAACGGTAGCCTAGCAGAAGGAGAGATAATAATTAGATCTGAAAATCTGACAGACAATGTCAAAACAATAA
TAGTACATCTTGATCAATCTGTAGAAATTGTGTGTACAAGACCCAACAATAATACAAGAAAAAGTATAAGGAT
AGGGCCAGGACAAACATTCTATGCAACAGGAGGCATAATAGGGAACATACGACAAGCACATTGTAACATTAG
TGAAGACAAATGGAATGAAACTTTACAAAGGGTGGGTAAAAAATTAGTAGAACACTTCCCTAATAAGACAAT
AAAATTTGCACCATCCTCAGGAGGGGACCTAGAAATTACAACACATAGCTTTAATTGTAGAGGAGAATTTTTC
TATTGCAGCACATCAAGACTGTTTAATAGTACATACATGCCTAATGATACAAAAAGTAAGTCAAACAAAACCA
TCACAATCCCATGCAGCATAAAACAAATTGTAAACATGTGGCAGGAGGTAGGACGAGCAATGTATGCCCCTC
CCATTGAAGGAAACATAACCTGTAGATCAAATATCACAGGAATACTATTGGTACGTGATGGAGGAGTAGATT
CAGAAGATCCAGAAAATAATAAGACAGAGACATTCCGACCTGGAGGAGGAGATATGAGGAACAATTGGAGA
AGTGAATTATATAAATATAAAGCGGCAGAAATTAAGCCATTGGGAGTAGCACCCACTCCAGCAAAAAGGAGA
GTGGTGGAGAGAGAAAAAAGAGCAGTAGGATTAGGAGCTGTGTTCCTTGGATTCTTGGGAGCAGCAGGAAG
CACTATGGGCGCAGCGTCAATAACGCTGACGGTACAGGCCAGACAATTGTTGTCTGGTATAGTGCAACAGCA
AAGCAATTTGCTGAGGGCTATCGAGGCGCAACAGCATCTGTTGCAACTCACGGTCTGGGGCATTAAGCAGCT
CCAGACAAGAGTCCTGGCTATCGAAAGATACCTAAAGGATCAACAGCTCCTAGGGCTTTGGGGCTGCTCTGG
AAAACTCATCTGCACCACTAATGTACCTTGGAACTCCAGTTGGAGTAACAAATCTCAAACAGATATTTGGGAA
AACATGACCTGGATGCAGTGGGATAAAGAAGTTAGTAATTACACAGACACAATATACAGGTTGCTTGAAGAC
TCGCAAACCCAGCAGGAAAGAAATGAAAAGGATTTATTAGCATTGGACAATTGGAAAAATCTGTGGAATTGG
TTTAGTATAACAAACTGGCTGTGGTATATAAAAATATTCATAATGATAGTAGGAGGCTTGATAGGCTTAAGAA
TAATTTTTGCTGTGCTTTCTATAGTGAATAGAGTTAGGCAGGGATACTCACCTTTGTCGTTTCAGACCCTTACCC
CAAACCCAAGGGGACCCGACAGGCTCGGAAGAATCGAAGAAGAAGGTGGAGGGCAAGACAGAGACAGATC
GATTCGATTAGTGAACGGATTCTTAGCACTTGCCTGGGACGACCTGTGGAGCCTGTGCCTCTTCAGCTACCAC
CGATTGAGAGACTTAATATTGGTGACAGCGAGAGCGGTGGAACTTCTGGGACACAGCAGTCTCAGGGGACT
ACAGAGGGGGTGGGAAGCCCTTAAGTATCTGGGAGGTATTGTGCAGTATTGGGGTCTGGAACTAAAAAAGA
GGGCTATTAGTCTGCTTGATACTGTAGCAATAGCAGTAGCTGAAGGCACAGATAGGATTATAgaattcctccaaag
aatttgtagagctatccgcaacatacctagaaggataagacagggctttgaagcagctttgcagtaa
(SEQ ID NO: 14)
Env HIV Clade C Protein Sequence (sequence encoded by GEO-D06)
MRVKGILRNYRQWWIWGILGFWMLMICNGNLWVTVYYGVPVWKEAKTTLFCASNAKAYEKEVHNVWATHAC
VPTDPNPQEMVLENVTENFNMWKNDMVNQMHEDVISLWDQSLKPCVKLTPLCVTLECRKVNATHNATNNGD
ATHNVTNNGQEIQNCSFNATTEIRDRKQRVYALFYRLDIVPLDKNNSSKNNSSEYYRLINCNTSAITQACPKVSFDPI
PIHYCAPAGYAILKCNNKTFNGTGPCNNVSTVQCTHGIKPVVSTQLLLNGSLAEGEIIIRSENLTDNVKTIIVHLDQSV
EIVCTRPNNNTRKSIRIGPGQTFYATGGIIGNIRQAHCNISEDKWNETLQRVGKKLVEHFPNKTIKFAPSSGGDLEIT
THSFNCRGEFFYCSTSRLFNSTYMPNDTKSKSNKTITIPCSIKQIVNMWQEVGRAMYAPPIEGNITCRSNITGILLVR
DGGVDSEDPENNKTETFRPGGGDMRNNWRSELYKYKAAEIKPLGVAPTPAKRRVVEREKRAVGLGAVFLGFLGA
AGSTMGAASITLTVQARQLLSGIVQQQSNLLRAIEAQQHLLQLTVWGIKQLQTRVLAIERYLKDQQLLGLWGCSG
KLICTTNVPWNSSWSNKSQTDIWENMTWMQWDKEVSNYTDTIYRLLEDSQTQQERNEKDLLALDNWKNLWN
WFSITNWLWYIKIFIMIVGGLIGLRIIFAVLSIVNRVRQGYSPLSFQTLTPNPRGPDRLGRIEEEGGGQDRDRSIRLVN
GFLALAWDDLWSLCLFSYHRLRDLILVTARAVELLGHSSLRGLQRGWEALKYLGGIVQYWGLELKKRAISLLDTVAI
AVAEGTDRIIEFLQRICRAIRNIPRRIRQGFEAALQ
(SEQ ID NO: 15)
gag HIV Clade B DNA Sequence (sequence present in GEO-03)
ATGGGTGCGAGAGCGTCAGTATTAAGCGGGGGAGAATTAGATCGATGGGAAAAAATTCGGTTAAGGCCAGG
GGGAAAGAAAAAATATAAATTAAAACATATAGTATGGGCAAGCAGGGAGCTAGAACGATTCGCAGTTAATCC
TGGCCTGTTAGAAACATCAGAAGGCTGTAGACAAATACTGGGACAGCTACAACCATCCCTTCAGACAGGATC
AGAAGAACTTAGATCATTATATAATACAGTAGCAACCCTCTATTGTGTGCATCAAAGGATAGAGATAAAAGAC
ACCAAGGAAGCTTTAGACAAGATAGAGGAAGAGCAAAACAAAAGTAAGAAAAAAGCACAGCAAGCAGCAG
CTGACACAGGACACAGCAATCAGGTCAGCCAAAATTACCCTATAGTGCAGAACATCCAGGGGCAAATGGTAC
ATCAGGCCATATCACCTAGAACTTTAAATGCATGGGTAAAAGTAGTAGAAGAGAAGGCTTTCAGCCCAGAAG
TGATACCCATGTTTTCAGCATTATCAGAAGGAGCCACCCCACAAGATTTAAACACCATGCTAAACACAGTGGG
GGGACATCAAGCAGCCATGCAAATGTTAAAAGAGACCATCAATGAGGAAGCTGCAGAATGGGATAGAGTGC
ATCCAGTGCATGCAGGGCCTATTGCACCAGGCCAGATGAGAGAACCAAGGGGAAGTGACATAGCAGGAACT
ACTAGTACCCTTCAGGAACAAATAGGATGGATGACAAATAATCCACCTATCCCAGTAGGAGAAATTTATAAAA
GATGGATAATCCTGGGATTAAATAAAATAGTAAGAATGTATAGCCCTACCAGCATTCTGGACATAAGACAAG
GACCAAAAGAACCCTTTAGAGACTATGTAGACCGGTTCTATAAAACTCTAAGAGCCGAGCAAGCTTCACAGG
AGGTAAAAAATTGGATGACAGAAACCTTGTTGGTCCAAAATGCGAACCCAGATTGTAAGACTATTTTAAAAGC
ATTGGGACCAGCGGCTACACTAGAAGAAATGATGACAGCATGTCAGGGAGTAGGAGGACCCGGCCATAAGG
CAAGAGTTTTGGCTGAAGCAATGAGCCAAGTAACAAATTCAGCTACCATAATGATGCAGAGAGGCAATTTTA
GGAACCAAAGAAAGATTGTTAAGAGCTTCAATAGCGGCAAAGAAGGGCACACAGCCAGAAATTGCAGGGCC
CCTAGGAAAAAGGGCAGCTGGAAAAGCGGAAAGGAAGGACACCAAATGAAAGATTGTACTGAGAGACAGG
CTAATTTTTTAGGGAAGATCTGGCCTTCCTACAAGGGAAGGCCAGGGAATTTTCTTCAGAGCAGACCAGAGCC
AACAGCCCCACCAGAAGAGAGCTTCAGGTCTGGGGTAGAGACAACAACTCCCCCTCAGAAGCAGGAGCCGA
TAGACAAGGAACTGTATCCTTTAACTTCCCTCAGATCACTCTTTGGCAACGACCCCTCGTCACAATAA
(SEQ ID NO: 16)
Gag HIV Clade B Protein Sequence (sequence encoded by GEO-D03)
MGARASVLSGGELDRWEKIRLRPGGKKKYKLKHIVWASRELERFAVNPGLLETSEGCRQILGQLQPSLQTGSEELRS
LYNTVATLYCVHQRIEIKDTKEALDKIEEEQNKSKKKAQQAAADTGHSNQVSQNYPIVQNIQGQMVHQAISPRTL
NAWVKVVEEKAFSPEVIPMFSALSEGATPQDLNTMLNTVGGHQAAMQMLKETINEEAAEWDRVHPVHAGPIA
PGQMREPRGSDIAGTTSTLQEQIGWMTNNPPIPVGEIYKRWIILGLNKIVRMYSPTSILDIRQGPKEPFRDYVDRFY
KTLRAEQASQEVKNWMTETLLVQNANPDCKTILKALGPAATLEEMMTACQGVGGPGHKARVLAEAMSQVTNS
ATIMMQRGNFRNQRKIVKSFNSGKEGHTARNCRAPRKKGSWKSGKEGHQMKDCTERQANFLGKIWPSYKGRP
GNFLQSRPEPTAPPEESFRSGVETTTPPQKQEPIDKELYPLTSLRSLFGNDPSSQ
(SEQ ID NO: 17)
gag HIV Clade C DNA Sequence (sequence present in GEO-D06)
ATGGGTGCGAGAGCGTCAATATTAAGAGGGGGAAAATTAGATAAATGGGAAAAGATTAGGTTAAGGCCAGG
GGGAAAGAAACACTATATGCTAAAACACCTAGTATGGGCAAGCAGGGAGCTGGAAAGATTTGCACTTAACCC
TGGCCTTTTAGAGACATCAGAAGGCTGTAAACAAATAATAAAACAGCTACAACCAGCTCTTCAGACAGGAAC
AGAGGAACTTAGGTCATTATTCAATGCAGTAGCAACTCTCTATTGTGTACATGCAGACATAGAGGTACGAGAC
ACCAAAGAAGCATTAGACAAGATAGAGGAAGAACAAAACAAAAGTCAGCAAAAAACGCAGCAGGCAAAAG
AGGCTGACAAAAAGGTCGTCAGTCAAAATTATCCTATAGTGCAGAATCTTCAAGGGCAAATGGTACACCAGG
CACTATCACCTAGAACTTTGAATGCATGGGTAAAAGTAATAGAAGAAAAAGCCTTTAGCCCGGAGGTAATAC
CCATGTTCACAGCATTATCAGAAGGAGCCACCCCACAAGATTTAAACACCATGTTAAATACCGTGGGGGGACA
TCAAGCAGCCATGCAAATGTTAAAAGATACCATCAATGAGGAGGCTGCAGAATGGGATAGATTACATCCAGT
ACATGCAGGGCCTGTTGCACCAGGCCAAATGAGAGAACCAAGGGGAAGTGACATAGCAGGAACTACTAGTA
ACCTTCAGGAACAAATAGCATGGATGACAAGTAACCCACCTATTCCAGTGGGAGATATCTATAAAAGATGGA
TAATTCTGGGGTTAAATAAAATAGTAAGAATGTATAGCCCTGTCAGCATTTTAGACATAAGACAAGGGCCAAA
GGAACCCTTTAGAGATTATGTAGACCGGTTCTTTAAAACTTTAAGAGCTGAACAAGCTTCACAAGATGTAAAA
AATTGGATGGCAGACACCTTGTTGGTCCAAAATGCGAACCCAGATTGTAAGACCATTTTAAGAGCATTAGGAC
CAGGAGCTACATTAGAAGAAATGATGACAGCATGTCAAGGAGTGGGAGGACCTAGCCACAAAGCAAGAGTG
TTGGCTGAGGCAATGAGCCAAACAGGCAGTACCATAATGATGCAGAGAAGCAATTTTAAAGGCTCTAAAAGA
ACTGTTAAATCCTTCAACTCTGGCAAGGAAGGGCACATAGCTAGAAATTGCAGGGCCCCTAGGAAAAAAGGC
TCTTGGAAATCTGGAAAGGAAGGACACCAAATGAAAGACTGTGCTGAGAGGCAGGCTAATTTTTTAGGGAAA
ATTTGGCCTTCCCACAAGGGGAGGCCAGGGAATTTCCTTCAGAACAGGCCAGAGCCAACAGCCCCACCAGCA
GAGAGCTTCAGGTTCGAGGAGACAACCCCTGCTCCGAAGCAGGAGCTGAAAGACAGGGAACCCTTAACCTCC
CTCAAATCACTCTTTGGCAGCGACCCCTTGTCTCAATAA
(SEQ ID NO: 18)
Gag HIV Clade C Protein Sequence (sequence encoded by GEO-D06)
MGARASILRGGKLDKWEKIRLRPGGKKHYMLKHLVWASRELERFALNPGLLETSEGCKQIIKQLQPALQTGTEELR
SLFNAVATLYCVHADIEVRDTKEALDKIEEEQNKSQQKTQQAKEADKKVVSQNYPIVQNLQGQMVHQALSPRTLN
AWVKVIEEKAFSPEVIPMFTALSEGATPQDLNTMLNTVGGHQAAMQMLKDTINEEAAEWDRLHPVHAGPVAP
GQMREPRGSDIAGTTSNLQEQIAWMTSNPPIPVGDIYKRWIILGLNKIVRMYSPVSILDIRQGPKEPFRDYVDRFFK
TLRAEQASQDVKNWMADTLLVQNANPDCKTILRALGPGATLEEMMTACQGVGGPSHKARVLAEAMSQTGSTI
MMQRSNFKGSKRTVKSFNSGKEGHIARNCRAPRKKGSWKSGKEGHQMKDCAERQANFLGKIWPSHKGRPGNF
LQNRPEPTAPPAESFRFEETTPAPKQELKDREPLTSLKSLFGSDPLSQ
(SEQ ID NO: 19)
pol HIV Clade B DNA Sequence (sequence present in GEO-D03)
TTTTTTAGGGAAGATCTGGCCTTCCTACAAGGGAAGGCCAGGGAATTTTCTTCAGAGCAGACCAGAGCCAAC
AGCCCCACCAGAAGAGAGCTTCAGGTCTGGGGTAGAGACAACAACTCCCCCTCAGAAGCAGGAGCCGATAG
ACAAGGAACTGTATCCTTTAACTTCCCTCAGATCACTCTTTGGCAACGACCCCTCGTCACAATAAAGATAGGGG
GGCAACTAAAGGAAGCTCTATTAGCCACAGGAGCAGATGATACAGTATTAGAAGAAATGAGTTTGCCAGGAA
GATGGAAACCAAAAATGATAGGGGGAATTGGAGGTTTTATCAAAGTAAGACAGTATGATCAGATACTCATAG
AAATCTGTGGACATAAAGCTATAGGTACAGTATTAGTAGGACCTACACCTGTCAACATAATTGGAAGAAATCT
GTTGACTCAGATTGGTTGCACTTTAAATTTTCCCATTAGCCCTATTGAGACTGTACCAGTAAAATTAAAGCCAG
GAATGGATGGCCCAAAAGTTAAACAATGGCCATTGACAGAAGAAAAGATAAAAGCATTAGTAGAAATTTGTA
CAGAGATGGAAAAGGAAGGGAAAATTTCAAAAATTGGGCCTGAAAATCCATACAATACTCCAGTATTTGCCA
TAAAGAAAAAAGACAGTACTAAATGGAGAAAATTAGTAGATTTCAGAGAACTTAATAAGAGAACTCAAGACT
TCTGGGAAGTTCAATTAGGAATACCACATCCCGCAGGGTTAAAAAAGAAAAAATCAGTAACAGTACTGGATG
TGGGTGATGCATATTTTTCAGTTCCCTTAGATGAAGACTTCAGGAAATATACTGCATTTACCATACCTAGTATA
AACAATGAGACACCAGGGATTAGATATCAGTACAATGTGCTTCCACAGGGATGGAAAGGATCACCAGCAATA
TTCCAAAGTAGCATGACAAAAATCTTAGAGCCTTTTAGAAAACAAAATCCAGACATAGTTATCTATCAATACAT
GAACGATTTGTATGTAGGATCTGACTTAGAAATAGGGCAGCATAGAACAAAAATAGAGGAGCTGAGACAAC
ATCTGTTGAGGTGGGGACTTACCACACCAGACAAAAAACATCAGAAAGAACCTCCATTCCTTTGGATGGGTTA
TGAACTCCATCCTGATAAATGGACAGTACAGCCTATAGTGCTGCCAGAAAAAGACAGCTGGACTGTCAATGA
CATACAGAAGTTAGTGGGGAAATTGAATACCGCAAGTCAGATTTACCCAGGGATTAAAGTAAGGCAATTATG
TAAACTCCTTAGAGGAACCAAAGCACTAACAGAAGTAATACCACTAACAGAAGAAGCAGAGCTAGAACTGGC
AGAAAACAGAGAGATTCTAAAAGAACCAGTACATGGAGTGTATTATGACCCATCAAAAGACTTAATAGCAGA
AATACAGAAGCAGGGGCAAGGCCAATGGACATATCAAATTTATCAAGAGCCATTTAAAAATCTGAAAACAGG
AAAATATGCAAGAATGAGGGGTGCCCACACTAATGATGTAAAACAATTAACAGAGGCAGTGCAAAAAATAAC
CACAGAAAGCATAGTAATATGGGGAAAGACTCCTAAATTTAAACTGCCCATACAAAAGGAAACATGGGAAAC
ATGGTGGACAGAGTATTGGCAAGCCACCTGGATTCCTGAGTGGGAGTTTGTTAATACCCCTCCTTTAGTGAAA
TTATGGTACCAGTTAGAGAAAGAACCCATAGTAGGAGCAGAAACCTTCTATGTAGATGGGGCAGCTAACAGG
GAGACTAAATTAGGAAAAGCAGGATATGTTACTAATAGAGGAAGACAAAAAGTTGTCACCCTAACTAACACA
ACAAATCAGAAAACTCAGTTACAAGCAATTTATCTAGCTTTGCAGGATTCGGGATTAGAAGTAAACATAGTAA
CAGACTCACAATATGCATTAGGAATCATTCAAGCACAACCAGATCAAAGTGAATCAGAGTTAGTCAATCAAAT
AATAGAGCAGTTAATAAAAAAGGAAAAGGTCTATCTGGCATGGGTACCAGCACACAAAGGAATTGGAGGAA
ATGAACAAGTAGATAAATTAGTCAGTGCTGGAATCAGGAAAGTACTATTTTTAGATGGAATAGATAAGGCCC
AAGATGAACATTAG
(SEQ ID NO: 20)
Pol HIV Clade B Protein Sequence (sequence encoded by GEO-D03)
FFREDLAFLQGKAREFSSEQTRANSPTRRELQVWGRDNNSPSEAGADRQGTVSFNFPQITLWQRPLVTIKIGGQL
KEALLATGADDTVLEEMSLPGRWKPKMIGGIGGFIKVRQYDQILIEICGHKAIGTVLVGPTPVNIIGRNLLTQIGCTL
NFPISPIETVPVKLKPGMDGPKVKQWPLTEEKIKALVEICTEMEKEGKISKIGPENPYNTPVFAIKKKDSTKWRKLVD
FRELNKRTQDFWEVQLGIPHPAGLKKKKSVTVLDVGDAYFSVPLDEDFRKYTAFTIPSINNETPGIRYQYNVLPQG
WKGSPAIFQSSMTKILEPFRKQNPDIVIYQYMNDLYVGSDLEIGQHRTKIEELRQHLLRWGLTTPDKKHQKEPPFL
WMGYELHPDKWTVQPIVLPEKDSWTVNDIQKLVGKLNTASQIYPGIKVRQLCKLLRGTKALTEVIPLTEEAELELAE
NREILKEPVHGVYYDPSKDLIAEIQKQGQGQWTYQIYQEPFKNLKTGKYARMRGAHTNDVKQLTEAVQKITTESIV
IWGKTPKFKLPIQKETWETWWTEYWQATWIPEWEFVNTPPLVKLWYQLEKEPIVGAETFYVDGAANRETKLGKA
GYVTNRGRQKVVTLTNTTNQKTQLQAIYLALQDSGLEVNIVTDSQYALGIIQAQPDQSESELVNQIIEQLIKKEKVYL
AWVPAHKGIGGNEQVDKLVSAGIRKVLFLDGIDKAQDEH
(SEQ ID NO: 21)
pol HIV Clade C DNA Sequence (sequence present in GEO-D06)
TTTTTTAGGGAAAATTTGGCCTTCCCACAAGGGGAGGCCAGGGAATTTCCTTCAGAACAGGCCAGAGCCAAC
AGCCCCACCAGCAGAGAGCTTCAGGTTCGAGGAGACAACCCCTGCTCCGAAGCAGGAGCTGAAAGACAGGG
AACCCTTAACCTCCCTCAAATCACTCTTTGGCAGCGACCCCTTGTCTCAATAAAAATAGGGGGCCAGATAAAG
GAGGCTCTCTTAGCCACAGGAGCAGATGATACAGTATTAGAAGAAATGAATTTGCCAGGAAAATGGAAACCA
AAAATGATAGGAGGAATTGGAGGTTTTATCAAAGTAAGACAGTATGATCAAATACTTATAGAAATTTGTGGA
AAAAAGGCTATAGGTACAGTATTAGTAGGACCCACACCTGTCAACATAATTGGAAGAAATATGCTGACTCAG
ATTGGATGCACGCTAAATTTTCCAATTAGTCCCATTGAAACTGTACCAGTAAAATTAAAGCCAGGAATGGATG
GCCCAAAGGTTAAACAATGGCCATTGACAGAGGAGAAAATAAAAGCATTAACAGCAATTTGTGATGAAATGG
AGAAGGAAGGAAAAATTACAAAAATTGGGCCTGAAAATCCATATAACACTCCAATATTCGCCATAAAAAAGA
AGGACAGTACTAAGTGGAGAAAATTAGTAGATTTCAGAGAACTTAATAAAAGAACTCAAGACTTCTGGGAAG
TTCAATTAGGAATACCACACCCAGCAGGGTTAAAAAAGAAAAAATCAGTGACAGTACTAGATGTGGGGGATG
CATATTTTTCAGTTCCTTTAGATGAAAGCTTTAGGAGGTATACTGCATTCACCATACCTAGTAGAAACAATGAA
ACACCAGGGATTAGATATCAATATAATGTGCTTCCACAAGGATGGAAAGGATCACCAGCAATATTCCAGAGT
AGCATGACAAAAATCTTAGAGCCCTTTAGAGCACAAAATCCAGAAATAGTCATCTATCAATATATGAATGACT
TGTATGTAGGATCTGACTTAGAAATAGGGCAACATAGAGCAAAGATAGAGGAATTAAGAGAACATCTATTAA
GGTGGGGATTTACCACACCAGACAAGAAACATCAGAAAGAACCCCCATTTCTTTGGATGGGGTATGAACTCC
ATCCTGACAAATGGACAGTACAGCCTATACAGCTGCCAGAAAAGGAGAGCTGGACTGTCAATGATATACAGA
AGTTAGTGGGAAAATTAAACACGGCAAGCCAGATTTACCCAGGGATTAAAGTAAGACAACTTTGTAGACTCC
TTAGAGGGGCCAAAGCACTAACAGACATAGTACCACTAACTGAAGAAGCAGAATTAGAATTGGCAGAGAAC
AGGGAAATTCTAAAAGAACCAGTACATGGAGTATATTATGACCCTTCAAAAGACTTGATAGCTGAAATACAG
AAACAGGGACATGACCAATGGACATATCAAATTTACCAAGAACCATTCAAAAATCTGAAAACAGGGAAGTAT
GCAAAAATGAGGACTGCCCACACTAATGATGTAAAACGGTTAACAGAGGCAGTGCAAAAAATAGCCTTAGAA
AGCATAGTAATATGGGGAAAGATTCCTAAACTTAGGTTACCCATCCAAAAAGAAACATGGGAGACATGGTGG
ACTGACTATTGGCAAGCCACCTGGATTCCTGAGTGGGAATTTGTTAATACTCCTCCCCTAGTAAAATTATGGTA
CCAGCTAGAGAAGGAACCCATAATAGGAGTAGAAACTTTCTATGTAGATGGAGCAGCTAATAGGGAAACCAA
AATAGGAAAAGCAGGGTATGTTACTGACAGAGGAAGGCAGAAAATTGTTTCTCTAACTGAAACAACAAATCA
GAAGACTCAATTACAAGCAATTTATCTAGCTTTGCAAGATTCAGGATCAGAAGTAAACATAGTAACAGACTCA
CAGTATGCATTAGGAATTATTCAAGCACAACCAGATAAGAGTGAATCAGGGTTAGTCAACCAAATAATAGAA
CAATTAATAAAAAAGGAAAGGGTCTACCTGTCATGGGTACCAGCACATAAAGGTATTGGAGGAAATGAACAA
GTAGACAAATTAGTAAGTAGTGGAATCAGGAGAGTGCTATAATAA
(SEQ ID NO: 22)
Pol HIV Clade C Protein Sequence (sequence encoded by GEO-D06)
FFRENLAFPQGEAREFPSEQARANSPTSRELQVRGDNPCSEAGAERQGTLNLPQITLWQRPLVSIKIGGQIKEALLA
TGADDTVLEEMNLPGKWKPKMIGGIGGFIKVRQYDQILIEICGKKAIGTVLVGPTPVNIIGRNMLTQIGCTLNFPISP
IETVPVKLKPGMDGPKVKQWPLTEEKIKALTAICDEMEKEGKITKIGPENPYNTPIFAIKKKDSTKWRKLVDFRELNK
RTQDFWEVQLGIPHPAGLKKKKSVTVLDVGDAYFSVPLDESFRRYTAFTIPSRNNETPGIRYQYNVLPQGWKGSPA
IFQSSMTKILEPFRAQNPEIVIYQYMNDLYVGSDLEIGQHRAKIEELREHLLRWGFTTPDKKHQKEPPFLWMGYEL
HPDKWTVQPIQLPEKESWTVNDIQKLVGKLNTASQIYPGIKVRQLCRLLRGAKALTDIVPLTEEAELELAENREILKE
PVHGVYYDPSKDLIAEIQKQGHDQWTYQIYQEPFKNLKTGKYAKMRTAHTNDVKRLTEAVQKIALESIVIWGKIPK
LRLPIQKETWETWWTDYWQATWIPEWEFVNTPPLVKLWYQLEKEPIIGVETFYVDGAANRETKIGKAGYVTDRG
RQKIVSLTETTNQKTQLQAIYLALQDSGSEVNIVTDSQYALGIIQAQPDKSESGLVNQIIEQLIKKERVYLSWVPAHK
GIGGNEQVDKLVSSGIRRVL
(SEQ ID NO: 23)
rev HIV Clade B DNA Sequence (sequence present in GEO-D03)
ATGGCAGGAAGAAGCGGAGACAGCGACGAAGAGCTCCTCAAGACAGTCAGACTCATCAAGTTTCTCTATCAA
AGCAACCCACCTCCCAGCCCCGAGGGGACCCGACAGGCCCGAAGGAATCGAAGAAGAAGGTGGAGACAGA
GACAGAGACAGATCCGTGCGATTAGTGGATggatccttagcacttatctgggacgatctgcggagcctgtgcctcttca
gctaccaccgcttgagagacttactcttgattgtaacgaggattgtggaacttctgggacgcagggggtgggaagccct
caaatattggtggaatctcctacagtattggagtcaggagctaaagaatag
(SEQ ID NO: 24)
Rev HIV Clade B Protein Sequence (sequence encoded by GEO-D03)
MAGRSGDSDEELLKTVRLIKFLYQSNPPPSPEGTRQARRNRRRRWRQRQRQIRAISGWILSTYLGRSAEPVPLQLP
PLERLTLDCNEDCGTSGTQGVGSPQILVESPTVLESGAKE
(SEQ ID NO: 25)
rev HIV Clade C DNA Sequence (sequence present in GEO-D06)
ATGGCAGGAAGAAGCGGAGACAGCGACGAAGCGCTCCTCAGAGCAGTGAGGATCATCAGAATTTTGTATCA
AAGCAACCCTTACCCCAAACCCAAGGGGACCCGACAGGCTCGGAAGAATCGAAGAAGAAGGTGGAGGGCAA
GACAGAGACAGATCGATTCGATTAGTGAACGGATTCTTAGCACTTGCCTGGGACGACCTGTGGAGCCTGTGC
CTCTTCAGCTACCACCGATTGAGAGACTTAATATTGGTGACAGCGAGAGCGGTGGAACTTCTGGGACACAGC
AGTCTCAGGGGACTACAGAGGGGGTGGGAAGCCCTTAA
(SEQ ID NO: 26)
Rev HIV Clade C Protein Sequence (sequence encoded by GEO-D06)
MAGRSGDSDEALLRAVRIIRILYQSNPYPKPKGTRQARKNRRRRWRARQRQIDSISERILSTCLGRPVEPVPLQLPPI
ERLNIGDSESGGTSGTQQSQGTTEGVGSP
(SEQ ID NO: 27)
tat HIV Clade B DNA Sequence (sequence present in GEO-D03)
ATGGAGCCAGTAGATCCTAGACTAGAGCCCTGGAAGCATCCAGGAAGTCAGCCTAAAACTGCTTGTACCAAT
TGCTATTGTAAAAAGTGTTGCTTTCATTGCCAAGTTTGTTTCATAACAAAAGCCTTAGGCATCTCCTATGGCAG
GAAGAAGCGGAGACAGCGACGAAGAGCTCCTCAAGACAGTCAGACTCATCAAGTTTCTCTATCAAAGCAACC
CACCTCCCAGCCCCGAGGGGACCCGACAGGCCCGAAGGAATCGAAGAAGAAGGTGGAGACAGAGACAGAG
ACAGATCCGTGCGATTAG
(SEQ ID NO: 28)
Tat HIV Clade B Protein Sequence (sequence encoded by GEO-D03)
MEPVDPRLEPWKHPGSQPKTACTNCYCKKCCFHCQVCFITKALGISYGRKKRRQRRRAPQDSQTHQVSLSKQPTS
QPRGDPTGPKESKKKVETETETDPCD
(SEQ ID NO: 29)
tat HIV Clade C DNA Sequence (sequence present in GEO-D06)
ATGGAGCCAGTAGATCCTAACCTAGAGCCCTGGAACCATCCAGGAAGTCAGCCTGAAACTGCTTGCAATAACT
GTTATTGTAAACGCTATAGCTACCATTGTCTAGTTTGCTTTCAGAGAAAAGGCTTAGGCATTTCCTATGGCAGG
AAGAAGCGGAGACAGCGACGAAGCGCTCCTCAGAGCAGTGAGGATCATCAGAATTTTGTATCAAAGCAACCC
TTACCCCAAACCCAAGGGGACCCGACAGGCTCGGAAGAATCGAAGAAGAAGGTGGAGGGCAAGACAGAGA
CAGATCGATTCGATTAG
(SEQ ID NO: 30)
Tat HIV Clade C Protein Sequence (sequence encoded by GEO-D06)
MEPVDPNLEPWNHPGSQPETACNNCYCKRYSYHCLVCFQRKGLGISYGRKKRRQRRSAPQSSEDHQNFVSKQPL
PQTQGDPTGSEESKKKVEGKTETDRFD
(SEQ ID NO: 31)
vpu HIV Clade B DNA Sequence (sequence present in GEO-D03)
ATGCAACCTTTACAAATATTAGCAATAGTAGCATTAGTAGTAGCAGCAATAATAGCAATAGTTGTGTGGACCA
TAGTATTCATAGAATATAGGAAAATATTAAGACAAAGAAAAATAGACAGGTTAATTGATAGGATAACAGAAA
GAGCAGAAGACAGTGGCAATGAAAGTGAAGGGGATCAGGAAGAATTATCAGCACTTGTGGAAATGGGGCA
TCATGCTCCTTGGGATGTTGATGATCTGTAG
(SEQ ID NO: 32)
Vpu HIV Clade B Protein Sequence (sequence encoded by GEO-D03)
MQPLQILAIVALVVAAIIAIVVWTIVFIEYRKILRQRKIDRLIDRITERAEDSGNESEGDQEELSALVEMGHHAPWDV
DDL
(SEQ ID NO: 33)
vpu HIV Clade C DNA Sequence (sequence present in GEO-D06)
ATGTTAGATTTAGATTATAAATTAGCAGTAGGAGCATTTATAGTAGCACTACTCATAGCAATAGTTGTGTGGA
CCATAGTATTTATAGAATATAGGAAATTGTTAAGACAAAGAAAAATAGACTGGTTAATTAAAAGAATTAGGG
AAAGAGCAGAAGACAGTGGCAATGAGAGTGAAGGGGATACTGAGGAATTATCGACAATGGTGGATATGGG
GCATCTTAGGCTTTTGGATGTTAATGATTTGTAA
(SEQ ID NO: 34)
Vpu HIV Clade C Protein Sequence (sequence encoded by GEO-D06)
MLDLDYKLAVGAFIVALLIAIVVWTIVFIEYRKLLRQRKIDWLIKRIRERAEDSGNESEGDTEELSTMVDMGHLRLLD
VNDL
(SEQ ID NO: 35)
env HIV Clade B DNA Sequence (sequence present in MVA62B)
ATGAAAGTGAAGGGGATCAGGAAGAATTATCAGCACTTGTGGAAATGGGGCATCATGCTCCTTGGGATGTTG
ATGATCTGTAGTGCTGTAGAAAATTTGTGGGTCACAGTTTATTATGGGGTACCTGTGTGGAAAGAAGCAACC
ACCACTCTATTTTGTGCATCAGATGCTAAAGCATATGATACAGAGGTACATAATGTTTGGGCCACACATGCCT
GTGTACCCACAGACCCCAACCCACAAGAAGTAGTATTGGAAAATGTGACAGAAAATTTTAACATGTGGAAAA
ATAACATGGTAGAACAGATGCATGAGGATATAATCAGTTTATGGGATCAAAGCCTAAAGCCATGTGTAAAAT
TAACCCCACTCTGTGTTACTTTAAATTGCACTGATTTGAGGAATGTTACTAATATCAATAATAGTAGTGAGGGA
ATGAGAGGAGAAATAAAAAACTGCTCTTTCAATATCACCACAAGCATAAGAGATAAGGTGAAGAAAGACTAT
GCACTTTTCTATAGACTTGATGTAGTACCAATAGATAATGATAATACTAGCTATAGGTTGATAAATTGTAATAC
CTCAACCATTACACAGGCCTGTCCAAAGGTATCCTTTGAGCCAATTCCCATACATTATTGTACCCCGGCTGGTT
TTGCGATTCTAAAGTGTAAAGACAAGAAGTTCAATGGAACAGGGCCATGTAAAAATGTCAGCACAGTACAAT
GTACACATGGAATTAGGCCAGTAGTGTCAACTCAACTGCTGTTAAATGGCAGTCTAGCAGAAGAAGAGGTAG
TAATTAGATCTAGTAATTTCACAGACAATGCAAAAAACATAATAGTACAGTTGAAAGAATCTGTAGAAATTAA
TTGTACAAGACCCAACAACAATACAAGGAAAAGTATACATATAGGACCAGGAAGAGCATTTTATACAACAGG
AGAAATAATAGGAGATATAAGACAAGCACATTGCAACATTAGTAGAACAAAATGGAATAACACTTTAAATCA
AATAGCTACAAAATTAAAAGAACAATTTGGGAATAATAAAACAATAGTCTTTAATCAATCCTCAGGAGGGGAC
CCAGAAATTGTAATGCACAGTTTTAATTGTGGAGGGGAATTCTTCTACTGTAATTCAACACAACTGTTTAATAG
TACTTGGAATTTTAATGGTACTTGGAATTTAACACAATCGAATGGTACTGAAGGAAATGACACTATCACACTCC
CATGTAGAATAAAACAAATTATAAATATGTGGCAGGAAGTAGGAAAAGCAATGTATGCCCCTCCCATCAGAG
GACAAATTAGATGCTCATCAAATATTACAGGGCTAATATTAACAAGAGATGGTGGAACTAACAGTAGTGGGT
CCGAGATCTTCAGACCTGGGGGAGGAGATATGAGGGACAATTGGAGAAGTGAATTATATAAATATAAAGTA
GTAAAAATTGAACCATTAGGAGTAGCACCCACCAAGGCAAAAAGAAGAGTGGTGCAGAGAGAAAAAAGAGC
AGTGGGAACGATAGGAGCTATGTTCCTTGGGTTCTTGGGAGCAGCAGGAAGCACTATGGGCGCAGCGTCAA
TAACGCTGACGGTACAGGCCAGACTATTATTGTCTGGTATAGTGCAACAGCAGAACAATTTGCTGAGGGCTAT
TGAGGCGCAACAGCATCTGTTGCAACTCACAGTCTGGGGCATCAAGCAGCTCCAGGCAAGAGTCCTGGCTGT
GGAAAGATACCTAAGGGATCAACAGCTCCTAGGGATTTGGGGTTGCTCTGGAAAACTCATCTGCACCACTGCT
GTGCCTTGGAATGCTAGTTGGAGTAATAAAACTCTGGATATGATTTGGGATAACATGACCTGGATGGAGTGG
GAAAGAGAAATCGAAAATTACACAGGCTTAATATACACCTTAATTGAGGAATCGCAGAACCAACAAGAAAAG
AATGAACAAGACTTATTAGCATTAGATAAGTGGGCAAGTTTGTGGAATTGGTTTGACATATCAAATTGGCTGT
GGTATGTAAAAATCTTCATAATGATAGTAGGAGGCTTGATAGGTTTAAGAATAGTTTTTACTGTACTTTCTATA
GTAAATAGAGTTAGGCAGGGATACTCACCATTGTCATTTCAGACCCACCTCCCAGCCCCGAGGGGACCCGACA
GGCCCGAAGGAATCGAAGAAGAAGGTGGAGACAGAGACTAA
(SEQ ID NO: 36)
Env HIV Clade B Protein Sequence (sequence encoded by MVA62B)
MKVKGIRKNYQHLWKWGIMLLGMLMICSAVENLWVTVYYGVPVWKEATTTLFCASDAKAYDTEVHNVWATH
ACVPTDPNPQEVVLENVTENFNMWKNNMVEQMHEDIISLWDQSLKPCVKLTPLCVTLNCTDLRNVTNINNSSE
GMRGEIKNCSFNITTSIRDKVKKDYALFYRLDVVPIDNDNTSYRLINCNTSTITQACPKVSFEPIPIHYCTPAGFAILKC
KDKKFNGTGPCKNVSTVQCTHGIRPVVSTQLLLNGSLAEEEVVIRSSNFTDNAKNIIVQLKESVEINCTRPNNNTRKS
IHIGPGRAFYTTGEIIGDIRQAHCNISRTKWNNTLNQIATKLKEQFGNNKTIVFNQSSGGDPEIVMHSFNCGGEFFY
CNSTQLFNSTWNFNGTWNLTQSNGTEGNDTITLPCRIKQIINMWQEVGKAMYAPPIRGQIRCSSNITGLILTRDG
GTNSSGSEIFRPGGGDMRDNWRSELYKYKVVKIEPLGVAPTKAKRRVVQREKRAVGTIGAMFLGFLGAAGSTMG
AASITLTVQARLLLSGIVQQQNNLLRAIEAQQHLLQLTVWGIKQLQARVLAVERYLRDQQLLGIWGCSGKLICTTAV
PWNASWSNKTLDMIWDNMTWMEWEREIENYTGLIYTLIEESQNQQEKNEQDLLALDKWASLWNWFDISNWL
WYVKIFIMIVGGLIGLRIVFTVLSIVNRVRQGYSPLSFQTHLPAPRGPDRPEGIEEEGGDRD
(SEQ ID NO: 37)
env HIV Clade C DNA Sequence (sequence present in MVA71C)
ATGAGAGTGAAGGGGATACTGAGGAATTATCGACAATGGTGGATATGGGGCATCTTAGGCTTTTGGATGTTA
ATGATTTGTAATGGAAACTTGTGGGTCACAGTCTATTATGGGGTACCTGTGTGGAAAGAAGCAAAAACTACTC
TATTCTGTGCATCAAATGCTAAAGCATATGAGAAAGAAGTACATAATGTCTGGGCTACACATGCCTGTGTACC
CACAGACCCCAACCCACAAGAAATGGTTTTGGAAAACGTAACAGAAAATTTTAACATGTGGAAAAATGACAT
GGTGAATCAGATGCATGAGGATGTAATCAGCTTATGGGATCAAAGCCTAAAGCCATGTGTAAAGTTGACCCC
ACTCTGTGTCACTTTAGAATGTAGAAAGGTTAATGCTACCCATAATGCTACCAATAATGGGGATGCTACCCAT
AATGTTACCAATAATGGGCAAGAAATACAAAATTGCTCTTTCAATGCAACCACAGAAATAAGAGATAGGAAG
CAGAGAGTGTATGCACTTTTCTATAGACTTGATATAGTACCACTTGATAAGAACAACTCTAGTAAGAACAACTC
TAGTGAGTATTATAGATTAATAAATTGTAATACCTCAGCCATAACACAAGCATGTCCAAAGGTCAGTTTTGATC
CAATTCCTATACACTATTGTGCTCCAGCTGGTTATGCGATTCTAAAGTGTAACAATAAGACATTCAATGGGACA
GGACCATGCAATAATGTCAGCACAGTACAATGTACACATGGAATTAAGCCAGTGGTATCAACTCAGCTATTGT
TAAACGGTAGCCTAGCAGAAGGAGAGATAATAATTAGATCTGAAAATCTGACAGACAATGTCAAAACAATAA
TAGTACATCTTGATCAATCTGTAGAAATTGTGTGTACAAGACCCAACAATAATACAAGAAAAAGTATAAGGAT
AGGGCCAGGACAAACATTCTATGCAACAGGAGGCATAATAGGGAACATACGACAAGCACATTGTAACATTAG
TGAAGACAAATGGAATGAAACTTTACAAAGGGTGGGTAAAAAATTAGTAGAACACTTCCCTAATAAGACAAT
AAAATTTGCACCATCCTCAGGAGGGGACCTAGAAATTACAACACATAGCTTTAATTGTAGAGGAGAATTCTTC
TATTGCAGCACATCAAGACTGTTTAATAGTACATACATGCCTAATGATACAAAAAGTAAGTCAAACAAAACCA
TCACAATCCCATGCAGCATAAAACAAATTGTAAACATGTGGCAGGAGGTAGGACGAGCAATGTATGCCCCTC
CCATTGAAGGAAACATAACCTGTAGATCAAATATCACAGGAATACTATTGGTACGTGATGGAGGAGTAGATT
CAGAAGATCCAGAAAATAATAAGACAGAGACATTCCGACCTGGAGGAGGAGATATGAGGAACAATTGGAGA
AGTGAATTATATAAATATAAAGCGGCAGAAATTAAGCCATTGGGAGTAGCACCCACTCCAGCAAAAAGGAGA
GTGGTGGAGAGAGAAAAAAGAGCAGTAGGATTAGGAGCTGTGTTCCTTGGATTCTTGGGAGCAGCAGGAAG
CACTATGGGCGCAGCGTCAATAACGCTGACGGTACAGGCCAGACAATTGTTGTCTGGTATAGTGCAACAGCA
AAGCAATTTGCTGAGGGCTATCGAGGCGCAACAGCATCTGTTGCAACTCACGGTCTGGGGCATTAAGCAGCT
CCAGACAAGAGTCCTGGCTATCGAAAGATACCTAAAGGATCAACAGCTCCTAGGGCTTTGGGGCTGCTCTGG
AAAACTCATCTGCACCACTAATGTACCTTGGAACTCCAGTTGGAGTAACAAATCTCAAACAGATATTTGGGAA
AACATGACCTGGATGCAGTGGGATAAAGAAGTTAGTAATTACACAGACACAATATACAGGTTGCTTGAAGAC
TCGCAAACCCAGCAGGAAAGAAATGAAAAGGATTTATTAGCATTGGACAATTGGAAAAATCTGTGGAATTGG
TTTAGTATAACAAACTGGCTGTGGTATATAAAAATATTCATAATGATAGTAGGAGGCTTGATAGGCTTAAGAA
TAATTTTTGCTGTGCTTTCTATAGTGAATAGAGTTAGGCAGGGATACTCACCTTTGTCGTTTCAGACCCTTACCC
CAAACCCAAGGGGACCCGACAGGCTCGGAAGAATCGAAGAAGAAGGTGGAGGGCAAGACAGAGACTAA
(SEQ ID NO: 38)
Env HIV Clade C Protein Sequence (sequence encoded by MVA71C)
MRVKGILRNYRQWWIWGILGFWMLMICNGNLWVTVYYGVPVWKEAKTTLFCASNAKAYEKEVHNVWATHAC
VPTDPNPQEMVLENVTENFNMWKNDMVNQMHEDVISLWDQSLKPCVKLTPLCVTLECRKVNATHNATNNGD
ATHNVTNNGQEIQNCSFNATTEIRDRKQRVYALFYRLDIVPLDKNNSSKNNSSEYYRLINCNTSAITQACPKVSFDPI
PIHYCAPAGYAILKCNNKTFNGTGPCNNVSTVQCTHGIKPVVSTQLLLNGSLAEGEIIIRSENLTDNVKTIIVHLDQSV
EIVCTRPNNNTRKSIRIGPGQTFYATGGIIGNIRQAHCNISEDKWNETLQRVGKKLVEHFPNKTIKFAPSSGGDLEIT
THSFNCRGEFFYCSTSRLFNSTYMPNDTKSKSNKTITIPCSIKQIVNMWQEVGRAMYAPPIEGNITCRSNITGILLVR
DGGVDSEDPENNKTETFRPGGGDMRNNWRSELYKYKAAEIKPLGVAPTPAKRRVVEREKRAVGLGAVFLGFLGA
AGSTMGAASITLTVQARQLLSGIVQQQSNLLRAIEAQQHLLQLTVWGIKQLQTRVLAIERYLKDQQLLGLWGCSG
KLICTTNVPWNSSWSNKSQTDIWENMTWMQWDKEVSNYTDTIYRLLEDSQTQQERNEKDLLALDNWKNLWN
WFSITNWLWYIKIFIMIVGGLIGLRIIFAVLSIVNRVRQGYSPLSFQTLTPNPRGPDRLGRIEEEGGGQDRD
(SEQ ID NO: 39)
gag HIV Clade B DNA Sequence (sequence present in MVA62B)
ATGGGTGCGAGAGCGTCAGTATTAAGCGGGGGAGAATTAGATCGATGGGAAAAAATTCGGTTAAGGCCAGG
GGGAAAGAAAAAATATAAATTAAAACATATAGTATGGGCAAGCAGGGAGCTAGAACGATTCGCAGTTAATCC
TGGCCTGTTAGAAACATCAGAAGGCTGTAGACAAATACTGGGACAGCTACAACCATCCCTTCAGACAGGATC
AGAAGAACTTAGATCATTATATAATACAGTAGCAACCCTCTATTGTGTGCATCAAAGGATAGAGATAAAAGAC
ACCAAGGAAGCTTTAGACAAGATAGAGGAAGAGCAAAACAAAAGTAAGAAAAAAGCACAGCAAGCAGCAG
CTGACACAGGACACAGCAATCAGGTCAGCCAAAATTACCCTATAGTGCAGAACATCCAGGGGCAAATGGTAC
ATCAGGCCATATCACCTAGAACTTTAAATGCATGGGTAAAAGTAGTAGAAGAGAAGGCTTTCAGCCCAGAAG
TGATACCCATGTTTTCAGCATTATCAGAAGGAGCCACCCCACAAGATTTAAACACCATGCTAAACACAGTGGG
GGGACATCAAGCAGCCATGCAAATGTTAAAAGAGACCATCAATGAGGAAGCTGCAGAATGGGATAGAGTGC
ATCCAGTGCATGCAGGGCCTATTGCACCAGGCCAGATGAGAGAACCAAGGGGAAGTGACATAGCAGGAACT
ACTAGTACCCTTCAGGAACAAATAGGATGGATGACAAATAATCCACCTATCCCAGTAGGAGAAATTTATAAAA
GATGGATAATCCTGGGATTAAATAAAATAGTAAGAATGTATAGCCCTACCAGCATTCTGGACATAAGACAAG
GACCAAAAGAACCCTTTAGAGACTATGTAGACCGGTTCTATAAAACTCTAAGAGCCGAGCAAGCTTCACAGG
AGGTAAAAAATTGGATGACAGAAACCTTGTTGGTCCAAAATGCGAACCCAGATTGTAAGACTATTTTAAAAGC
ATTGGGACCAGCGGCTACACTAGAAGAAATGATGACAGCATGTCAGGGAGTAGGAGGACCCGGCCATAAGG
CAAGAGTTTTGGCTGAAGCAATGAGCCAAGTAACAAATTCAGCTACCATAATGATGCAGAGAGGCAATTTTA
GGAACCAAAGAAAGATTGTTAAGTGTTTCAATTGTGGCAAAGAAGGGCACACAGCCAGAAATTGCAGGGCC
CCTAGGAAAAAGGGCTGTTGGAAATGTGGAAAGGAAGGACACCAAATGAAAGATTGTACTGAGAGACAGGC
TAATTTTTTAGGGAAGATCTGGCCTTCCTACAAGGGAAGGCCAGGGAATTTTCTTCAGAGCAGACCAGAGCCA
ACAGCCCCACCAGAAGAGAGCTTCAGGTCTGGGGTAGAGACAACAACTCCCCCTCAGAAGCAGGAGCCGAT
AGACAAGGAACTGTATCCTTTAACTTCCCTCAGATCACTCTTTGGCAACGACCCCTCGTCACAATAA
(SEQ ID NO: 40)
Gag HIV Clade B Protein Sequence (sequence encoded by MVA62B)
MGARASVLSGGELDRWEKIRLRPGGKKKYKLKHIVWASRELERFAVNPGLLETSEGCRQILGQLQPSLQTGSEELRS
LYNTVATLYCVHQRIEIKDTKEALDKIEEEQNKSKKKAQQAAADTGHSNQVSQNYPIVQNIQGQMVHQAISPRTL
NAWVKVVEEKAFSPEVIPMFSALSEGATPQDLNTMLNTVGGHQAAMQMLKETINEEAAEWDRVHPVHAGPIA
PGQMREPRGSDIAGTTSTLQEQIGWMTNNPPIPVGEIYKRWIILGLNKIVRMYSPTSILDIRQGPKEPFRDYVDRFY
KTLRAEQASQEVKNWMTETLLVQNANPDCKTILKALGPAATLEEMMTACQGVGGPGHKARVLAEAMSQVTNS
ATIMMQRGNFRNQRKIVKCFNCGKEGHTARNCRAPRKKGCWKCGKEGHQMKDCTERQANFLGKIWPSYKGRP
GNFLQSRPEPTAPPEESFRSGVETTTPPQKQEPIDKELYPLTSLRSLFGNDPSSQ
(SEQ ID NO: 41)
gag HIV Clade C DNA Sequence (sequence present in MVA71C)
ATGGGTGCGAGAGCGTCAATATTAAGAGGGGGAAAATTAGATAAATGGGAAAAGATTAGGTTAAGGCCAGG
GGGAAAGAAACACTATATGCTAAAACACCTAGTATGGGCAAGCAGGGAGCTGGAAAGATTTGCACTTAACCC
TGGCCTTTTAGAGACATCAGAAGGCTGTAAACAAATAATAAAACAGCTACAACCAGCTCTTCAGACAGGAAC
AGAGGAACTTAGGTCATTATTCAATGCAGTAGCAACTCTCTATTGTGTACATGCAGACATAGAGGTACGAGAC
ACCAAAGAAGCATTAGACAAGATAGAGGAAGAACAAAACAAAAGTCAGCAAAAAACGCAGCAGGCAAAAG
AGGCTGACAAAAAGGTCGTCAGTCAAAATTATCCTATAGTGCAGAATCTTCAAGGGCAAATGGTACACCAGG
CACTATCACCTAGAACTTTGAATGCATGGGTAAAAGTAATAGAAGAAAAAGCCTTTAGCCCGGAGGTAATAC
CCATGTTCACAGCATTATCAGAAGGAGCCACCCCACAAGATTTAAACACCATGTTAAATACCGTGGGGGGACA
TCAAGCAGCCATGCAAATGTTAAAAGATACCATCAATGAGGAGGCTGCAGAATGGGATAGATTACATCCAGT
ACATGCAGGGCCTGTTGCACCAGGCCAAATGAGAGAACCAAGGGGAAGTGACATAGCAGGAACTACTAGTA
ACCTTCAGGAACAAATAGCATGGATGACAAGTAACCCACCTATTCCAGTGGGAGATATCTATAAAAGATGGA
TAATTCTGGGGTTAAATAAAATAGTAAGAATGTATAGCCCTGTCAGCATTTTAGACATAAGACAAGGGCCAAA
GGAACCCTTTAGAGATTATGTAGACCGGTTCTTTAAAACTTTAAGAGCTGAACAAGCTTCACAAGATGTAAAA
AATTGGATGGCAGACACCTTGTTGGTCCAAAATGCGAACCCAGATTGTAAGACCATTTTAAGAGCATTAGGAC
CAGGAGCTACATTAGAAGAAATGATGACAGCATGTCAAGGAGTGGGAGGACCTAGCCACAAAGCAAGAGTG
TTGGCTGAGGCAATGAGCCAAACAGGCAGTACCATAATGATGCAGAGAAGCAATTTTAAAGGCTCTAAAAGA
ACTGTTAAATGCTTCAACTGTGGCAAGGAAGGGCACATAGCTAGAAATTGCAGGGCCCCTAGGAAAAAAGGC
TGTTGGAAATGTGGAAAGGAAGGACACCAAATGAAAGACTGTGCTGAGAGGCAGGCTAATTTTTTAGGGAA
AATTTGGCCTTCCCACAAGGGGAGGCCAGGGAATTTCCTTCAGAACAGGCCAGAGCCAACAGCCCCACCAGC
AGAGAGCTTCAGGTTCGAGGAGACAACCCCTGCTCCGAAGCAGGAGCTGAAAGACAGGGAACCCTTAACCTC
CCTCAAATCACTCTTTGGCAGCGACCCCTTGTCTCAATAA
(SEQ ID NO: 42)
Gag HIV Clade C Protein Sequence (sequence encoded by MVA71C)
MGARASILRGGKLDKWEKIRLRPGGKKHYMLKHLVWASRELERFALNPGLLETSEGCKQIIKQLQPALQTGTEELR
SLFNAVATLYCVHADIEVRDTKEALDKIEEEQNKSQQKTQQAKEADKKVVSQNYPIVQNLQGQMVHQALSPRTLN
AWVKVIEEKAFSPEVIPMFTALSEGATPQDLNTMLNTVGGHQAAMQMLKDTINEEAAEWDRLHPVHAGPVAP
GQMREPRGSDIAGTTSNLQEQIAWMTSNPPIPVGDIYKRWIILGLNKIVRMYSPVSILDIRQGPKEPFRDYVDRFFK
TLRAEQASQDVKNWMADTLLVQNANPDCKTILRALGPGATLEEMMTACQGVGGPSHKARVLAEAMSQTGSTI
MMQRSNFKGSKRTVKCFNCGKEGHIARNCRAPRKKGCWKCGKEGHQMKDCAERQANFLGKIWPSHKGRPGN
FLQNRPEPTAPPAESFRFEETTPAPKQELKDREPLTSLKSLFGSDPLSQ
(SEQ ID NO: 43)
pol HIV Clade B DNA Sequence (sequence present in MVA62B)
TTTTTTAGGGAAGATCTGGCCTTCCTACAAGGGAAGGCCAGGGAATTTTCTTCAGAGCAGACCAGAGCCAAC
AGCCCCACCAGAAGAGAGCTTCAGGTCTGGGGTAGAGACAACAACTCCCCCTCAGAAGCAGGAGCCGATAG
ACAAGGAACTGTATCCTTTAACTTCCCTCAGATCACTCTTTGGCAACGACCCCTCGTCACAATAAAGATAGGGG
GGCAACTAAAGGAAGCTCTATTAGATACAGGAGCAGATGATACAGTATTAGAAGAAATGAGTTTGCCAGGAA
GATGGAAACCAAAAATGATAGGGGGAATTGGAGGTTTTATCAAAGTAAGACAGTATGATCAGATACTCATAG
AAATCTGTGGACATAAAGCTATAGGTACAGTATTAGTAGGACCTACACCTGTCAACATAATTGGAAGAAATCT
GTTGACTCAGATTGGTTGCACTTTAAATTTTCCCATTAGCCCTATTGAGACTGTACCAGTAAAATTAAAGCCAG
GAATGGATGGCCCAAAAGTTAAACAATGGCCATTGACAGAAGAAAAAATAAAAGCATTAGTAGAAATTTGTA
CAGAAATGGAAAAGGAAGGGAAAATTTCAAAAATTGGGCCTGAGAATCCATACAATACTCCAGTATTTGCCA
TAAAGAAAAAAGACAGTACTAAATGGAGGAAATTAGTAGATTTCAGAGAACTTAATAAGAGAACTCAAGACT
TCTGGGAAGTTCAATTAGGAATACCACATCCCGCAGGGTTAAAAAAGAAAAAATCAGTAACAGTACTGGATG
TGGGTGATGCATATTTTTCAGTTCCCTTAGATGAAGACTTCAGGAAGTATACTGCATTTACCATACCTAGTATA
AACAATGAGACACCAGGGATTAGATATCAGTACAATGTGCTTCCACAGGGATGGAAAGGATCACCAGCAATA
TTCCAAAGTAGCATGACAAAAATCTTAGAGCCTTTTAAAAAACAAAATCCAGACATAGTTATCTATCAATACAT
GAACGATTTGTATGTAGGATCTGACTTAGAAATAGGGCAGCATAGAACAAAAATAGAGGAGCTGAGACAAC
ATCTGTTGAGGTGGGGACTTACCACACCAGACAAAAAACATCAGAAAGAACCTCCATTCCTTTGGATGGGTTA
TGAACTCCATCCTGATAAATGGACAGTACAGCCTATAGTGCTGCCAGAAAAAGACAGCTGGACTGTCAATGA
CATACAGAAGTTAGTGGGGAAATTGAATACCGCAAGTCAGATTTACCCAGGGATTAAAGTAAGGCAATTATG
TAAACTCCTTAGAGGAACCAAAGCACTAACAGAAGTAATACCACTAACAGAAGAAGCAGAGCTAGAACTGGC
AGAAAACAGAGAGATTCTAAAAGAACCAGTACATGGAGTGTATTATGACCCATCAAAAGACTTAATAGCAGA
AATACAGAAGCAGGGGCAAGGCCAATGGACATATCAAATTTATCAAGAGCCATTTAAAAATCTGAAAACAGG
AAAATATGCAAGAATGAGGGGTGCCCACACTAATGATGTAAAACAATTAACAGAGGCAGTGCAAAAAATAAC
CACAGAAAGCATAGTAATATGGGGAAAGACTCCTAAATTTAAACTACCCATACAAAAGGAAACATGGGAAAC
ATGGTGGACAGAGTATTGGCAAGCCACCTGGATTCCTGAGTGGGAGTTTGTTAATACCCCTCCTTTAGTGAAA
TTATGGTACCAGTTAGAGAAAGAACCCATAGTAGGAGCAGAAACCTTCTATGTAGATGGGGCAGCTAACAGG
GAGACTAAATTAGGAAAAGCAGGATATGTTACTAACAAAGGAAGACAAAAGGTTGTCCCCCTAACTAACACA
ACAAATCAGAAAACTCAGTTACAAGCAATTTATCTAGCTTTGCAGGATTCAGGATTAGAAGTAAACATAGTAA
CAGACTCACAATATGCATTAGGAATCATTCAAGCACAACCAGATAAAAGTGAATCAGAGTTAGTCAATCAAAT
AATAGAGCAGTTAATAAAAAAGGAAAAGGTCTATCTGGCATGGGTACCAGCACACAAAGGAATTGGAGGAA
ATGAACAAGTAGATAAATTAGTCAGTGCTGGAATCAGGAAAATACTATTTTTAGATGGAATAGATAAGGCCC
AAGATGAACATTAG
(SEQ ID NO: 44)
Pol HIV Clade B Protein Sequence (sequence encoded by MVA62B)
FFREDLAFLQGKAREFSSEQTRANSPTRRELQVWGRDNNSPSEAGADRQGTVSFNFPQITLWQRPLVTIKIGGQL
KEALLDTGADDTVLEEMSLPGRWKPKMIGGIGGFIKVRQYDQILIEICGHKAIGTVLVGPTPVNIIGRNLLTQIGCTL
NFPISPIETVPVKLKPGMDGPKVKQWPLTEEKIKALVEICTEMEKEGKISKIGPENPYNTPVFAIKKKDSTKWRKLVD
FRELNKRTQDFWEVQLGIPHPAGLKKKKSVTVLDVGDAYFSVPLDEDFRKYTAFTIPSINNETPGIRYQYNVLPQG
WKGSPAIFQSSMTKILEPFKKQNPDIVIYQYMNDLYVGSDLEIGQHRTKIEELRQHLLRWGLTTPDKKHQKEPPFL
WMGYELHPDKWTVQPIVLPEKDSWTVNDIQKLVGKLNTASQIYPGIKVRQLCKLLRGTKALTEVIPLTEEAELELAE
NREILKEPVHGVYYDPSKDLIAEIQKQGQGQWTYQIYQEPFKNLKTGKYARMRGAHTNDVKQLTEAVQKITTESIV
IWGKTPKFKLPIQKETWETWWTEYWQATWIPEWEFVNTPPLVKLWYQLEKEPIVGAETFYVDGAANRETKLGKA
GYVTNKGRQKVVPLTNTTNQKTQLQAIYLALQDSGLEVNIVTDSQYALGIIQAQPDKSESELVNQIIEQLIKKEKVYL
AWVPAHKGIGGNEQVDKLVSAGIRKILFLDGIDKAQDEH
(SEQ ID NO: 45)
pol HIV Clade C DNA Sequence (sequence present in MVA71C)
TTTTTTAGGGAAAATTTGGCCTTCCCACAAGGGGAGGCCAGGGAATTTCCTTCAGAACAGGCCAGAGCCAAC
AGCCCCACCAGCAGAGAGCTTCAGGTTCGAGGAGACAACCCCTGCTCCGAAGCAGGAGCTGAAAGACAGGG
AACCCTTAACCTCCCTCAAATCACTCTTTGGCAGCGACCCCTTGTCTCAATAAAAATAGGGGGCCAGATAAAG
GAGGCTCTCTTAGACACAGGAGCAGATGATACAGTATTAGAAGAAATGAATTTGCCAGGAAAATGGAAACCA
AAAATGATAGGAGGAATTGGAGGTTTTATCAAAGTAAGACAGTATGATCAAATACTTATAGAAATTTGTGGA
AAAAAGGCTATAGGTACAGTATTAGTAGGACCCACACCTGTCAACATAATTGGAAGAAATATGCTGACTCAG
ATTGGATGCACGCTAAATTTTCCAATTAGTCCCATTGAAACTGTACCAGTAAAATTAAAGCCAGGAATGGATG
GCCCAAAGGTTAAACAATGGCCATTGACAGAGGAGAAAATAAAAGCATTAACAGCAATTTGTGATGAAATGG
AGAAGGAAGGAAAAATTACAAAAATTGGGCCTGAAAATCCATATAACACTCCAATATTCGCCATAAAAAAGA
AGGACAGTACTAAGTGGAGAAAATTAGTAGATTTCAGAGAACTTAATAAAAGAACTCAAGACTTCTGGGAAG
TTCAATTAGGAATACCACACCCAGCAGGGTTAAAAAAGAAAAAATCAGTGACAGTACTAGATGTGGGGGATG
CATATTTTTCAGTTCCTTTAGATGAAAGCTTTAGGAGGTATACTGCATTCACCATACCTAGTAGAAACAATGAA
ACACCAGGGATTAGATATCAATATAATGTGCTTCCACAAGGATGGAAAGGATCACCAGCAATATTCCAGAGT
AGCATGACAAAAATCTTAGAGCCCTTTAGAGCACAAAATCCAGAAATAGTCATCTATCAATATATGAATGACT
TGTATGTAGGATCTGACTTAGAAATAGGGCAACATAGAGCAAAGATAGAGGAATTAAGAGAACATCTATTAA
GGTGGGGATTTACCACACCAGACAAGAAACATCAGAAAGAACCCCCATTTCTTTGGATGGGGTATGAACTCC
ATCCTGACAAATGGACAGTACAGCCTATACAGCTGCCAGAAAAGGAGAGCTGGACTGTCAATGATATACAGA
AGTTAGTGGGAAAATTAAACACGGCAAGCCAGATTTACCCAGGGATTAAAGTAAGACAACTTTGTAGACTCC
TTAGAGGGGCCAAAGCACTAACAGACATAGTACCACTAACTGAAGAAGCAGAATTAGAATTGGCAGAGAAC
AGGGAAATTCTAAAAGAACCAGTACATGGAGTATATTATGACCCTTCAAAAGACTTGATAGCTGAAATACAG
AAACAGGGACATGACCAATGGACATATCAAATTTACCAAGAACCATTCAAAAATCTGAAAACAGGGAAGTAT
GCAAAAATGAGGACTGCCCACACTAATGATGTAAAACGGTTAACAGAGGCAGTGCAAAAAATAGCCTTAGAA
AGCATAGTAATATGGGGAAAGATTCCTAAACTTAGGTTACCCATCCAAAAAGAAACATGGGAGACATGGTGG
ACTGACTATTGGCAAGCCACCTGGATTCCTGAGTGGGAATTTGTTAATACTCCTCCCCTAGTAAAATTATGGTA
CCAGCTAGAGAAGGAACCCATAATAGGAGTAGAAACTTTCTATGTAGATGGAGCAGCTAATAGGGAAACCAA
AATAGGAAAAGCAGGGTATGTTACTGACAGAGGAAGGCAGAAAATTGTTTCTCTAACTGAAACAACAAATCA
GAAGACTCAATTACAAGCAATTTATCTAGCTTTGCAAGATTCAGGATCAGAAGTAAACATAGTAACAGACTCA
CAGTATGCATTAGGAATTATTCAAGCACAACCAGATAAGAGTGAATCAGGGTTAGTCAACCAAATAATAGAA
CAATTAATAAAAAAGGAAAGGGTCTACCTGTCATGGGTACCAGCACATAAAGGTATTGGAGGAAATGAACAA
GTAGACAAATTAGTAAGTAGTGGAATCAGGAGAGTGCTATAG
(SEQ ID NO: 46)
Pol HIV Clade C Protein Sequence (sequence encoded by MVA71C)
FFRENLAFPQGEAREFPSEQARANSPTSRELQVRGDNPCSEAGAERQGTLNLPQITLWQRPLVSIKIGGQIKEALLD
TGADDTVLEEMNLPGKWKPKMIGGIGGFIKVRQYDQILIEICGKKAIGTVLVGPTPVNIIGRNMLTQIGCTLNFPISP
IETVPVKLKPGMDGPKVKQWPLTEEKIKALTAICDEMEKEGKITKIGPENPYNTPIFAIKKKDSTKWRKLVDFRELNK
RTQDFWEVQLGIPHPAGLKKKKSVTVLDVGDAYFSVPLDESFRRYTAFTIPSRNNETPGIRYQYNVLPQGWKGSPA
IFQSSMTKILEPFRAQNPEIVIYQYMNDLYVGSDLEIGQHRAKIEELREHLLRWGFTTPDKKHQKEPPFLWMGYEL
HPDKWTVQPIQLPEKESWTVNDIQKLVGKLNTASQIYPGIKVRQLCRLLRGAKALTDIVPLTEEAELELAENREILKE
PVHGVYYDPSKDLIAEIQKQGHDQWTYQIYQEPFKNLKTGKYAKMRTAHTNDVKRLTEAVQKIALESIVIWGKIPK
LRLPIQKETWETWWTDYWQATWIPEWEFVNTPPLVKLWYQLEKEPIIGVETFYVDGAANRETKIGKAGYVTDRG
RQKIVSLTETTNQKTQLQAIYLALQDSGSEVNIVTDSQYALGIIQAQPDKSESGLVNQIIEQLIKKERVYLSWVPAHK
GIGGNEQVDKLVSSGIRRVL

A number of embodiments of the disclosure have been described. Nevertheless, it will be understood that various modifications may be made without departing from the spirit and scope of the disclosure. Accordingly, other embodiments are within the scope of the following claims.

1. A vector comprising:
a prokaryotic origin of replication;
a promoter sequence;
a eukaryotic transcription cassette comprising a vaccine insert encoding one or more immunogens and GM-CSF;
a polyadenylation sequence; and a
a transcription termination sequence.

2. The vector of claim 1, wherein the vaccine insert comprises a sequence that encodes one or more immunogens selected from the group consisting of: gag, gp120, pol, env, Tat, Rev, Vpu, Nef, Vif, and Vpr.

3.-9. (canceled)

10. The vector of claim 2, wherein the insert comprises a sequence that encodes gag, pol, Tat, Rev, and env.

11.-16. (canceled)

17. The vector of claim 1, wherein the encoded GM-CSF is a full-length human GM-CSF.

18. The vector of claim 17, wherein the sequence encoding GM-CSF comprises the sequence of: nucleotides 6633-7068 of SEQ ID NO: 7, nucleotides 6648-7082 of SEQ ID NO: 8, or nucleotides 7336-7770 of SEQ ID NO: 9.

19.-21. (canceled)

22. The vector of claim 1, comprising the sequence of GEO-D03 (SEQ ID NO: 7), GEO-D06 (SEQ ID NO: 8) or GEO-D07 (SEQ ID NO: 9).

23.-24. (canceled)

25. A vaccine insert encoding one or more immunogens and GM-CSF.

26.-43. (canceled)

44. A method of inducing an immune response in a subject comprising administering to a subject one or more doses of the vector of claim 1.

45. The method of claim 44, wherein the vaccine insert comprises a sequence that encodes one or more immunogens selected from the group consisting of: gag, gp120, pol, env, Tat, Rev, Vpu, Nef, Vif, and Vpr.

46. The method of claim 44, wherein the vector comprises the sequence of GEO-D03 (SEQ ID NO: 7), GEO-D06 (SEQ ID NO: 8) or GEO-D07 (SEQ ID NO: 9).

47.-48. (canceled)

49. The method of claim 44, wherein at least two doses of the vector are administered to the subject.

50. The method of claim 49, wherein at least two doses of the vector are administered at least two months apart.

51. The method of claim 44, further comprising the step of administering one or more doses of a MVA vaccine encoding one or more immunogens.

52.-54. (canceled)

55. The method of claim 51, wherein at least one dose of the MVA vaccine is administered to the subject after the administration of at least one dose of the vector of claim 1 to the subject.

56. (canceled)

57. The method of claim 51, wherein at least one dose of the MVA vaccine is administered to the subject at the same time as administration of a dose of the vector of claim 1 to the subject.

58-59. (canceled)

60. The method of claim 51, wherein said administering results in an increase in the avidity of immunogen-specific antibodies, an increase in immunogen-specific antibody titers, an increase in immunogen specific IgA levels, or an increase in resistance to HIV infection.

61. A method of treating a subject having HIV, comprising administering to the subject one or more doses of the vector of claim 1.

62. (canceled)

63. The method of claim 61, wherein the vector comprises the sequence of GEO-D03 (SEQ ID NO: 7), GEO-D06 (SEQ ID NO: 8) or GEO-D07 (SEQ ID NO: 9).

64.-67. (canceled)

68. The method of claim 61, further comprising the step of administering one or more doses of a MVA vaccine encoding one or more immunogens.

69.-75. (canceled)

76. The method of claim 61, wherein said administering results in an increase in the avidity of immunogen-specific antibodies, an increase in immunogen-specific antibody titers, or an increase in immunogen specific IgA levels.

77.-81. (canceled)


SEQUENCE LISTING (46 sequences)

SEQ ID NO: 1-6 - skipped (6 entries)

SEQ ID NO: 7 - DNA, 9940 bp, Artificial Sequence
  Description of Artificial Sequence: Synthetic GEO-D03 vector polynucleotide
atcgatgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagagcttcaatagcggcaaagaagggcacacagccagaaattgcagggcccctaggaaaaagggcagctggaaaagcggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataaagataggggggcaactaaaggaagctctattagccacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaagataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaaatatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatgaacgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaataccgcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcctttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactaacacaacaaatcagaaaactcagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacattagaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcctcaagacagtcagactcatcaagtttctctatcaaagcagtaagtagtaaatgtaatgcaacctttacaaatattagcaatagtagcattagtagtagcagcaataatagcaatagttgtgtggaccatagtattcatagaatataggaaaatattaagacaaagaaaaatagacaggttaattgataggataacagaaagagcagaagacagtggcaatgaaagtgaaggggatcaggaagaattatcagcacttgtggaaatggggcatcatgctccttgggatgttgatgatctgtagtgctgtagaaaatttgtgggtcacagtttattatggggtacctgtgtggaaagaagcaaccaccactctattttgtgcatcagatgctaaagcatatgatacagaggtacataatgtttgggccacacatgcctgtgtacccacagaccccaacccacaagaagtagtattggaaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatgttactaatatcaataatagtagtgagggaatgagaggagaaataaaaaactgctctttcaatatcaccacaagcataagagataaggtgaagaaagactatgcacttttttatagacttgatgtagtaccaatagataatgataatactagctataggttgataaattgtaatacctcaaccattacacaggcctgtccaaaggtatcctttgagccaattcccatacattattgtaccccggctggttttgcgattctaaagtgtaaagacaagaagttcaatggaacagggccatgtaaaaatgtcagcacagtacaatgtacacatggaattaggccagtagtgtcaactcaactgctgttaaatggcagtctagcagaagaagaggtagtaattagatctagtaatttcacagacaatgcaaaaaacataatagtacagttgaaagaatctgtagaaattaattgtacaagacccaacaacaatacaaggaaaagtatacatataggaccaggaagagcattttatacaacaggagaaataataggagatataagacaagcacattgcaacattagtagaacaaaatggaataacactttaaatcaaatagctacaaaattaaaagaacaatttgggaataataaaacaatagtctttaatcaatcctcaggaggggacccagaaattgtaatgcacagttttaattgtggaggggaatttttctactgtaattcaacacaactgtttaatagtacttggaattttaatggtacttggaatttaacacaatcgaatggtactgaaggaaatgacactatcacactcccatgtagaataaaacaaattataaatatgtggcaggaagtaggaaaagcaatgtatgcccctcccatcagaggacaaattagatgctcatcaaatattacagggctaatattaacaagagatggtggaactaacagtagtgggtccgagatcttcagacctgggggaggagatatgagggacaattggagaagtgaattatataaatataaagtagtaaaaattgaaccattaggagtagcacccaccaaggcaaaaagaagagtggtgcagagagaaaaaagagcagtgggaacgataggagctatgttccttgggttcttgggagcagcaggaagcactatgggcgcagcgtcaataacgctgacggtacaggccagactattattgtctggtatagtgcaacagcagaacaatttgctgagggctattgaggcgcaacagcatctgttgcaactcacagtctggggcatcaagcagctccaggcaagagtcctggctgtggaaagatacctaagggatcaacagctcctagggatttggggttgctctggaaaactcatctgcaccactgctgtgccttggaatgctagttggagtaataaaactctggatatgatttgggataacatgacctggatggagtgggaaagagaaatcgaaaattacacaggcttaatatacaccttaattgaagaatcgcagaaccaacaagaaaagaatgaacaagacttattagcattagataagtgggcaagtttgtggaattggtttgacatatcaaattggctgtggtatgtaaaaatcttcataatgatagtaggaggcttgataggtttaagaatagtttttactgtactttctatagtaaatagagttaggcagggatactcaccattgtcatttcagacccacctcccagccccgaggggacccgacaggcccgaaggaatcgaagaagaaggtggagacagagacagagacagatccgtgcgattagtggatggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggagctaaagaatagtgctgttagcttgctcaatgccacagctatagcagtagctgaggggacagatagggttatagaagtagtacaaggagcttatagagctattcgccacatacctagaagaataagacagggcttggaaaggattttgctataactcgagatgtggctgcaaggcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgaggctagccccgggtgataaacggaccgcgcaatccctaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatataaaaaacgcccggcggcaaccgagcgttctgaacgctagagtcgacaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtctgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcccttttacggttcctggccttttgctggccttttgctcacatgttgtcgacaatattggctattggccattgcatacgttgtatctatatcataatatgtacatttatattggctcatgtccaatatgaccgccatgttgacattgattattgactagttattaatagtaatcaattacgggttcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgc

SEQ ID NO: 8 - DNA, 10900 bp, Artificial Sequence
  Description of Artificial Sequence: Synthetic GEO-D06 vector polynucleotide
ggatccggcttgctgaagtgcactcggcaagaggcgaggggtggcggctggtgagtacgccaaattttatttgactagcggaggctagaaggagagagatgggtgcgagagcgtcaatattaagagggggaaaattagataaatgggaaaagattaggttaaggccagggggaaagaaacactatatgctaaaacacctagtatgggcaagcagggagctggaaagatttgcacttaaccctggccttttagagacatcagaaggctgtaaacaaataataaaacagctacaaccagctcttcagacaggaacagaggaacttaggtcattattcaatgcagtagcaactctctattgtgtacatgcagacatagaggtacgagacaccaaagaagcattagacaagatagaggaagaacaaaacaaaagtcagcaaaaaacgcagcaggcaaaagaggctgacaaaaaggtcgtcagtcaaaattatcctatagtgcagaatcttcaagggcaaatggtacaccaggcactatcacctagaactttgaatgcatgggtaaaagtaatagaagaaaaagcctttagcccggaggtaatacccatgttcacagcattatcagaaggagccaccccacaagatttaaacaccatgttaaataccgtggggggacatcaagcagccatgcaaatgttaaaagataccatcaatgaggaggctgcagaatgggatagattacatccagtacatgcagggcctgttgcaccaggccaaatgagagaaccaaggggaagtgacatagcaggaactactagtaaccttcaggaacaaatagcatggatgacaagtaacccacctattccagtgggagatatctataaaagatggataattctggggttaaataaaatagtaagaatgtatagccctgtcagcattttagacataagacaagggccaaaggaaccctttagagattatgtagaccggttctttaaaactttaagagctgaacaagcttcacaagatgtaaaaaattggatggcagacaccttgttggtccaaaatgcgaacccagattgtaagaccattttaagagcattaggaccaggagctacattagaagaaatgatgacagcatgtcaaggagtgggaggacctagccacaaagcaagagtgttggctgaggcaatgagccaaacaggcagtaccataatgatgcagagaagcaattttaaaggctctaaaagaactgttaaatccttcaactctggcaaggaagggcacatagctagaaattgcagggcccctaggaaaaaaggctcttggaaatctggaaaggaaggacaccaaatgaaagactgtgctgagaggcaggctaattttttagggaaaatttggccttcccacaaggggaggccagggaatttccttcagaacaggccagagccaacagccccaccagcagagagcttcaggttcgaggagacaacccctgctccgaagcaggagctgaaagacagggaacccttaacctccctcaaatcactctttggcagcgaccccttgtctcaataaaaatagggggccagataaaggaggctctcttagccacaggagcagatgatacagtattagaagaaatgaatttgccaggaaaatggaaaccaaaaatgataggaggaattggaggttttatcaaagtaagacagtatgatcaaatacttatagaaatttgtggaaaaaaggctataggtacagtattagtaggacccacacctgtcaacataattggaagaaatatgctgactcagattggatgcacgctaaattttccaattagtcccattgaaactgtaccagtaaaattaaagccaggaatggatggcccaaaggttaaacaatggccattgacagaggagaaaataaaagcattaacagcaatttgtgatgaaatggagaaggaaggaaaaattacaaaaattgggcctgaaaatccatataacactccaatattcgccataaaaaagaaggacagtactaagtggagaaaattagtagatttcagagaacttaataaaagaactcaagacttctgggaagttcaattaggaataccacacccagcagggttaaaaaagaaaaaatcagtgacagtactagatgtgggggatgcatatttttcagttcctttagatgaaagctttaggaggtatactgcattcaccatacctagtagaaacaatgaaacaccagggattagatatcaatataatgtgcttccacaaggatggaaaggatcaccagcaatattccagagtagcatgacaaaaatcttagagccctttagagcacaaaatccagaaatagtcatctatcaatatatgaatgacttgtatgtaggatctgacttagaaatagggcaacatagagcaaagatagaggaattaagagaacatctattaaggtggggatttaccacaccagacaagaaacatcagaaagaacccccatttctttggatggggtatgaactccatcctgacaaatggacagtacagcctatacagctgccagaaaaggagagctggactgtcaatgatatacagaagttagtgggaaaattaaacacggcaagccagatttacccagggattaaagtaagacaactttgtagactccttagaggggccaaagcactaacagacatagtaccactaactgaagaagcagaattagaattggcagagaacagggaaattctaaaagaaccagtacatggagtatattatgacccttcaaaagacttgatagctgaaatacagaaacagggacatgaccaatggacatatcaaatttaccaagaaccattcaaaaatctgaaaacagggaagtatgcaaaaatgaggactgcccacactaatgatgtaaaacggttaacagaggcagtgcaaaaaatagccttagaaagcatagtaatatggggaaagattcctaaacttaggttacccatccaaaaagaaacatgggagacatggtggactgactattggcaagccacctggattcctgagtgggaatttgttaatactcctcccctagtaaaattatggtaccagctagagaaggaacccataataggagtagaaactttctatgtagatggagcagctaatagggaaaccaaaataggaaaagcagggtatgttactgacagaggaaggcagaaaattgtttctctaactgaaacaacaaatcagaagactcaattacaagcaatttatctagctttgcaagattcaggatcagaagtaaacatagtaacagactcacagtatgcattaggaattattcaagcacaaccagataagagtgaatcagggttagtcaaccaaataatagaacaattaataaaaaaggaaagggtctacctgtcatgggtaccagcacataaaggtattggaggaaatgaacaagtagacaaattagtaagtagtggaatcaggagagtgctataataagctcgagatacttggacaggagttgaaactatcataagaatgctgcaacaactactgtttattcatttcagaattgggtgccagcatagcagaataggcattatgagacagagaagagcaagaaatggagccagtagatcctaacctagagccctggaaccatccaggaagtcagcctgaaactgcttgcaataactgttattgtaaacgctatagctaccattgtctagtttgctttcagagaaaaggcttaggcatttcctatggcaggaagaagcggagacagcgacgaagcgctcctcagagcagtgaggatcatcagaattttgtatcaaagcagtaagtatctgtaatgttagatttagattataaattagcagtaggagcatttatagtagcactactcatagcaatagttgtgtggaccatagtatttatagaatataggaaattgttaagacaaagaaaaatagactggttaattaaaagaattagggaaagagcagaagacagtggcaatgagagtgaaggggatactgaggaattatcgacaatggtggatatggggcatcttaggcttttggatgttaatgatttgtaatggaaacttgtgggtcacagtctattatggggtacctgtgtggaaagaagcaaaaactactctattctgtgcatcaaatgctaaagcatatgagaaagaagtacataatgtctgggctacacatgcctgtgtacccacagaccccaacccacaagaaatggttttggaaaacgtaacagaaaattttaacatgtggaaaaatgacatggtgaatcagatgcatgaggatgtaatcagcttatgggatcaaagcctaaagccatgtgtaaagttgaccccactctgtgtcactttagaatgtagaaaggttaatgctacccataatgctaccaataatggggatgctacccataatgttaccaataatgggcaagaaatacaaaattgctctttcaatgcaaccacagaaataagagataggaagcagagagtgtatgcacttttttatagacttgatatagtaccacttgataagaacaactctagtaagaacaactctagtgagtattatagattaataaattgtaatacctcagccataacacaagcatgtccaaaggtcagttttgatccaattcctatacactattgtgctccagctggttatgcgattctaaagtgtaacaataagacattcaatgggacaggaccatgcaataatgtcagcacagtacaatgtacacatggaattaagccagtggtatcaactcagctattgttaaacggtagcctagcagaaggagagataataattagatctgaaaatctgacagacaatgtcaaaacaataatagtacatcttgatcaatctgtagaaattgtgtgtacaagacccaacaataatacaagaaaaagtataaggatagggccaggacaaacattctatgcaacaggaggcataatagggaacatacgacaagcacattgtaacattagtgaagacaaatggaatgaaactttacaaagggtgggtaaaaaattagtagaacacttccctaataagacaataaaatttgcaccatcctcaggaggggacctagaaattacaacacatagctttaattgtagaggagaatttttctattgcagcacatcaagactgtttaatagtacatacatgcctaatgatacaaaaagtaagtcaaacaaaaccatcacaatcccatgcagcataaaacaaattgtaaacatgtggcaggaggtaggacgagcaatgtatgcccctcccattgaaggaaacataacctgtagatcaaatatcacaggaatactattggtacgtgatggaggagtagattcagaagatccagaaaataataagacagagacattccgacctggaggaggagatatgaggaacaattggagaagtgaattatataaatataaagcggcagaaattaagccattgggagtagcacccactccagcaaaaaggagagtggtggagagagaaaaaagagcagtaggattaggagctgtgttccttggattcttgggagcagcaggaagcactatgggcgcagcgtcaataacgctgacggtacaggccagacaattgttgtctggtatagtgcaacagcaaagcaatttgctgagggctatcgaggcgcaacagcatctgttgcaactcacggtctggggcattaagcagctccagacaagagtcctggctatcgaaagatacctaaaggatcaacagctcctagggctttggggctgctctggaaaactcatctgcaccactaatgtaccttggaactccagttggagtaacaaatctcaaacagatatttgggaaaacatgacctggatgcagtgggataaagaagttagtaattacacagacacaatatacaggttgcttgaagactcgcaaacccagcaggaaagaaatgaaaaggatttattagcattggacaattggaaaaatctgtggaattggtttagtataacaaactggctgtggtatataaaaatattcataatgatagtaggaggcttgataggcttaagaataatttttgctgtgctttctatagtgaatagagttaggcagggatactcacctttgtcgtttcagacccttaccccaaacccaaggggacccgacaggctcggaagaatcgaagaagaaggtggagggcaagacagagacagatcgattcgattagtgaacggattcttagcacttgcctgggacgacctgtggagcctgtgcctcttcagctaccaccgattgagagacttaatattggtgacagcgagagcggtggaacttctgggacacagcagtctcaggggactacagagggggtgggaagcccttaagtatctgggaggtattgtgcagtattggggtctggaactaaaaaagagggctattagtctgcttgatactgtagcaatagcagtagctgaaggcacagataggattatagaattcctccaaagaatttgtagagctatccgcaacatacctagaaggataagacagggctttgaagcagctttgcagtaatctagatgtggctgcaaggcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgaggctagccccgggtgataaacggaccgcgcaatccctaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatataaaaaacgcccggcggcaaccgagcgttctgaacgctagagtcgacaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtctgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcccttttacggttcctggccttttgctggccttttgctcacatgttgtcgacaatattggctattggccattgcatacgttgtatctatatcataatatgtacatttatattggctcatgtccaatatgaccgccatgttgacattgattattgactagttattaatagtaatcaattacgggttcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccat

SEQ ID NO: 9 - DNA, 9944 bp, Artificial Sequence
  Description of Artificial Sequence: Synthetic GEO-D07 vector polynucleotide
cgacaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaactgatccggcttgctgaagtgcactcggcaagaggcgaggggtggcggctggtgagtacgccaaattttatttgactagcggaggctagaaggagagagatgggtgcgagagcgtcaatattaagagggggaaaattagataaatgggaaaagattaggttaaggccagggggaaagaaacactatatgctaaaacacctagtatgggcaagcagggagctggaaagatttgcacttaaccctggccttttagagacatcagaaggctgtaaacaaataataaaacagctacaaccagctcttcagacaggaacagaggaacttaggtcattattcaatgcagtagcaactctctattgtgtacatgcagacatagaggtacgagacaccaaagaagcattagacaagatagaggaagaacaaaacaaaagtcagcaaaaaacgcagcaggcaaaagaggctgacaaaaaggtcgtcagtcaaaattatcctatagtgcagaatcttcaagggcaaatggtacaccaggcactatcacctagaactttgaatgcatgggtaaaagtaatagaagaaaaagcctttagcccggaggtaatacccatgttcacagcattatcagaaggagccaccccacaagatttaaacaccatgttaaataccgtggggggacatcaagcagccatgcaaatgttaaaagataccatcaatgaggaggctgcagaatgggatagattacatccagtacatgcagggcctgttgcaccaggccaaatgagagaaccaaggggaagtgacatagcaggaactactagtaaccttcaggaacaaatagcatggatgacaagtaacccacctattccagtgggagatatctataaaagatggataattctggggttaaataaaatagtaagaatgtatagccctgtcagcattttagacataagacaagggccaaaggaaccctttagagattatgtagaccggttctttaaaactttaagagctgaacaagcttcacaagatgtaaaaaattggatggcagacaccttgttggtccaaaatgcgaacccagattgtaagaccattttaagagcattaggaccaggagctacattagaagaaatgatgacagcatgtcaaggagtgggaggacctagccacaaagcaagagtgttggctgaggcaatgagccaaacaggcagtaccataatgatgcagagaagcaattttaaaggctctaaaagaactgttaaatccttcaactctggcaaggaagggcacatagctagaaattgcagggcccctaggaaaaaaggctcttggaaatctggaaaggaaggacaccaaatgaaagactgtgctgagaggcaggctaattttttagggaaaatttggccttcccacaaggggaggccagggaatttccttcagaacaggccagagccaacagccccaccagcagagagcttcaggttcgaggagacaacccctgctccgaagcaggagctgaaagacagggaacccttaacctccctcaaatcactctttggcagcgaccccttgtctcaataaaaatagggggccagataaaggaggctctcttagccacaggagcagatgatacagtattagaagaaatgaatttgccaggaaaatggaaaccaaaaatgataggaggaattggaggttttatcaaagtaagacagtatgatcaaatacttatagaaatttgtggaaaaaaggctataggtacagtattagtaggacccacacctgtcaacataattggaagaaatatgctgactcagattggatgcacgctaaattttccaattagtcccattgaaactgtaccagtaaaattaaagccaggaatggatggcccaaaggttaaacaatggccattgacagaggagaaaataaaagcattaacagcaatttgtgatgaaatggagaaggaaggaaaaattacaaaaattgggcctgaaaatccatataacactccaatattcgccataaaaaagaaggacagtactaagtggagaaaattagtagatttcagagaacttaataaaagaactcaagacttctgggaagttcaattaggaataccacacccagcagggttaaaaaagaaaaaatcagtgacagtactagatgtgggggatgcatatttttcagttcctttagatgaaagctttaggaggtatactgcattcaccatacctagtagaaacaatgaaacaccagggattagatatcaatataatgtgcttccacaaggatggaaaggatcaccagcaatattccagagtagcatgacaaaaatcttagagccctttagagcacaaaatccagaaatagtcatctatcaatatatgaatgacttgtatgtaggatctgacttagaaatagggcaacatagagcaaagatagaggaattaagagaacatctattaaggtggggatttaccacaccagacaagaaacatcagaaagaacccccatttctttggatggggtatgaactccatcctgacaaatggacagtacagcctatacagctgccagaaaaggagagctggactgtcaatgatatacagaagttagtgggaaaattaaacacggcaagccagatttacccagggattaaagtaagacaactttgtagactccttagaggggccaaagcactaacagacatagtaccactaactgaagaagcagaattagaattggcagagaacagggaaattctaaaagaaccagtacatggagtatattatgacccttcaaaagacttgatagctgaaatacagaaacagggacatgaccaatggacatatcaaatttaccaagaaccattcaaaaatctgaaaacagggaagtatgcaaaaatgaggactgcccacactaatgatgtaaaacggttaacagaggcagtgcaaaaaatagccttagaaagcatagtaatatggggaaagattcctaaacttaggttacccatccaaaaagaaacatgggagacatggtggactgactattggcaagccacctggattcctgagtgggaatttgttaatactcctcccctagtaaaattatggtaccagctagagaaggaacccataataggagtagaaactttctatgtagatggagcagctaatagggaaaccaaaataggaaaagcagggtatgttactgacagaggaaggcagaaaattgtttctctaactgaaacaacaaatcagaagactcaattacaagcaatttatctagctttgcaagattcaggatcagaagtaaacatagtaacagactcacagtatgcattaggaattattcaagcacaaccagataagagtgaatcagggttagtcaaccaaataatagaacaattaataaaaaaggaaagggtctacctgtcatgggtaccagcacataaaggtattggaggaaatgaacaagtagacaaattagtaagtagtggaatcaggagagtgctataataagctcgagatacttggacaggagttgaaactatcataagaatgctgcaacaactactgtttattcatttcagaattgggtgccagcatagcagaataggcattatgagacagagaagagcaagaaatggagccagtagatcctaacctagagccctggaaccatccaggaagtcagcctgaaactgcttgcaataactgttattgtaaacgctatagctaccattgtctagtttgctttcagagaaaaggcttaggcatttcctatggcaggaagaagcggagacagcgacgaagcgctcctcagagcagtgaggatcatcagaattttgtatcaaagcagtaagtatctgtaatgttagatttagattataaattagcagtaggagcatttatagtagcactactcatagcaatagttgtgtggaccatagtatttatagaatataggaaattgttaagacaaagaaaaatagactggttaattaaaagaattagggaaagagcagaagacagtggcaatgagagtgaaggggatactgaggaattatcgacaatggtggatatggggcatcttaggcttttggatgttaatgatttgtaatggaaacttgtgggtcacagtctattatggggtacctgtgtggaaagaagcaaaaactactctattctgtgcatcaaatgctaaagcatatgagaaagaagtacataatgtctgggctacacatgcctgtgtacccacagaccccaacccacaagaaatggttttggaaaacgtaacagaaaattttaacatgtggaaaaatgacatggtgaatcagatgcatgaggatgtaatcagcttatgggatcaaagcctaaagccatgtgtaaagttgaccccactctgtgtcactttagaatgtagaaaggttaatgctacccataatgctaccaataatggggatgctacccataatgttaccaataatgggcaagaaatacaaaattgctctttcaatgcaaccacagaaataagagataggaagcagagagtgtatgcacttttttatagacttgatatagtaccacttgataagaacaactctagtaagaacaactctagtgagtattatagattaataaattgtaatacctcagccataacacaagcatgtccaaaggtcagttttgatccaattcctatacactattgtgctccagctggttatgcgattctaaagtgtaacaataagacattcaatgggacaggaccatgcaataatgtcagcacagtacaatgtacacatggaattaagccagtggtatcaactcagctattgttaaacggtagcctagcagaaggagagataataattagatctgaaaatctgacagacaatgtcaaaacaataatagtacatcttgatcaatctgtagaaattgtgtgtacaagacccaacaataatacaagaaaaagtataaggatagggccaggacaaacattctatgcaacaggaggcataatagggaacatacgacaagcacattgtaacattagtgaagacaaatggaatgaaactttacaaagggtgggtaaaaaattagtagaacacttccctaataagacaataaaatttgcaccatcctcaggaggggacctagaaattacaacacatagctttaattgtagaggagaatttttctattgcagcacatcaagactgtttaatagtacatacatgcctaatgatacaaaaagtaagtcaaacaaaaccatcacaatcccatgcagcataaaacaaattgtaaacatgtggcaggaggtaggacgagcaatgtatgcccctcccattgaaggaaacataacctgtagatcaaatatcacaggaatactattggtacgtgatggaggagtagattcagaagatccagaaaataataagacagagacattccgacctggaggaggagatatgaggaacaattggagaagtgaattatataaatataaagcggcagaaattaagccattgggagtagcacccactccagcaaaaaggagagtggtggagagagaaaaaagagcagtaggattaggagctgtgttccttggattcttgggagcagcaggaagcactatgggcgcagcgtcaataacgctgacggtacaggccagacaattgttgtctggtatagtgcaacagcaaagcaatttgctgagggctatcgaggcgcaacagcatctgttgcaactcacggtctggggcattaagcagctccagacaagagtcctggctatcgaaagatacctaaaggatcaacagctcctagggctttggggctgctctggaaaactcatctgcaccactaatgtaccttggaactccagttggagtaacaaatctcaaacagatatttgggaaaacatgacctggatgcagtgggataaagaagttagtaattacacagacacaatatacaggttgcttgaagactcgcaaacccagcaggaaagaaatgaaaaggatttattagcattggacaattggaaaaatctgtggaattggtttagtataacaaactggctgtggtatataaaaatattcataatgatagtaggaggcttgataggcttaagaataatttttgctgtgctttctatagtgaatagagttaggcagggatactcacctttgtcgtttcagacccttaccccaaacccaaggggacccgacaggctcggaagaatcgaagaagaaggtggagggcaagacagagacagatcgattcgattagtgaacggattcttagcacttgcctgggacgacctgtggagcctgtgcctcttcagctaccaccgattgagagacttaatattggtgacagcgagagcggtggaacttctgggacacagcagtctcaggggactacagagggggtgggaagcccttaagtatctgggaggtattgtgcagtattggggtctggaactaaaaaagagggctattagtctgcttgatactgtagcaatagcagtagctgaaggcacagataggattatagaattcctccaaagaatttgtagagctatccgcaacatacctagaaggataagacagggctttgaagcagctttgcagtaatctagatgtggctgcaaggcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgaggctagccccgggtgataaacggaccgcgcaatccctaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatataaaaaacgcccggcggcaaccgagcgttctgaacgctagagtcgacaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtctgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcccttttacggttcctggccttttgctggccttttgctcacatgttgt

SEQ ID NO: 10 - protein, 144 aa, Homo sapiens
  Human GM-CSF
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 11 - DNA, 2562 bp, Human immunodeficiency virus
  HIV Clade B Env DNA sequence
atgaaagtgaaggggatcaggaagaattatcagcacttgtggaaatggggcatcatgctccttgggatgttgatgatctgtagtgctgtagaaaatttgtgggtcacagtttattatggggtacctgtgtggaaagaagcaaccaccactctattttgtgcatcagatgctaaagcatatgatacagaggtacataatgtttgggccacacatgcctgtgtacccacagaccccaacccacaagaagtagtattggaaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatgttactaatatcaataatagtagtgagggaatgagaggagaaataaaaaactgctctttcaatatcaccacaagcataagagataaggtgaagaaagactatgcacttttttatagacttgatgtagtaccaatagataatgataatactagctataggttgataaattgtaatacctcaaccattacacaggcctgtccaaaggtatcctttgagccaattcccatacattattgtaccccggctggttttgcgattctaaagtgtaaagacaagaagttcaatggaacagggccatgtaaaaatgtcagcacagtacaatgtacacatggaattaggccagtagtgtcaactcaactgctgttaaatggcagtctagcagaagaagaggtagtaattagatctagtaatttcacagacaatgcaaaaaacataatagtacagttgaaagaatctgtagaaattaattgtacaagacccaacaacaatacaaggaaaagtatacatataggaccaggaagagcattttatacaacaggagaaataataggagatataagacaagcacattgcaacattagtagaacaaaatggaataacactttaaatcaaatagctacaaaattaaaagaacaatttgggaataataaaacaatagtctttaatcaatcctcaggaggggacccagaaattgtaatgcacagttttaattgtggaggggaatttttctactgtaattcaacacaactgtttaatagtacttggaattttaatggtacttggaatttaacacaatcgaatggtactgaaggaaatgacactatcacactcccatgtagaataaaacaaattataaatatgtggcaggaagtaggaaaagcaatgtatgcccctcccatcagaggacaaattagatgctcatcaaatattacagggctaatattaacaagagatggtggaactaacagtagtgggtccgagatcttcagacctgggggaggagatatgagggacaattggagaagtgaattatataaatataaagtagtaaaaattgaaccattaggagtagcacccaccaaggcaaaaagaagagtggtgcagagagaaaaaagagcagtgggaacgataggagctatgttccttgggttcttgggagcagcaggaagcactatgggcgcagcgtcaataacgctgacggtacaggccagactattattgtctggtatagtgcaacagcagaacaatttgctgagggctattgaggcgcaacagcatctgttgcaactcacagtctggggcatcaagcagctccaggcaagagtcctggctgtggaaagatacctaagggatcaacagctcctagggatttggggttgctctggaaaactcatctgcaccactgctgtgccttggaatgctagttggagtaataaaactctggatatgatttgggataacatgacctggatggagtgggaaagagaaatcgaaaattacacaggcttaatatacaccttaattgaagaatcgcagaaccaacaagaaaagaatgaacaagacttattagcattagataagtgggcaagtttgtggaattggtttgacatatcaaattggctgtggtatgtaaaaatcttcataatgatagtaggaggcttgataggtttaagaatagtttttactgtactttctatagtaaatagagttaggcagggatactcaccattgtcatttcagacccacctcccagccccgaggggacccgacaggcccgaaggaatcgaagaagaaggtggagacagagacagagacagatccgtgcgattagtggatggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggagctaaagaatagtgctgttagcttgctcaatgccacagctatagcagtagctgaggggacagatagggttatagaagtagtacaaggagcttatagagctattcgccacatacctagaagaataagacagggcttggaaaggattttgctataa

SEQ ID NO: 12 - protein, 853 aa, Human immunodeficiency virus
  HIV Clade B Env protein sequence
MKVKGIRKNYQHLWKWGIMLLGMLMICSAVENLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNVTNINNSSEGMRGEIKNCSFNITTSIRDKVKKDYALFYRLDVVPIDNDNTSYRLINCNTSTITQACPKVSFEPIPIHYCTPAGFAILKCKDKKFNGTGPCKNVSTVQCTHGIRPVVSTQLLLNGSLAEEEVVIRSSNFTDNAKNIIVQLKESVEINCTRPNNNTRKSIHIGPGRAFYTTGEIIGDIRQAHCNISRTKWNNTLNQIATKLKEQFGNNKTIVFNQSSGGDPEIVMHSFNCGGEFFYCNSTQLFNSTWNFNGTWNLTQSNGTEGNDTITLPCRIKQIINMWQEVGKAMYAPPIRGQIRCSSNITGLILTRDGGTNSSGSEIFRPGGGDMRDNWRSELYKYKVVKIEPLGVAPTKAKRRVVQREKRAVGTIGAMFLGFLGAAGSTMGAASITLTVQARLLLSGIVQQQNNLLRAIEAQQHLLQLTVWGIKQLQARVLAVERYLRDQQLLGIWGCSGKLICTTAVPWNASWSNKTLDMIWDNMTWMEWEREIENYTGLIYTLIEESQNQQEKNEQDLLALDKWASLWNWFDISNWLWYVKIFIMIVGGLIGLRIVFTVLSIVNRVRQGYSPLSFQTHLPAPRGPDRPEGIEEEGGDRDRDRSVRLVDGSLALIWDDLRSLCLFSYHRLRDLLLIVTRIVELLGRRGWEALKYWWNLLQYWSQELKNSAVSLLNATAIAVAEGTDRVIEVVQGAYRAIRHIPRRIRQGLERILL

SEQ ID NO: 13 - DNA, 2604 bp, Human immunodeficiency virus
  HIV Clade C Env DNA sequence
atgagagtgaaggggatactgaggaattatcgacaatggtggatatggggcatcttaggcttttggatgttaatgatttgtaatggaaacttgtgggtcacagtctattatggggtacctgtgtggaaagaagcaaaaactactctattctgtgcatcaaatgctaaagcatatgagaaagaagtacataatgtctgggctacacatgcctgtgtacccacagaccccaacccacaagaaatggttttggaaaacgtaacagaaaattttaacatgtggaaaaatgacatggtgaatcagatgcatgaggatgtaatcagcttatgggatcaaagcctaaagccatgtgtaaagttgaccccactctgtgtcactttagaatgtagaaaggttaatgctacccataatgctaccaataatggggatgctacccataatgttaccaataatgggcaagaaatacaaaattgctctttcaatgcaaccacagaaataagagataggaagcagagagtgtatgcacttttttatagacttgatatagtaccacttgataagaacaactctagtaagaacaactctagtgagtattatagattaataaattgtaatacctcagccataacacaagcatgtccaaaggtcagttttgatccaattcctatacactattgtgctccagctggttatgcgattctaaagtgtaacaataagacattcaatgggacaggaccatgcaataatgtcagcacagtacaatgtacacatggaattaagccagtggtatcaactcagctattgttaaacggtagcctagcagaaggagagataataattagatctgaaaatctgacagacaatgtcaaaacaataatagtacatcttgatcaatctgtagaaattgtgtgtacaagacccaacaataatacaagaaaaagtataaggatagggccaggacaaacattctatgcaacaggaggcataatagggaacatacgacaagcacattgtaacattagtgaagacaaatggaatgaaactttacaaagggtgggtaaaaaattagtagaacacttccctaataagacaataaaatttgcaccatcctcaggaggggacctagaaattacaacacatagctttaattgtagaggagaatttttctattgcagcacatcaagactgtttaatagtacatacatgcctaatgatacaaaaagtaagtcaaacaaaaccatcacaatcccatgcagcataaaacaaattgtaaacatgtggcaggaggtaggacgagcaatgtatgcccctcccattgaaggaaacataacctgtagatcaaatatcacaggaatactattggtacgtgatggaggagtagattcagaagatccagaaaataataagacagagacattccgacctggaggaggagatatgaggaacaattggagaagtgaattatataaatataaagcggcagaaattaagccattgggagtagcacccactccagcaaaaaggagagtggtggagagagaaaaaagagcagtaggattaggagctgtgttccttggattcttgggagcagcaggaagcactatgggcgcagcgtcaataacgctgacggtacaggccagacaattgttgtctggtatagtgcaacagcaaagcaatttgctgagggctatcgaggcgcaacagcatctgttgcaactcacggtctggggcattaagcagctccagacaagagtcctggctatcgaaagatacctaaaggatcaacagctcctagggctttggggctgctctggaaaactcatctgcaccactaatgtaccttggaactccagttggagtaacaaatctcaaacagatatttgggaaaacatgacctggatgcagtgggataaagaagttagtaattacacagacacaatatacaggttgcttgaagactcgcaaacccagcaggaaagaaatgaaaaggatttattagcattggacaattggaaaaatctgtggaattggtttagtataacaaactggctgtggtatataaaaatattcataatgatagtaggaggcttgataggcttaagaataatttttgctgtgctttctatagtgaatagagttaggcagggatactcacctttgtcgtttcagacccttaccccaaacccaaggggacccgacaggctcggaagaatcgaagaagaaggtggagggcaagacagagacagatcgattcgattagtgaacggattcttagcacttgcctgggacgacctgtggagcctgtgcctcttcagctaccaccgattgagagacttaatattggtgacagcgagagcggtggaacttctgggacacagcagtctcaggggactacagagggggtgggaagcccttaagtatctgggaggtattgtgcagtattggggtctggaactaaaaaagagggctattagtctgcttgatactgtagcaatagcagtagctgaaggcacagataggattatagaattcctccaaagaatttgtagagctatccgcaacatacctagaaggataagacagggctttgaagcagctttgcagtaa

SEQ ID NO: 14 - protein, 867 aa, Human immunodeficiency virus
  HIV Clade C Env protein sequence
MRVKGILRNYRQWWIWGILGFWMLMICNGNLWVTVYYGVPVWKEAKTTLFCASNAKAYEKEVHNVWATHACVPTDPNPQEMVLENVTENFNMWKNDMVNQMHEDVISLWDQSLKPCVKLTPLCVTLECRKVNATHNATNNGDATHNVTNNGQEIQNCSFNATTEIRDRKQRVYALFYRLDIVPLDKNNSSKNNSSEYYRLINCNTSAITQACPKVSFDPIPIHYCAPAGYAILKCNNKTFNGTGPCNNVSTVQCTHGIKPVVSTQLLLNGSLAEGEIIIRSENLTDNVKTIIVHLDQSVEIVCTRPNNNTRKSIRIGPGQTFYATGGIIGNIRQAHCNISEDKWNETLQRVGKKLVEHFPNKTIKFAPSSGGDLEITTHSFNCRGEFFYCSTSRLFNSTYMPNDTKSKSNKTITIPCSIKQIVNMWQEVGRAMYAPPIEGNITCRSNITGILLVRDGGVDSEDPENNKTETFRPGGGDMRNNWRSELYKYKAAEIKPLGVAPTPAKRRVVEREKRAVGLGAVFLGFLGAAGSTMGAASITLTVQARQLLSGIVQQQSNLLRAIEAQQHLLQLTVWGIKQLQTRVLAIERYLKDQQLLGLWGCSGKLICTTNVPWNSSWSNKSQTDIWENMTWMQWDKEVSNYTDTIYRLLEDSQTQQERNEKDLLALDNWKNLWNWFSITNWLWYIKIFIMIVGGLIGLRIIFAVLSIVNRVRQGYSPLSFQTLTPNPRGPDRLGRIEEEGGGQDRDRSIRLVNGFLALAWDDLWSLCLFSYHRLRDLILVTARAVELLGHSSLRGLQRGWEALKYLGGIVQYWGLELKKRAISLLDTVAIAVAEGTDRIIEFLQRICRAIRNIPRRIRQGFEAALQ

SEQ ID NO: 15 - DNA, 1503 bp, Human immunodeficiency virus
  HIV Clade B Gag DNA sequence
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagagcttcaatagcggcaaagaagggcacacagccagaaattgcagggcccctaggaaaaagggcagctggaaaagcggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataa

SEQ ID NO: 16 - protein, 500 aa, Human immunodeficiency virus
  HIV Clade B Gag protein sequence
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKSFNSGKEGHTARNCRAPRKKGSWKSGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 17 - DNA, 1479 bp, Human immunodeficiency virus
  HIV Clade C Gag DNA sequence
atgggtgcgagagcgtcaatattaagagggggaaaattagataaatgggaaaagattaggttaaggccagggggaaagaaacactatatgctaaaacacctagtatgggcaagcagggagctggaaagatttgcacttaaccctggccttttagagacatcagaaggctgtaaacaaataataaaacagctacaaccagctcttcagacaggaacagaggaacttaggtcattattcaatgcagtagcaactctctattgtgtacatgcagacatagaggtacgagacaccaaagaagcattagacaagatagaggaagaacaaaacaaaagtcagcaaaaaacgcagcaggcaaaagaggctgacaaaaaggtcgtcagtcaaaattatcctatagtgcagaatcttcaagggcaaatggtacaccaggcactatcacctagaactttgaatgcatgggtaaaagtaatagaagaaaaagcctttagcccggaggtaatacccatgttcacagcattatcagaaggagccaccccacaagatttaaacaccatgttaaataccgtggggggacatcaagcagccatgcaaatgttaaaagataccatcaatgaggaggctgcagaatgggatagattacatccagtacatgcagggcctgttgcaccaggccaaatgagagaaccaaggggaagtgacatagcaggaactactagtaaccttcaggaacaaatagcatggatgacaagtaacccacctattccagtgggagatatctataaaagatggataattctggggttaaataaaatagtaagaatgtatagccctgtcagcattttagacataagacaagggccaaaggaaccctttagagattatgtagaccggttctttaaaactttaagagctgaacaagcttcacaagatgtaaaaaattggatggcagacaccttgttggtccaaaatgcgaacccagattgtaagaccattttaagagcattaggaccaggagctacattagaagaaatgatgacagcatgtcaaggagtgggaggacctagccacaaagcaagagtgttggctgaggcaatgagccaaacaggcagtaccataatgatgcagagaagcaattttaaaggctctaaaagaactgttaaatccttcaactctggcaaggaagggcacatagctagaaattgcagggcccctaggaaaaaaggctcttggaaatctggaaaggaaggacaccaaatgaaagactgtgctgagaggcaggctaattttttagggaaaatttggccttcccacaaggggaggccagggaatttccttcagaacaggccagagccaacagccccaccagcagagagcttcaggttcgaggagacaacccctgctccgaagcaggagctgaaagacagggaacccttaacctccctcaaatcactctttggcagcgaccccttgtctcaataa

SEQ ID NO: 18 - protein, 492 aa, Human immunodeficiency virus
  HIV Clade C Gag protein sequence
MGARASILRGGKLDKWEKIRLRPGGKKHYMLKHLVWASRELERFALNPGLLETSEGCKQIIKQLQPALQTGTEELRSLFNAVATLYCVHADIEVRDTKEALDKIEEEQNKSQQKTQQAKEADKKVVSQNYPIVQNLQGQMVHQALSPRTLNAWVKVIEEKAFSPEVIPMFTALSEGATPQDLNTMLNTVGGHQAAMQMLKDTINEEAAEWDRLHPVHAGPVAPGQMREPRGSDIAGTTSNLQEQIAWMTSNPPIPVGDIYKRWIILGLNKIVRMYSPVSILDIRQGPKEPFRDYVDRFFKTLRAEQASQDVKNWMADTLLVQNANPDCKTILRALGPGATLEEMMTACQGVGGPSHKARVLAEAMSQTGSTIMMQRSNFKGSKRTVKSFNSGKEGHIARNCRAPRKKGSWKSGKEGHQMKDCAERQANFLGKIWPSHKGRPGNFLQNRPEPTAPPAESFRFEETTPAPKQELKDREPLTSLKSLFGSDPLSQ

SEQ ID NO: 19 - DNA, 2184 bp, Human immunodeficiency virus
  HIV Clade B Pol DNA sequence
ttttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataaagataggggggcaactaaaggaagctctattagccacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaagataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaaatatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatgaacgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaataccgcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcctttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactaacacaacaaatcagaaaactcagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacattag

SEQ ID NO: 20 - protein, 727 aa, Human immunodeficiency virus
  HIV Clade B Pol protein sequence
FFREDLAFLQGKAREFSSEQTRANSPTRRELQVWGRDNNSPSEAGADRQGTVSFNFPQITLWQRPLVTIKIGGQLKEALLATGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMNDLYVGSDLEIGQHRTKIEELRQHLLRWGLTTPDKKHQKEPPFLWMGYELHPDKWTVQPIVLPEKDSWTVNDIQKLVGKLNTASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTNTTNQKTQLQAIYLALQDSGLEVNIVTDSQYALGIIQAQPDQSESELVNQIIEQLIKKEKVYLAWVPAHKGIGGNEQVDKLVSAGIRKVLFLDGIDKAQDEH

SEQ ID NO: 21 - DNA, 2139 bp, Human immunodeficiency virus
  HIV Clade C Pol DNA sequence
ttttttagggaaaatttggccttcccacaaggggaggccagggaatttccttcagaacaggccagagccaacagccccaccagcagagagcttcaggttcgaggagacaacccctgctccgaagcaggagctgaaagacagggaacccttaacctccctcaaatcactctttggcagcgaccccttgtctcaataaaaatagggggccagataaaggaggctctcttagccacaggagcagatgatacagtattagaagaaatgaatttgccaggaaaatggaaaccaaaaatgataggaggaattggaggttttatcaaagtaagacagtatgatcaaatacttatagaaatttgtggaaaaaaggctataggtacagtattagtaggacccacacctgtcaacataattggaagaaatatgctgactcagattggatgcacgctaaattttccaattagtcccattgaaactgtaccagtaaaattaaagccaggaatggatggcccaaaggttaaacaatggccattgacagaggagaaaataaaagcattaacagcaatttgtgatgaaatggagaaggaaggaaaaattacaaaaattgggcctgaaaatccatataacactccaatattcgccataaaaaagaaggacagtactaagtggagaaaattagtagatttcagagaacttaataaaagaactcaagacttctgggaagttcaattaggaataccacacccagcagggttaaaaaagaaaaaatcagtgacagtactagatgtgggggatgcatatttttcagttcctttagatgaaagctttaggaggtatactgcattcaccatacctagtagaaacaatgaaacaccagggattagatatcaatataatgtgcttccacaaggatggaaaggatcaccagcaatattccagagtagcatgacaaaaatcttagagccctttagagcacaaaatccagaaatagtcatctatcaatatatgaatgacttgtatgtaggatctgacttagaaatagggcaacatagagcaaagatagaggaattaagagaacatctattaaggtggggatttaccacaccagacaagaaacatcagaaagaacccccatttctttggatggggtatgaactccatcctgacaaatggacagtacagcctatacagctgccagaaaaggagagctggactgtcaatgatatacagaagttagtgggaaaattaaacacggcaagccagatttacccagggattaaagtaagacaactttgtagactccttagaggggccaaagcactaacagacatagtaccactaactgaagaagcagaattagaattggcagagaacagggaaattctaaaagaaccagtacatggagtatattatgacccttcaaaagacttgatagctgaaatacagaaacagggacatgaccaatggacatatcaaatttaccaagaaccattcaaaaatctgaaaacagggaagtatgcaaaaatgaggactgcccacactaatgatgtaaaacggttaacagaggcagtgcaaaaaatagccttagaaagcatagtaatatggggaaagattcctaaacttaggttacccatccaaaaagaaacatgggagacatggtggactgactattggcaagccacctggattcctgagtgggaatttgttaatactcctcccctagtaaaattatggtaccagctagagaaggaacccataataggagtagaaactttctatgtagatggagcagctaatagggaaaccaaaataggaaaagcagggtatgttactgacagaggaaggcagaaaattgtttctctaactgaaacaacaaatcagaagactcaattacaagcaatttatctagctttgcaagattcaggatcagaagtaaacatagtaacagactcacagtatgcattaggaattattcaagcacaaccagataagagtgaatcagggttagtcaaccaaataatagaacaattaataaaaaaggaaagggtctacctgtcatgggtaccagcacataaaggtattggaggaaatgaacaagtagacaaattagtaagtagtggaatcaggagagtgctataataa

SEQ ID NO: 22 - protein, 711 aa, Human immunodeficiency virus
  HIV Clade C Pol protein sequence
FFRENLAFPQGEAREFPSEQARANSPTSRELQVRGDNPCSEAGAERQGTLNLPQITLWQRPLVSIKIGGQIKEALLATGADDTVLEEMNLPGKWKPKMIGGIGGFIKVRQYDQILIEICGKKAIGTVLVGPTPVNIIGRNMLTQIGCTLNFPISPIETVPVKLKPGMDGPKVKQWPLTEEKIKALTAICDEMEKEGKITKIGPENPYNTPIFAIKKKDSTKWRKLVDFRELNKRTQDFWEVQLGIPHPAGLKKKKSVTVLDVGDAYFSVPLDESFRRYTAFTIPSRNNETPGIRYQYNVLPQGWKGSPAIFQSSMTKILEPFRAQNPEIVIYQYMNDLYVGSDLEIGQHRAKIEELREHLLRWGFTTPDKKHQKEPPFLWMGYELHPDKWTVQPIQLPEKESWTVNDIQKLVGKLNTASQIYPGIKVRQLCRLLRGAKALTDIVPLTEEAELELAENREILKEPVHGVYYDPSKDLIAEIQKQGHDQWTYQIYQEPFKNLKTGKYAKMRTAHTNDVKRLTEAVQKIALESIVIWGKIPKLRLPIQKETWETWWTDYWQATWIPEWEFVNTPPLVKLWYQLEKEPIIGVETFYVDGAANRETKIGKAGYVTDRGRQKIVSLTETTNQKTQLQAIYLALQDSGSEVNIVTDSQYALGIIQAQPDKSESGLVNQIIEQLIKKERVYLSWVPAHKGIGGNEQVDKLVSSGIRRVL

SEQ ID NO: 23 - DNA, 351 bp, Human immunodeficiency virus
  HIV Clade B Rev DNA sequence
atggcaggaagaagcggagacagcgacgaagagctcctcaagacagtcagactcatcaagtttctctatcaaagcaacccacctcccagccccgaggggacccgacaggcccgaaggaatcgaagaagaaggtggagacagagacagagacagatccgtgcgattagtggatggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggagctaaagaatag

SEQ ID NO: 24 - protein, 116 aa, Human immunodeficiency virus
  HIV Clade B Rev protein sequence
MAGRSGDSDEELLKTVRLIKFLYQSNPPPSPEGTRQARRNRRRRWRQRQRQIRAISGWILSTYLGRSAEPVPLQLPPLERLTLDCNEDCGTSGTQGVGSPQILVESPTVLESGAKE

SEQ ID NO: 25 - DNA, 324 bp, Human immunodeficiency virus
  HIV Clade C Rev DNA sequence
atggcaggaagaagcggagacagcgacgaagcgctcctcagagcagtgaggatcatcagaattttgtatcaaagcaacccttaccccaaacccaaggggacccgacaggctcggaagaatcgaagaagaaggtggagggcaagacagagacagatcgattcgattagtgaacggattcttagcacttgcctgggacgacctgtggagcctgtgcctcttcagctaccaccgattgagagacttaatattggtgacagcgagagcggtggaacttctgggacacagcagtctcaggggactacagagggggtgggaagcccttaa

SEQ ID NO: 26 - protein, 107 aa, Human immunodeficiency virus
  HIV Clade C Rev protein sequence
MAGRSGDSDEALLRAVRIIRILYQSNPYPKPKGTRQARKNRRRRWRARQRQIDSISERILSTCLGRPVEPVPLQLPPIERLNIGDSESGGTSGTQQSQGTTEGVGSP

SEQ ID NO: 27 - DNA, 306 bp, Human immunodeficiency virus
  HIV Clade B Tat DNA sequence
atggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcctcaagacagtcagactcatcaagtttctctatcaaagcaacccacctcccagccccgaggggacccgacaggcccgaaggaatcgaagaagaaggtggagacagagacagagacagatccgtgcgattag

SEQ ID NO: 28 - protein, 101 aa, Human immunodeficiency virus
  HIV Clade B Tat protein sequence
MEPVDPRLEPWKHPGSQPKTACTNCYCKKCCFHCQVCFITKALGISYGRKKRRQRRRAPQDSQTHQVSLSKQPTSQPRGDPTGPKESKKKVETETETDPCD

SEQ ID NO: 29 - DNA, 306 bp, Human immunodeficiency virus
  HIV Clade C Tat DNA sequence
atggagccagtagatcctaacctagagccctggaaccatccaggaagtcagcctgaaactgcttgcaataactgttattgtaaacgctatagctaccattgtctagtttgctttcagagaaaaggcttaggcatttcctatggcaggaagaagcggagacagcgacgaagcgctcctcagagcagtgaggatcatcagaattttgtatcaaagcaacccttaccccaaacccaaggggacccgacaggctcggaagaatcgaagaagaaggtggagggcaagacagagacagatcgattcgattag

SEQ ID NO: 30 - protein, 101 aa, Human immunodeficiency virus
  HIV Clade C Tat protein sequence
MEPVDPNLEPWNHPGSQPETACNNCYCKRYSYHCLVCFQRKGLGISYGRKKRRQRRSAPQSSEDHQNFVSKQPLPQTQGDPTGSEESKKKVEGKTETDRFD

SEQ ID NO: 31 - DNA, 246 bp, Human immunodeficiency virus
  HIV Clade B Vpu DNA sequence
atgcaacctttacaaatattagcaatagtagcattagtagtagcagcaataatagcaatagttgtgtggaccatagtattcatagaatataggaaaatattaagacaaagaaaaatagacaggttaattgataggataacagaaagagcagaagacagtggcaatgaaagtgaaggggatcaggaagaattatcagcacttgtggaaatggggcatcatgctccttgggatgttgatgatctgtag

SEQ ID NO: 32 - protein, 81 aa, Human immunodeficiency virus
  HIV Clade B Vpu protein sequence
MQPLQILAIVALVVAAIIAIVVWTIVFIEYRKILRQRKIDRLIDRITERAEDSGNESEGDQEELSALVEMGHHAPWDVDDL

SEQ ID NO: 33 - DNA, 249 bp, Human immunodeficiency virus
  HIV Clade C Vpu DNA sequence
atgttagatttagattataaattagcagtaggagcatttatagtagcactactcatagcaatagttgtgtggaccatagtatttatagaatataggaaattgttaagacaaagaaaaatagactggttaattaaaagaattagggaaagagcagaagacagtggcaatgagagtgaaggggatactgaggaattatcgacaatggtggatatggggcatcttaggcttttggatgttaatgatttgtaa

SEQ ID NO: 34 - protein, 82 aa, Human immunodeficiency virus
  HIV Clade C Vpu protein sequence
MLDLDYKLAVGAFIVALLIAIVVWTIVFIEYRKLLRQRKIDWLIKRIRERAEDSGNESEGDTEELSTMVDMGHLRLLDVNDL

SEQ ID NO: 35 - DNA, 2217 bp, Human immunodeficiency virus
  HIV Clade B Env DNA sequence
atgaaagtgaaggggatcaggaagaattatcagcacttgtggaaatggggcatcatgctccttgggatgttgatgatctgtagtgctgtagaaaatttgtgggtcacagtttattatggggtacctgtgtggaaagaagcaaccaccactctattttgtgcatcagatgctaaagcatatgatacagaggtacataatgtttgggccacacatgcctgtgtacccacagaccccaacccacaagaagtagtattggaaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatgttactaatatcaataatagtagtgagggaatgagaggagaaataaaaaactgctctttcaatatcaccacaagcataagagataaggtgaagaaagactatgcacttttctatagacttgatgtagtaccaatagataatgataatactagctataggttgataaattgtaatacctcaaccattacacaggcctgtccaaaggtatcctttgagccaattcccatacattattgtaccccggctggttttgcgattctaaagtgtaaagacaagaagttcaatggaacagggccatgtaaaaatgtcagcacagtacaatgtacacatggaattaggccagtagtgtcaactcaactgctgttaaatggcagtctagcagaagaagaggtagtaattagatctagtaatttcacagacaatgcaaaaaacataatagtacagttgaaagaatctgtagaaattaattgtacaagacccaacaacaatacaaggaaaagtatacatataggaccaggaagagcattttatacaacaggagaaataataggagatataagacaagcacattgcaacattagtagaacaaaatggaataacactttaaatcaaatagctacaaaattaaaagaacaatttgggaataataaaacaatagtctttaatcaatcctcaggaggggacccagaaattgtaatgcacagttttaattgtggaggggaattcttctactgtaattcaacacaactgtttaatagtacttggaattttaatggtacttggaatttaacacaatcgaatggtactgaaggaaatgacactatcacactcccatgtagaataaaacaaattataaatatgtggcaggaagtaggaaaagcaatgtatgcccctcccatcagaggacaaattagatgctcatcaaatattacagggctaatattaacaagagatggtggaactaacagtagtgggtccgagatcttcagacctgggggaggagatatgagggacaattggagaagtgaattatataaatataaagtagtaaaaattgaaccattaggagtagcacccaccaaggcaaaaagaagagtggtgcagagagaaaaaagagcagtgggaacgataggagctatgttccttgggttcttgggagcagcaggaagcactatgggcgcagcgtcaataacgctgacggtacaggccagactattattgtctggtatagtgcaacagcagaacaatttgctgagggctattgaggcgcaacagcatctgttgcaactcacagtctggggcatcaagcagctccaggcaagagtcctggctgtggaaagatacctaagggatcaacagctcctagggatttggggttgctctggaaaactcatctgcaccactgctgtgccttggaatgctagttggagtaataaaactctggatatgatttgggataacatgacctggatggagtgggaaagagaaatcgaaaattacacaggcttaatatacaccttaattgaggaatcgcagaaccaacaagaaaagaatgaacaagacttattagcattagataagtgggcaagtttgtggaattggtttgacatatcaaattggctgtggtatgtaaaaatcttcataatgatagtaggaggcttgataggtttaagaatagtttttactgtactttctatagtaaatagagttaggcagggatactcaccattgtcatttcagacccacctcccagccccgaggggacccgacaggcccgaaggaatcgaagaagaaggtggagacagagactaa

SEQ ID NO: 36 - protein, 738 aa, Human immunodeficiency virus
  HIV Clade B Env Protein sequence
MKVKGIRKNYQHLWKWGIMLLGMLMICSAVENLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNVTNINNSSEGMRGEIKNCSFNITTSIRDKVKKDYALFYRLDVVPIDNDNTSYRLINCNTSTITQACPKVSFEPIPIHYCTPAGFAILKCKDKKFNGTGPCKNVSTVQCTHGIRPVVSTQLLLNGSLAEEEVVIRSSNFTDNAKNIIVQLKESVEINCTRPNNNTRKSIHIGPGRAFYTTGEIIGDIRQAHCNISRTKWNNTLNQIATKLKEQFGNNKTIVFNQSSGGDPEIVMHSFNCGGEFFYCNSTQLFNSTWNFNGTWNLTQSNGTEGNDTITLPCRIKQIINMWQEVGKAMYAPPIRGQIRCSSNITGLILTRDGGTNSSGSEIFRPGGGDMRDNWRSELYKYKVVKIEPLGVAPTKAKRRVVQREKRAVGTIGAMFLGFLGAAGSTMGAASITLTVQARLLLSGIVQQQNNLLRAIEAQQHLLQLTVWGIKQLQARVLAVERYLRDQQLLGIWGCSGKLICTTAVPWNASWSNKTLDMIWDNMTWMEWEREIENYTGLIYTLIEESQNQQEKNEQDLLALDKWASLWNWFDISNWLWYVKIFIMIVGGLIGLRIVFTVLSIVNRVRQGYSPLSFQTHLPAPRGPDRPEGIEEEGGDRD

SEQ ID NO: 37 - DNA, 2244 bp, Human immunodeficiency virus
  HIV Clade C Env DNA sequence
atgagagtgaaggggatactgaggaattatcgacaatggtggatatggggcatcttaggcttttggatgttaatgatttgtaatggaaacttgtgggtcacagtctattatggggtacctgtgtggaaagaagcaaaaactactctattctgtgcatcaaatgctaaagcatatgagaaagaagtacataatgtctgggctacacatgcctgtgtacccacagaccccaacccacaagaaatggttttggaaaacgtaacagaaaattttaacatgtggaaaaatgacatggtgaatcagatgcatgaggatgtaatcagcttatgggatcaaagcctaaagccatgtgtaaagttgaccccactctgtgtcactttagaatgtagaaaggttaatgctacccataatgctaccaataatggggatgctacccataatgttaccaataatgggcaagaaatacaaaattgctctttcaatgcaaccacagaaataagagataggaagcagagagtgtatgcacttttctatagacttgatatagtaccacttgataagaacaactctagtaagaacaactctagtgagtattatagattaataaattgtaatacctcagccataacacaagcatgtccaaaggtcagttttgatccaattcctatacactattgtgctccagctggttatgcgattctaaagtgtaacaataagacattcaatgggacaggaccatgcaataatgtcagcacagtacaatgtacacatggaattaagccagtggtatcaactcagctattgttaaacggtagcctagcagaaggagagataataattagatctgaaaatctgacagacaatgtcaaaacaataatagtacatcttgatcaatctgtagaaattgtgtgtacaagacccaacaataatacaagaaaaagtataaggatagggccaggacaaacattctatgcaacaggaggcataatagggaacatacgacaagcacattgtaacattagtgaagacaaatggaatgaaactttacaaagggtgggtaaaaaattagtagaacacttccctaataagacaataaaatttgcaccatcctcaggaggggacctagaaattacaacacatagctttaattgtagaggagaattcttctattgcagcacatcaagactgtttaatagtacatacatgcctaatgatacaaaaagtaagtcaaacaaaaccatcacaatcccatgcagcataaaacaaattgtaaacatgtggcaggaggtaggacgagcaatgtatgcccctcccattgaaggaaacataacctgtagatcaaatatcacaggaatactattggtacgtgatggaggagtagattcagaagatccagaaaataataagacagagacattccgacctggaggaggagatatgaggaacaattggagaagtgaattatataaatataaagcggcagaaattaagccattgggagtagcacccactccagcaaaaaggagagtggtggagagagaaaaaagagcagtaggattaggagctgtgttccttggattcttgggagcagcaggaagcactatgggcgcagcgtcaataacgctgacggtacaggccagacaattgttgtctggtatagtgcaacagcaaagcaatttgctgagggctatcgaggcgcaacagcatctgttgcaactcacggtctggggcattaagcagctccagacaagagtcctggctatcgaaagatacctaaaggatcaacagctcctagggctttggggctgctctggaaaactcatctgcaccactaatgtaccttggaactccagttggagtaacaaatctcaaacagatatttgggaaaacatgacctggatgcagtgggataaagaagttagtaattacacagacacaatatacaggttgcttgaagactcgcaaacccagcaggaaagaaatgaaaaggatttattagcattggacaattggaaaaatctgtggaattggtttagtataacaaactggctgtggtatataaaaatattcataatgatagtaggaggcttgataggcttaagaataatttttgctgtgctttctatagtgaatagagttaggcagggatactcacctttgtcgtttcagacccttaccccaaacccaaggggacccgacaggctcggaagaatcgaagaagaaggtggagggcaagacagagactaa

SEQ ID NO: 38 - protein, 747 aa, Human immunodeficiency virus
  HIV Clade C Env protein sequence
MRVKGILRNYRQWWIWGILGFWMLMICNGNLWVTVYYGVPVWKEAKTTLFCASNAKAYEKEVHNVWATHACVPTDPNPQEMVLENVTENFNMWKNDMVNQMHEDVISLWDQSLKPCVKLTPLCVTLECRKVNATHNATNNGDATHNVTNNGQEIQNCSFNATTEIRDRKQRVYALFYRLDIVPLDKNNSSKNNSSEYYRLINCNTSAITQACPKVSFDPIPIHYCAPAGYAILKCNNKTFNGTGPCNNVSTVQCTHGIKPVVSTQLLLNGSLAEGEIIIRSENLTDNVKTIIVHLDQSVEIVCTRPNNNTRKSIRIGPGQTFYATGGIIGNIRQAHCNISEDKWNETLQRVGKKLVEHFPNKTIKFAPSSGGDLEITTHSFNCRGEFFYCSTSRLFNSTYMPNDTKSKSNKTITIPCSIKQIVNMWQEVGRAMYAPPIEGNITCRSNITGILLVRDGGVDSEDPENNKTETFRPGGGDMRNNWRSELYKYKAAEIKPLGVAPTPAKRRVVEREKRAVGLGAVFLGFLGAAGSTMGAASITLTVQARQLLSGIVQQQSNLLRAIEAQQHLLQLTVWGIKQLQTRVLAIERYLKDQQLLGLWGCSGKLICTTNVPWNSSWSNKSQTDIWENMTWMQWDKEVSNYTDTIYRLLEDSQTQQERNEKDLLALDNWKNLWNWFSITNWLWYIKIFIMIVGGLIGLRIIFAVLSIVNRVRQGYSPLSFQTLTPNPRGPDRLGRIEEEGGGQDRD

SEQ ID NO: 39 - DNA, 1503 bp, Human immunodeficiency virus
  HIV Clade B Gag DNA sequence
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataa

SEQ ID NO: 40 - protein, 500 aa, Human immunodeficiency virus
  HIV Clade B Gag protein sequence
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 41 - DNA, 1479 bp, Human immunodeficiency virus
  HIV Clade C Gag DNA sequence
atgggtgcgagagcgtcaatattaagagggggaaaattagataaatgggaaaagattaggttaaggccagggggaaagaaacactatatgctaaaacacctagtatgggcaagcagggagctggaaagatttgcacttaaccctggccttttagagacatcagaaggctgtaaacaaataataaaacagctacaaccagctcttcagacaggaacagaggaacttaggtcattattcaatgcagtagcaactctctattgtgtacatgcagacatagaggtacgagacaccaaagaagcattagacaagatagaggaagaacaaaacaaaagtcagcaaaaaacgcagcaggcaaaagaggctgacaaaaaggtcgtcagtcaaaattatcctatagtgcagaatcttcaagggcaaatggtacaccaggcactatcacctagaactttgaatgcatgggtaaaagtaatagaagaaaaagcctttagcccggaggtaatacccatgttcacagcattatcagaaggagccaccccacaagatttaaacaccatgttaaataccgtggggggacatcaagcagccatgcaaatgttaaaagataccatcaatgaggaggctgcagaatgggatagattacatccagtacatgcagggcctgttgcaccaggccaaatgagagaaccaaggggaagtgacatagcaggaactactagtaaccttcaggaacaaatagcatggatgacaagtaacccacctattccagtgggagatatctataaaagatggataattctggggttaaataaaatagtaagaatgtatagccctgtcagcattttagacataagacaagggccaaaggaaccctttagagattatgtagaccggttctttaaaactttaagagctgaacaagcttcacaagatgtaaaaaattggatggcagacaccttgttggtccaaaatgcgaacccagattgtaagaccattttaagagcattaggaccaggagctacattagaagaaatgatgacagcatgtcaaggagtgggaggacctagccacaaagcaagagtgttggctgaggcaatgagccaaacaggcagtaccataatgatgcagagaagcaattttaaaggctctaaaagaactgttaaatgcttcaactgtggcaaggaagggcacatagctagaaattgcagggcccctaggaaaaaaggctgttggaaatgtggaaaggaaggacaccaaatgaaagactgtgctgagaggcaggctaattttttagggaaaatttggccttcccacaaggggaggccagggaatttccttcagaacaggccagagccaacagccccaccagcagagagcttcaggttcgaggagacaacccctgctccgaagcaggagctgaaagacagggaacccttaacctccctcaaatcactctttggcagcgaccccttgtctcaataa

SEQ ID NO: 42 - protein, 492 aa, Human immunodeficiency virus
  HIV Clade C Gag protein sequence
MGARASILRGGKLDKWEKIRLRPGGKKHYMLKHLVWASRELERFALNPGLLETSEGCKQIIKQLQPALQTGTEELRSLFNAVATLYCVHADIEVRDTKEALDKIEEEQNKSQQKTQQAKEADKKVVSQNYPIVQNLQGQMVHQALSPRTLNAWVKVIEEKAFSPEVIPMFTALSEGATPQDLNTMLNTVGGHQAAMQMLKDTINEEAAEWDRLHPVHAGPVAPGQMREPRGSDIAGTTSNLQEQIAWMTSNPPIPVGDIYKRWIILGLNKIVRMYSPVSILDIRQGPKEPFRDYVDRFFKTLRAEQASQDVKNWMADTLLVQNANPDCKTILRALGPGATLEEMMTACQGVGGPSHKARVLAEAMSQTGSTIMMQRSNFKGSKRTVKCFNCGKEGHIARNCRAPRKKGCWKCGKEGHQMKDCAERQANFLGKIWPSHKGRPGNFLQNRPEPTAPPAESFRFEETTPAPKQELKDREPLTSLKSLFGSDPLSQ

SEQ ID NO: 43 - DNA, 2184 bp, Human immunodeficiency virus
  HIV Clade B Pol DNA sequence
ttttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgagaatccatacaatactccagtatttgccataaagaaaaaagacagtactaaatggaggaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttaaaaaacaaaatccagacatagttatctatcaatacatgaacgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaataccgcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactacccatacaaaaggaaacatgggaaacatggtggacagagtattggcaagccacctggattcctgagtgggagtttgttaatacccctcctttagtgaaattatggtaccagttagagaaagaacccatagtaggagcagaaaccttctatgtagatggggcagctaacagggagactaaattaggaaaagcaggatatgttactaacaaaggaagacaaaaggttgtccccctaactaacacaacaaatcagaaaactcagttacaagcaatttatctagctttgcaggattcaggattagaagtaaacatagtaacagactcacaatatgcattaggaatcattcaagcacaaccagataaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaaatactatttttagatggaatagataaggcccaagatgaacattag

SEQ ID NO: 44 - protein, 727 aa, Human immunodeficiency virus
  HIV Clade B Pol protein sequence
FFREDLAFLQGKAREFSSEQTRANSPTRRELQVWGRDNNSPSEAGADRQGTVSFNFPQITLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFKKQNPDIVIYQYMNDLYVGSDLEIGQHRTKIEELRQHLLRWGLTTPDKKHQKEPPFLWMGYELHPDKWTVQPIVLPEKDSWTVNDIQKLVGKLNTASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNKGRQKVVPLTNTTNQKTQLQAIYLALQDSGLEVNIVTDSQYALGIIQAQPDKSESELVNQIIEQLIKKEKVYLAWVPAHKGIGGNEQVDKLVSAGIRKILFLDGIDKAQDEH

SEQ ID NO: 45 - DNA, 2136 bp, Human immunodeficiency virus
  HIV Clade C Pol DNA sequence
ttttttagggaaaatttggccttcccacaaggggaggccagggaatttccttcagaacaggccagagccaacagccccaccagcagagagcttcaggttcgaggagacaacccctgctccgaagcaggagctgaaagacagggaacccttaacctccctcaaatcactctttggcagcgaccccttgtctcaataaaaatagggggccagataaaggaggctctcttagacacaggagcagatgatacagtattagaagaaatgaatttgccaggaaaatggaaaccaaaaatgataggaggaattggaggttttatcaaagtaagacagtatgatcaaatacttatagaaatttgtggaaaaaaggctataggtacagtattagtaggacccacacctgtcaacataattggaagaaatatgctgactcagattggatgcacgctaaattttccaattagtcccattgaaactgtaccagtaaaattaaagccaggaatggatggcccaaaggttaaacaatggccattgacagaggagaaaataaaagcattaacagcaatttgtgatgaaatggagaaggaaggaaaaattacaaaaattgggcctgaaaatccatataacactccaatattcgccataaaaaagaaggacagtactaagtggagaaaattagtagatttcagagaacttaataaaagaactcaagacttctgggaagttcaattaggaataccacacccagcagggttaaaaaagaaaaaatcagtgacagtactagatgtgggggatgcatatttttcagttcctttagatgaaagctttaggaggtatactgcattcaccatacctagtagaaacaatgaaacaccagggattagatatcaatataatgtgcttccacaaggatggaaaggatcaccagcaatattccagagtagcatgacaaaaatcttagagccctttagagcacaaaatccagaaatagtcatctatcaatatatgaatgacttgtatgtaggatctgacttagaaatagggcaacatagagcaaagatagaggaattaagagaacatctattaaggtggggatttaccacaccagacaagaaacatcagaaagaacccccatttctttggatggggtatgaactccatcctgacaaatggacagtacagcctatacagctgccagaaaaggagagctggactgtcaatgatatacagaagttagtgggaaaattaaacacggcaagccagatttacccagggattaaagtaagacaactttgtagactccttagaggggccaaagcactaacagacatagtaccactaactgaagaagcagaattagaattggcagagaacagggaaattctaaaagaaccagtacatggagtatattatgacccttcaaaagacttgatagctgaaatacagaaacagggacatgaccaatggacatatcaaatttaccaagaaccattcaaaaatctgaaaacagggaagtatgcaaaaatgaggactgcccacactaatgatgtaaaacggttaacagaggcagtgcaaaaaatagccttagaaagcatagtaatatggggaaagattcctaaacttaggttacccatccaaaaagaaacatgggagacatggtggactgactattggcaagccacctggattcctgagtgggaatttgttaatactcctcccctagtaaaattatggtaccagctagagaaggaacccataataggagtagaaactttctatgtagatggagcagctaatagggaaaccaaaataggaaaagcagggtatgttactgacagaggaaggcagaaaattgtttctctaactgaaacaacaaatcagaagactcaattacaagcaatttatctagctttgcaagattcaggatcagaagtaaacatagtaacagactcacagtatgcattaggaattattcaagcacaaccagataagagtgaatcagggttagtcaaccaaataatagaacaattaataaaaaaggaaagggtctacctgtcatgggtaccagcacataaaggtattggaggaaatgaacaagtagacaaattagtaagtagtggaatcaggagagtgctatag

SEQ ID NO: 46 - protein, 711 aa, Human immunodeficiency virus
  HIV Clade C Pol protein sequence
FFRENLAFPQGEAREFPSEQARANSPTSRELQVRGDNPCSEAGAERQGTLNLPQITLWQRPLVSIKIGGQIKEALLDTGADDTVLEEMNLPGKWKPKMIGGIGGFIKVRQYDQILIEICGKKAIGTVLVGPTPVNIIGRNMLTQIGCTLNFPISPIETVPVKLKPGMDGPKVKQWPLTEEKIKALTAICDEMEKEGKITKIGPENPYNTPIFAIKKKDSTKWRKLVDFRELNKRTQDFWEVQLGIPHPAGLKKKKSVTVLDVGDAYFSVPLDESFRRYTAFTIPSRNNETPGIRYQYNVLPQGWKGSPAIFQSSMTKILEPFRAQNPEIVIYQYMNDLYVGSDLEIGQHRAKIEELREHLLRWGFTTPDKKHQKEPPFLWMGYELHPDKWTVQPIQLPEKESWTVNDIQKLVGKLNTASQIYPGIKVRQLCRLLRGAKALTDIVPLTEEAELELAENREILKEPVHGVYYDPSKDLIAEIQKQGHDQWTYQIYQEPFKNLKTGKYAKMRTAHTNDVKRLTEAVQKIALESIVIWGKIPKLRLPIQKETWETWWTDYWQATWIPEWEFVNTPPLVKLWYQLEKEPIIGVETFYVDGAANRETKIGKAGYVTDRGRQKIVSLTETTNQKTQLQAIYLALQDSGSEVNIVTDSQYALGIIQAQPDKSESGLVNQIIEQLIKKERVYLSWVPAHKGIGGNEQVDKLVSSGIRRVL